(19) United States
(12) Patent Application Publication
(10) Pub. No.: US 2020/0172595 A1
(43) Pub. Date: Jun. 4, 2020

(54) ANTIGEN-PRESENTING POLYPEPTIDES AND METHODS OF USE THEREOF

(71) Applicant: Cue Biopharma, Inc., Cambridge, MA (US)

(72) Inventors: Ronald D. Seidel, III, Cambridge, MA (US); Rodolfo J. Chaparro, Cambridge, MA (US); John F. Ross, Cambridge, MA (US)

(21) Appl. No.: 16/789,057

(22) Filed: Feb. 12, 2020

Related U.S. Application Data
(63) Continuation of application No. PCT/US2018/049760, filed on Sep. 6, 2018.
(60) Provisional application No. 62/692,314, filed on Jun. 29, 2018.
(60) Provisional application No. 62/555,526, filed on Sep. 7, 2017.

(51) Int. Cl. C07K 14/725; C07K 14/74; C12N 15/85

(52) U.S. Cl. CPC C07K 14/7051; C07K 14/70539; A61K 38/00; C07K 2317/31; C07K 2317/34; C12N 15/85

(57) ABSTRACT

The present disclosure provides antigen-presenting polypeptides, including single-chain antigen-presenting polypeptides and multimeric antigen-presenting polypeptides. The present disclosure provides nucleic acids comprising nucleotide sequences encoding antigen-presenting polypeptides of the present disclosure, as well as cells genetically modified with the nucleic acids. An antigen-presenting polypeptide of the present disclosure is useful for modulating activity of a T cell. Thus, the present disclosure provides methods of modulating activity of a T cell.

### CROSS-REFERENCE

This application claims the benefit of U.S. Provisional Patent Application No. 62/555,526, filed Sep. 7, 2017, and of U.S. Provisional Patent Application No. 62/692,314, filed Jun. 29, 2018, which applications are incorporated herein by reference in their entirety.

### INTRODUCTION

Central to the proper functioning of the mammalian immune system are the coordinated activities and communications between two specialized cell types, antigen-presenting cells (“APCs”) and T cells. APCs serve to capture and break the proteins from foreign organisms, or abnormal proteins (e.g., from genetic mutation in cancer cells), into smaller fragments suitable as signals for scrutiny by the larger immune system, including T cells. In particular, APCs break down proteins into small peptide fragments, which are then paired with proteins of the major histocompatibility complex (“MHC”) and displayed on the cell surface. Cell surface display of an MHC together with a peptide fragment, also known as a T cell epitope, provides the underlying scaffold surveilled by T cells, allowing for specific recognition. The peptide fragments can be pathogen-derived, tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T cells through additional costimulatory signals in order to generate a more effective response.

T cells recognize peptide-major histocompatibility complex (“pMHC”) complexes through a specialized cell surface receptor, the T cell receptor (“TCR”). The TCR is unique to each T cell; as a consequence, each T cell is highly specific for a particular pMHC target. In order to adequately address the universe of potential threats, a very large number (˜10,000,000) of distinct T cells with distinct TCRs exist in the human body. Further, any given T cell, specific for a particular T cell peptide, is initially a very small fraction of the total T cell population. Although normally dormant and in limited numbers, T cells bearing specific TCRs can be readily activated and amplified by APCs to generate highly potent T cell responses that involve many millions of T cells. Such activated T cell responses are capable of attacking and clearing viral infections, bacterial infections, and other cellular threats including tumors, as illustrated below. Conversely, the broad, non-specific activation of overly active T cell responses against self or shared antigens can give rise to T cells inappropriately attacking and destroying healthy tissues or cells.

MHC proteins are referred to as human leukocyte antigens (HLA) in humans. HLA class II gene loci include HLA-DM (HLA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

### SUMMARY

The present disclosure provides antigen-presenting polypeptides, including single-chain antigen-presenting polypeptides and multimeric antigen-presenting polypeptides. The present disclosure provides nucleic acids comprising nucleotide sequences encoding antigen-presenting polypeptides of the present disclosure, as well as cells genetically modified with the nucleic acids. An antigen-presenting polypeptide of the present disclosure is useful for modulating activity of a T cell. Thus, the present disclosure provides methods of modulating activity of a T cell.

### BRIEF DESCRIPTION OF THE DRAWINGS

FIG. 1 provides a schematic depiction of MHC Class II alpha- and beta-chains with a peptide.

FIG. 2A-2C provide schematic depictions of examples of antigen-presenting polypeptides (APPs).

FIG. 3A-3B provide a schematic depiction of an example of an antigen-presenting polypeptide (FIG. 3A); and a crystal structure of the human Class II MHC protein HLA-DR1 complexed with an influenza virus peptide (FIG. 3B).

FIG. 4A-4C depict gel analysis (FIG. 4A), expression levels (FIG. 4B), and descriptions (FIG. 4C) of APPs of the present disclosure.

FIG. 5A-5B provide schematic depictions of APPs without immunomodulatory (MOD) polypeptides (FIG. 5A) and with a MOD polypeptide (FIG. 5B). The unmarked rectangle in FIG. 5A represents a dimerization domain (e.g., a bZIP polypeptide). In FIG. 5B, the arrows pointing to the dashed lines indicate possible positions of a MOD polypeptide(s).

FIG. 6 provides an amino acid sequence of an HLA Class II DRA α chain.

FIG. 7A-7J provide amino acid sequences of HLA Class II DRB1 β chains.

FIG. 8A-8C provide amino acid sequences of HLA Class II DRB3 β chains.

FIG. 9 provides an amino acid sequence of an HLA Class II DRB4 β chain.

FIG. 10 provides an amino acid sequence of an HLA Class II DRB5 β chain.

FIG. 11 provides an amino acid sequence of an HLA Class II DMA α chain.

FIG. 12 provides an amino acid sequence of an HLA Class II DMB β chain.

FIG. 13 provides an amino acid sequence of an HLA Class II DOA α chain.

FIG. 14 provides an amino acid sequence of an HLA Class II DOB β chain.

FIG. 15 provides an amino acid sequence of an HLA Class II DPA1 α chain.

FIG. 16 provides an amino acid sequence of an HLA Class II DPB1 β chain.

FIG. 17 provides an amino acid sequence of an HLA Class II DQA1 α chain.

FIG. 18 provides an amino acid sequence of an HLA Class II DQA2 α chain.

FIG. 19A-19B provide amino acid sequences of HLA Class II DQB1 β chains.

FIG. 20A-20B provide amino acid sequence of HLA Class II DQB2 β chains.

FIG. 21A-21G provide amino acid sequences of immunoglobulin Fc polypeptides.

FIG. 22A-22L provide schematic depictions of exemplary multimeric T-cell modulatory antigen-presenting polypeptides (TMAPPs) of the present disclosure.

FIG. 23A-23I provide schematic depictions of exemplary single-chain TMAPPs of the present disclosure.

FIG. 24 depicts production of exemplary APPs of the present disclosure.

FIG. 25A-25B provide the amino acid sequence (FIG. 25A) of an exemplary polypeptide chain of a multimeric TMAPP, and a nucleotide sequence (FIG. 25B) encoding same.

FIG. 26A-26B provide the amino acid sequence (FIG. 26A) of an exemplary polypeptide chain of a multimeric TMAPP, and a nucleotide sequence (FIG. 26B) encoding same.

FIG. 27A-27B provide the amino acid sequence (FIG. 27A) of an exemplary single-chain APP, and a nucleotide sequence (FIG. 27B) encoding same.

FIG. 28A-28B provide the amino acid sequence (FIG. 28A) of an exemplary single-chain TMAPP, and a nucleotide sequence (FIG. 28B) encoding same.

FIG. 29A-29B provide the amino acid sequence (FIG. 29A) of an exemplary single-chain TMAPP, and a nucleotide sequence (FIG. 29B) encoding same.

FIG. 30A-30B provide the amino acid sequence (FIG. 30A) of an exemplary polypeptide chain of a multimeric TMAPP, and a nucleotide sequence (FIG. 30B) encoding same.

FIG. 31A-31B provide the amino acid sequence (FIG. 31A) of an exemplary polypeptide chain of a multimeric TMAPP, and a nucleotide sequence (FIG. 31B) encoding same.

FIG. 32A-32B provide the amino acid sequence (FIG. 32A) of an exemplary polypeptide chain of a multimeric TMAPP, and a nucleotide sequence (FIG. 32B) encoding same.

FIG. 33A-33B provide the amino acid sequence (FIG. 33A) of an exemplary polypeptide chain of a multimeric TMAPP, and a nucleotide sequence (FIG. 33B) encoding same.

FIG. 34A-34B provide the amino acid sequence (FIG. 34A) of an exemplary polypeptide chain of a multimeric TMAPP, and a nucleotide sequence (FIG. 34B) encoding same.

FIG. 35A-35B provide the amino acid sequence (FIG. 35A) of an exemplary polypeptide chain of a multimeric TMAPP, and a nucleotide sequence (FIG. 35B) encoding same.

FIG. 36 provides a schematic depiction of an exemplary TMAPP of the present disclosure, and provides gel analysis of expression.

FIGS. 37A and 37B provide the amino acid sequence (FIG. 37A) of an exemplary polypeptide chain of a multimeric TMAPP, and a nucleotide sequence (FIG. 37B) encoding same.

FIGS. 38A and 38B provide the amino acid sequence (FIG. 38A) of an exemplary polypeptide chain of a multimeric TMAPP, and a nucleotide sequence (FIG. 38B) encoding same.

FIG. 39 depicts production of an exemplary APP of the present disclosure.

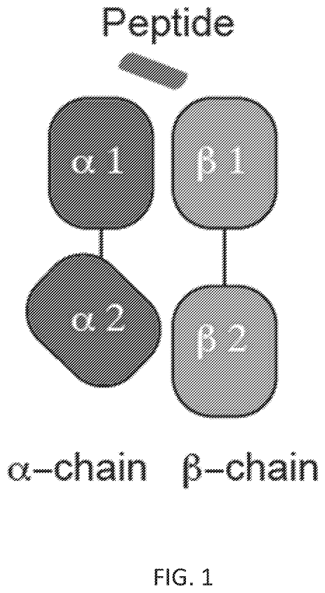

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “binding,” as used herein (e.g. with reference to binding of a T-cell modulatory antigen-presenting polypeptide to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of non-covalent binding, increased binding affinity being correlated with a lower K_{D}. “Specific binding” generally refers to binding with an affinity of at least about 10⁻⁷ M or greater, e.g., 5×10⁻⁷ M, 10⁻⁸ M, 5×10⁻⁸ M, 10⁻⁹ M, and greater. “Non-specific binding” generally refers to binding (e.g., the binding of a ligand to a moiety other than its designated binding site or receptor) with an affinity of less than about 10⁻⁷ M (e.g., binding with an affinity of 10⁻⁶ M, 10⁻⁵ M, 10⁻⁴ M). However, in some contexts, e.g., binding between a TCR and a peptide/MHC complex, “specific binding” can be in the range of from 1 μM to 100 μM, or from 100 μM to 1 mM. “Covalent binding” or “covalent bond,” as used herein, refers to the formation of one or more covalent chemical binds between two different molecules.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. 2001; 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “co-stimulatory polypeptide”), as used herein, includes a polypeptide on an antigen presenting cell (APC) (e.g., a dendritic cell, a B cell, and the like), or a portion of the polypeptide on an APC, that specifically binds a cognate co-immunomodulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. An immunomodulatory polypeptide can include, but is not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory polypeptide also encompasses, inter alia, an antibody that specifically binds with a cognate co-stimulatory molecule present on a T cell, such as, but not limited to, IL-2, CD27, CD28, 4-1BB, OX40, CD30, CD40, PD-1, ICOS, lymphocyte function-associated antigen-1 (LFA-1), CD2, LIGHT, NKG2C, B7-H3, and a ligand that specifically binds to CD83.

As noted above, an “immunomodulatory polypeptide” (also referred to herein as a “MOD”) specifically binds a cognate co-immunomodulatory polypeptide on a T cell.

An “immunomodulatory domain” (“MOD”) of a TMAPP of the present disclosure binds a cognate co-immunomodulatory polypeptide, which may be present on a target T cell.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

The term “binding” refers to a direct association between two molecules, due to, for example, covalent, electrostatic, hydrophobic, and ionic and/or hydrogen-bond interactions, including interactions such as salt bridges and water bridges. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Treg” includes a plurality of such Tregs and reference to “the MHC Class II alpha chain” includes reference to one or more MHC Class II alpha chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides antigen-presenting polypeptides, including single-chain antigen-presenting polypeptides and multimeric antigen-presenting polypeptides. The present disclosure provides nucleic acids comprising nucleotide sequences encoding antigen-presenting polypeptides of the present disclosure, as well as cells genetically modified with the nucleic acids. An antigen-presenting polypeptide of the present disclosure is useful for modulating activity of a T cell. Thus, the present disclosure provides methods of modulating activity of a T cell.

An antigen-presenting polypeptide (APP) of the present disclosure can be a single-chain polypeptide or a multi-chain (multimeric) polypeptide. An APP of the present disclosure will in some cases include an immunomodulatory polypeptide. In other instances, an APP of the present disclosure does not include an immunomodulatory polypeptide; in these instances, the APP may be referred to herein as a T-cell modulatory APP or “TMAPP.” In some cases, a TMAPP of the present disclosure forms a higher order complex; for example, in some cases a TMAPP of the present disclosure forms a homodimer. Thus, the term “APP” includes: a multimeric APP; a single-chain APP; a multimeric TMAPP; and a single-chain TMAPP. The term further includes higher-order complexes of an APP.

### Antigen-Presenting Polypeptides

The present disclosure provides antigen-presenting polypeptides (APPs), including single-chain APPs and multimeric APPs.

Naturally occurring Class II MHC polypeptides comprise an α chain and a β chain. “Class II MHC polypeptides” include human leukocyte antigen (HLA) α- and β-chains. MHC Class II polypeptides include MCH Class II DP α and β polypeptides, DM α and β polypeptides, DOA α and β polypeptides, DOB α and β polypeptides, DQ α and β polypeptides, and DR α and β polypeptides. As used herein, a “Class II MHC polypeptide” can comprise a class II MHC α chain polypeptide, a class II MHC β chain polypeptide, or only a portion of a class II MHC α or β chain polypeptide. For example, a “Class II MHC polypeptide” can be a polypeptide that includes: i) only the α1 domain of a class II MHC α chain polypeptide; ii) only the α2 domain of a class II MHC α chain; iii) only the α1 domain and an α2 domain of a class II MHC α chain; iv) only the β1 domain of a class II MHC β chain; v) only the β2 domain of a class II MHC β chain; vi) only the β1 domain and the β2 domain of a class II MHC β chain; vii) the α1 domain of a class II MHC α chain, the β1 domain of a class II MHC β chain, and the β2 domain of a class II MHC; and the like.

Class II MHC polypeptides include allelic forms. The HLA locus is highly polymorphic in nature. As disclosed in the Nomenclature for Factors of the HLA System 2000 (Hum. Immunol.; 62(4):419-68, 2001) there are 221 HLA-DRB 1 alleles, 19 DRB3 alleles, 89 DRB4 alleles, 14 DRB5 alleles, 19 DQA1 alleles and 39 DQB1 alleles, with new alleles being discovered continuously. A 2007 update by the WHO nomenclature Committee for Factors of the HLA System (www.anthonynolan.com/HIG/) showed there are 3 DRA alleles, 494 DRB 1 alleles, 1 DRB2 alleles, 44 DRB3 alleles, 13 DRB4 alleles, 18 DRB5 alleles, 3 DRB6 alleles, 2 DRB7 alleles, 10 DRB8 alleles, 1 DRB9 alleles, 34 DQA1 alleles, 83 DQB1 alleles, 23 DPA1, 126 DPB1 alleles, 4 DMA alleles, 7 DMB alleles, 12 DOA alleles and 9 DOB alleles. As used herein, the term “Class II MHC polypeptide” includes allelic forms of any known Class II MHC polypeptide.

### Multimeric Antigen-Presenting Polypeptides

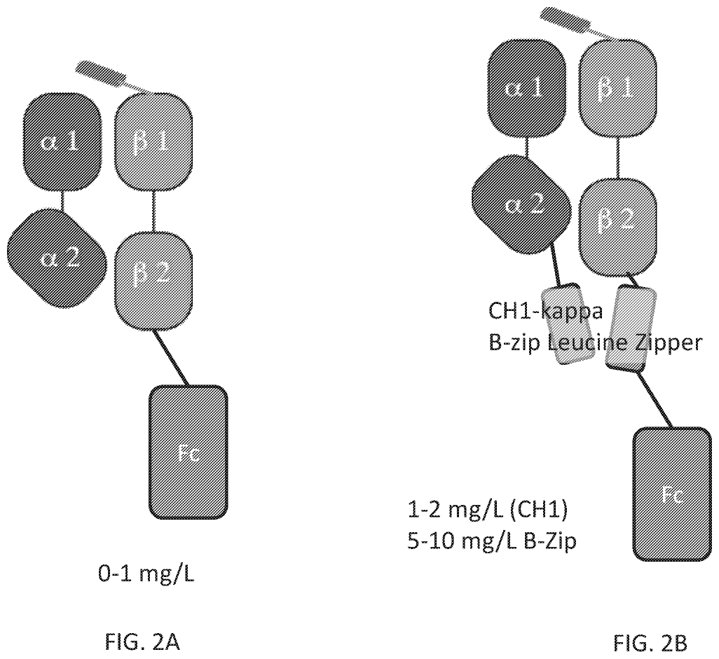

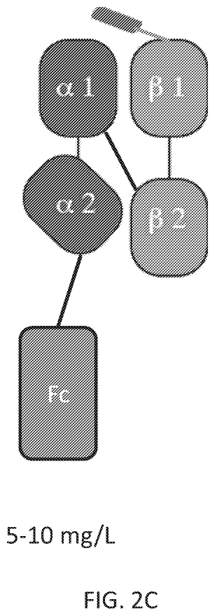

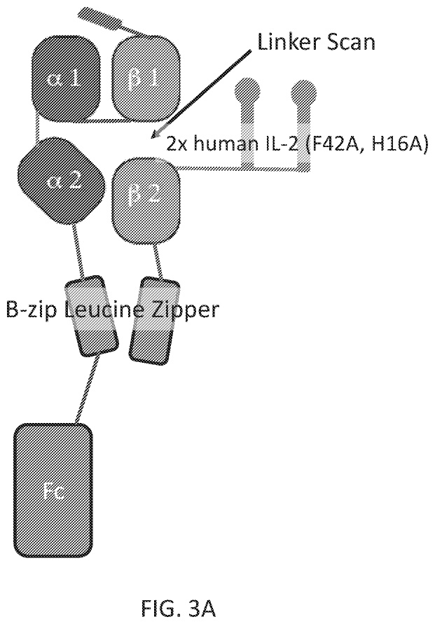

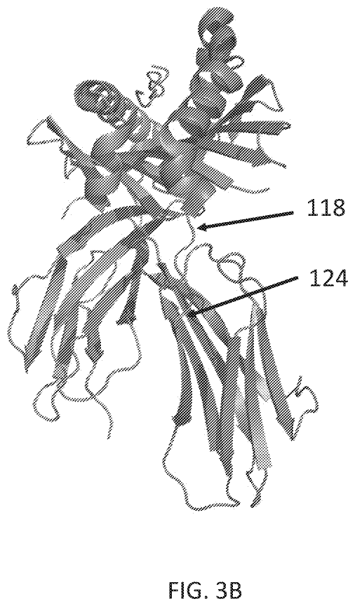

In some cases, an APP of the present disclosure comprises two polypeptide chains. In some cases, the two polypeptide chains are covalently linked to one another, e.g., via a disulfide bond. In other instances, the two polypeptide chains are not covalently linked to one another. In some cases, the two polypeptide chains are not covalently linked to one another; and in some of these cases, each of the two polypeptide chains comprises a member of a dimerization pair. Examples of multimeric APPs of the present disclosure are depicted schematically in FIG. 2A and FIG. 2B.

In some cases, an antigen-presenting multimeric polypeptide (multimeric APP) of the present disclosure comprises: a) a first polypeptide comprising, in order from N-terminus to C-terminus: i) an MHC Class II α1 polypeptide; and ii) an MHC Class II α2 polypeptide; and b) a second polypeptide comprising, in order from N-terminus to C-terminus: i) a peptide antigen (an “epitope”) that is recognized (e.g., is capable of being recognized and bound) by a T-cell receptor (TCR); ii) an MHC Class II pi polypeptide; and iii) an MHC Class II β2 polypeptide. In some cases, an APP of the present disclosure comprises: a) a first polypeptide comprising, in order from N-terminus to C-terminus: i) an MHC Class II α1 polypeptide; and ii) an MHC Class II α2 polypeptide; and b) a second polypeptide comprising, in order from N-terminus to C-terminus: i) a peptide antigen (an “epitope”) that is recognized (e.g., is capable of being recognized and bound) by a T-cell receptor (TCR); ii) an MHC Class II β1 polypeptide; iii) an MHC Class II β2 polypeptide; and iv) an immunoglobulin or non-immunoglobulin scaffold polypeptide. In some cases, an APP of the present disclosure comprises: a) a first polypeptide comprising, in order from N-terminus to C-terminus: i) an MHC Class II α1 polypeptide; and ii) an MHC Class II α2 polypeptide; and b) a second polypeptide comprising, in order from N-terminus to C-terminus: i) a peptide antigen (an “epitope”) that is recognized (e.g., is capable of being recognized and bound) by a T-cell receptor (TCR); ii) an MHC Class II β1 polypeptide; iii) an MHC Class II β2 polypeptide; and iv) an immunoglobulin (Ig) Fc polypeptide. In some cases, the second polypeptide comprises a linker between the peptide antigen and the MHC Class II β1 polypeptide. In some cases, the second polypeptide comprises a linker between the MHC Class II β1 polypeptide and the immunoglobulin or non-immunoglobulin scaffold polypeptide.

In some cases, an antigen-presenting multimeric polypeptide (a multimeric APP) of the present disclosure comprises: a) a first polypeptide comprising, in order from N-terminus to C-terminus: i) an MHC Class II α1 polypeptide; ii) an MHC Class II α2 polypeptide; and iii) a first member of a dimerizer pair; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) a second member of the dimerizer pair. The first and the second members of the dimerizer pair bind to one another non-covalently. In some cases, the first and the second members of the dimerizer pair bind to one another non-covalently without the need for a dimerization agent. In some cases, the first and the second members of the dimerizer pair bind to one another non-covalently in the presence of a dimerizer agent. In some cases, an APP of the present disclosure comprises: a) a first polypeptide comprising, in order from N-terminus to C-terminus: i) an MHC Class II α1 polypeptide; ii) an MHC Class II α2 polypeptide; and iii) a first member of a dimerizer pair; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) a second member of the dimerizer pair; and v) an immunoglobulin or non-immunoglobulin scaffold polypeptide. In some cases, an APP of the present disclosure comprises: a) a first polypeptide comprising, in order from N-terminus to C-terminus: i) an MHC Class II α1 polypeptide; ii) an MHC Class II α2 polypeptide; and iii) a first member of a dimerizer pair; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) a second member of the dimerizer pair; and v) an Ig Fc polypeptide. In some cases, an APP of the present disclosure comprises: a) a first polypeptide comprising, in order from N-terminus to C-terminus: i) an MHC Class II α1 polypeptide; ii) an MHC Class II α2 polypeptide; and iii) a first leucine zipper polypeptide; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) a second leucine zipper polypeptide; and v) an Ig Fc polypeptide. In some cases, the second polypeptide comprises a linker between the peptide antigen and the MHC Class II 11 polypeptide. In some cases, the second polypeptide comprises a linker between the MHC Class II β1 polypeptide and the second member of the dimerizing pair. In some cases, the first polypeptide comprises a linker between the MHC Class II α2 polypeptide and the first member of the dimerizing pair.

In some cases, an antigen-presenting multimeric polypeptide (a multimeric APP) of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first member of a dimerizing pair; and b) a second polypeptide comprising, in order from N-terminus to C-terminus: i) an MHC Class II β2 polypeptide; and ii) a second member of the dimerizing pair. In some cases, an APP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first member of a dimerizing pair; vi) an immunoglobulin or non-immunoglobulin scaffold polypeptide; and b) a second polypeptide comprising, in order from N-terminus to C-terminus: i) an MHC Class II β2 polypeptide; and ii) a second member of the dimerizing pair. In some cases, an APP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first member of a dimerizing pair; vi) an Ig Fc polypeptide; and b) a second polypeptide comprising, in order from N-terminus to C-terminus: i) an MHC Class II β2 polypeptide; and ii) a second member of the dimerizing pair. In some cases, an APP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first leucine zipper polypeptide; vi) an Ig Fc polypeptide; and b) a second polypeptide comprising, in order from N-terminus to C-terminus: i) an MHC Class II β2 polypeptide; and ii) a second leucine zipper polypeptide. In some cases, the first polypeptide comprises a linker between the peptide antigen and the MHC Class II β1 polypeptide. In some cases, the first polypeptide comprises a linker between the MHC Class II β1 polypeptide and the MHC Class II α1 polypeptide. In some cases, the first polypeptide comprises a linker between the MHC Class II α2 polypeptide and the first member of the dimerizing pair. In some cases, the second polypeptide comprises a linker between the MHC Class II β2 polypeptide and the second member of the dimerizing pair.

### Monomeric Antigen-Presenting Polypeptides

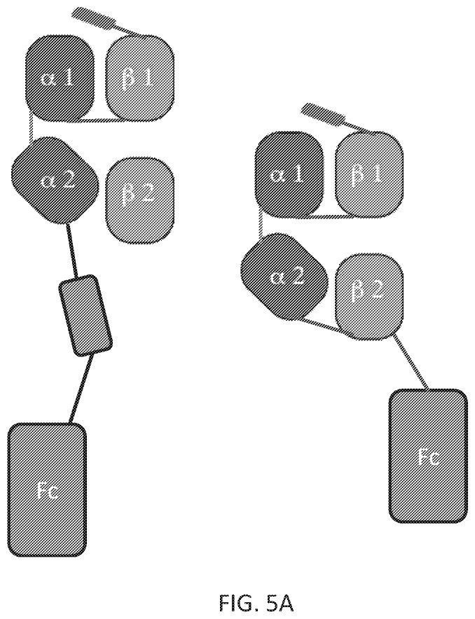

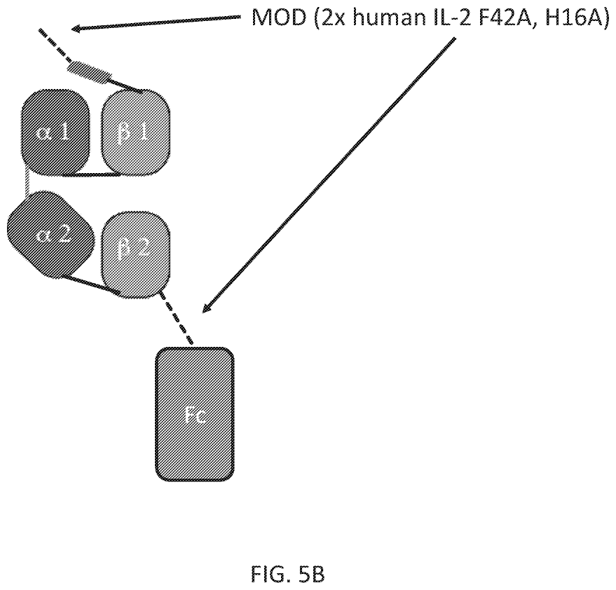

In some cases, an APP of the present disclosure is a single polypeptide chain. Examples are depicted schematically in FIG. 2C and FIG. 5A.

In some cases, an APP (e.g., a single-chain APP) of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; and v) an MHC Class II α2 polypeptide. In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; and vi) an immunoglobulin or non-immunoglobulin scaffold polypeptide. In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; and vi) an Ig Fc polypeptide. In some cases, the APP comprises a linker between the peptide antigen and the MHC Class II β1 polypeptide. In some cases, the APP comprises a linker between the MHC Class II β2 polypeptide and the MHC Class II α1 polypeptide. In some cases, the APP comprises a linker between the MHC Class II α2 polypeptide and the immunoglobulin or non-immunoglobulin scaffold.

In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an MHC Class II β2 polypeptide. In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; and vi) an immunoglobulin or non-immunoglobulin scaffold polypeptide. In some cases, an APP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; and vi) an Ig Fc polypeptide. In some cases, the APP comprises a linker between the peptide antigen and the MHC Class II β1 polypeptide. In some cases, the APP comprises a linker between the MHC Class II β1 polypeptide and the MHC Class II α1 polypeptide. In some cases, the APP comprises a linker between the MHC Class II α2 polypeptide and the MHC Class II β2 polypeptide. In some cases, the APP comprises a linker between the MHC Class II β2 polypeptide and the Ig or non-Ig scaffold.

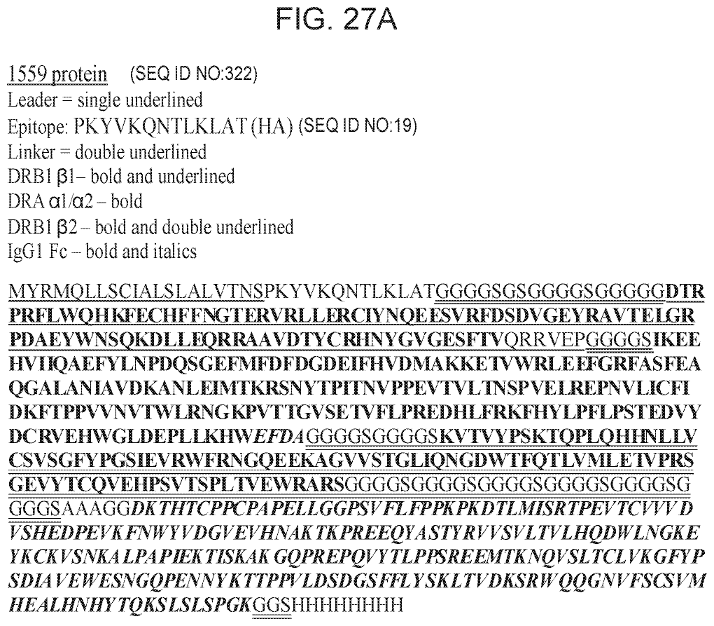

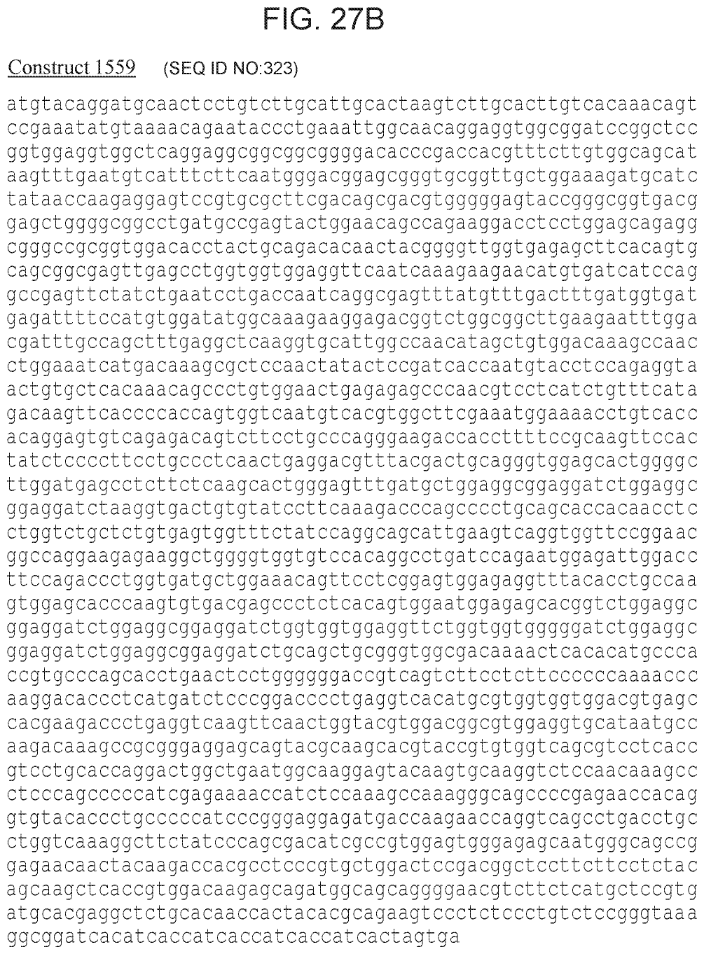

In some cases, a single-chain APP of the present disclosure comprises, in order from N-terminus to C-terminus: i) an epitope; ii) an HLA 31 polypeptide; iii) an HLA α1 polypeptide; iv) an HLA α2 polypeptide; v) an HLA β2 polypeptide; and vi) an Ig Fc polypeptide. As one non-limiting example, a single-chain APP of the present disclosure can comprise, in order from N-terminus to C-terminus: i) an epitope; ii) an HLA DRB1 β1 polypeptide; iii) an HLA DRA α1 polypeptide; iv) an HLA DRA α2 polypeptide; v) an HLA DRB β2 polypeptide; and vi) an IgG1 Fc polypeptide. In some cases, the epitope is a hemagglutinin epitope (PKYVKQNTLKLAT; SEQ ID NO:19). In other instances, the epitope is not PKYVKQNTLKLAT (SEQ ID NO:19); instead, the epitope is substituted with a different epitope. In some cases, the single-chain polypeptide comprises the 1559 amino acid sequence depicted in FIG. 27A, without the leader peptide and without the C-terminal linker and histidine tag. For example, in some cases, the single-chain polypeptide comprises amino acids 21-700 of the amino acid sequence depicted in FIG. 27A.

### MHC Class II Alpha Chains

MHC Class II alpha chains comprise an α1 domain and an α2 domain. In some cases, the α1 domain and the α2 domain present in an antigen-presenting cell are from the same MHC Class II α chain polypeptide. In some cases, the α1 domain and the α2 domain present in an antigen-presenting cell are from two different MHC Class II α chain polypeptides.

MHC Class II alpha chains suitable for inclusion in an APP (e.g., a multimeric APP; a single-chain APP; a multimeric TMAPP; a single-chain TMAPP) of the present disclosure lack a signal peptide. An MHC Class II alpha chain suitable for inclusion in a multimeric polypeptide of the present disclosure can have a length of from about 60 amino acids to about 190 amino acids; for example, an MHC Class II alpha chain suitable for inclusion in an APP of the present disclosure can have a length of from about 60 amino acids to about 80 amino acids, from about 80 amino acids to about 100 amino acids, from about 100 amino acids to about 120 amino acids, from about 120 amino acids to about 140 amino acids, from about 140 amino acids to about 160 amino acids, from about 160 amino acids to about 180 amino acids, or from about 180 amino acids to about 200 amino acids. An MHC Class II α1 domain suitable for inclusion in an APP of the present disclosure can have a length of from about 30 amino acids to about 95 amino acids; for example, an MHC Class II α1 domain suitable for inclusion in an APP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, or from about 90 amino acids to about 95 amino acids. An MHC Class II α2 domain suitable for inclusion in an APP of the present disclosure can have a length of from about 30 amino acids to about 95 amino acids; for example, an MHC Class II α2 domain suitable for inclusion in an APP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, or from about 90 amino acids to about 95 amino acids.

### DRA

In some cases, a suitable MHC Class II α chain polypeptide is a DRA polypeptide. A DRA polypeptide can have at least 85%, at least 90%, at least 95%, at least 98%, at least 99%, or 100%, amino acid sequence identity with amino acids 26-203 of the DRA amino acid sequence depicted in FIG. 6. In some cases, the DRA polypeptide has a length of about 178 amino acids (e.g., 175, 176, 177, 178, 179, or 180 amino acids).

A “DRA polypeptide” includes allelic variants, e.g., naturally occurring allelic variants. Thus, in some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLEIMTK RSNYTPITNV PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO:20), or an allelic variant thereof.

A suitable DRA α1 domain comprises an amino acid sequence having at least 85%, at least 90%, at least 95%, at least 98%, at least 99%, or 100%, amino acid sequence identity to the following amino acid sequence: VIIQAEFYLN PDQSGEFMFD FDGDEIFHVD MAKKETVWRL EEFGRFASFE AQGALANIAV DKANLEIMTK RSNYTPITN (SEQ ID NO:21); and can have a length of about 84 amino acids (e.g., 80, 81, 82, 83, 84, 85, or 86 amino acids). A suitable DRA α1 domain can comprise the following amino acid sequence: VIIQAEFYLN PDQSGEFMFD FDGDEIFHVD MAKKETVWRL EEFGRFASFE AQGALANIAV DKANLEIMTK RSNYTPITN (SEQ ID NO:21), or a naturally-occurring allelic variant.

A suitable DRA α2 domain comprises an amino acid sequence having at least 85%, at least 90%, at least 95%, at least 98%, at least 99%, or 100%, amino acid sequence identity to the following amino acid sequence: V PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO:22); and can have a length of about 94 amino acids (e.g., 90, 91, 92, 93, 94, 95, 96, 97, or 98 amino acids).

### DMA

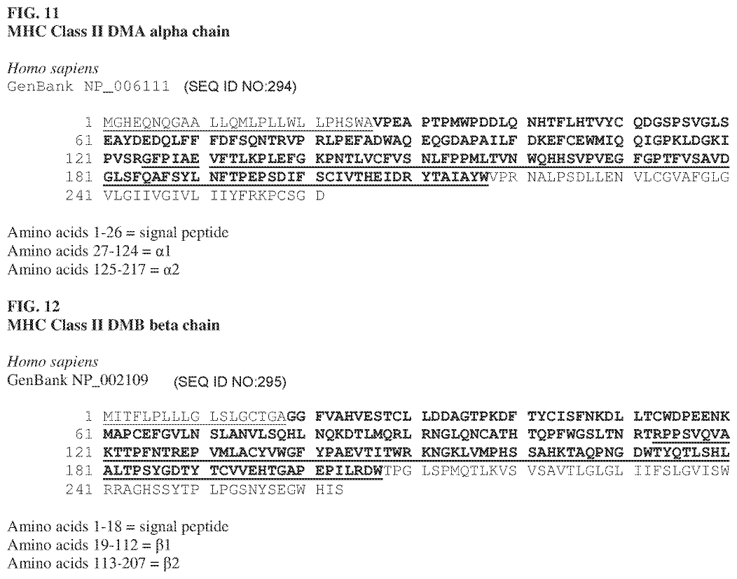

In some cases, a suitable MHC Class II α chain polypeptide is a DMA polypeptide. A DMA polypeptide can have at least 85%, at least 90%, at least 95%, at least 98%, at least 99%, or 100%, amino acid sequence identity with amino acids 27-217 of the DMA amino acid sequence depicted in FIG. 11. In some cases, the DMA polypeptide has a length of about 191 amino acids (e.g., 188, 189, 190, 191, 192, or 193 amino acids).

A “DMAA polypeptide” includes allelic variants, e.g., naturally occurring allelic variants. Thus, in some cases, a suitable DMAA polypeptide comprises the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSRGFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO:23), or an allelic variant thereof.

A suitable DMA α1 domain comprises an amino acid sequence having at least 85%, at least 90%, at least 95%, at least 98%, at least 99%, or 100%, amino acid sequence identity to the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSR (SEQ ID NO:24); and can have a length of about 98 amino acids (e.g., 94, 95, 96, 97, 98, 99, 100, or 101 amino acids). A suitable DMA α1 domain can comprise the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSR (SEQ ID NO:24), or a naturally-occurring allelic variant thereof.

A suitable DMA α2 domain comprises an amino acid sequence having at least 85%, at least 90%, at least 95%, at least 98%, at least 99%, or 100%, amino acid sequence identity to the following amino acid sequence: GFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO:25); and can have a length of about 93 amino acids (e.g., 90, 91, 92, 93, 94, 95, 96, or 97 amino acids). A suitable DMA α2 domain can comprise the following amino acid sequence: GFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO:25), or a naturally-occurring allelic variant thereof.

### DOA

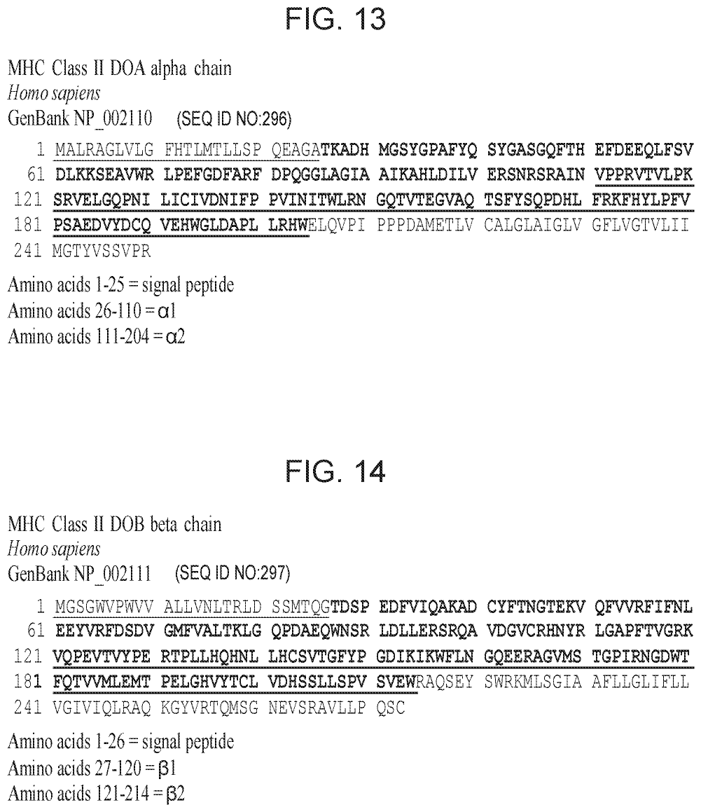

In some cases, a suitable MHC Class II α chain polypeptide is a DOA polypeptide. A DOA polypeptide can have at least 85%, at least 90%, at least 95%, at least 98%, at least 99%, or 100%, amino acid sequence identity with amino acids 26-204 of the DOA amino acid sequence depicted in FIG. 13. In some cases, the DOA polypeptide has a length of about 179 amino acids (e.g., 175, 176, 177, 178, 179, 180, 181, or 182 amino acids).

A “DOA polypeptide” includes allelic variants, e.g., naturally occurring allelic variants. Thus, in some cases, a suitable DOA polypeptide comprises the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO:26), or an allelic variant thereof.

A suitable DOA α1 domain comprises an amino acid sequence having at least 85%, at least 90%, at least 95%, at least 98%, at least 99%, or 100%, amino acid sequence identity to the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN (SEQ ID NO:27); and can have a length of about 85 amino acids (e.g., 83, 84, 85, 86, 87, or 88 amino acids). A suitable DOA α1 domain can comprise the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN (SEQ ID NO:27), or a naturally-occurring allelic variant.

A suitable DOA α2 domain comprises an amino acid sequence having at least 85%, at least 90%, at least 95%, at least 98%, at least 99%, or 100%, amino acid sequence identity to the following amino acid sequence: VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO:28); and can have a length of about 94 amino acids (e.g., 91, 92, 93, 94, 95, 96, or 97 amino acids). A suitable DOA α2 domain can comprise the following amino acid sequence: VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO:28), or a naturally-occurring allelic variant thereof.

### DPA1

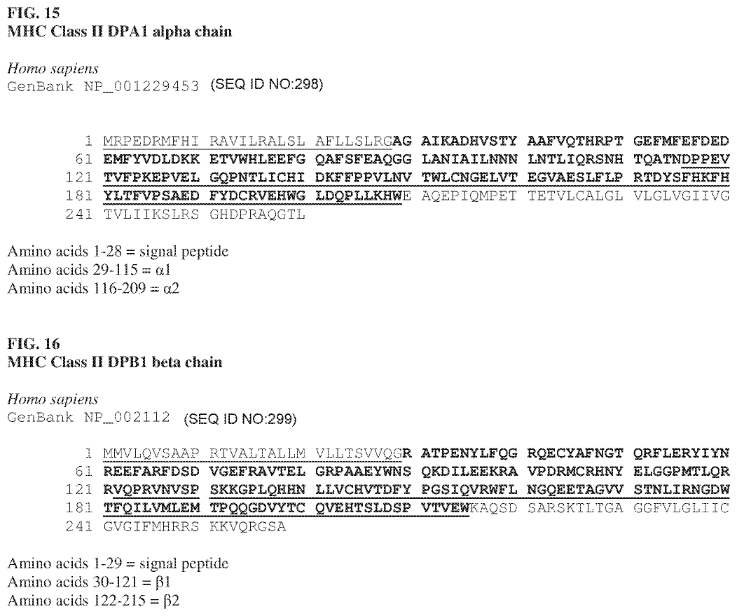

In some cases, a suitable MHC Class II α chain polypeptide is a DPA1 polypeptide. A DPA1 polypeptide can have at least 85%, at least 90%, at least 95%, at least 98%, at least 99%, or 100%, amino acid sequence identity with amino acids 29-209 of the DPA1 amino acid sequence depicted in FIG. 15. In some cases, the DPA1 polypeptide has a length of about 181 amino acids (e.g., 178, 179, 180, 181, 182, 183, or 184 amino acids).

A “DPA1 polypeptide” includes allelic variants, e.g., naturally occurring allelic variants. Thus, in some cases, a suitable DPA1 polypeptide comprises the following amino acid sequence: AG AIKADHVSTY AAFVQTHRPT GEFMFEFDED EMFYVDLDKK ETVWHLEEFG QAFSFEAQGG LANIAILNNN LNTLIQRSNH TQATNDPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:29), or an allelic variant thereof.

A suitable DPA1 α1 domain comprises an amino acid sequence having at least 85%, at least 90%, at least 95%, at least 98%, at least 99%, or 100%, amino acid sequence identity to the following amino acid sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO:30); and can have a length of about 87 amino acids (e.g., 84, 85, 86, 87, 88, or 89 amino acids). A suitable DPA1 α1 domain can comprise the following amino acid sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO:30), or a naturally-occurring allelic variant.

A suitable DPA1 α2 domain comprises an amino acid sequence having at least 85%, at least 90%, at least 95%, at least 98%, at least 99%, or 100%, amino acid sequence identity to the following amino acid sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:31); and can have a length of about 97 amino acids (e.g., 91, 92, 93, 94, 95, 96, or 97 amino acids). A suitable DPA1 α2 domain can comprise the following amino acid sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:31), or a naturally-occurring allelic variant thereof.

### DQA1

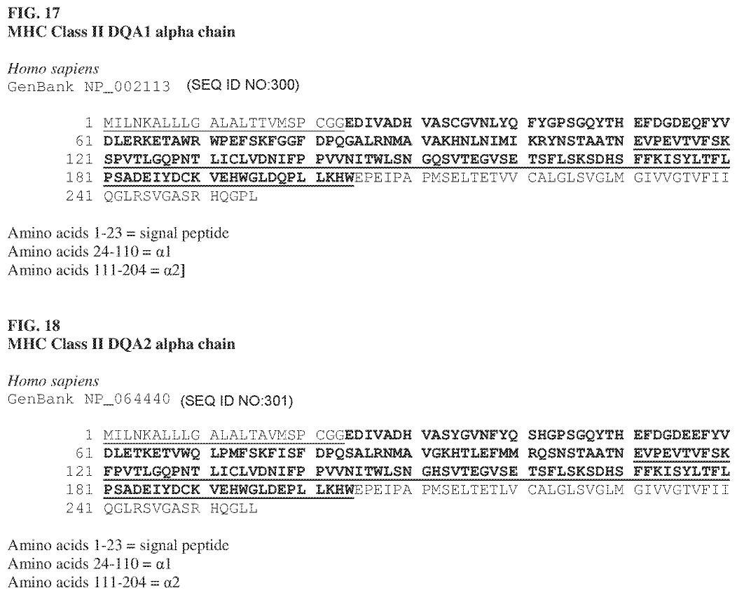

In some cases, a suitable MHC Class II α chain polypeptide is a DQA1 polypeptide. A DQA1 polypeptide can have at least 85%, at least 90%, at least 95%, at least 98%, at least 99%, or 100%, amino acid sequence identity with amino acids 24-204 of the DQA1 amino acid sequence depicted in FIG. 17. In some cases, the DQA1 polypeptide has a length of about 181 amino acids (e.g., 177, 178, 179, 180, 181, 182, or 183 amino acids).

A “DQA1 polypeptide” includes allelic variants, e.g., naturally occurring allelic variants. Thus, in some cases, a suitable DQA1 polypeptide comprises the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:32), or an allelic variant thereof.

A suitable DQA1 α1 domain comprises an amino acid sequence having at least 85%, at least 90%, at least 95%, at least 98%, at least 99%, or 100%, amino acid sequence identity to the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO:33); and can have a length of about 87 amino acids (e.g., 84, 85, 86, 87, 88, or 89 amino acids). A suitable DQA1 α1 domain can comprise the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO:33), or a naturally-occurring allelic variant.

A suitable DQA1 α2 domain comprises an amino acid sequence having at least 85%, at least 90%, at least 95%, at least 98%, at least 99%, or 100%, amino acid sequence identity to the following amino acid sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:34); and can have a length of about 94 amino acids (e.g., 91, 92, 93, 94, 95, 96, or 97 amino acids). A suitable DQA1 α2 domain can comprise the following amino acid sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:34), or a naturally-occurring allelic variant thereof.

### DQA2

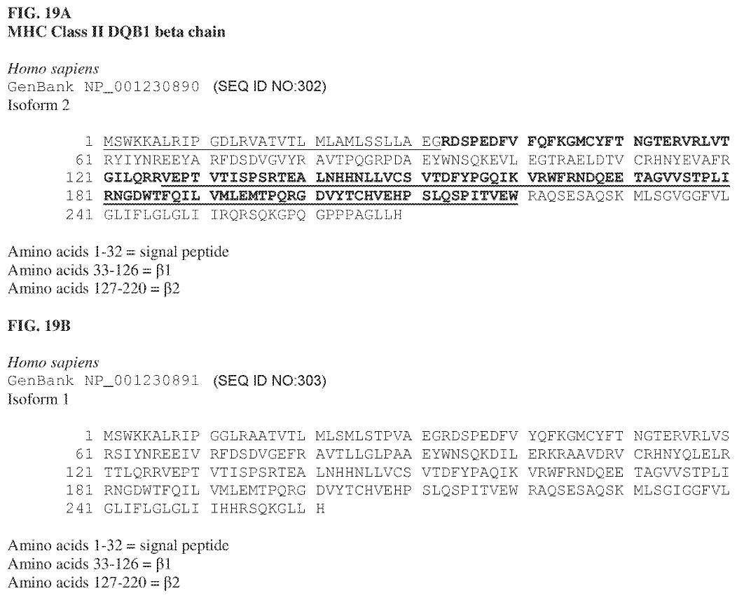

In some cases, a suitable MHC Class II α chain polypeptide is a DQA2 polypeptide. A DQA2 polypeptide can have at least 85%, at least 90%, at least 95%, at least 98%, at least 99%, or 100%, amino acid sequence identity with amino acids 24-204 of the DQA2 amino acid sequence depicted in FIG. 18. In some cases, the DQA2 polypeptide has a length of about 181 amino acids (e.g., 177, 178, 179, 180, 181, 182, or 183 amino acids).

A “DQA2 polypeptide” includes allelic variants, e.g., naturally occurring allelic variants. Thus, in some cases, a suitable DQA2 polypeptide comprises the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:35), or an allelic variant thereof.

A suitable DQA2 α1 domain comprises an amino acid sequence having at least 85%, at least 90%, at least 95%, at least 98%, at least 99%, or 100%, amino acid sequence identity to the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN (SEQ ID NO:36); and can have a length of about 87 amino acids (e.g., 84, 85, 86, 87, 88, or 89 amino acids). A suitable DQA2 α1 domain can comprise the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN (SEQ ID NO:36), or a naturally-occurring allelic variant.

A suitable DQA2 α2 domain comprises an amino acid sequence having at least 85%, at least 90%, at least 95%, at least 98%, at least 99%, or 100%, amino acid sequence identity to the following amino acid sequence: EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:37); and can have a length of about 94 amino acids (e.g., 91, 92, 93, 94, 95, 96, or 97 amino acids). A suitable DQA2 α2 domain can comprise the following amino acid sequence: EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:37), or a naturally-occurring allelic variant thereof.

### MHC Class II Beta Chains

MHC Class II beta chains comprise a β1 domain and a β2 domain. In some cases, the β1 domain and the β2 domain present in an antigen-presenting cell are from the same MHC Class II β chain polypeptide. In some cases, the β1 domain and the β2 domain present in an antigen-presenting cell are from two different MHC Class II β chain polypeptides.

MHC Class II beta chains suitable for inclusion in an APP (e.g., a multimeric APP; a single-chain APP; a multimeric TMAPP; a single-chain TMAPP) of the present disclosure lack a signal peptide. An MHC Class II beta chain suitable for inclusion in an APP of the present disclosure can have a length of from about 60 amino acids to about 210 amino acids; for example, an MHC Class II beta chain suitable for inclusion in an APP of the present disclosure can have a length of from about 60 amino acids to about 80 amino acids, from about 80 amino acids to about 100 amino acids, from about 100 amino acids to about 120 amino acids, from about 120 amino acids to about 140 amino acids, from about 140 amino acids to about 160 amino acids, from about 160 amino acids to about 180 amino acids, from about 180 amino acids to about 200 amino acids, or from about 200 amino acids to about 210 amino acids. An MHC Class II domain suitable for inclusion in an APP of the present disclosure can have a length of from about 30 amino acids to about 105 amino acids; for example, an MHC Class II domain suitable for inclusion in an APP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, from about 90 amino acids to about 95 amino acids, from about 95 amino acids to about 100 amino acids, or from about 100 amino acids to about 105 amino acids. An MHC Class II 2 domain suitable for inclusion in an APP of the present disclosure can have a length of from about 30 amino acids to about 105 amino acids; for example, an MHC Class II 2 domain suitable for inclusion in an APP of the present disclosure can have a length of from about 30 amino acids to about 40 amino acids, from about 40 amino acids to about 50 amino acids, from about 50 amino acids to about 60 amino acids, from about 60 amino acids to about 70 amino acids, from about 70 amino acids to about 80 amino acids, from about 80 amino acids to about 90 amino acids, from about 90 amino acids to about 95 amino acids, from about 95 amino acids to about 100 amino acids, or from about 100 amino acids to about 105 amino acids.

### DRB1

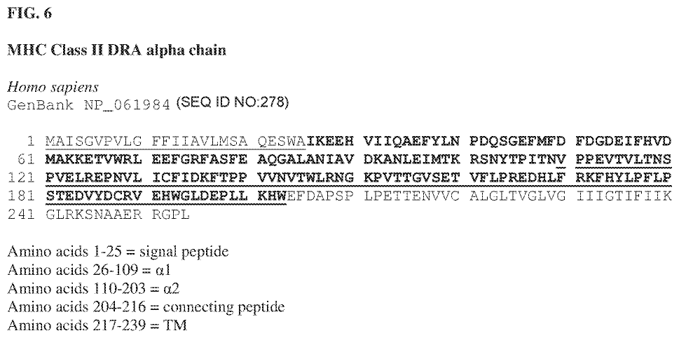

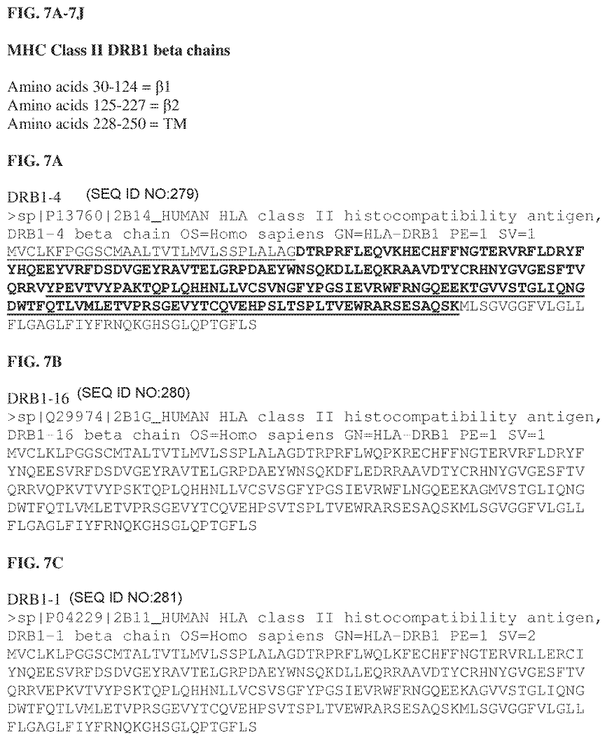

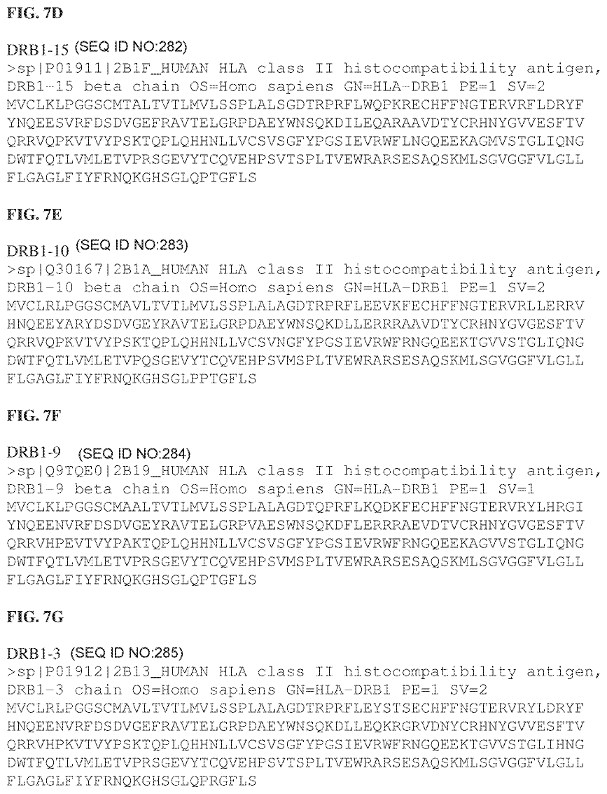

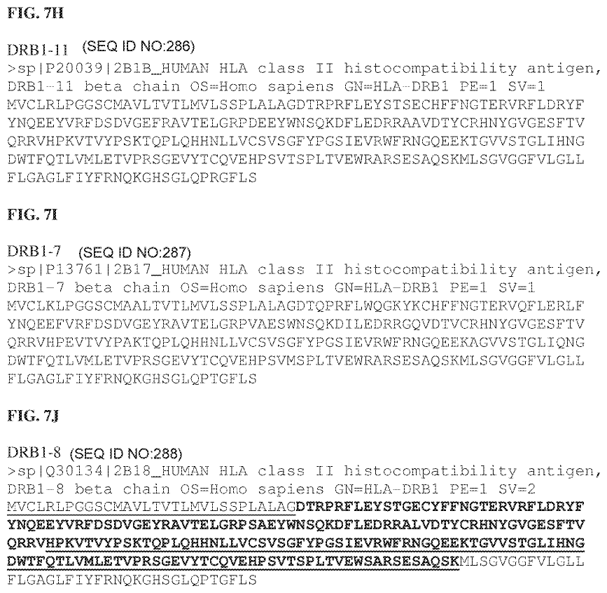

In some cases, a suitable MHC Class II chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in any one of FIG. 7A-7J. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7A. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7B. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7C. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7D. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7E. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7F. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7G. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7H. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7I. In some cases, a suitable MHC Class II β chain polypeptide is a DRB1 polypeptide. A DRB1 polypeptide can have at least 85%, at least 90%, at least 95%, at least 98%, at least 99%, or 100%, amino acid sequence identity with amino acids 30-227 of the DRB1 amino acid sequence depicted in FIG. 7J. In some cases, the DRB1 polypeptide has a length of about 198 amino acids (e.g., 195, 196, 197, 198, 199, 200, 201, or 202 amino acids).

A “DRB1 polypeptide” includes allelic variants, e.g., naturally occurring allelic variants. Thus, in some cases, a suitable DRB1 polypeptide comprises the following amino acid sequence: DTRPRFLEQVKHECHFFNGTERVRFLDRYFYHQEEYVRFDSDVGEYRAVTELGRPDAE YWNSQKDLLEQKRAAVDTYCRHNYGVGESFTVQRRVYPEVTVYPAKTQPLQHHNLLV CSVNGFYPGSIEVRWFRNGQEEKTGVVSTGLIQNGDWTFQTLVMLETVPRSGEVYTCQ VEHPSLTSPLTVEWRARSESAQSK (SEQ ID NO:38), or an allelic variant thereof.

A suitable DRB1 β1 domain comprises an amino acid sequence having at least 85%, at least 90%, at least 95%, at least 98%, at least 99%, or 100%, amino acid sequence identity to the following amino acid sequence: DTRPRFLEQVKHECHFFNGTERVRFLDRYFYHQEEYVRFDSDVGEYRAVTELGRPDAE YWNSQKDLLEQKRAAVDTYCRHNYGVGESFTVQRRV (SEQ ID NO:39); and can have a length of about 95 amino acids (e.g., 92, 93, 94, 95, 96, 97, or 98 amino acids). A suitable DRB1 β1 domain can comprise the following amino acid sequence: DTRPRFLEQVKHECHFFNGTERVRFLDRYFYHQEEYVRFDSDVGEYRAVTELGRPDAE YWNSQKDLLEQKRAAVDTYCRHNYGVGESFTVQRRV (SEQ ID NO:39), or a naturally-occurring allelic variant.

A suitable DRB1 β2 domain comprises an amino acid sequence having at least 85%, at least 90%, at least 95%, at least 98%, at least 99%, or 100%, amino acid sequence identity to the following amino acid sequence: YPEVTVYPAKTQPLQHHNLLVCSVNGFYPGSIEVRWFRNGQEEKTGVVSTGLIQNGDW TFQTLVMLETVPRSGEVYTCQVEHPSLTSPLTVEWRARSESAQSK (SEQ ID NO:40); and can have a length of about 103 amino acids (e.g., 100, 101, 102, 103, 104, 105, or 106 amino acids). A suitable DRB1 β2 domain can comprise the following amino acid sequence: YPEVTVYPAKTQPLQHHNLLVCSVNGFYPGSIEVRWFRNGQEEKTGVVSTGLIQNGDW TFQTLVMLETVPRSGEVYTCQVEHPSLTSPLTVEWRARSESAQSK (SEQ ID NO:40), or a naturally-occurring allelic variant thereof.

### DRB3

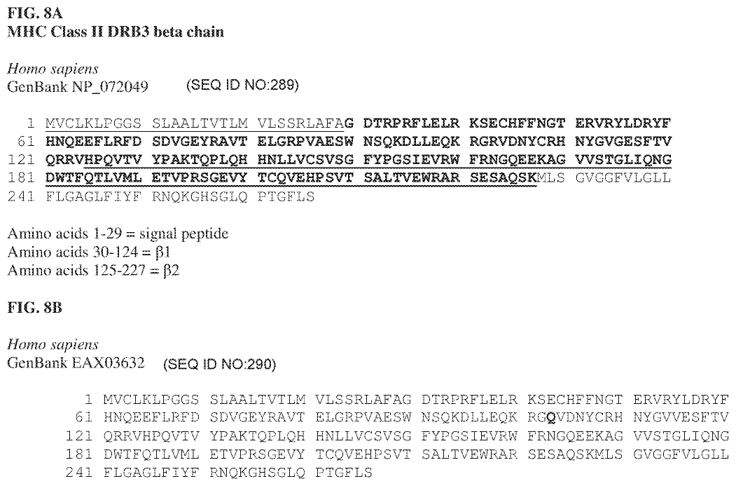

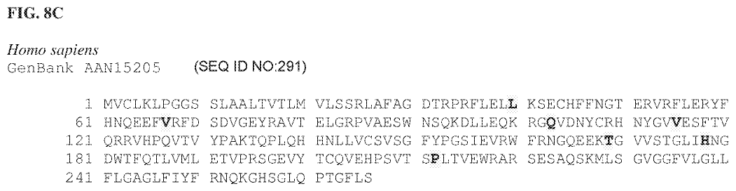

In some cases, a suitable MHC Class II β chain polypeptide is a DRB3 polypeptide. A DRB3 polypeptide can have at least 85%, at least 90%, at least 95%, at least 98%, at least 99%, or 100%, amino acid sequence identity with amino acids 30-227 of the DRB3 amino acid sequence depicted in any one of FIG. 8A-8C. In some cases, a suitable MHC Class II β chain polypeptide is a DRB3 polypeptide. A DRB3 polypeptide can have at least 85%, at least 90%, at least 95%, at least 98%, at least 99%, or 100%, amino acid sequence identity with amino acids 30-227 of the DRB3 amino acid sequence depicted in FIG. 8A. In some cases, a suitable MHC Class II β chain polypeptide is a DRB3 polypeptide. A DRB3 polypeptide can have at least 85%, at least 90%, at least 95%, at least 98%, at least 99%, or 100%, amino acid sequence identity with amino acids 30-227 of the DRB3 amino acid sequence depicted in FIG. 8B. In some cases, a suitable MHC Class II β chain polypeptide is a DRB3 polypeptide. A DRB3 polypeptide can have at least 85%, at least 90%, at least 95%, at least 98%, at least 99%, or 100%, amino acid sequence identity with amino acids 30-227 of the DRB3 amino acid sequence depicted in FIG. 8C. In some cases, the DRB3 polypeptide has a length of about 198 amino acids (e.g., 195, 196, 197, 198, 199, 200, 201, or 202 amino acids).

A “DRB3 polypeptide” includes allelic variants, e.g., naturally occurring allelic variants. Thus, in some cases, a suitable DRB3 polypeptide comprises the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRVHPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:41), or an allelic variant thereof.

A suitable DRB3 β1 domain comprises an amino acid sequence having at least 85%, at least 90%, at least 95%, at least 98%, at least 99%, or 100%, amino acid sequence identity to the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:42); and can have a length of about 95 amino acids (e.g., 93, 94, 95, 96, 97, or 98 amino acids). A suitable DRB3 β1 domain can comprise the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:42), or a naturally-occurring allelic variant.

A suitable DRB3 β2 domain comprises an amino acid sequence having at least 85%, at least 90%, at least 95%, at least 98%, at least 99%, or 100%, amino acid sequence identity to the following amino acid sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:43); and can have a length of about 103 amino acids (e.g., 100, 101, 102, 103, 104, or 105 amino acids). A suitable DRB3 β2 domain can comprise the following amino acid sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:43), or a naturally-occurring allelic variant thereof.

### DRB4

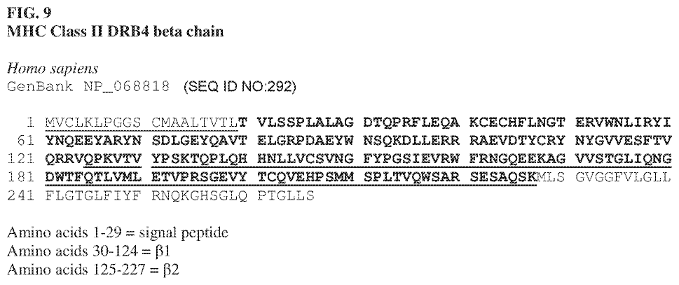

In some cases, a suitable MHC Class II β chain polypeptide is a DRB4 polypeptide. A DRB4 polypeptide can have at least 85%, at least 90%, at least 95%, at least 98%, at least 99%, or 100%, amino acid sequence identity with amino acids 30-227 of the DRB4 amino acid sequence depicted in FIG. 9. In some cases, the DRB4 polypeptide has a length of about 198 amino acids (e.g., 195, 196, 197, 198, 199, 200, 201, or 202 amino acids).

A “DRB4 polypeptide” includes allelic variants, e.g., naturally occurring allelic variants. Thus, in some cases, a suitable CDR4 polypeptide comprises the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRVQPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:44), or an allelic variant thereof.

A suitable DRB4 β1 domain comprises an amino acid sequence having at least 85%, at least 90%, at least 95%, at least 98%, at least 99%, or 100%, amino acid sequence identity to the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:45); and can have a length of about 95 amino acids (e.g., 93, 94, 95, 96, 97, or 98 amino acids). A suitable DRB4 β1 domain can comprise the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:45), or a naturally-occurring allelic variant.

A suitable DRB4 β2 domain comprises an amino acid sequence having at least 85%, at least 90%, at least 95%, at least 98%, at least 99%, or 100%, amino acid sequence identity to the following amino acid sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:46); and can have a length of about 103 amino acids (e.g., 100, 101, 102, 103, 104, or 105 amino acids). A suitable DRB4 β2 domain can comprise the following amino acid sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:46), or a naturally-occurring allelic variant thereof.

### DRB5

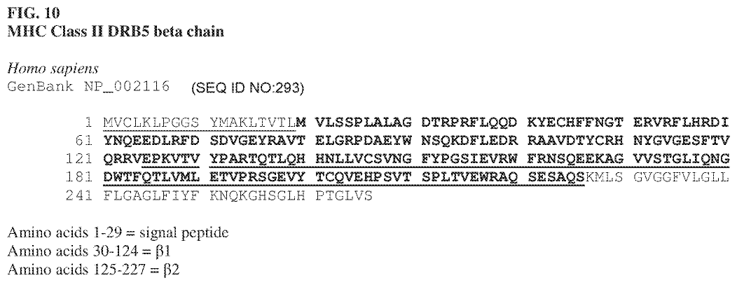

In some cases, a suitable MHC Class II β chain polypeptide is a DRB5 polypeptide. A DRB5 polypeptide can have at least 85%, at least 90%, at least 95%, at least 98%, at least 99%, or 100%, amino acid sequence identity with amino acids 30-227 of the DRB5 amino acid sequence depicted in FIG. 10. In some cases, the DRB5 polypeptide has a length of about 198 amino acids (e.g., 195, 196, 197, 198, 199, 200, 201, or 202 amino acids).

A “DRB5 polypeptide” includes allelic variants, e.g., naturally occurring allelic variants. Thus, in some cases, a suitable DRB5 polypeptide comprises the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRVEPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:47), or an allelic variant thereof.

A suitable DRB5 β1 domain comprises an amino acid sequence having at least 85%, at least 90%, at least 95%, at least 98%, at least 99%, or 100%, amino acid sequence identity to the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO:48); and can have a length of about 95 amino acids (e.g., 93, 94, 95, 96, 97, or 98 amino acids). A suitable DRB5 β1 domain can comprise the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO:48), or a naturally-occurring allelic variant.

A suitable DRB5 β2 domain comprises an amino acid sequence having at least 85%, at least 90%, at least 95%, at least 98%, at least 99%, or 100%, amino acid sequence identity to the following amino acid sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:49); and can have a length of about 103 amino acids (e.g., 100, 101, 102, 103, 104, or 105 amino acids). A suitable DRB5 β2 domain can comprise the following amino acid sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:49), or a naturally-occurring allelic variant thereof.

### DMB

In some cases, a suitable MHC Class II β chain polypeptide is a DMB polypeptide. A DMB polypeptide can have at least 85%, at least 90%, at least 95%, at least 98%, at least 99%, or 100%, amino acid sequence identity with amino acids 19-207 of the DMB amino acid sequence depicted in FIG. 12. In some cases, the DMB polypeptide has a length of about 189 amino acids (e.g., 187, 188, 189, 190, or 191 amino acids).

A “DMB polypeptide” includes allelic variants, e.g., naturally occurring allelic variants. Thus, in some cases, a suitable DMB polypeptide comprises the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RTRPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO:50), or an allelic variant thereof.

A suitable DMB β1 domain comprises an amino acid sequence having at least 85%, at least 90%, at least 95%, at least 98%, at least 99%, or 100%, amino acid sequence identity to the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RT (SEQ ID NO:51); and can have a length of about 94 amino acids (e.g., 92, 93, 94, 95, 96, or 97 amino acids). A suitable DMB β1 domain can comprise the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RT (SEQ ID NO:51), or a naturally-occurring allelic variant.

A suitable DMB β2 domain comprises an amino acid sequence having at least 85%, at least 90%, at least 95%, at least 98%, at least 99%, or 100%, amino acid sequence identity to the following amino acid sequence: RPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO:52); and can have a length of about 95 amino acids (e.g., 93, 94, 95, 96, 97, or 98 amino acids). A suitable DMB β2 domain can comprise the following amino acid sequence: RPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO:52), or a naturally-occurring allelic variant thereof.

### DOB

In some cases, a suitable MHC Class II β chain polypeptide is a DOB polypeptide. A DOB polypeptide can have at least 85%, at least 90%, at least 95%, at least 98%, at least 99%, or 100%, amino acid sequence identity with amino acids 27-214 of the DOB amino acid sequence depicted in FIG. 14. In some cases, the DOB polypeptide has a length of about 188 amino acids (e.g., 186, 187, 188, 189, or 190 amino acids).

A “DOB polypeptide” includes allelic variants, e.g., naturally occurring allelic variants. Thus, in some cases, a suitable DOB polypeptide comprises the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO:53), or an allelic variant thereof.

A suitable DOB β1 domain comprises an amino acid sequence having at least 85%, at least 90%, at least 95%, at least 98%, at least 99%, or 100%, amino acid sequence identity to the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK (SEQ ID NO:54); and can have a length of about 94 amino acids (e.g., 92, 93, 94, 95, 96, or 97 amino acids). A suitable DOB β1 domain can comprise the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK (SEQ ID NO:54), or a naturally-occurring allelic variant.

A suitable DOB β2 domain comprises an amino acid sequence having at least 85%, at least 90%, at least 95%, at least 98%, at least 99%, or 100%, amino acid sequence identity to the following amino acid sequence: VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO:55); and can have a length of about 94 amino acids (e.g., 92, 93, 94, 95, 96, or 97 amino acids). A suitable DOB β2 domain can comprise the following amino acid sequence: VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO:55), or a naturally-occurring allelic variant thereof.

### DPB1

In some cases, a suitable MHC Class II β chain polypeptide is a DPB1 polypeptide. A DPB1 polypeptide can have at least 85%, at least 90%, at least 95%, at least 98%, at least 99%, or 100%, amino acid sequence identity with amino acids 30-215 of the DPB1 amino acid sequence depicted in FIG. 16. In some cases, the DPB1 polypeptide has a length of about 186 amino acids (e.g., 184, 185, 186, 187, or 188 amino acids).

A “DPB1 polypeptide” includes allelic variants, e.g., naturally occurring allelic variants. Thus, in some cases, a suitable DPB1 polypeptide comprises the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR RVQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:56), or an allelic variant thereof.

A suitable DPB1 β1 domain comprises an amino acid sequence having at least 85%, at least 90%, at least 95%, at least 98%, at least 99%, or 100%, amino acid sequence identity to the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO:57); and can have a length of about 92 amino acids (e.g., 90, 91, 92, 93, or 94 amino acids). A suitable DPB1 β1 domain can comprise the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO:57), or a naturally-occurring allelic variant.

A suitable DPB1 β2 domain comprises an amino acid sequence having at least 85%, at least 90%, at least 95%, at least 98%, at least 99%, or 100%, amino acid sequence identity to the following amino acid sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:58); and can have a length of about 94 amino acids (e.g., 92, 93, 94, 95, 96, or 97 amino acids). A suitable DPB1 β2 domain can comprise the following amino acid sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:58), or a naturally-occurring allelic variant thereof.

### DQB1

In some cases, a suitable MHC Class II β chain polypeptide is a DQB1 polypeptide. A DQB1 polypeptide can have at least 85%, at least 90%, at least 95%, at least 98%, at least 99%, or 100%, amino acid sequence identity with amino acids 33-220 of the DQB1 amino acid sequence depicted in FIG. 19A or FIG. 19B. In some cases, the DQB1 polypeptide has a length of about 188 amino acids (e.g., 186, 187, 188, 190, 191, or 192 amino acids).

A “DQB1 polypeptide” includes allelic variants, e.g., naturally occurring allelic variants. Thus, in some cases, a suitable DQB1 polypeptide comprises the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRRVEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:59), or an allelic variant thereof.

A suitable DQB1 β1 domain comprises an amino acid sequence having at least 85%, at least 90%, at least 95%, at least 98%, at least 99%, or 100%, amino acid sequence identity to the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO:60); and can have a length of about 94 amino acids (e.g., 92, 93, 94, 95, or 96 amino acids). A suitable DQB1 β1 domain can comprise the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO:60), or a naturally-occurring allelic variant.

A suitable DQB1 β2 domain comprises an amino acid sequence having at least 85%, at least 90%, at least 95%, at least 98%, at least 99%, or 100%, amino acid sequence identity to the following amino acid sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:61); and can have a length of about 94 amino acids (e.g., 92, 93, 94, 95, or 96 amino acids). A suitable DQB1 β2 domain can comprise the following amino acid sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:61), or a naturally-occurring allelic variant thereof.

### DQB2

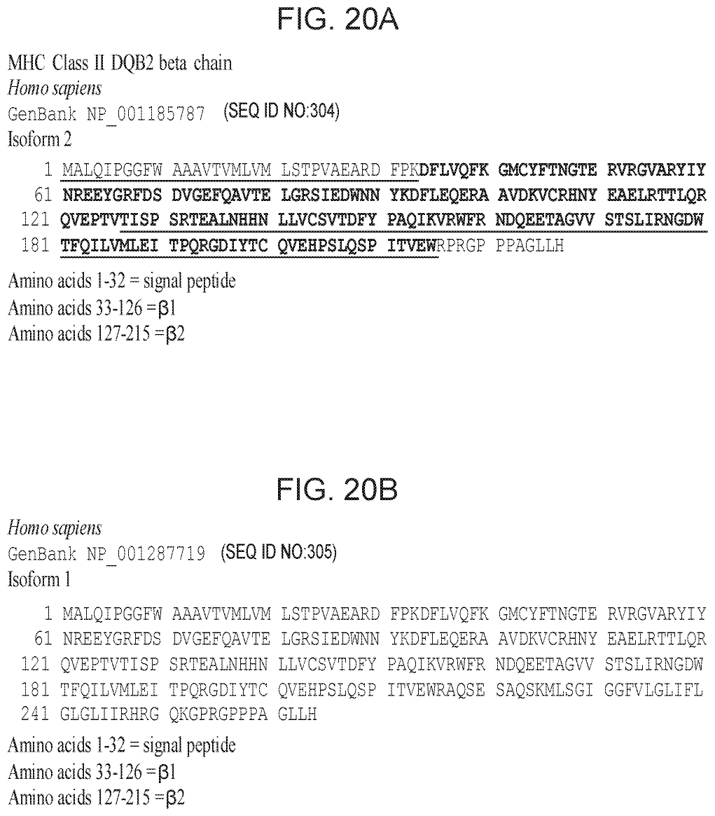

In some cases, a suitable MHC Class II chain polypeptide is a DQB2 polypeptide. A DQB2 polypeptide can have at least 85%, at least 90%, at least 95%, at least 98%, at least 99%, or 100%, amino acid sequence identity with amino acids 33-215 of the DQB2 amino acid sequence depicted in FIG. 20A or FIG. 20. In some cases, the DQB2 polypeptide has a length of about 182 amino acids (e.g., 175, 176, 177, 178, 179, 180, 181, or 182 amino acids).

A “DQB2 polypeptide” includes allelic variants, e.g., naturally occurring allelic variants. Thus, in some cases, a suitable DQB2 polypeptide comprises the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTVTISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:62), or an allelic variant thereof.

A suitable DQB2 β1 domain comprises an amino acid sequence having at least 85%, at least 90%, at least 95%, at least 98%, at least 99%, or 100%, amino acid sequence identity to the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTV (SEQ ID NO:63); and can have a length of about 94 amino acids (e.g., 92 93, 94, 95, 96, or 97 amino acids). A suitable DQB2 β1 domain can comprise the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTV (SEQ ID NO:63), or a naturally-occurring allelic variant.

A suitable DQB2 β2 domain comprises an amino acid sequence having at least 85%, at least 90%, at least 95%, at least 98%, at least 99%, or 100%, amino acid sequence identity to the following amino acid sequence: TISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:64); and can have a length of about 94 amino acids (e.g., 92 93, 94, 95, 96, or 97 amino acids). A suitable DQB2 β2 domain can comprise the following amino acid sequence: TISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:64), or a naturally-occurring allelic variant thereof.

### Scaffold Polypeptides

An APP of the present disclosure, whether multimeric or monomeric, can comprise an immunoglobulin or non-immunoglobulin scaffold. An APP polypeptide of the present disclosure, whether multimeric or monomeric, can comprise an Fc polypeptide, or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:65), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the multimeric polypeptide, compared to a control multimeric polypeptide lacking the scaffold polypeptide. For example, in some cases, a scaffold polypeptide increases the in vivo half-life (e.g., the serum half-life) of the multimeric polypeptide, compared to a control multimeric polypeptide lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the multimeric polypeptide, compared to a control multimeric polypeptide lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

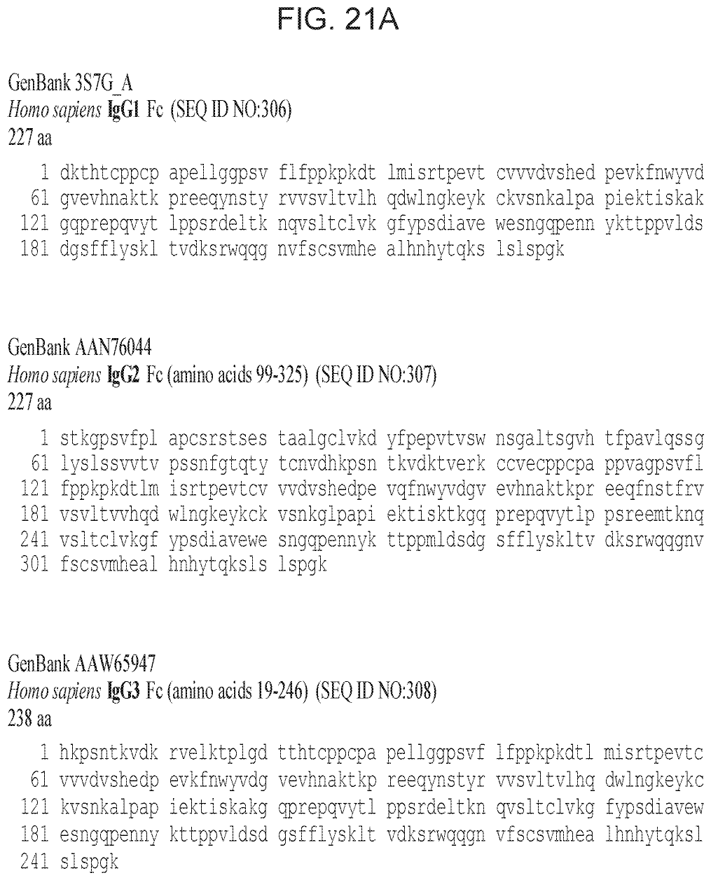

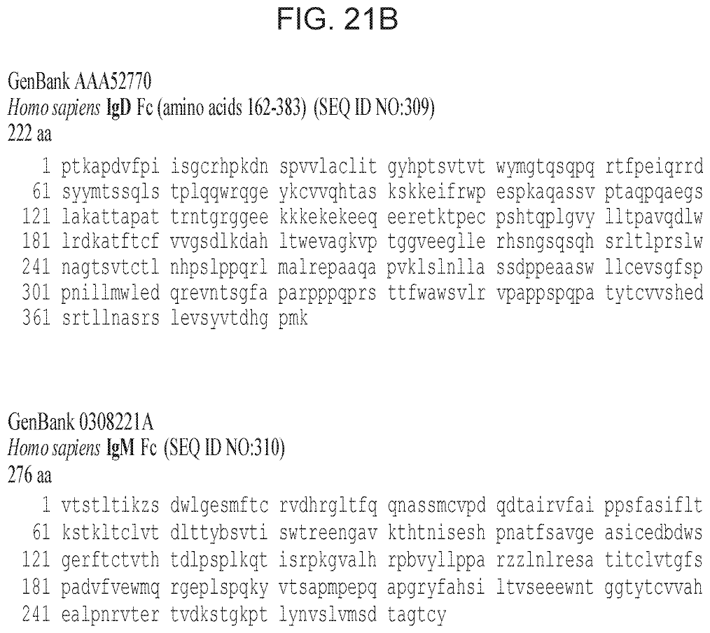

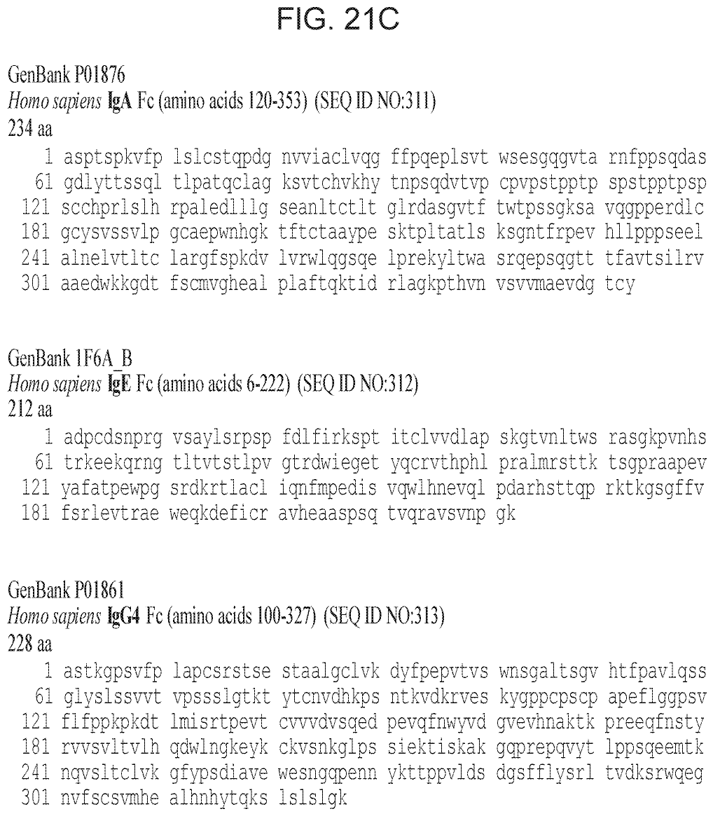

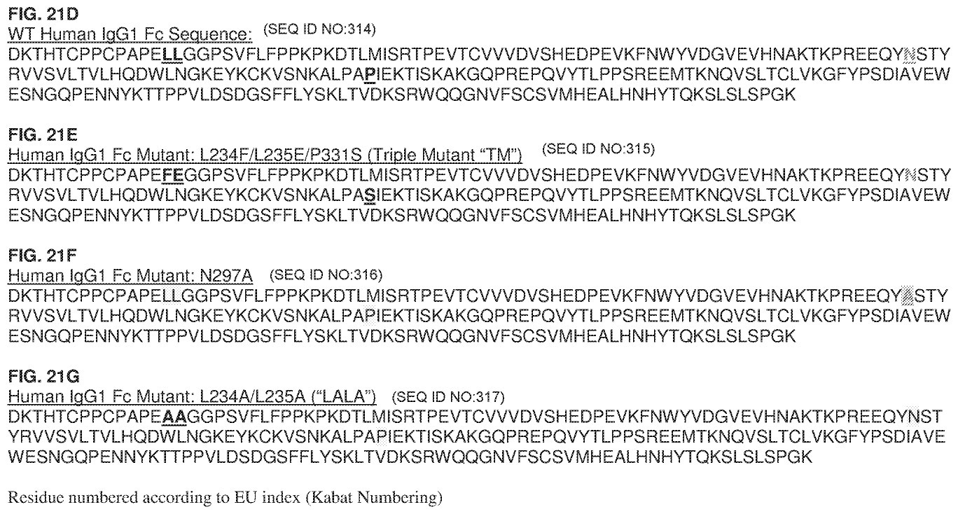

As noted above, in some cases, an APP of the present disclosure can comprise an Ig Fc polypeptide. For example, where the APP is a multimeric polypeptide, in some cases, the first and/or the second polypeptide chain of a multimeric polypeptide comprises an Fc polypeptide. In some cases, an APP of the present disclosure is a monomeric polypeptide and comprises an Ig Fc polypeptide. The Fc polypeptide can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to an amino acid sequence of an Fc region depicted in FIG. 21A-21G. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 21A. In some cases, the Fc region comprises an amino acid sequence having at least about 70%, at least about 75%, at least about 80%, at least about 85%, at least about 90%, at least about 95%, at least about 98%, at least about 99%, or 100%, amino acid sequence identity to the human IgG1 Fc polypeptide depicted in FIG. 21A; and comprises a substitution of N77; e.g., the Fc polypeptide comprises an N77A substitution. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG2 Fc polypeptide depicted in FIG. 21A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 99-325 of the human IgG2 Fc polypeptide depicted in FIG. 21A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG3 Fc polypeptide depicted in FIG. 21A; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 19-246 of the human IgG3 Fc polypeptide depicted in FIG. 21A. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgM Fc polypeptide depicted in FIG. 21B; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-276 to the human IgM Fc polypeptide depicted in FIG. 21B. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgA Fc polypeptide depicted in FIG. 21C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 1-234 to the human IgA Fc polypeptide depicted in FIG. 21C. In some cases, the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to the human IgG4 Fc polypeptide depicted in FIG. 21C; e.g., the Fc polypeptide comprises an amino acid sequence having at least about 70%, at least about 75%, at least about 80%, at least about 85%, at least about 90%, at least about 95%, at least about 98%, at least about 99%, or 100%, amino acid sequence identity to amino acids 100-327 of the human IgG4 Fc polypeptide depicted in FIG. 21C.

In some cases, the Fc polypeptide present in an APP of the present disclosure comprises the amino acid sequence depicted in FIG. 21A (human IgG1 Fc). In some cases, the Fc polypeptide present in an APP of the present disclosure comprises the amino acid sequence depicted in FIG. 21A (human IgG1 Fc), except for a substitution of N297 with an amino acid other than asparagine. In some cases, the Fc polypeptide present in an APP of the present disclosure comprises the amino acid sequence depicted in FIG. 21C (human IgG1 Fc comprising an N297A substitution). In some cases, the Fc polypeptide present in an APP of the present disclosure comprises the amino acid sequence depicted in FIG. 21A (human IgG1 Fc), except for a substitution of L234 with an amino acid other than leucine. In some cases, the Fc polypeptide present in an APP of the present disclosure comprises the amino acid sequence depicted in FIG. 21A (human IgG1 Fc), except for a substitution of L235 with an amino acid other than leucine.

In some cases, the Fc polypeptide present in an APP of the present disclosure comprises the amino acid sequence depicted in FIG. 21E. In some cases, the Fc polypeptide present in an APP of the present disclosure comprises the amino acid sequence depicted in FIG. 21F. In some cases, the Fc polypeptide present in an APP of the present disclosure comprises the amino acid sequence depicted in FIG. 21G (human IgG1 Fc comprising an L234A substitution and an L235A substitution). In some cases, the Fc polypeptide present in an APP of the present disclosure comprises the amino acid sequence depicted in FIG. 21A (human IgG1 Fc), except for a substitution of P331 with an amino acid other than proline; in some cases, the substitution is a P331S substitution. In some cases, the Fc polypeptide present in an APP of the present disclosure comprises the amino acid sequence depicted in FIG. 21A (human IgG1 Fc), except for substitutions at L234 and L235 with amino acids other than leucine. In some cases, the Fc polypeptide present in an APP of the present disclosure comprises the amino acid sequence depicted in FIG. 21A (human IgG1 Fc), except for substitutions at L234 and L235 with amino acids other than leucine, and a substitution of P331 with an amino acid other than proline. In some cases, the Fc polypeptide present in an APP of the present disclosure comprises the amino acid sequence depicted in FIG. 21B (human IgG1 Fc comprising L234F, L235E, and P331S substitutions). In some cases, the Fc polypeptide present in an APP of the present disclosure is an IgG1 Fc polypeptide that comprises L234A and L235A substitutions.

### Linkers

As noted above, an APP of the present disclosure can include a linker peptide interposed between, e.g., an epitope and an MHC polypeptide; between an MHC polypeptide and an Ig Fc polypeptide; between a first MHC polypeptide and a second MHC polypeptide; etc.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. A suitable linker can be from 25 to 35 amino acids in length. A suitable linker can be 25, 26, 27, 28, 29, 30, 31, 32, 33, 34, or 35 amino acids in length. A suitable linker can be from 35 to 45 amino acids in length. A suitable linker can be 35, 36, 37, 38, 39, 40, 41, 42, 43, 44, or 45 amino acids in length. A suitable linker can be from 45 to 50 amino acids in length. A suitable linker can be 45, 46, 47, 48, 49, or 50 amino acids in length.

Exemplary linkers include glycine polymers (G)ₙ, glycine-serine polymers (including, for example, (GS)ₙ, (GSGGS)ₙ (SEQ ID NO:66) and (GGGS)ₙ (SEQ ID NO:67), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:68), GGSGG (SEQ ID NO:69), GSGSG (SEQ ID NO:70), GSGGG (SEQ ID NO:71), GGGSG (SEQ ID NO:72), GSSSG (SEQ ID NO:73), and the like. Exemplary linkers can include, e.g., Gly(Ser₄)n, (SEQ ID NO:344) where n is 1, 2, 3, 4, 5, 6, 7, 8, 9, or 10. In some cases, a linker comprises the amino acid sequence (GSSSS)n (SEQ ID NO:74), where n is 4. In some cases, a linker comprises the amino acid sequence (GSSSS)n (SEQ ID NO:74), where n is 5. Exemplary linkers can include, e.g., (GlyGlyGlyGlySer)n (SEQ ID NO:75), where n is 1, 2, 3, 4, 5, 6, 7, 8, 9, or 10. In some cases, a linker comprises the amino acid sequence (GGGGS)n (SEQ ID NO:75), where n is 1. In some cases, a linker comprises the amino acid sequence (GGGGS)n (SEQ ID NO:345), where n is 2. In some cases, a linker comprises the amino acid sequence (GGGGS)n (SEQ ID NO:346), where n is 3. In some cases, a linker comprises the amino acid sequence (GGGGS)n (SEQ ID NO:347), where n is 4. In some cases, a linker comprises the amino acid sequence (GGGGS)n (SEQ ID NO:348), where n is 5. In some cases, a linker comprises the amino acid sequence (GGGGS)n (SEQ ID NO:349), where n is 6. In some cases, a linker comprises the amino acid sequence (GGGGS)n (SEQ ID NO:350), where n is 7. In some cases, a linker comprises the amino acid sequence (GGGGS)n (SEQ ID NO:351), where n is 8. In some cases, a linker comprises the amino acid sequence (GGGGS)n (SEQ ID NO:352), where n is 9. In some cases, a linker comprises the amino acid sequence (GGGGS)n (SEQ ID NO:353), where n is 10. In some cases, a linker comprises the amino acid sequence AAAGG (SEQ ID NO:76).

In some cases, a linker polypeptide present in an APP of the present disclosure includes a cysteine residue that can form a disulfide bond with a cysteine residue present in a second polypeptide of the APP. In some cases, for example, a suitable linker comprises the amino acid sequence GCGASGGGGSGGGGS (SEQ ID NO:77).

### Epitope-Presenting Peptides

A peptide epitope (also referred to herein as a “peptide antigen” or “epitope-presenting peptide” or “epitope”) present in an APP of the present disclosure presents an epitope to a TCR on the surface of a T cell. An epitope-presenting peptide can have a length of from about 4 amino acids to about 25 amino acids, e.g., the epitope can have a length of from 4 amino acids (aa) to 10 aa, from 10 aa to 15 aa, from 15 aa to 20 aa, or from 20 aa to 25 aa. For example, an epitope present in an APP of the present disclosure can have a length of 4 amino acids (aa), 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa. In some cases, an epitope-presenting peptide present in an APP of the present disclosure has a length of from 5 amino acids to 10 amino acids, e.g., 5 aa, 6 aa, 7 aa, 8 aa, 9 aa, or 10 aa.

An epitope-presenting peptide present in an APP of the present disclosure is specifically bound by a T-cell, i.e., the epitope is specifically bound by an epitope-specific T cell. An epitope-specific T cell binds an epitope-presenting peptide having a reference amino acid sequence, but does not substantially bind an epitope that differs from the reference amino acid sequence. For example, an epitope-specific T cell binds an epitope-presenting peptide having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope-presenting peptide for which it is specific with an affinity of at least 10⁻⁷ M, at least 10⁻⁸ M, at least 10⁻⁹ M, or at least 10⁻¹⁰ M.

Suitable epitope-presenting peptides include, but are not limited to, epitope-presenting peptides present in a cancer-associated antigen. Cancer-associated antigens include, but are not limited to, α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-α2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); and vascular endothelial growth factor receptor-2 (VEGF-R2). See, e.g., Vigneron et al. (2013) Cancer Immunity 13:15; and Vigneron (2015) BioMed Res. Int'l Article ID 948501. In some cases, the epitope is a human papilloma virus E7 antigen epitope; see, e.g., Ramos et al. (2013) J. Immunother. 36:66.

In some cases, a suitable peptide epitope is a peptide fragment of from about 4 amino acids to about 20 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a MUC1 polypeptide, a human papillomavirus (HPV) E6 polypeptide, an LMP2 polypeptide, an HPV E7 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an alpha-fetoprotein (AFP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosin kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGF3) polypeptide, a MAD-CT-2 polypeptide, a Fos-related antigen-1 (FOSL) polypeptide, and a Wilms tumor-1 (WT1) polypeptide.

Amino acid sequences of cancer-associated antigens are known in the art; see, e.g., MUC1 (GenBank CAA56734); LMP2 (GenBank CAA47024); HPV E6 (GenBank AAD33252); HPV E7 (GenBank AHG99480); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); AFP (GenBank NP_001125); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CA099273); MYCN (GenBank NP_001280157); RhoC (GenBank AAH52808); TRP-2 (GenBank AAC60627); mesothelin (GenBank AAH09272); PSCA (GenBank AAH65183); MAGE A1 (GenBank NP_004979); CYP1B1 (GenBank AAM50512); PLAC1 (GenBank AAG22596); BORIS (GenBank NP_001255969); ETV6 (GenBank NP_001978); NY-BR1 (GenBank NP_443723); SART3 (GenBank NP_055521); carbonic anhydrase IX (GenBank EAW58359); PAX5 (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGF3 (GenBank NP_002600); MAD-CT-2 (GenBank NP_001138574); FOSL (GenBank NP_005429); and WT-1 (GenBank NP_000369). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; Zhang et al. (1996) Nature 383:168.

In some cases, the epitope is HPV16E7/82-90 (LLMGTLGIV; SEQ ID NO:78). In some cases, the epitope is HPV16E7/86-93 (TLGIVCPI; SEQ ID NO:79). In some cases, the epitope is HPV16E7/11-20 (YMLDLQPETT; SEQ ID NO:80). In some cases, the epitope is HPV16E7/11-19 (YMLDLQPET; SEQ ID NO:81). See, e.g., Ressing et al. ((1995) J. Immunol. 154:5934) for additional suitable HPV epitopes.

In some cases, the peptide epitope is an epitope associated with or present in a “self” antigen (an autoantigen). Autoantigens include, e.g., aggrecan, alanyl-tRNA syntetase (PL-12), alpha beta crystallin, alpha fodrin (Sptan 1), alpha-actinin, α1 antichymotrypsin, α1 antitripsin, α1 microglobulin, alsolase, aminoacyl-tRNA synthetase, an amyloid, an annexin, an apolipoprotein, aquaporin, bactericidal/permeability-increasing protein (BPI), β-globin precursor BP1, β-actin, β-lactoglobulin A, β-2-glycoprotein I, .beta.2-microglobulin, a blood group antigen, C reactive protein (CRP), calmodulin, calreticulin, cardiolipin, catalase, cathepsin B, a centromere protein, chondroitin sulfate, chromatin, collagen, a complement component, cytochrome C, cytochrome P450 2D6, cytokeratins, decorin, dermatan sulfate, DNA, DNA topoisomerase I, elastin, Epstein-Barr nuclear antigen 1 (EBNA1), elastin, entaktin, an extractable nuclear antigen, Factor I, Factor P, Factor B, Factor D, Factor H, Factor X, fibrinogen, fibronectin, formiminotransferase cyclodeaminase (LC-1), gliadin and amidated gliadin peptides (DGPs), gp210 nuclear envelope protein, GP2 (major zymogen granule membrane glycoprotein), a glutenin, glycoprotein gpIIb/IIIa, glial fibrillary acidic protein (GFAP), glycated albumin, glyceraldehyde 3-phosphate dehydrogenase (GAPDH), haptoglobin A2, heat shock proteins, hemocyanin, heparin, a histone, histidyl-tRNA synthetase (Jo-1), a hordein, hyaluronidase, immunoglobulins, insulin, insulin receptor, an integrin, interstitial retinol-binding protein 3, intrinsic factor, Ku (p70/p80), lactate dehydrogenase, laminin, liver cytosol antigen type 1 (LC1), liver/kidney microsomal antigen 1 (LKM1), lysozyme, melanoma differentiation-associated protein 5 (MDAS), Mi-2 (chromodomain helicase DNA binding protein 4), a mitochondrial protein, muscarinic receptors, myelin-associated glycoprotein, myosin, myelin basic protein, myelin oligodendrocyte glycoprotein, myeloperoxidase (MPO), rheumatoid factor (IgM anti-IgG), neuron-specific enolase, nicotinic acetylcholine receptor A chain, nucleolin, a nucleoporin, nucleosome antigen, PM/Sc1100, PM/Scl 75, pancreatic β-cell antigen, pepsinogen, peroxiredoxin 1, phosphoglucose isomerase, phospholipids, phosphotidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, a ribonucleoprotein, ribosomes, a ribosomal phosphoprotein, RNA, an Sm protein, Sp100 nuclear protein, SRP54 (signal recognition particle 54 kDa), a secalin, selectin, smooth muscle proteins, sphingomyelin, streptococcal antigens, superoxide dismutase, synovial joint proteins, T1F1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis alpha, topoisomerase, UI-dnRNP 68/70 kDa, UI-snRNP A, UI-snRNP C, U-snRNP B/B′, ubiquitin, vascular endothelial growth factor, vimentin, and vitronectin.

Antigens associated with type 1 diabetes (T1D) include, e.g., preproinsulin, proinsulin, insulin, insulin β chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose6-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529. An antigen “associated with” a particular autoimmune disorder is an antigen that is a target of autoantibodies and/or autoreactive T cells present in individuals with that autoimmune disorder, where such autoantibodies and/or autoreactive T cells mediate a pathological state associated with the autoimmune disorder. A suitable epitope-presenting peptide for inclusion in an antigen-presenting polypeptide of the present disclosure can be an epitope-presenting peptide of from 4 amino acids to about 25 amino acids in length of any one of the aforementioned T1D-associated antigens. As one non-limiting example, an epitope-presenting peptide is proinsulin 73-90 (GAGSLQPLALEGSLQKR; SEQ ID NO:82). As another non-limiting example, an epitope-presenting peptide is the following insulin (InsA (1-15) peptide: GIVDQCCTSICSLYQ (SEQ ID NO:83). As another non-limiting example, an epitope-presenting peptide is the following insulin (InsA(1-15; D4E) peptide: GIVEQCCTSICSLYQ (SEQ ID NO:84). As another non-limiting example, an epitope-presenting peptide is the following GAD65 (555-567) peptide; NFFRMVISNPAAT (SEQ ID NO:85). As another non-limiting example, an epitope-presenting peptide is the following GAD65 (555-567; F557I) peptide; NFIRMVISNPAAT (SEQ ID NO: 86). As another non-limiting example, an epitope-presenting peptide is the following islet antigen 2 (IA2) peptide: SFYLKNVQTQETRTLTQFHF (SEQ ID NO:87).

Antigens associated with Grave's disease include, for example, thyroglobulin, thyroid peroxidase, and thyrotropin receptor (TSH-R). A suitable epitope-presenting peptide for inclusion in an A{{of the present disclosure can be an epitope-presenting peptide of from 4 amino acids to about 25 amino acids in length of any one of the aforementioned Grave's disease-associated antigens.

Antigens associated with autoimmune polyendocrine syndrome include, 17-alpha hydroxylase, histidine decarboxylase, tryptophan hydroxylase, and tyrosine hydroxylase. A suitable epitope-presenting peptide for inclusion in an antigen-presenting polypeptide of the present disclosure can be an epitope-presenting peptide of from 4 amino acids to about 25 amino acids in length of any one of the aforementioned autoimmune polyendocrine syndrome-associated antigens.

Antigens associated with rheumatoid arthritis include, e.g., collagen, vimentin, aggregan, and fibrinogen. A suitable epitope-presenting peptide for inclusion in an antigen-presenting polypeptide of the present disclosure can be an epitope-presenting peptide of from 4 amino acids to about 25 amino acids in length of any one of the aforementioned rheumatoid arthritis-associated antigens.

Antigens associated with Parkinson's disease include, e.g., α-synuclein. A suitable epitope-presenting peptide for inclusion in an APP of the present disclosure can be an epitope-presenting peptide of from 4 amino acids to about 25 amino acids in length of any one of the aforementioned Parkinson's disease-associated antigens.

Antigens associated with multiple sclerosis include, e.g., myelin basic protein, myelin oligodendrocyte glycoprotein, and proteolipid protein. A suitable epitope-presenting peptide for inclusion in an APP of the present disclosure can be an epitope-presenting peptide of from 4 amino acids to about 25 amino acids in length of any one of the aforementioned multiple sclerosis-associated antigens.

Antigens associated with celiac disease include, e.g., tissue transglutaminase and gliadin. A suitable epitope-presenting peptide for inclusion in an APP of the present disclosure can be an epitope-presenting peptide of from 4 amino acids to about 25 amino acids in length of any one of the aforementioned celiac-associated antigens. Other antigens associated with celiac disease include, e.g., secalins, hordeins, avenins, and glutenins. Examples of secalins include rye secalins. Examples of hordeins include barley hordeins. Examples of glutenins include wheat glutenins. See, e.g., U.S. 2016/0279233.

### Antigen-Presenting Polypeptides Comprising an Immunomodulatory Domain

In some cases, an APP of the present disclosure is a T-cell modulatory antigen-presenting polypeptide (TMAPP). Thus, the present disclosure provides TMAPPs. In some cases, a TMAPP of the present disclosure comprises two polypeptide chains and is sometimes referred to herein as a “multimeric T-cell modulatory antigen-presenting polypeptide.” In some cases, a TMAPP of the present disclosure comprises a single polypeptide chain. A TMAPP of the present disclosure is also referred to as a “synTac polypeptide.”

A TMAPP of the present disclosure comprises one or more immunomodulatory polypeptides. In some cases, a TMAPP of the present disclosure comprises a single immunomodulatory polypeptide. In some cases, a TMAPP of the present disclosure comprises two or more immunomodulatory polypeptides (e.g., 2, 3, 4, or 5 immunomodulatory polypeptides).

In some cases, a TMAPP of the present disclosure comprises two or more immunomodulatory polypeptides. In some cases, where a TMAPP of the present disclosure comprises a first polypeptide and a second polypeptide, the two or more immunomodulatory polypeptides are present in the first polypeptide chain only. In some cases, where a TMAPP of the present disclosure comprises a first polypeptide and a second polypeptide, the two or more immunomodulatory polypeptides are present in the second polypeptide chain only. In some cases, where a TMAPP of the present disclosure comprises a first polypeptide and a second polypeptide, at least one of the two or more immunomodulatory polypeptides are present in the first polypeptide chain; and at least one of the two or more immunomodulatory polypeptides are present in the second polypeptide chain.

In some cases, where a TMAPP of the present disclosure comprises two immunomodulatory polypeptides, the two immunomodulatory polypeptides have the same amino acid sequence, i.e., the TMAPP comprises two copies of an immunomodulatory polypeptide. In some cases, where a TMAPP of the present disclosure comprises two immunomodulatory polypeptides, the two immunomodulatory polypeptides do not have the same amino acid sequence; e.g., one of the two immunomodulatory polypeptides comprises a first amino acid sequence and the second of the two immunomodulatory polypeptides comprises a second amino acid sequence, where the first and the second amino acid sequences are not identical. In some cases, the first and the second amino acid sequences differ from one another in amino acid sequence by from 1 amino acid to 10 amino acids, from 10 amino acids to 25 amino acids, or more than 25 amino acids. In some cases, the first and the second amino acid sequences share less than 98%, less than 95%, less than 90%, less than 85%, less than 80%, less than 75%, or less than 70%, amino acid sequence identity with one another.

A TMAPP of the present disclosure modulates activity of a T cell. In some cases, a TMAPP of the present disclosure activates a CD8⁺ T cell response, e.g., a CD8⁺ T cell response to a cancer cell. In some cases, a TMAPP of the present disclosure reduces activity of an autoreactive T cell and/or an autoreactive B cell. In some cases, a TMAPP of the present disclosure increases the number and/or activity of a regulator T cell (Treg), resulting in reduced activity of an autoreactive T cell and/or an autoreactive B cell.

Immunomodulatory polypeptides that are suitable for inclusion in a TMAPP of the present disclosure include, but are not limited to, IL-2, transforming growth factor-beta (TGFβ), JAG1, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM. In some cases, an immunomodulatory polypeptide suitable for inclusion in a TMAPP of the present disclosure is a variant that comprises from 1 to 10 amino acid substitutions relative to a wild-type or naturally-occurring immunomodulatory polypeptide, and that exhibits reduced affinity to its cognate co-immunomodulatory polypeptide (e.g., a co-immunomodulatory polypeptide present on the surface of a T cell), compared to the affinity of the wild-type or naturally-occurring immunomodulatory polypeptide for the cognate co-immunomodulatory polypeptide.

### T-Cell Modulatory Antigen-Presenting Polypeptides

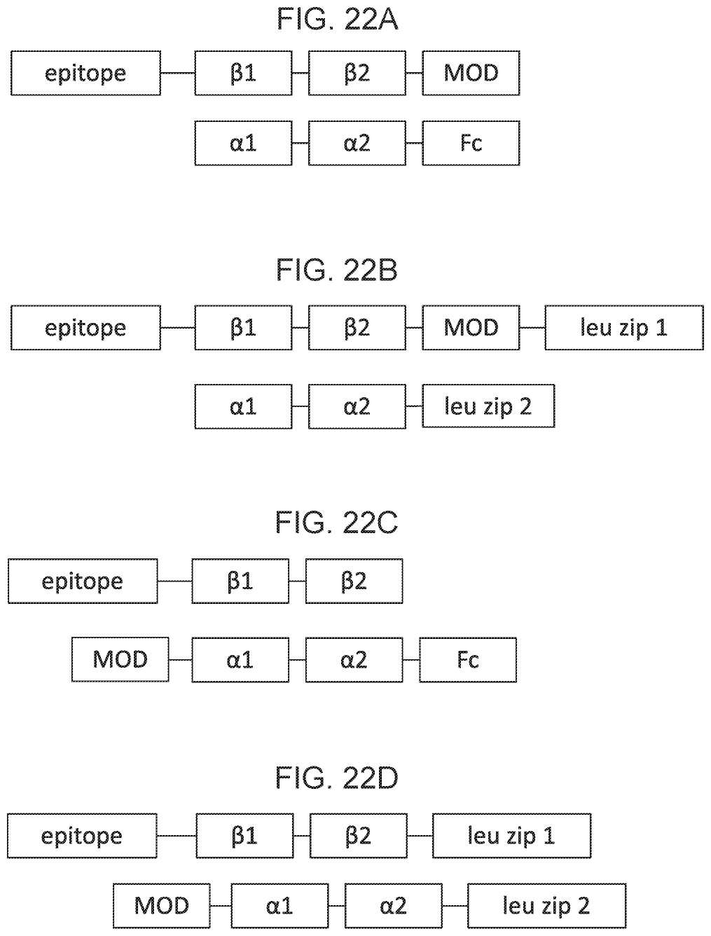

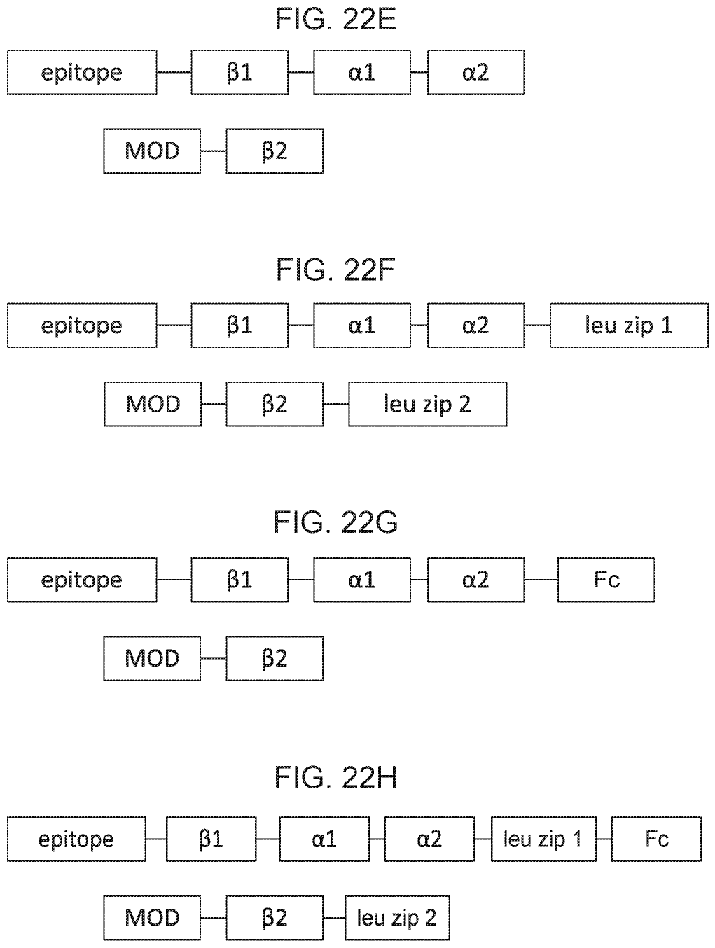

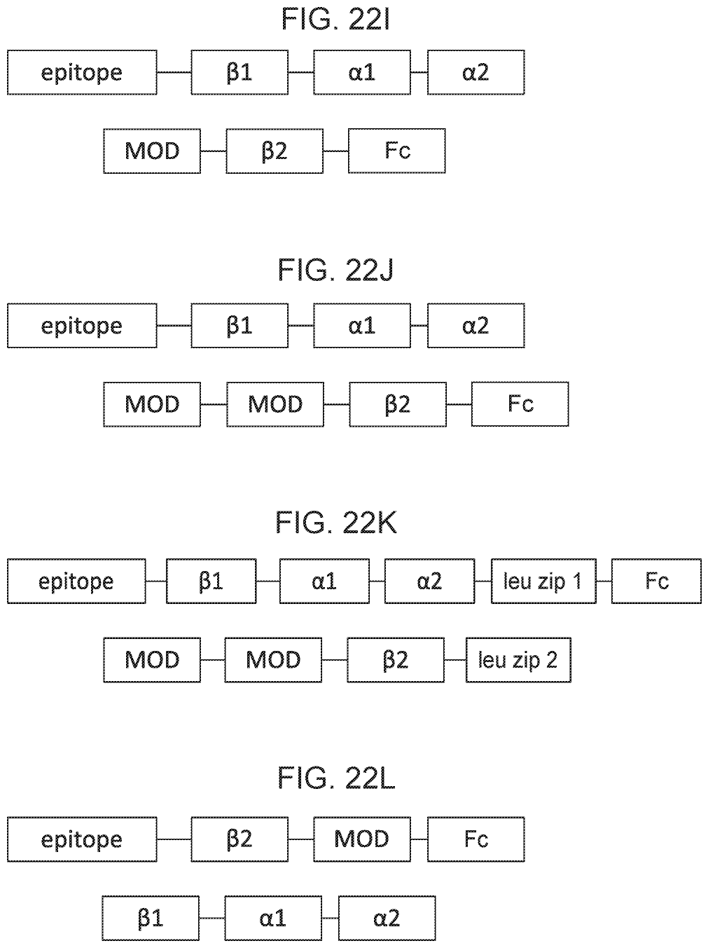

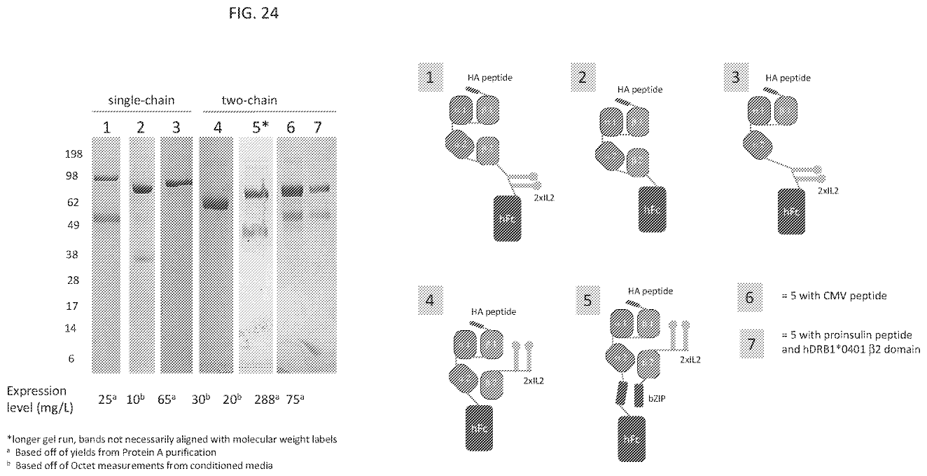

A TMAPP of the present disclosure comprises: i) a peptide epitope (a peptide recognized and bound by a TCR); ii) an MHC Class II α chain polypeptide; iii) an MHC Class II β chain polypeptide; and iv) an immunomodulatory polypeptide (also referred to herein as a “MOD polypeptide” or a “MOD domain”). A TMAPP of the present disclosure can further include one or both of: a dimerizer polypeptide; and an immunoglobulin scaffold (e.g., an Ig Fc polypeptide) or a non-immunoglobulin scaffold. Non-limiting example of multimeric TMAPPs of the present disclosure is schematically depicted in FIG. 22A-22J and FIG. 24.

In some cases, a TMAPP of the present disclosure comprises a single immunomodulatory polypeptide. In some cases, a TMAPP of the present disclosure comprises 2 copies of an immunomodulatory polypeptide. In some cases, a TMAPP of the present disclosure comprises 3 copies of an immunomodulatory polypeptide. Where a TMAPP of the present disclosure comprises 2 or 3 copies of an immunomodulatory polypeptide, in some cases, the 2 or 3 copies are in tandem. Where a TMAPP of the present disclosure comprises 2 or 3 copies of an immunomodulatory polypeptide, in some cases, the 2 or 3 copies are separated from one another by a linker.

A TMAPP of the present disclosure can include one or more linkers, where the one or more linkers are between one or more of: i) an MHC Class II polypeptide and an Ig Fc polypeptide, where such a linker is referred to herein as “L1”; ii) an immunomodulatory polypeptide and an MHC Class II polypeptide, where such a linker is referred to herein as “L2”; iii) a first immunomodulatory polypeptide and a second immunomodulatory polypeptide, where such a linker is referred to herein as “L3”; iv) a peptide antigen (“epitope”) and an MHC Class II polypeptide; v) an MHC Class II polypeptide and a dimerization polypeptide (e.g., a first or a second member of a dimerizing pair); and vi) a dimerization polypeptide (e.g., a first or a second member of a dimerizing pair) and an IgFc polypeptide. In some cases, an L1 linker comprises (GGGGS)n (SEQ ID NO:75), where n is 1, 2, 3, 4, 5, 6, 7, or 8. In some cases, an L2 linker comprises (GGGGS)n (SEQ ID NO:75), where n is 1, 2, 3, 4, 5, 6, 7, or 8. In some cases, an L3 linker comprises (GGGGS)n (SEQ ID NO:75), where n is 1, 2, 3, 4, 5, 6, 7, or 8.

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II pi polypeptide; iii) an MHC Class II α1 polypeptide; and iv) an MHC Class II (2 polypeptide; and b) a second polypeptide comprising: i) an immunomodulatory polypeptide; and ii) an MHC Class II β2 polypeptide. In some cases, the second polypeptide comprises, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an immunoglobulin or non-immunoglobulin scaffold polypeptide; and b) a second polypeptide comprising: i) an immunomodulatory polypeptide; and ii) an MHC Class II β2 polypeptide. In some cases, the second polypeptide comprises, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an Ig Fc polypeptide; and b) a second polypeptide comprising: i) an immunomodulatory polypeptide; and ii) an MHC Class II β2 polypeptide. In some cases, the second polypeptide comprises, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first member of a dimerizer pair; and b) a second polypeptide comprising: i) an immunomodulatory polypeptide; ii) an MHC Class II β2 polypeptide; iii) a second member of the dimerizer pair. In some cases, the second polypeptide comprises, in order from N-terminus to C-terminus: i) an immunomodulatory polypeptide; ii) an MHC Class II β2 polypeptide; iii) a second member of the dimerizer pair. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first leucine zipper polypeptide; and b) a second polypeptide comprising: i) an immunomodulatory polypeptide; ii) an MHC Class II β2 polypeptide; and iii) a second leucine zipper polypeptide. In some cases, the second polypeptide comprises, in order from N-terminus to C-terminus: i) an immunomodulatory polypeptide; ii) an MHC Class II β2 polypeptide; and iii) a second leucine zipper polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first leucine zipper polypeptide; and vi) an Ig Fc polypeptide; and b) a second polypeptide comprising: i) an immunomodulatory polypeptide; ii) an MHC Class II β2 polypeptide; and iii) a second leucine zipper polypeptide. In some cases, the second polypeptide comprises, in order from N-terminus to C-terminus: i) an immunomodulatory polypeptide; ii) an MHC Class II β2 polypeptide; and iii) a second leucine zipper polypeptide. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. For example, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first leucine zipper polypeptide; and vi) an Ig Fc polypeptide; and b) a second polypeptide comprising: i) a first immunomodulatory polypeptide; ii) a second immunomodulatory polypeptide; iii) an MHC Class II β2 polypeptide; and iv) a second leucine zipper polypeptide. In some cases, the second polypeptide comprises, in order from N-terminus to C-terminus: i) a first immunomodulatory polypeptide; ii) a second immunomodulatory polypeptide; iii) an MHC Class II β2 polypeptide; and iv) a second leucine zipper polypeptide. In some cases, the first and the second immunomodulatory polypeptides comprise the same amino acid sequences. As another example, in some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an Ig Fc polypeptide; and b) a second polypeptide comprising: i) a first immunomodulatory polypeptide; ii) a second immunomodulatory polypeptide; and iii) an MHC Class II β2 polypeptide. In some cases, the second polypeptide comprises, in order from N-terminus to C-terminus: i) a first immunomodulatory polypeptide; ii) a second immunomodulatory polypeptide; and iii) an MHC Class II β2 polypeptide. In some cases, the first and the second immunomodulatory polypeptides comprise the same amino acid sequences. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; and iv) an MHC Class II α2 polypeptide; and b) a second polypeptide comprising: i) an immunomodulatory polypeptide; ii) an MHC Class II β2 polypeptide; and iii) an Ig Fc polypeptide. In some cases, the second polypeptide comprises, in order from N-terminus to C-terminus: i) an immunomodulatory polypeptide; ii) an MHC Class II β2 polypeptide; and iii) an Ig Fc polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; and iv) an MHC Class II α2 polypeptide; and b) a second polypeptide comprising: i) a first immunomodulatory polypeptide; ii) a second immunomodulatory polypeptide; iii) an MHC Class II β2 polypeptide; iv) an Ig Fc polypeptide. In some cases, the second polypeptide comprises, in order from N-terminus to C-terminus: i) a first immunomodulatory polypeptide; ii) a second immunomodulatory polypeptide; iii) an MHC Class II β2 polypeptide; iv) an Ig Fc polypeptide. In some cases, the first and the second immunomodulatory polypeptides comprise the same amino acid sequence. In some cases, a TMAPP of the present disclosure comprises: a) a first polypeptide comprising, in order from N-terminus to C-terminus: i) an immunomodulatory polypeptide; ii) an MHC Class II β1 polypeptide; iii) an MHC Class II α1 polypeptide; and iv) an MHC Class II α2 polypeptide; and b) a second polypeptide comprising: i) a peptide antigen (an “epitope”) that is recognized (e.g., is capable of being recognized and bound) by a TCR; ii) an MHC Class II β2 polypeptide; and iii) an Ig Fc polypeptide. In some cases, the second polypeptide comprises, in order from N-terminus to C-terminus: i) a peptide antigen (an “epitope”) that is recognized (e.g., is capable of being recognized and bound) by a TCR; ii) an MHC Class II β2 polypeptide; and iii) an Ig Fc polypeptide. In some cases, a TMAPP of the present disclosure comprises: a) a first polypeptide comprising, in order from N-terminus to C-terminus: i) a first immunomodulatory polypeptide; ii) a second immunomodulatory polypeptide; iii) an MHC Class II β1 polypeptide; iv) an MHC Class II α1 polypeptide; and v) an MHC Class II α2 polypeptide; and b) a second polypeptide comprising, in order from N-terminus to C-terminus: i) a peptide antigen (an “epitope”) that is recognized (e.g., is capable of being recognized and bound) by a TCR; ii) an MHC Class II β2 polypeptide; and iii) an Ig Fc polypeptide. In some cases, the second polypeptide comprises, in order from N-terminus to C-terminus: i) a peptide antigen (an “epitope”) that is recognized (e.g., is capable of being recognized and bound) by a TCR; ii) an MHC Class II β2 polypeptide; and iii) an Ig Fc polypeptide. In some cases, the first and the second immunomodulatory polypeptides comprise the same amino acid sequence. Where a TMAPP of the present disclosure comprises two immunomodulatory polypeptides, in some cases, the first immunomodulatory polypeptide is linked to the second immunomodulatory polypeptide by a linker (an “L3” linker); e.g., a linker of from about 2 amino acids to 50 amino acids in length. Suitable L3 linkers include (GGGGS)n (SEQ ID NO:75), where n is 1, 2, 3, 4, 5, 6, 7, or 8. In some cases, the TMAPP comprises a linker (an “L1”) between the MHC polypeptide and the Ig Fc polypeptide; where exemplary suitable linkers include (GGGGS)n (SEQ ID NO:75), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO:75), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO:75), where n is 1, 2, 3, 4, 5, 6, 7, or 8.

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II α1 polypeptide; iii) an MHC Class II α2 polypeptide; and iv) an immunoglobulin or non-immunoglobulin scaffold polypeptide; and b) a second polypeptide comprising, in order from N-terminus to C-terminus: i) an immunomodulatory polypeptide; ii) an MHC Class II β1 polypeptide; and iii) an MHC Class II β2 polypeptide. In some cases, a TMAPP of the present disclosure comprises: a) a first polypeptide comprising, in order from N-terminus to C-terminus: i) an immunomodulatory polypeptide; ii) an MHC Class II α1 polypeptide; and iii) an MHC Class II α2 polypeptide; and iv) an immunoglobulin or non-immunoglobulin scaffold polypeptide; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II α1 polypeptide; and iii) an MHC Class II α2 polypeptide; and b) a second polypeptide comprising, in order from N-terminus to C-terminus: i) an immunomodulatory polypeptide; ii) an MHC Class II β1 polypeptide; iii) an MHC Class II β2 polypeptide; and iv) an immunoglobulin or non-immunoglobulin scaffold polypeptide. In some cases, a TMAPP of the present disclosure comprises: a) a first polypeptide comprising, in order from N-terminus to C-terminus: i) an immunomodulatory polypeptide; ii) an MHC Class II α1 polypeptide; and iii) an MHC Class II α2 polypeptide; and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an immunoglobulin or non-immunoglobulin scaffold polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II α1 polypeptide; iii) an MHC Class II α2 polypeptide; iv) an immunoglobulin or non-immunoglobulin scaffold polypeptide; and v) a first member of a dimerizer pair (e.g., a first leucine zipper polypeptide); and b) a second polypeptide comprising, in order from N-terminus to C-terminus: i) an immunomodulatory polypeptide; ii) an MHC Class II β1 polypeptide; iii) an MHC Class II β2 polypeptide; and iv) a second member of a dimerizer pair (e.g., a second leucine zipper polypeptide). In some cases, a TMAPP of the present disclosure comprises: a) a first polypeptide comprising, in order from N-terminus to C-terminus: i) an immunomodulatory polypeptide; ii) an MHC Class II α1 polypeptide; iii) an MHC Class II α2 polypeptide; iv) an immunoglobulin or non-immunoglobulin scaffold polypeptide; and v) and v) a first member of a dimerizer pair (e.g., a first leucine zipper polypeptide); and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) a second member of a dimerizer pair (e.g., a second leucine zipper polypeptide). In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II α1 polypeptide; iii) an MHC Class II α2 polypeptide; and iv) a first member of a dimerizer pair (e.g., a first leucine zipper polypeptide); and b) a second polypeptide comprising, in order from N-terminus to C-terminus: i) an immunomodulatory polypeptide; ii) an MHC Class II β1 polypeptide; iii) an MHC Class II β2 polypeptide; iv) an immunoglobulin or non-immunoglobulin scaffold polypeptide; and v) a second member of a dimerizer pair (e.g., a second leucine zipper polypeptide). In some cases, a TMAPP of the present disclosure comprises: a) a first polypeptide comprising, in order from N-terminus to C-terminus: i) an immunomodulatory polypeptide; ii) an MHC Class II α1 polypeptide; iii) an MHC Class II α2 polypeptide; and iv) a first member of a dimerizer pair (e.g., a first leucine zipper polypeptide); and b) a second polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunoglobulin or non-immunoglobulin scaffold polypeptide; and v) a second member of a dimerizer pair (e.g., a second leucine zipper polypeptide). In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO:75), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO:75), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO:75), where n is 1, 2, 3, 4, 5, 6, 7, or 8.

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II P31 polypeptide; iii) an MHC Class II β2 polypeptide; and iv) an immunomodulatory polypeptide; and b) a second polypeptide comprising, in order from N-terminus to C-terminus: i) an MHC Class II α1 polypeptide; and ii) an MHC Class II α2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an immunomodulatory polypeptide; and b) a second polypeptide comprising, in order from N-terminus to C-terminus: i) an MHC Class II α1 polypeptide; ii) an MHC Class II α2 polypeptide; and iii) an immunoglobulin or non-immunoglobulin scaffold polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) an immunomodulatory polypeptide; and b) a second polypeptide comprising, in order from N-terminus to C-terminus: i) an MHC Class II α1 polypeptide; ii) an MHC Class II α2 polypeptide; and iii) an Ig Fc polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunomodulatory polypeptide; and v) a first member of a dimerizer pair; and b) a second polypeptide comprising, in order from N-terminus to C-terminus: i) an MHC Class II α1 polypeptide; ii) an MHC Class II α2 polypeptide; and iii) a second member of the dimerizer pair. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an immunomodulatory polypeptide; and v) a first leucine zipper polypeptide; and b) a second polypeptide comprising, in order from N-terminus to C-terminus: i) an MHC Class II α1 polypeptide; ii) an MHC Class II α2 polypeptide; and iii) a second leucine zipper polypeptide. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO:75), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO:75), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO:75), where n is 1, 2, 3, 4, 5, 6, 7, or 8.

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an immunomodulatory polypeptide; ii) an MHC Class II α1 polypeptide; and iii) an MHC Class II α2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an immunomodulatory polypeptide; ii) an MHC Class II α1 polypeptide; iii) an MHC Class II α2 polypeptide; and iv) an immunoglobulin or non-immunoglobulin scaffold polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; and iii) an MHC Class II β2 polypeptide; and b) a second polypeptide comprising, in order from N-terminus to C-terminus: i) an immunomodulatory polypeptide; ii) an MHC Class II α1 polypeptide; iii) an MHC Class II α2 polypeptide; and iv) an Ig Fc polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) a first member of a dimerizer pair; and b) a second polypeptide comprising, in order from N-terminus to C-terminus: i) an immunomodulatory polypeptide; ii) an MHC Class II α1 polypeptide; iii) an MHC Class II α2 polypeptide; and iv) a second member of the dimerizer pair. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; and iv) a first leucine zipper polypeptide; and b) a second polypeptide comprising, in order from N-terminus to C-terminus: i) an immunomodulatory polypeptide; ii) an MHC Class II α1 polypeptide; iii) an MHC Class II α2 polypeptide; and iv) a second leucine zipper polypeptide. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO:75), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO:75), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO:75), where n is 1, 2, 3, 4, 5, 6, 7, or 8.

In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; and iv) an MHC Class II α2 polypeptide; and b) a second polypeptide comprising, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an immunoglobulin or non-immunoglobulin scaffold polypeptide; and b) a second polypeptide comprising, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) an Ig Fc polypeptide; and b) a second polypeptide comprising, in order from N-terminus to C-terminus: i) an immunomodulatory polypeptide; and ii) an MHC Class II β2 polypeptide. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first member of a dimerizer pair; and b) a second polypeptide comprising, in order from N-terminus to C-terminus: i) an immunomodulatory polypeptide; ii) an MHC Class II β2 polypeptide; and iii) a second member of the dimerizer pair. In some cases, a TMAPP of the present disclosure comprises: a) a first polypeptide comprising, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) a first leucine zipper polypeptide; and b) a second polypeptide comprising, in order from N-terminus to C-terminus: i) an immunomodulatory polypeptide; ii) an MHC Class II β2 polypeptide; and iii) a second leucine zipper polypeptide. In any one of the above embodiments, the TMAPP can include a single immunomodulatory polypeptide. In any one of the above embodiments, the TMAPP can include 2 copies of the immunomodulatory polypeptide; the 2 copies can be in tandem, or can be separated by a linker. In any one of the above embodiments, the TMAPP can include 3 copies of the immunomodulatory polypeptide; the 3 copies can be in tandem, or can be separated by a linker. In some cases, the TMAPP comprises a linker (an “L1”) between the MHC polypeptide and the Ig Fc polypeptide; where exemplary suitable linkers include (GGGGS)n (SEQ ID NO:75), where n is 1, 2, 3, 4, 5, 6, 7, or 8. In some cases, the TMAPP comprises a linker (an “L2”) between the immunomodulatory polypeptide and the MHC polypeptide, where exemplary suitable linkers include (GGGGS)n (SEQ ID NO:75), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO:75), where n is 1, 2, 3, 4, 5, 6, 7, or 8.

### Exemplary Multimeric T-Cell Modulatory Antigen-Presenting Polypeptides

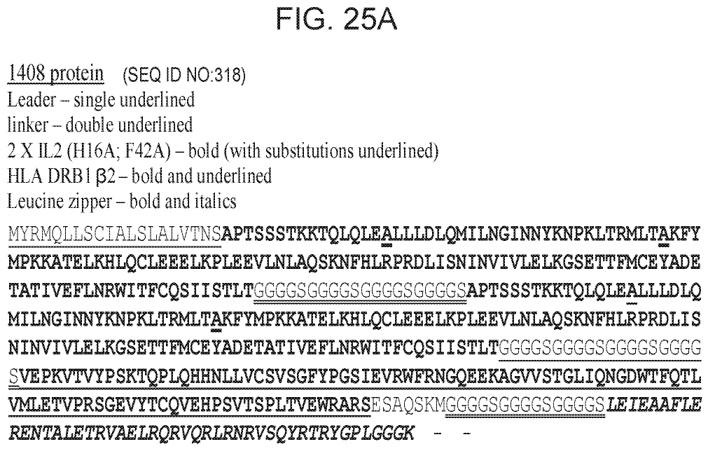

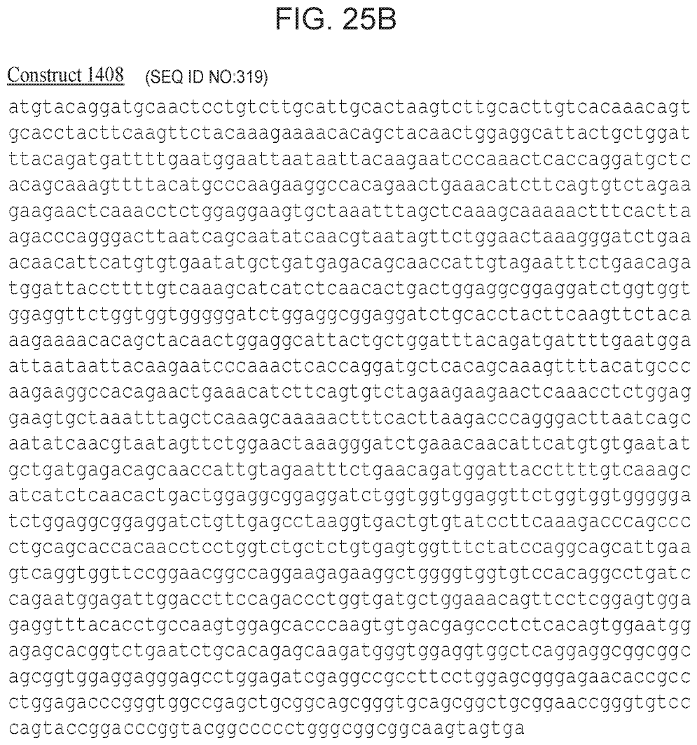

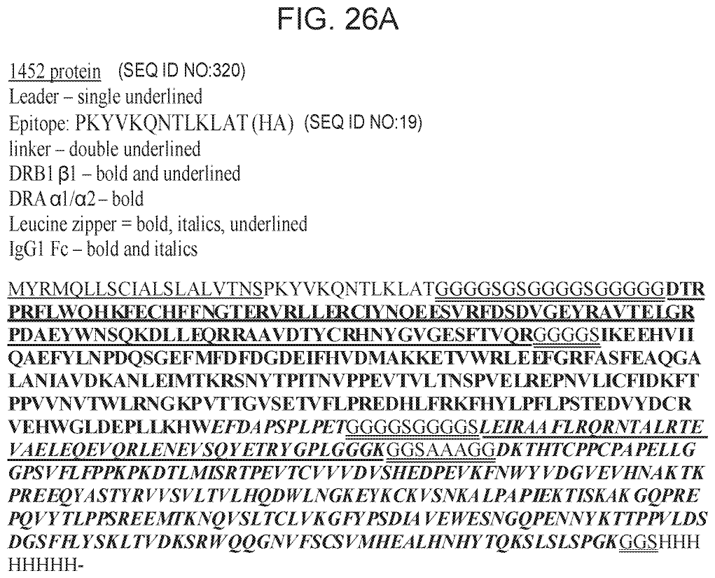

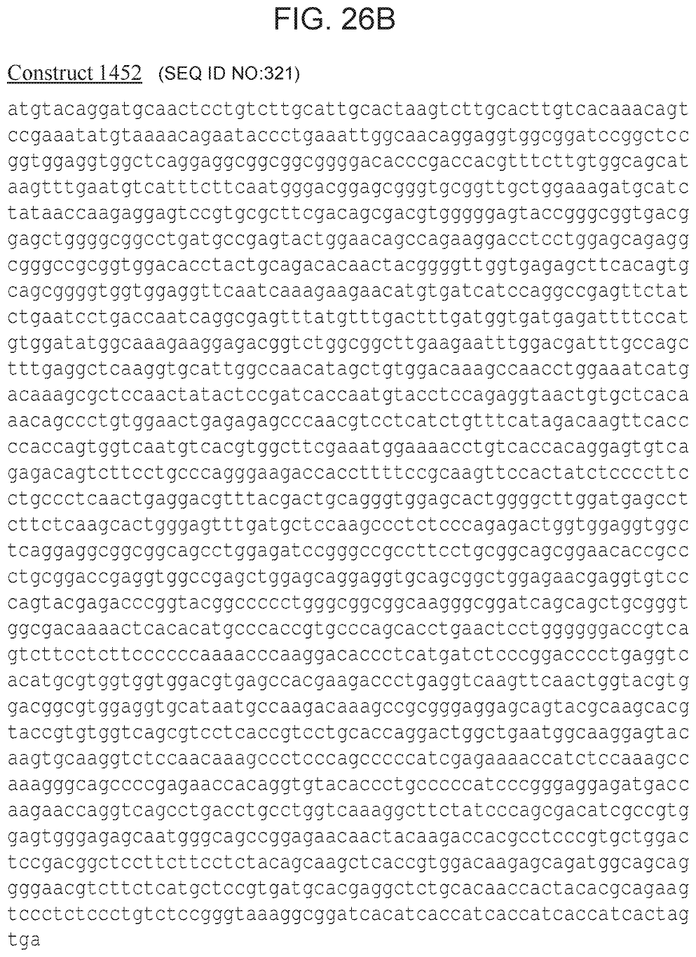

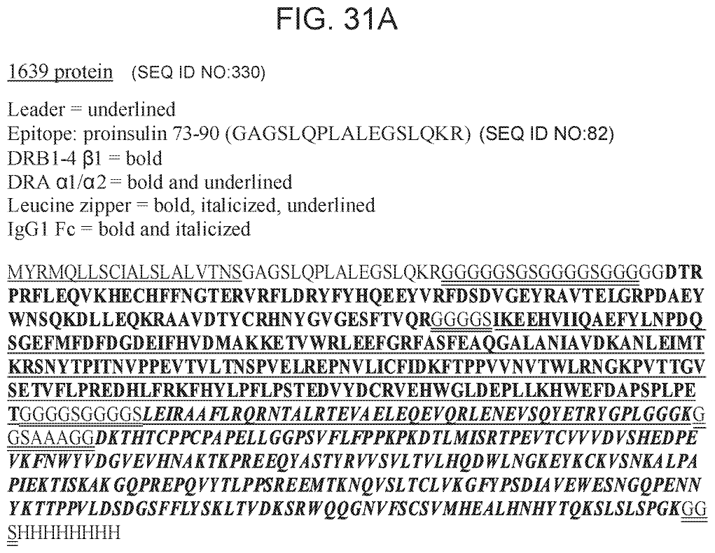

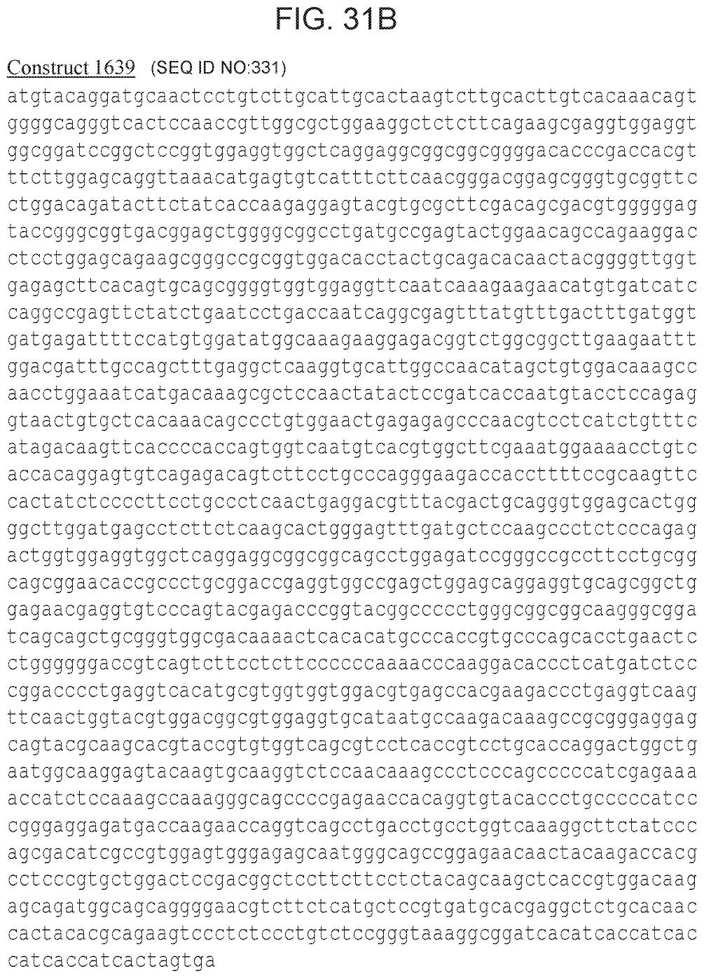

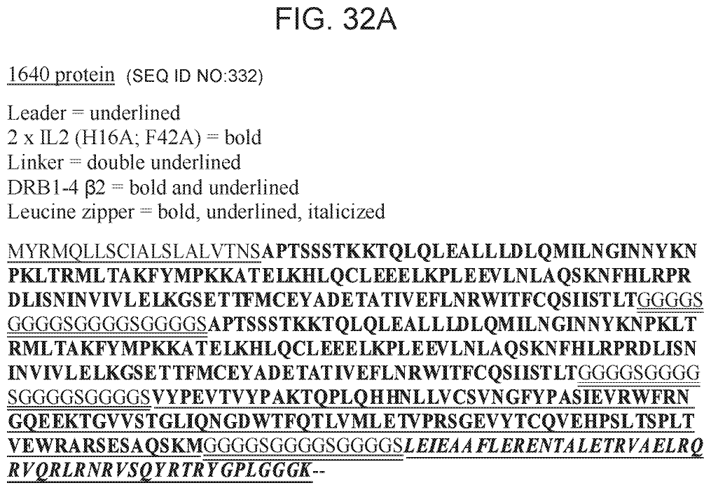

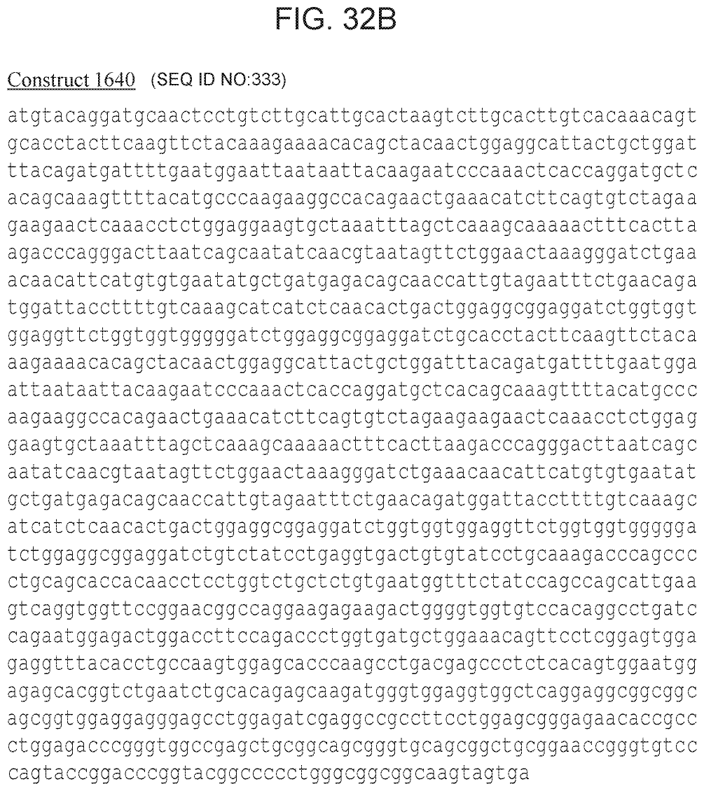

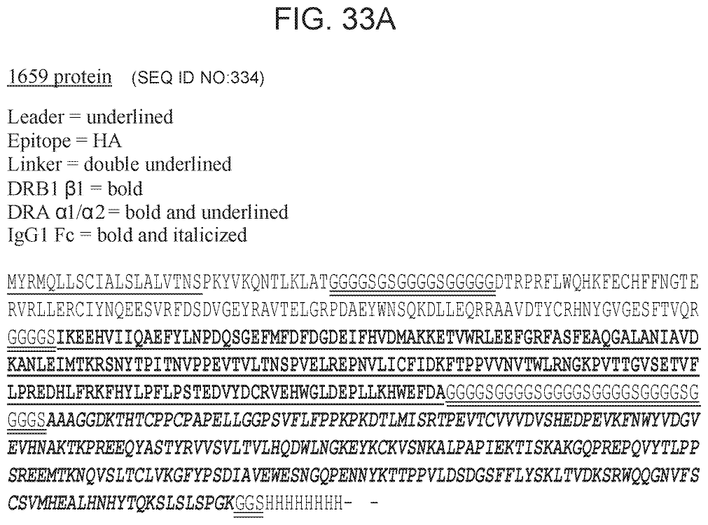

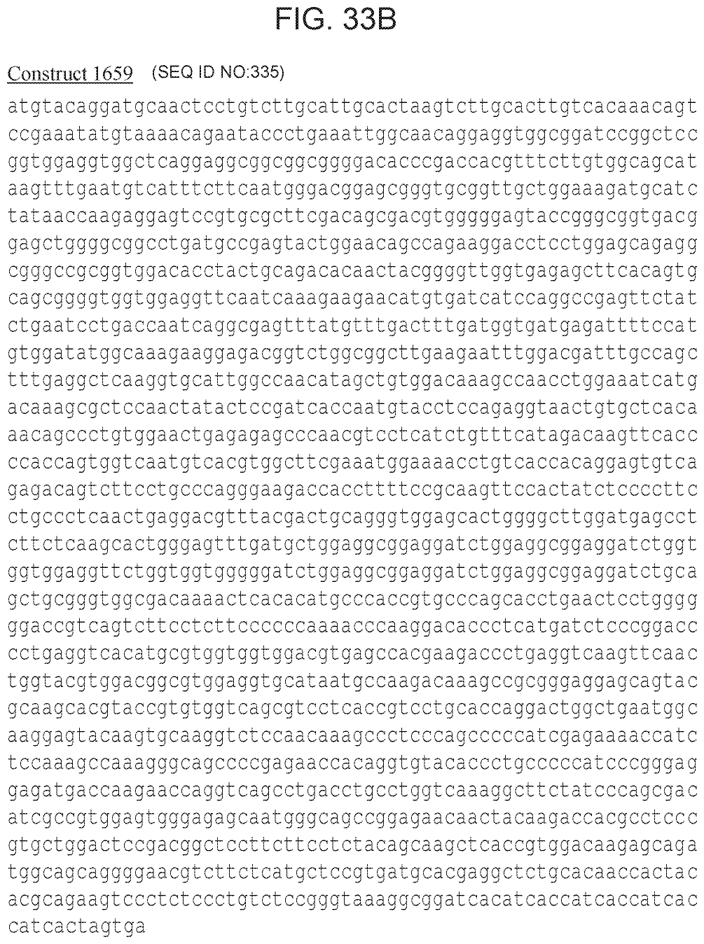

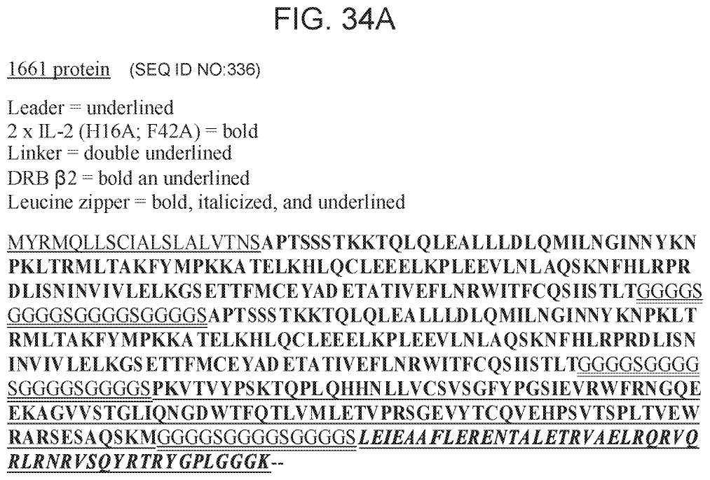

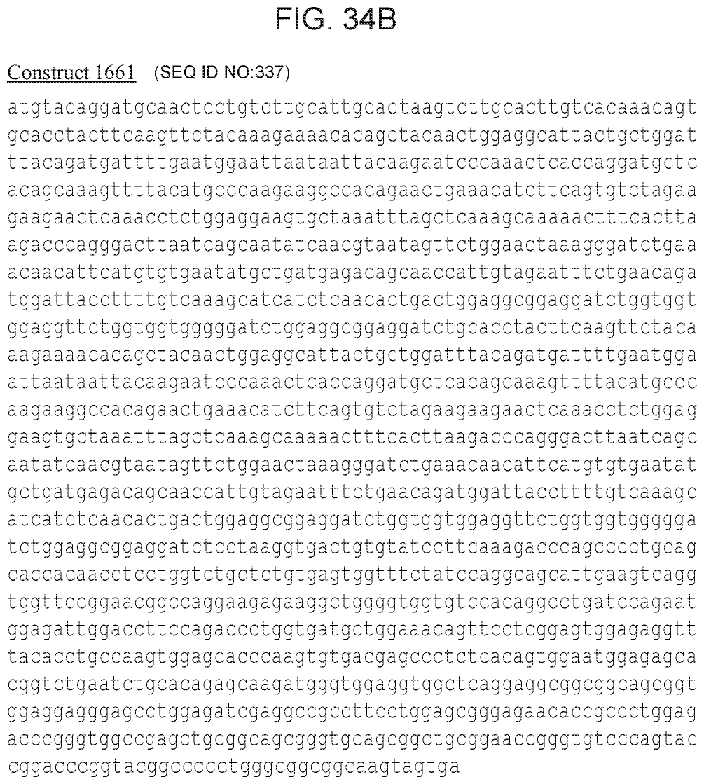

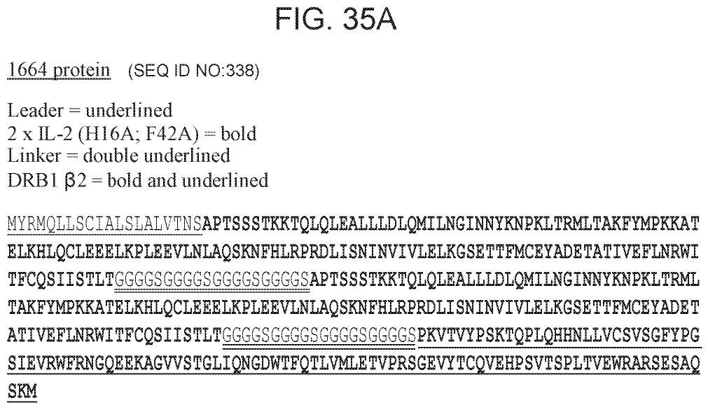

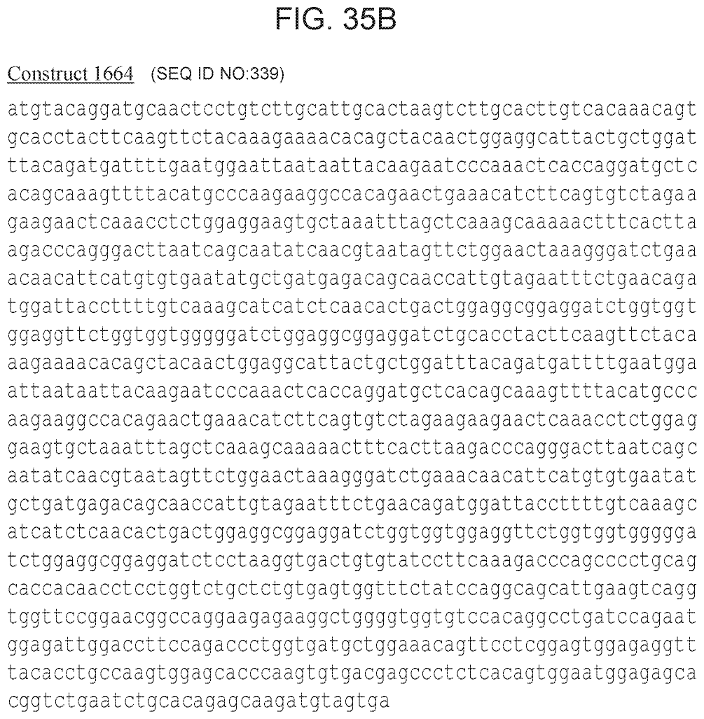

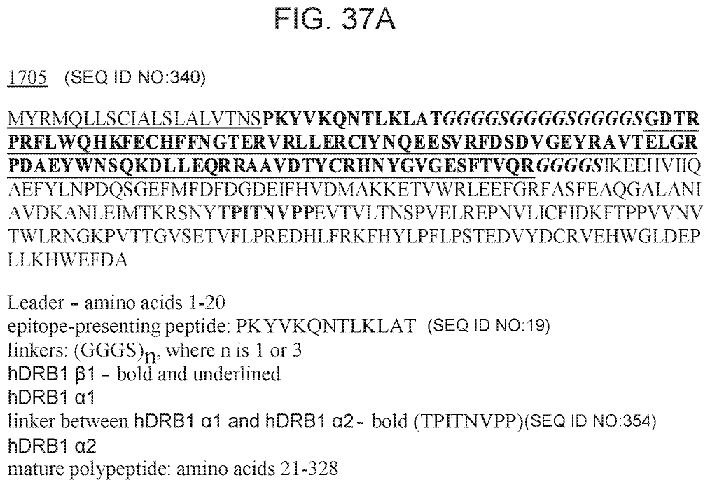

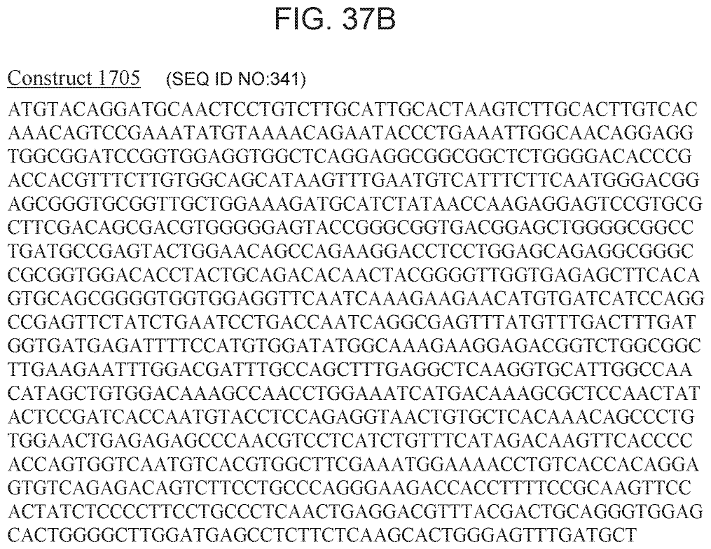

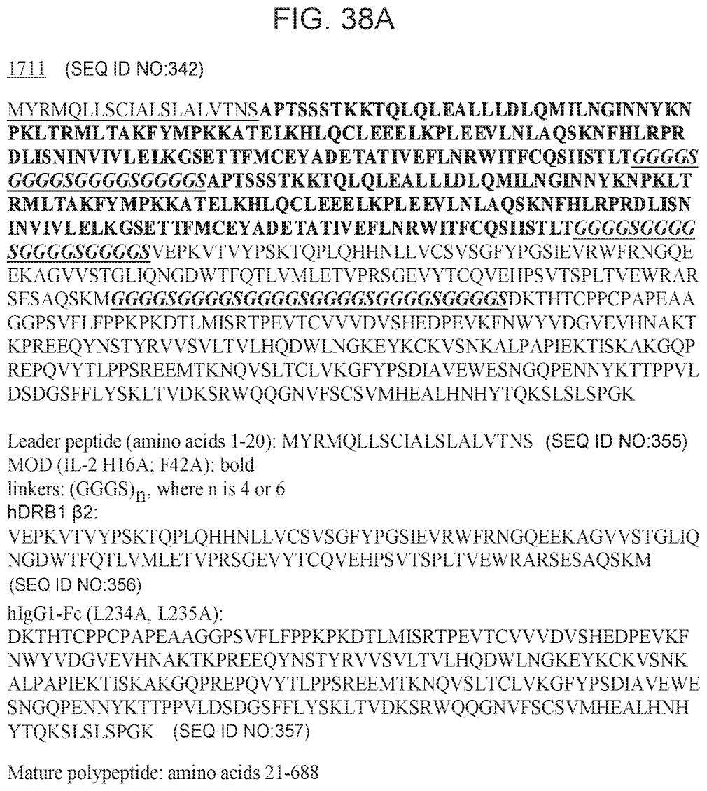

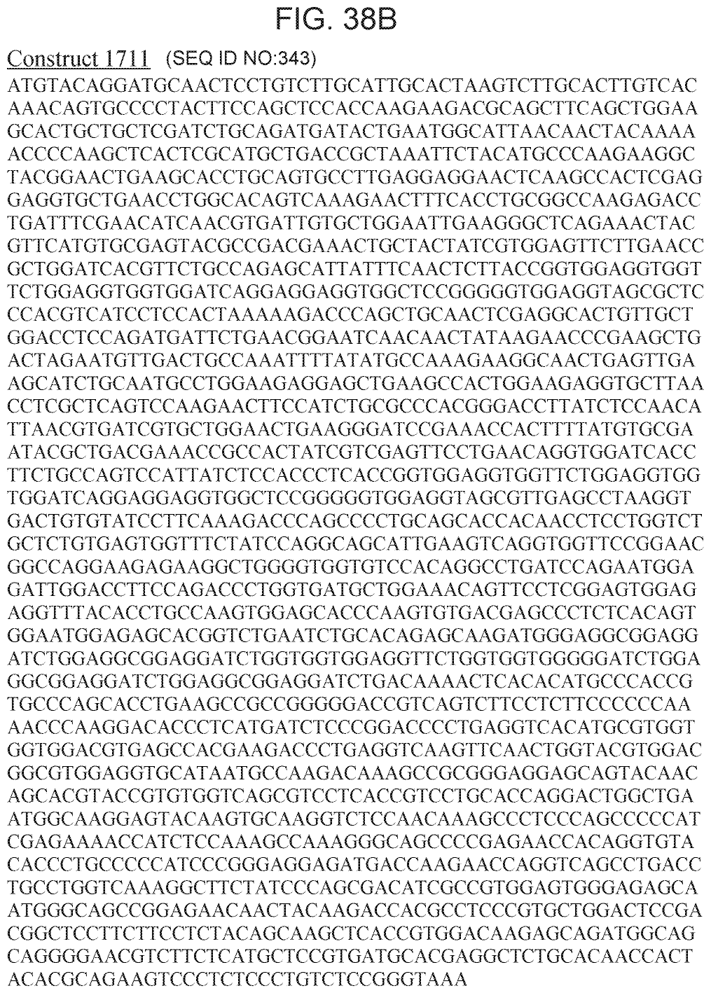

The following are non-limiting examples of multimeric TMAPPs of the present disclosure employing the following pairs of polypeptides: 1) the 1452 polypeptide depicted in FIG. 26A and the 1661 polypeptide depicted in FIG. 34A; 2) the 1659 polypeptide depicted in FIG. 33A and the 1664 polypeptide depicted in FIG. 35A; 3) the 1637 polypeptide depicted in FIG. and the 1408 polypeptide depicted in FIG. 25A; the 1639 polypeptide depicted in FIG. 31A and the 1640 polypeptide depicted in FIG. 32A; and 5) the 1711 polypeptide depicted in FIG. 37A and the 1705 polypeptide depicted in FIG. 38A. A TMAPP to be administered to an individual in need thereof will generally not include a leader sequence or a histidine tag as depicted in the aforementioned figures.

1) 1452+1661. In some cases, a multimeric TMAPP of the present disclosure comprises: a) a first polypeptide comprising, in order from N-terminus to C-terminus: i) an epitope; ii) a linker; iii) an HLA β1 polypeptide; iv) an HLA α1 polypeptide; v) an HLA α2 polypeptide; vi) a dimerizer polypeptide; and vii) an Ig Fc polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second immunomodulatory polypeptide (e.g., a variant immunomodulatory polypeptide with reduced affinity for its cognate co-immunomodulatory polypeptide); iii) an HLA β2 polypeptide; and iv) a dimerizer polypeptide. As one non-limiting example, a multimeric TMAPP of the present disclosure can comprise: a) a first polypeptide comprising, in order from N-terminus to C-terminus: i) an epitope; ii) a linker; iii) an HLA DRB1 β1 polypeptide; iv) an HLA DRA α1 polypeptide; v) an HLA DRA α2 polypeptide; vi) a leucine zipper dimerizer polypeptide; and vii) an IgG1 Fc polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) an HLA DRB β2 polypeptide; and iv) a leucine zipper dimerizer polypeptide. In some cases, the epitope is a hemagglutinin epitope, e.g., PKYVKQNTLKLAT (SEQ ID NO: 19). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:88), where the H16A and F42A substitutions are underlined. In some cases, the HLA-DRB1 β1 polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQR (SEQ ID NO:89). In some cases, the HLA DRA α1 polypeptide comprises the following amino acid sequence IKEEHVIIQAEFYLNPDQSGEFMFDFDGDEIFHVDMAKKETVWRLEEFGRFASFEAQGAL ANIAVDKANLEIMTKRSNYTPITN (SEQ ID NO:90). In some cases, the HLA DRA α2 polypeptide comprises the following amino acid sequence VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDAPSPLPET (SEQ ID NO:91). In some cases, the leucine zipper dimerizer polypeptide comprises the following amino acid sequence: LEIRAAFLRQRNTALRTEVAELEQEVQRLENEVSQYETRYGPLGGGK (SEQ ID NO:92). In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO:93). In some cases, the first polypeptide comprises the 1452 amino acid sequence depicted in FIG. 26A, without the leader sequence and without the C-terminal linker and histidine tag. For example, in some cases, the first polypeptide comprises amino acids 21 to 628 of the 1452 amino acid sequence depicted in FIG. 26A. In some cases, the second polypeptide comprises the 1661 amino acid sequence depicted in FIG. 34A, without the leader sequence. For example, in some cases, the second polypeptide comprises amino acids 21 to 491 of the amino acid sequence depicted in FIG. 34A. In some cases, the epitope of the first polypeptide is not PKYVKQNTLKLAT (SEQ ID NO: 19), but instead is substituted with a different epitope.

2) 1659+1664. In some cases, a multimeric TMAPP of the present disclosure comprises: a) a first polypeptide comprising, in order from N-terminus to C-terminus: i) an epitope; ii) an HLA β1 polypeptide; iii) an HLA α1 polypeptide; iv) an HLA α2 polypeptide; and v) an Ig Fc polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second immunomodulatory polypeptide (e.g., a variant immunomodulatory polypeptide with reduced affinity for its cognate co-immunomodulatory polypeptide); and iii) an HLA 32 polypeptide. As one non-limiting example, a multimeric TMAPP of the present disclosure can comprise: a) a first polypeptide comprising, in order from N-terminus to C-terminus: i) an epitope; ii) an HLA DRB1 β1 polypeptide; iii) an HLA DRA α1 polypeptide; iv) an HLA DRA α2 polypeptide; and v) an IgG1 Fc polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); and iii) an HLA DRB1 β2 polypeptide. In some cases, the epitope is a hemagglutinin epitope, e.g., PKYVKQNTLKLAT (SEQ ID NO:19). In some cases, the HLA DRB1 β1 polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQR (SEQ ID NO:89). In some cases, the DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO:90). In some cases, the DRA α2 polypeptide comprises the following amino acid sequence: VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDA (SEQ ID NO:94). In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO:93). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:88), where the H16A and F42A substitutions are underlined. In some cases, the HLA DRB1 β2 polypeptide comprises the following amino acid sequence: PKVTVYPSKTQPLQHHNLLVCSVSGFYPGSIEVRWFRNGQEEKAGVVSTGLIQNGDWTF QTLVMLETVPRSGEVYTCQVEHPSVTSPLTVEWRARSESAQSKM (SEQ ID NO:95). In some cases, the first polypeptide comprises the 1659 amino acid sequence depicted in FIG. 33A, without the leader peptide and without the C-terminal linker and histidine tag. For example, in some cases, the first polypeptide comprises amino acids 21 to 591 of the 1659 amino acid sequence depicted in FIG. 33A. In some cases, the epitope is not PKYVKQNTLKLAT (SEQ ID NO: 19), but instead is substituted with a different epitope. In some cases, the second polypeptide comprises the 1664 amino acid sequence depicted in FIG. 35A, without the leader sequence. For example in some cases, the second polypeptide comprises amino acids 21 to 429 of the 1664 amino acid sequence depicted in FIG. 35A.

3) 1637-1408. In some cases, a multimeric TMAPP of the present disclosure comprises: a) a first polypeptide comprising, in order from N-terminus to C-terminus: i) an epitope; ii) an HLA 31 polypeptide; iii) an HLA α1 polypeptide; iv) an HLA α2 polypeptide; v) a dimerizer polypeptide; and vi) an Ig Fc polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second immunomodulatory polypeptide (e.g., a variant immunomodulatory polypeptide with reduced affinity for its cognate co-immunomodulatory polypeptide); iii) an HLA β2 polypeptide; and iv) a dimerizer polypeptide. As one non-limiting example, a multimeric TMAPP of the present disclosure can comprise: a) a first polypeptide comprising, in order from N-terminus to C-terminus: i) an epitope; ii) an HLA DRB1 β1 polypeptide; iii) an HLA DRA α1 polypeptide; iv) an HLA DRA α2 polypeptide; v) a leucine zipper dimerizer polypeptide; and vi) an IgG1 Fc polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) an HLA DRB1 β2 polypeptide; and iv) a leucine zipper dimerizer polypeptide. In some cases, the epitope is a cytomegalovirus (CMV) pp65 epitope (LPLKMLNIPSINVH; SEQ ID NO:96). In some cases, the HLA DRB 31 polypeptide comprises the following amino acid sequence: DTRPRFLWQHKFECHFFNGTERVRLLERCIYNQEESVRFDSDVGEYRAVTELGRPAAEY WNSQKDLLEQRRAAVDTYCRHNYGVGESFTVQR (SEQ ID NO:97). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO:90). In some cases, the HLA DRA α2 polypeptide comprises the following amino acid sequence: VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDAPSPLPET (SEQ ID NO:91). In some cases, the leucine zipper polypeptide comprises the following amino acid sequence: LEIRAAFLRQRNTALRTEVAELEQEVQRLENEVSQYETRYGPLGGGK (SEQ ID NO:92). In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO:93). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:88), where the H16A and F42A substitutions are underlined. In some cases, the HLA DRB1 β2 polypeptide comprises the following amino acid sequence:

(SEQ ID NO: 98)
VEPKVTVYPSKTQPLQHHNLLVCSVSGFYPGSIEVRWFRNGQEEKAGV
VSTGLIQNGDWTFQTLVMLETVPRSGEVYTCQVEHPSVTSPLTVEWRA
RSESAQSKM.

In some cases, the leucine zipper polypeptide comprises the following amino acid sequence:

(SEQ ID NO: 99)
LEIEAAFLERENTALETRVAELRQRVQRLRNRVSQYRTRYGPLGGGK.

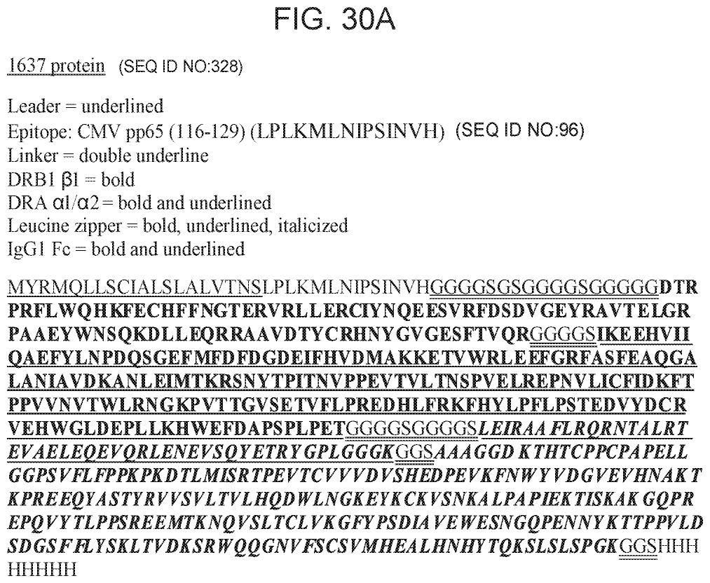

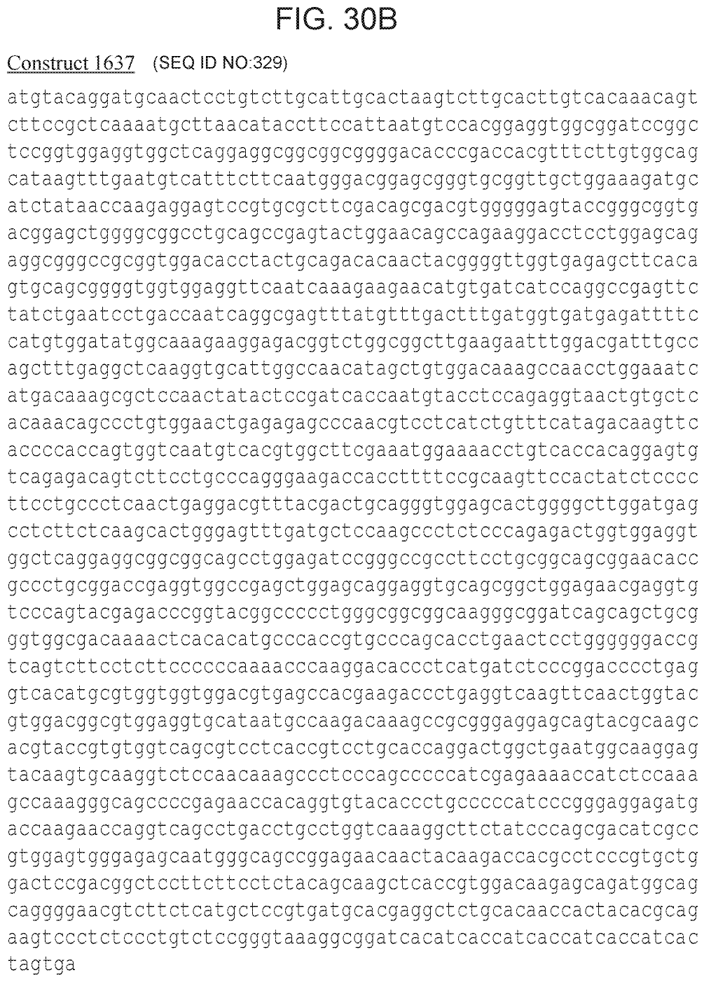

In some cases, the first polypeptide comprises the 1637 amino acid sequence depicted in FIG. 30A, without the leader sequence and without the C-terminal linker and histidine tag. For example, in some cases, the first polypeptide comprises amino acids 21-629 of the 1637 amino acid sequence depicted in FIG. 30A. In some cases, the first polypeptide does not include the epitope LPLKMLNIPSINVH (SEQ ID NO:96); instead, the epitope is substituted with a different epitope. In some cases, the second polypeptide comprises the amino acid sequence depicted in FIG. 25A, but without the leader peptide. Thus, for example, in some cases, the second polypeptide comprises amino acids 21-493 of the amino acid sequence depicted in FIG. 25A.

4) 1639+1640. In some cases, a multimeric TMAPP of the present disclosure comprises: a) a first polypeptide comprising, in order from N-terminus to C-terminus: i) an epitope; ii) an HLA β1 polypeptide; iii) an HLA α1 polypeptide; iv) an HLA α2 polypeptide; v) a dimerizer polypeptide; and vi) an Ig Fc polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second immunomodulatory polypeptide (e.g., a variant immunomodulatory polypeptide with reduced affinity for its cognate co-immunomodulatory polypeptide); iii) an HLA β2 polypeptide; and iv) a dimerizer polypeptide. As one non-limiting example, a multimeric TMAPP of the present disclosure can comprise: a) a first polypeptide comprising, in order from N-terminus to C-terminus: i) an epitope; ii) an HLA DRB1-4 β1 polypeptide; iii) an HLA DRA α1 polypeptide; iv) an HLA DRA α2 polypeptide; v) a leucine zipper dimerizer polypeptide; and vi) an IgG1 Fc polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) an HLA DRB1-4 β2 polypeptide; and iv) a leucine zipper dimerizer polypeptide. In some cases, the epitope is proinsulin 73-90 (GAGSLQPLALEGSLQKR; SEQ ID NO:82). In some cases, the HLA DRB1-4 β1 polypeptide comprises the following amino acid sequence: DTRPRFLEQVKHECHFFNGTERVRFLDRYFYHQEEYVRFDSDVGEYRAVTELGRPDAE YWNSQKDLLEQKRAAVDTYCRHNYGVGESFTVQR (SEQ ID NO:100). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO:90). In some cases, the HLA DRA α2 polypeptide comprises the following amino acid sequence: VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDAPSPLPET (SEQ ID NO:91). In some cases, the leucine zipper polypeptide comprises the following amino acid sequence: LEIRAAFLRQRNTALRTEVAELEQEVQRLENEVSQYETRYGPLGGGK (SEQ ID NO:92). In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO:93). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:88), where the H16A and F42A substitutions are underlined. In some cases, the HLA DRB1-4 β2 polypeptide comprises the following amino acid sequence: VYPEVTVYPAKTQPLQHHNLLVCSVNGFYPASIEVRWFRNGQEEKTGVVSTGLIQNGD WTFQTLVMLETVPRSGEVYTCQVEHPSLTSPLTVEWRARSESAQSKM (SEQ ID NO:101). In some cases, the leucine zipper polypeptide comprises the following amino acid sequence: LEIEAAFLERENTALETRVAELRQRVQRLRNRVSQYRTRYGPLGGGK (SEQ ID NO:99). In some cases, the first polypeptide comprises the amino acid sequence depicted in FIG. 31A, without the leader peptide and without the C-terminal linker and histidine tag. For example, in some cases, the first polypeptide comprises amino acids 21-633 of the amino acid sequence depicted in FIG. 31A. In some cases, the epitope is not proinsulin 73-90 (GAGSLQPLALEGSLQKR; SEQ ID NO:82); instead, the epitope is substituted with a different epitope. In some cases, the second polypeptide comprises the amino acid sequence depicted in FIG. 32A, without the leader peptide. For example, in some cases, the second polypeptide comprises amino acids 21-493 of the amino acid sequence depicted in FIG. 32A.

5) 1711+1705. In some cases, a multimeric TMAPP of the present disclosure comprises: a) a first polypeptide comprising, in order from N-terminus to C-terminus: i) an epitope; ii) an HLA β1 polypeptide; iii) an HLA α1 polypeptide; and iv) an HLA α2 polypeptide; and b) a second polypeptide comprising, in order from N-terminus to C-terminus: i) a first immunomodulatory polypeptide (e.g., a variant immunomodulatory polypeptide with reduced affinity for its cognate co-immunomodulatory polypeptide); ii) a second immunomodulatory polypeptide (e.g., a variant immunomodulatory polypeptide with reduced affinity for its cognate co-immunomodulatory polypeptide); iii) an HLA β2 polypeptide; and iv) an Ig Fc polypeptide. As one non-limiting example, a multimeric TMAPP of the present disclosure can comprise: a) a first polypeptide comprising, in order from N-terminus to C-terminus: i) an epitope; ii) an HLA DRB1 β1 polypeptide; iii) an HLA DRA α1 polypeptide; and iv) an HLA DRA α2 polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) an HLA DRB1 β2 polypeptide; and iv) an IgG Fc polypeptide. The multimeric TMAPP can include a variant IgG Fc polypeptide. For example, a multimeric TMAPP of the present disclosure can comprise: a) a first polypeptide comprising, in order from N-terminus to C-terminus: i) an epitope; ii) an HLA DRB1 β1 polypeptide; iii) an HLA DRA α1 polypeptide; and iv) an HLA DRA α2 polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) an HLA DRB1 β2 polypeptide; and iv) an IgG1 Fc polypeptide comprising L234A and L235A substitutions. The multimeric TMAPP can include one or more linkers. For example, a multimeric TMAPP of the present disclosure can comprise: a) a first polypeptide comprising, in order from N-terminus to C-terminus: i) an epitope; ii) a peptide linker; iii) an HLA DRB1 β1 polypeptide; iv) a peptide linker; v) an HLA DRA α1 polypeptide; and vi) an HLA DRA α2 polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) a peptide linker; iv) an HLA DRB1 β2 polypeptide; v) a peptide linker; and vi) an Ig Fc polypeptide (e.g., an IgG1 Fc polypeptide comprising L234A and L235A substitutions). For example, a multimeric TMAPP of the present disclosure can comprise: a) a first polypeptide comprising, in order from N-terminus to C-terminus: i) an epitope; ii) the peptide linker (GGGGS)₃ (SEQ ID NO:346); iii) an HLA DRB1 β1 polypeptide; iv) the peptide linker GGGGS (SEQ ID NO:75); v) an HLA DRA α1 polypeptide; and vi) an HLA DRA α2 polypeptide; and b) a second polypeptide comprising, in order from N-terminus to C-terminus: i) a first immunomodulatory polypeptide (e.g., a variant IL-2 polypeptide comprising H16A and F42A substitutions); ii) a second immunomodulatory polypeptide (e.g., a variant IL-2 polypeptide comprising H16A and F42A substitutions); iii) the peptide linker (GGGGS)₄ (SEQ ID NO:347); iv) an HLA DRB1 β2 polypeptide; v) the peptide linker (GGGGS)₆ (SEQ ID NO:349); and vi) an Ig Fc polypeptide (e.g., an IgG1 Fc polypeptide comprising L234A and L235A substitutions). For example, a multimeric TMAPP of the present disclosure can comprise: a) a first polypeptide comprising, in order from N-terminus to C-terminus: i) an epitope; ii) the peptide linker (GGGGS)₃ (SEQ ID NO:346); iii) an HLA DRB1 β1 polypeptide; iv) the peptide linker GGGGS (SEQ ID NO:75); v) an HLA DRA α1 polypeptide; and vi) an HLA DRA α2 polypeptide; and b) a second polypeptide comprising, in order from N-terminus to C-terminus: i) a first variant IL-2 polypeptide comprising H16A and F42A substitutions; ii) a second variant IL-2 polypeptide comprising H16A and F42A substitutions (e.g., where the first and the second variant IL-2 polypeptides comprise the same amino acid sequence); iii) the peptide linker (GGGGS)₄ (SEQ ID NO:347); iv) an HLA DRB1 β2 polypeptide; v) the peptide linker (GGGGS)₆ (SEQ ID NO:349); and vi) an IgG1 Fc polypeptide comprising L234A and L235A substitutions. In some cases, the HLA DRB1 β polypeptide comprises the following amino acid sequence: GDTRPRFLWQHKFECHFFNGTERVRLLERCIYNQEESVRFDSDVGEYRAVTELGRPDAE YWNSQKDLLEQRRAAVDTYCRHNYGVGESFTVQR (SEQ ID NO:102). In some cases, the HLA DRB1 β1p polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQRRVEP (SEQ ID NO:106). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNY (SEQ ID NO: 103). In some cases, the HLA DRA α2 polypeptide comprises the following amino acid sequence: EVTVLTNSPVELREPNVLICFIDKFTPPVVNVTWLRNGKPVTTGVSETVFLPREDHLFRK FHYLPFLPSTEDVYDCRVEHWGLDEPLLKHWEFDA (SEQ ID NO: 104). In some cases, the HLA DRB1 β2 polypeptide comprises the following amino acid sequence:

(SEQ ID NO: 98)
VEPKVTVYPSKTQPLQHHNLLVCSVSGFYPGSIEVRWFRNGQEEKAGV
VSTGLIQNGDWTFQTLVMLETVPRSGEVYTCQVEHPSVTSPLTVEWRA
RSESAQSKM.

In some cases, the first and the second immunomodulatory polypeptides are variant IL-2 polypeptides, both comprising the amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:88). In some cases, the Fc polypeptide is an IgG1 Fc polypeptide comprising L234A and L235A substitutions, and comprises the amino acid sequence: DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 105). In some cases, a TMAPP of the present disclosure comprises: a) a first polypeptide comprising amino acids 21-328 of the amino acid sequence depicted in FIG. 37A; and b) a second polypeptide comprising amino acids 21-688 of the amino acid sequence depicted in FIG. 38A. In some cases, a TMAPP of the present disclosure comprises: a) a first polypeptide encoded by the nucleotide sequence depicted in FIG. 37B; and b) a second polypeptide encoded by the nucleotide sequence depicted in FIG. 38B.

### Single Polypeptide Chain T-Cell Modulatory Antigen-Presenting Polypeptides

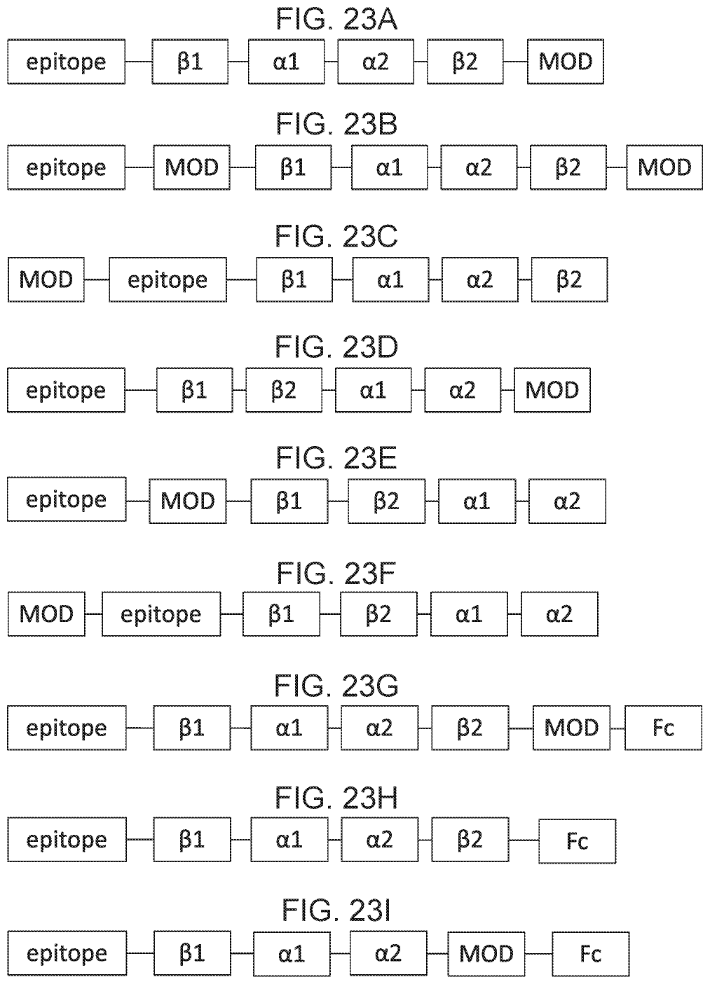

As noted above, in some cases, a TMAPP of the present disclosure comprises a single polypeptide chain. Non-limiting examples are depicted schematically in FIG. 23A-23I. A single-chain TMAPP of the present disclosure can include one or more linkers between any two adjacent polypeptides, e.g., between a peptide antigen and an immunomodulatory polypeptide, between an immunomodulatory polypeptide and an MHC Class II polypeptide, between two MHC Class II polypeptides, between an immunomodulatory polypeptide and an Ig Fc polypeptide, etc.

In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; and vi) an immunomodulatory polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; vi) an immunomodulatory polypeptide; and vii) an Ig Fc polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; and vi) 2 copies of an immunomodulatory polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; vi) 2 copies of an immunomodulatory polypeptide; and v) an Ig Fc polypeptide.

In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II α1 polypeptide; v) an MHC Class II α2 polypeptide; vi) an MHC Class II β2 polypeptide; and v) a second copy of the immunomodulatory polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II α1 polypeptide; v) an MHC Class II α2 polypeptide; vi) an MHC Class II β2 polypeptide; vii) a second copy of the immunomodulatory polypeptide; and viii) an immunoglobulin or non-immunoglobulin scaffold polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II α1 polypeptide; v) an MHC Class II α2 polypeptide; vi) an MHC Class II β2 polypeptide; vii) a second copy of the immunomodulatory polypeptide; and viii) an Ig Fc polypeptide.

In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II α1 polypeptide; v) an MHC Class II α2 polypeptide; and vi) an MHC Class II β2 polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II α1 polypeptide; v) an MHC Class II α2 polypeptide; vi) an MHC Class II β2 polypeptide; and vii) an immunoglobulin or non-immunoglobulin scaffold polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II α1 polypeptide; v) an MHC Class II α2 polypeptide; vi) an MHC Class II β2 polypeptide; and vii) an Ig Fc polypeptide.

In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II α1 polypeptide; v) an MHC Class II α2 polypeptide; vi) an MHC Class II β2 polypeptide; and vii) a second copy of the immunomodulatory polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II α1 polypeptide; v) an MHC Class II α2 polypeptide; vi) an MHC Class II β2 polypeptide; vii) a second copy of the immunomodulatory polypeptide; and viii) an immunoglobulin or non-immunoglobulin scaffold polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II α1 polypeptide; v) an MHC Class II α2 polypeptide; vi) an MHC Class II β2 polypeptide; vii) a second copy of the immunomodulatory polypeptide; and viii) an Ig Fc polypeptide.

In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; and vi) an immunomodulatory polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; vi) an immunomodulatory polypeptide; and vii) an immunoglobulin or non-immunoglobulin scaffold polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II β2 polypeptide; iv) an MHC Class II α1 polypeptide; v) an MHC Class II α2 polypeptide; vi) an immunomodulatory polypeptide; and vii) an Ig Fc polypeptide. In any one of the above embodiments, the TMAPP can comprise one or more linkers, where the linker may be between one or more of: i) the peptide antigen and the MHC Class II β1 polypeptide; ii) the MHC Class II β2 polypeptide and the MHC Class II α1 polypeptide; iii) the MHC Class II α2 polypeptide and the immunomodulatory polypeptide; and iv) the immunomodulatory polypeptide and the Ig Fc polypeptide.

In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II β2 polypeptide; v) an MHC Class II α1 polypeptide; and v) an MHC Class II α2 polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II β2 polypeptide; v) an MHC Class II α1 polypeptide; vi) an MHC Class II α2 polypeptide; and vii) an immunoglobulin or non-immunoglobulin scaffold polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II β2 polypeptide; v) an MHC Class II α1 polypeptide; vi) an MHC Class II α2 polypeptide; and vii) an Ig Fc polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II β2 polypeptide; v) an MHC Class II α1 polypeptide; vi) an MHC Class II α2 polypeptide; and vii) a second copy of the immunomodulatory polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II β2 polypeptide; v) an MHC Class II α1 polypeptide; vi) an MHC Class II α2 polypeptide; vii) a second copy of the immunomodulatory polypeptide; and viii) an immunoglobulin or non-immunoglobulin scaffold polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an immunomodulatory polypeptide; iii) an MHC Class II β1 polypeptide; iv) an MHC Class II β2 polypeptide; v) an MHC Class II α1 polypeptide; vi) an MHC Class II α2 polypeptide; vii) a second copy of the immunomodulatory polypeptide; and viii) an Ig Fc polypeptide. In any one of the above embodiments, the TMAPP can comprise one or more linkers, where the linker may be between one or more of: i) the peptide antigen and the immunomodulatory polypeptide; ii) the immunomodulatory polypeptide and the MHC Class II β1 polypeptide; iii) the MHC Class II α2 polypeptide and the Ig Fc polypeptide; and iv) the MHC Class II α2 polypeptide and the second copy of the immunomodulatory polypeptide.

In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II β2 polypeptide; v) an MHC Class II α1 polypeptide; and vi) an MHC Class II α2 polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II β2 polypeptide; v) an MHC Class II α1 polypeptide; vi) an MHC Class II α2 polypeptide; and vii) an immunoglobulin or non-immunoglobulin scaffold polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II β2 polypeptide; v) an MHC Class II α1 polypeptide; vi) an MHC Class II α2 polypeptide; and vii) an Ig Fc polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II β2 polypeptide; v) an MHC Class II α1 polypeptide; vi) an MHC Class II α2 polypeptide; and vii) a second copy of the immunomodulatory polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II β2 polypeptide; v) an MHC Class II α1 polypeptide; vi) an MHC Class II α2 polypeptide; vii) a second copy of the immunomodulatory polypeptide; and viii) an immunoglobulin or non-immunoglobulin scaffold polypeptide. In some cases, a TMAPP of the present disclosure comprises, in order from N-terminus to C-terminus: i) an immunomodulatory polypeptide; ii) a peptide antigen (an “epitope”) that is recognized (e.g., is capable of being recognized and bound) by a TCR; iii) an MHC Class II β1 polypeptide; iv) an MHC Class II β2 polypeptide; v) an MHC Class II α1 polypeptide; vi) an MHC Class II α2 polypeptide; vii) a second copy of the immunomodulatory polypeptide; and viii) an Ig Fc polypeptide. In any one of the above embodiments, the TMAPP can comprise one or more linkers, where the linker may be between one or more of: i) the immunomodulatory polypeptide and the peptide antigen; ii) the peptide antigen and the MHC Class II β1 polypeptide; iii) the MHC Class II α2 polypeptide and the Ig Fc polypeptide; and iv) the MHC Class II α2 polypeptide and the second copy of the immunomodulatory polypeptide.

In some cases, a TMAPP of the present disclosure comprises a single polypeptide chain. For example, in some cases, a TMAPP of the present disclosure comprises a single polypeptide chain comprising: i) a peptide antigen (an “epitope”) that is recognized (e.g., is capable of being recognized and bound) by a TCR; ii) an MHC Class II α1 polypeptide; iii) an MHC Class II α2 polypeptide; iv) an MHC Class II β1 polypeptide; and v) one or more immunomodulatory polypeptides. In some cases, a TMAPP of the present disclosure comprises a single polypeptide chain. For example, in some cases, a TMAPP of the present disclosure comprises a single polypeptide chain comprising: i) a peptide antigen (an “epitope”) that is recognized (e.g., is capable of being recognized and bound) by a TCR; ii) an MHC Class II α1 polypeptide; iii) an MHC Class II α2 polypeptide; iv) an MHC Class II β1 polypeptide; v) an MHC Class II β2 polypeptide; and vi) one or more immunomodulatory polypeptides. In some cases, a TMAPP of the present disclosure comprises a single polypeptide chain comprising: i) a peptide antigen (an “epitope”) that is recognized (e.g., is capable of being recognized and bound) by a TCR; ii) an MHC Class II α1 polypeptide; iii) an MHC Class II α2 polypeptide; iv) an MHC Class II β1 polypeptide; v) an MHC Class II β2 polypeptide; vi) one or more immunomodulatory polypeptides; and vii) an Ig or a non-Ig scaffold polypeptide. In some cases, a TMAPP of the present disclosure comprises a single polypeptide chain comprising: i) a peptide antigen (an “epitope”) that is recognized (e.g., is capable of being recognized and bound) by a TCR; ii) an MHC Class II α1 polypeptide; iii) an MHC Class II α2 polypeptide; iv) an MHC Class II β1 polypeptide; v) an MHC Class II β2 polypeptide; vi) one or more immunomodulatory polypeptides; and vii) a dimerizing polypeptide. In some cases, the TMAPP comprises a linker (an “L1”) between an MHC polypeptide and an Ig Fc polypeptide; where exemplary suitable linkers include (GGGGS)n (SEQ ID NO:75), where n is 1, 2, 3, 4, 5, 6, 7, or 8. In some cases, the TMAPP comprises a linker (an “L2”) between an immunomodulatory polypeptide and an MHC polypeptide, where exemplary suitable linkers include (GGGGS)n (SEQ ID NO:75), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO:75), where n is 1, 2, 3, 4, 5, 6, 7, or 8.

In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; and vi) one or more immunomodulatory polypeptides. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; and v) one or more immunomodulatory polypeptides. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; vi) one or more immunomodulatory polypeptides; and vii) an Ig Fc polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; vi) a first immunomodulatory polypeptide; vii) a second immunomodulatory polypeptide; and viii) an Ig Fc polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) a first immunomodulatory polypeptide; vi) a second immunomodulatory polypeptide; and vii) an Ig Fc polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; vi) one or more immunomodulatory polypeptides; and vii) a dimerizing polypeptide. In some cases, a TMAPP of the present disclosure comprises, in order from N-terminus to C-terminus: i) a peptide antigen (an “epitope”) that is recognized (e.g., is capable of being recognized and bound) by a TCR; ii) an MHC Class II β1 polypeptide; iii) an MHC Class II α1 polypeptide; iv) an MHC Class II α2 polypeptide; v) an MHC Class II β2 polypeptide; vi) one or more immunomodulatory polypeptides; vii) a dimerizing polypeptide; and viii) a dimerizing polypeptide. In some cases, the TMAPP comprises a linker (an “L1”) between an MHC polypeptide and an Ig Fc polypeptide; where exemplary suitable linkers include (GGGGS)n (SEQ ID NO:75), where n is 1, 2, 3, 4, 5, 6, 7, or 8. In some cases, the TMAPP comprises a linker (an “L2”) between an immunomodulatory polypeptide and an MHC polypeptide, where exemplary suitable linkers include (GGGGS)n (SEQ ID NO:75), where n is 1, 2, 3, 4, 5, 6, 7, or 8. In some cases, where the TMAPP comprises two immunomodulatory polypeptides, in some cases, the two immunomodulatory polypeptides are separated by a linker (an “L3); where exemplary suitable linkers include (GGGGS)n (SEQ ID NO:75), where n is 1, 2, 3, 4, 5, 6, 7, or 8.

### Exemplary Single-Chain T-Cell Modulatory Antigen-Presenting Polypeptides

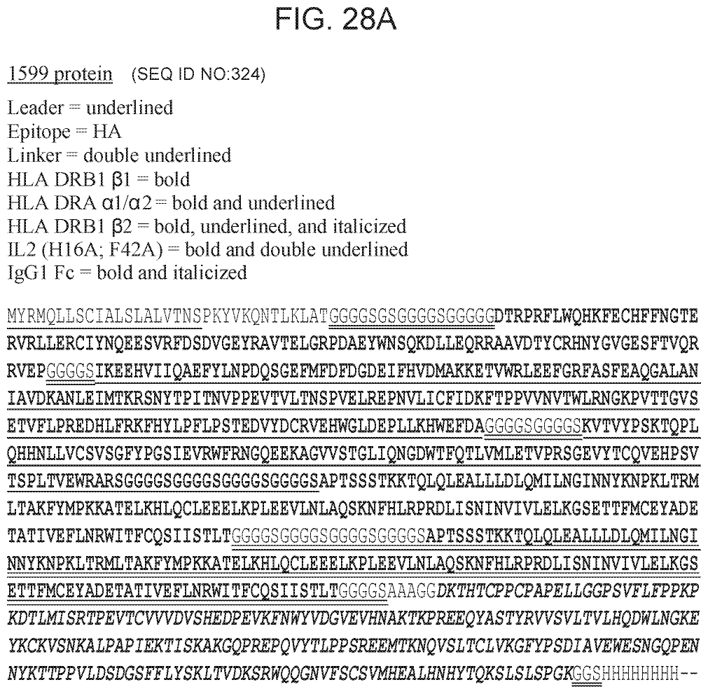

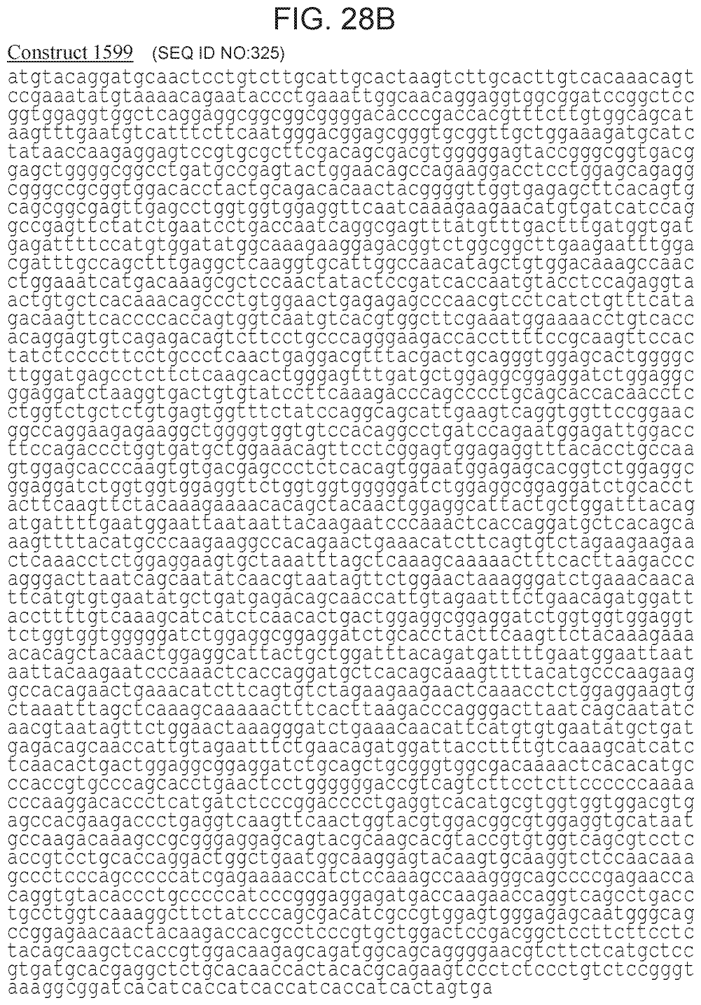

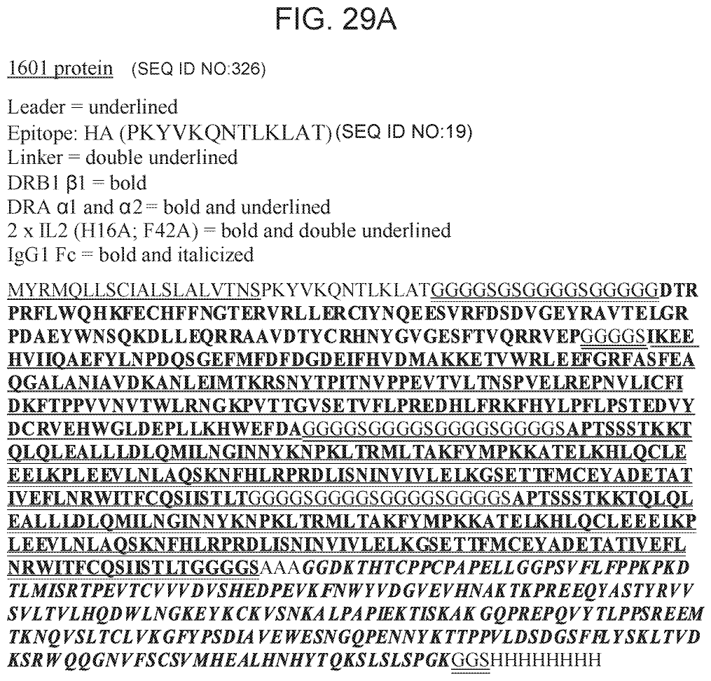

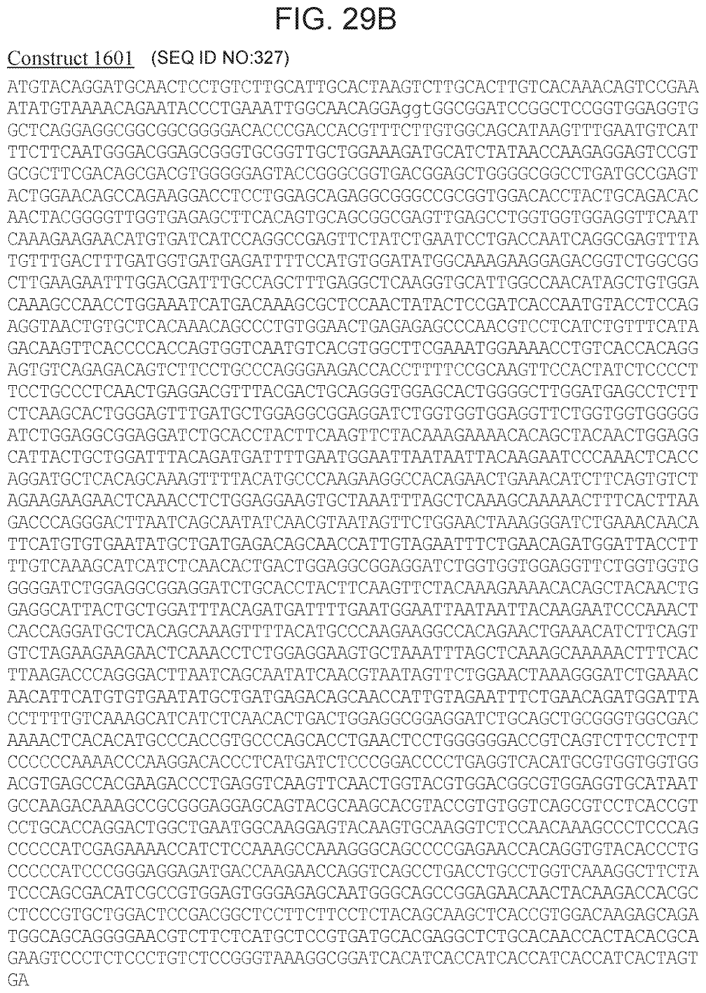

The following are non-limiting examples of single-chain TMAPPs of the present disclosure. See, e.g., FIG. 28A (1599 polypeptide); and FIG. 29A (1601 polypeptide). A TMAPP to be administered to an individual in need thereof will generally not include a leader sequence or a histidine tag as depicted in the aforementioned figures.

1) 1599. In some cases, a single-chain TMAPP of the present disclosure comprises, in order from N-terminus to C-terminus: i) an epitope; ii) an HLA β1 polypeptide; iii) an HLA α1 polypeptide; iv) an HLA α2 polypeptide; v) an HLA β2 polypeptide; vi) an immunomodulatory polypeptide (e.g., a variant immunomodulatory polypeptide with reduced affinity for its cognate co-immunomodulatory polypeptide); and vii) an Ig Fc polypeptide. As one non-limiting example, a single-chain TMAPP of the present disclosure can comprise, in order from N-terminus to C-terminus: i) an epitope; ii) an HLA DRB1 β1 polypeptide; iii) an HLA DRA α1 polypeptide; iv) an HLA DRA α2 polypeptide; v) an HLA DRB β2 polypeptide; vi) an immunomodulatory polypeptide (e.g., a variant IL-2 polypeptide comprising H16A and F42A substitutions); and vii) an IgG1 Fc polypeptide. In some cases, the epitope is a hemagglutinin epitope (e.g., PKYVKQNTLKLAT; SEQ ID NO:19). In some cases, the HLA DRB1 β1 polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQRRVEP (SEQ ID NO:106). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO:90). In some cases, the HLA DRB β2 polypeptide comprises the following amino acid sequence: KVTVYPSKTQPLQHHNLLVCSVSGFYPGSIEVRWFRNGQEEKAGVVSTGLIQNGDWTF QTLVMLETVPRSGEVYTCQVEHPSVTSPLTVEWRARS (SEQ ID NO: 107). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:88), where the H16A and F42A substitutions are underlined. In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO:93). In some cases, the single-chain polypeptide comprises the amino acid sequence depicted in FIG. 28A, without the leader peptide and without the C-terminal linker and histidine tag. For example, in some cases, the single-chain polypeptide comprises amino acids 21-981 of the amino acid sequence depicted in FIG. 28A. In some cases, the single-chain polypeptide does not include a hemagglutinin epitope (e.g., PKYVKQNTLKLAT; SEQ ID NO:19); instead, the epitope is substituted with a different epitope.

2) 1601. In some cases, a single-chain TMAPP of the present disclosure comprises, in order from N-terminus to C-terminus: i) an epitope; ii) an HLA 31 polypeptide; iii) an HLA α1 polypeptide; iv) an HLA α2 polypeptide; v) a first immunomodulatory polypeptide (e.g., a variant immunomodulatory polypeptide with reduced affinity for its cognate co-immunomodulatory polypeptide); vi) a second immunomodulatory polypeptide (e.g., a variant immunomodulatory polypeptide with reduced affinity for its cognate co-immunomodulatory polypeptide); and vii) an Ig Fc polypeptide. As one non-limiting example, a single-chain TMAPP of the present disclosure can comprise, in order from N-terminus to C-terminus: i) an epitope; ii) an HLA DRB1 β1 polypeptide; iii) an HLA DRA α1 polypeptide; iv) an HLA DRA α2 polypeptide; v) a first immunomodulatory polypeptide (e.g., a variant IL-2 polypeptide comprising H16A and F42A substitutions); vi) a second immunomodulatory polypeptide (e.g., a variant IL-2 polypeptide comprising H16A and F42A substitutions); and vii) an IgG1 Fc polypeptide. In some cases, the epitope is a hemagglutinin epitope (e.g., PKYVKQNTLKLAT; SEQ ID NO:19). In some cases, the HLA DRB1 β1 polypeptide comprises the following amino acid sequence: DTRPRFLWQHKFECHFFNGTERVRLLERCIYNQEESVRFDSDVGEYRAVTELGRPDAEY WNSQKDLLEQRRAAVDTYCRHNYGVGESFTVQRRVEP (SEQ ID NO:106). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGAL ANIAVDKANLEIMTKRSNYTPITN (SEQ ID NO:90). In some cases, the HLA DRA α2 polypeptide comprises the following amino acid sequence: VPPEVTVLTNSPVELREPNVLICFIDKFTPPVVNVTWLRNGKPVTTGVSETVFLPREDHL FRKFHYLPFLPSTEDVYDCRVEHWGLDEPLLKHWEFDA (SEQ ID NO:94). In some cases, the variant IL-2 polypeptide comprises the following amino acid sequence: APTSSSTKKTQLQLEALLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:88), where the H16A and F42A substitutions are underlined. In some cases, the IgG1 Fc polypeptide comprises the following amino acid sequence: DKTHTCPPCPAPELLGGPSVFLFPPKPKDTLMISRTPEVTCVVVDVSHEDPEVKFNWYV DGVEVHNAKTKPREEQYASTYRVVSVLTVLHQDWLNGKEYKCKVSNKALPAPIEKTIS KAKGQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO:93). In some cases, the single-chain polypeptide comprises the amino acid sequence depicted in FIG. 29A, without the leader peptide and without the C-terminal linker and histidine tag. For example, in some cases, the single-chain polypeptide comprises amino acids 21-876 of the amino acid sequence depicted in FIG. 29A.

### Immunomodulatory Polypeptides

Immunomodulatory polypeptides that are suitable for inclusion in a TMAPP of the present disclosure include, but are not limited to, IL-2, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, and HVEM.

In some cases, the immunomodulatory polypeptide is selected from a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, and a PD-L2 polypeptide. The immunomodulatory polypeptide can comprise only the extracellular portion of a full-length immunomodulatory polypeptide. Thus, for example, the immunomodulatory polypeptide can in some cases exclude one or more of a signal peptide, a transmembrane domain, and an intracellular domain normally found in a naturally-occurring immunomodulatory polypeptide.

In some cases, an immunomodulatory polypeptide suitable for inclusion in a TMAPP of the present disclosure comprises all or a portion of (e.g., an extracellular portion of) the amino acid sequence of a naturally-occurring immunomodulatory polypeptide. In other instances, an immunomodulatory polypeptide suitable for inclusion in a TMAPP of the present disclosure is a variant immunomodulatory polypeptide that comprises at least one amino acid substitution compared to the amino acid sequence of a naturally-occurring immunomodulatory polypeptide. In some instances, a variant immunomodulatory polypeptide exhibits a binding affinity for a co-immunomodulatory polypeptide that is lower than the affinity of a corresponding naturally-occurring immunomodulatory polypeptide (e.g., an immunomodulatory polypeptide not comprising the amino acid substitution(s) present in the variant) for the co-immunomodulatory polypeptide.

Variant Immunomodulatory Polypeptides with Reduced Affinity

Suitable immunomodulatory domains that exhibit reduced affinity for a co-immunomodulatory domain can have from 1 amino acid (aa) to 20 aa differences from a wild-type immunomodulatory domain. For example, in some cases, a variant immunomodulatory polypeptide present in a TMAPP of the present disclosure differs in amino acid sequence by 1 aa, 2 aa, 3 aa, 4 aa, 5 aa, 6 aa, 7 aa, 8 aa, 9 aa, or 10 aa, from a corresponding wild-type immunomodulatory polypeptide. As another example, in some cases, a variant immunomodulatory polypeptide present in a TMAPP of the present disclosure differs in amino acid sequence by 11 aa, 12 aa, 13 aa, 14 aa, 15 aa, 16 aa, 17 aa, 18 aa, 19 aa, or 20 aa, from a corresponding wild-type immunomodulatory polypeptide. As an example, in some cases, a variant immunomodulatory polypeptide present in a TMAPP of the present disclosure includes 1, 2, 3, 4, 5, 6, 7, 8, 9, or 10 amino acid substitutions, compared to a corresponding reference (e.g., wild-type) immunomodulatory polypeptide. In some cases, a variant immunomodulatory polypeptide present in a TMAPP of the present disclosure includes a single amino acid substitution compared to a corresponding reference (e.g., wild-type) immunomodulatory polypeptide. In some cases, a variant immunomodulatory polypeptide present in a TMAPP of the present disclosure includes 2 amino acid substitutions (e.g., no more than 2 amino acid substitutions) compared to a corresponding reference (e.g., wild-type) immunomodulatory polypeptide. In some cases, a variant immunomodulatory polypeptide present in a TMAPP of the present disclosure includes 3 amino acid substitutions (e.g., no more than 3 amino acid substitutions) compared to a corresponding reference (e.g., wild-type) immunomodulatory polypeptide. In some cases, a variant immunomodulatory polypeptide present in a TMAPP of the present disclosure includes 4 amino acid substitutions (e.g., no more than 4 amino acid substitutions) compared to a corresponding reference (e.g., wild-type) immunomodulatory polypeptide. In some cases, a variant immunomodulatory polypeptide present in a TMAPP of the present disclosure includes 5 amino acid substitutions (e.g., no more than 5 amino acid substitutions) compared to a corresponding reference (e.g., wild-type) immunomodulatory polypeptide. In some cases, a variant immunomodulatory polypeptide present in a TMAPP of the present disclosure includes 6 amino acid substitutions (e.g., no more than 6 amino acid substitutions) compared to a corresponding reference (e.g., wild-type) immunomodulatory polypeptide. In some cases, a variant immunomodulatory polypeptide present in a TMAPP of the present disclosure includes 7 amino acid substitutions (e.g., no more than 7 amino acid substitutions) compared to a corresponding reference (e.g., wild-type) immunomodulatory polypeptide. In some cases, a variant immunomodulatory polypeptide present in a TMAPP of the present disclosure includes 8 amino acid substitutions (e.g., no more than 8 amino acid substitutions) compared to a corresponding reference (e.g., wild-type) immunomodulatory polypeptide. In some cases, a variant immunomodulatory polypeptide present in a TMAPP of the present disclosure includes 9 amino acid substitutions (e.g., no more than 9 amino acid substitutions) compared to a corresponding reference (e.g., wild-type) immunomodulatory polypeptide. In some cases, a variant immunomodulatory polypeptide present in a TMAPP of the present disclosure includes 10 amino acid substitutions (e.g., no more than 10 amino acid substitutions) compared to a corresponding reference (e.g., wild-type) immunomodulatory polypeptide.

In some cases, a variant immunomodulatory polypeptide present in a TMAPP of the present disclosure includes 11 amino acid substitutions (e.g., no more than 11 amino acid substitutions) compared to a corresponding reference (e.g., wild-type) immunomodulatory polypeptide. In some cases, a variant immunomodulatory polypeptide present in a TMAPP of the present disclosure includes 12 amino acid substitutions (e.g., no more than 12 amino acid substitutions) compared to a corresponding reference (e.g., wild-type) immunomodulatory polypeptide. In some cases, a variant immunomodulatory polypeptide present in a TMAPP of the present disclosure includes 13 amino acid substitutions (e.g., no more than 13 amino acid substitutions) compared to a corresponding reference (e.g., wild-type) immunomodulatory polypeptide. In some cases, a variant immunomodulatory polypeptide present in a TMAPP of the present disclosure includes 14 amino acid substitutions (e.g., no more than 14 amino acid substitutions) compared to a corresponding reference (e.g., wild-type) immunomodulatory polypeptide. In some cases, a variant immunomodulatory polypeptide present in a TMAPP of the present disclosure includes 15 amino acid substitutions (e.g., no more than 15 amino acid substitutions) compared to a corresponding reference (e.g., wild-type) immunomodulatory polypeptide. In some cases, a variant immunomodulatory polypeptide present in a TMAPP of the present disclosure includes 16 amino acid substitutions (e.g., no more than 16 amino acid substitutions) compared to a corresponding reference (e.g., wild-type) immunomodulatory polypeptide. In some cases, a variant immunomodulatory polypeptide present in a TMAPP of the present disclosure includes 17 amino acid substitutions (e.g., no more than 17 amino acid substitutions) compared to a corresponding reference (e.g., wild-type) immunomodulatory polypeptide. In some cases, a variant immunomodulatory polypeptide present in a TMAPP of the present disclosure includes 18 amino acid substitutions (e.g., no more than 18 amino acid substitutions) compared to a corresponding reference (e.g., wild-type) immunomodulatory polypeptide. In some cases, a variant immunomodulatory polypeptide present in a TMAPP of the present disclosure includes 19 amino acid substitutions (e.g., no more than 19 amino acid substitutions) compared to a corresponding reference (e.g., wild-type) immunomodulatory polypeptide. In some cases, a variant immunomodulatory polypeptide present in a TMAPP of the present disclosure includes 20 amino acid substitutions (e.g., no more than 20 amino acid substitutions) compared to a corresponding reference (e.g., wild-type) immunomodulatory polypeptide.

As discussed above, a variant immunomodulatory polypeptide suitable for inclusion in a TMAPP of the present disclosure exhibits reduced affinity for a cognate co-immunomodulatory polypeptide, compared to the affinity of a corresponding wild-type immunomodulatory polypeptide for the cognate co-immunomodulatory polypeptide.

Exemplary pairs of immunomodulatory polypeptide and cognate co-immunomodulatory polypeptide include, but are not limited to:

a) 4-1BBL (immunomodulatory polypeptide) and 4-1BB (cognate co-immunomodulatory polypeptide);

b) PD-L1 (immunomodulatory polypeptide) and PD1 (cognate co-immunomodulatory polypeptide);

c) IL-2 (immunomodulatory polypeptide) and IL-2 receptor (cognate co-immunomodulatory polypeptide);

d) CD80 (immunomodulatory polypeptide) and CD28 (cognate co-immunomodulatory polypeptide);

e) CD86 (immunomodulatory polypeptide) and CD28 (cognate co-immunomodulatory polypeptide);

f) OX40L (CD252) (immunomodulatory polypeptide) and OX40 (CD134) (cognate co-immunomodulatory polypeptide);

g) Fas ligand (immunomodulatory polypeptide) and Fas (cognate co-immunomodulatory polypeptide);

h) ICOS-L (immunomodulatory polypeptide) and ICOS (cognate co-immunomodulatory polypeptide);

i) ICAM (immunomodulatory polypeptide) and LFA-1 (cognate co-immunomodulatory polypeptide);

j) CD30L (immunomodulatory polypeptide) and CD30 (cognate co-immunomodulatory polypeptide);

k) CD40 (immunomodulatory polypeptide) and CD40L (cognate co-immunomodulatory polypeptide);

1) CD83 (immunomodulatory polypeptide) and CD83L (cognate co-immunomodulatory polypeptide);

m) HVEM (CD270) (immunomodulatory polypeptide) and CD160 (cognate co-immunomodulatory polypeptide);

n) JAG1 (CD339) (immunomodulatory polypeptide) and Notch (cognate co-immunomodulatory polypeptide);

o) JAG1 (immunomodulatory polypeptide) and CD46 (cognate co-immunomodulatory polypeptide);

p) CD80 (immunomodulatory polypeptide) and CTLA4 (cognate co-immunomodulatory polypeptide);

q) CD86 (immunomodulatory polypeptide) and CTLA4 (cognate co-immunomodulatory polypeptide); and

r) CD70 (immunomodulatory polypeptide) and CD27 (cognate co-immunomodulatory polypeptide).

In some cases, a variant immunomodulatory polypeptide present in a TMAPP of the present disclosure has a binding affinity for a cognate co-immunomodulatory polypeptide that is from 100 nM to 100 μM. For example, in some cases, a variant immunomodulatory polypeptide present in a TMAPP of the present disclosure has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

### Determining Binding Affinity

Binding affinity between an immunomodulatory polypeptide and its cognate co-immunomodulatory polypeptide can be determined by bio-layer interferometry (BLI) using purified immunomodulatory polypeptide and purified cognate co-immunomodulatory polypeptide. Binding affinity between a TMAPP and its cognate co-immunomodulatory polypeptide can also be determined by BLI using purified TMAPP and the cognate co-immunomodulatory polypeptide. BLI methods are well known to those skilled in the art. See, e.g., Lad et al. (2015) J. Biomol. Screen. 20(4):498-507; and Shah and Duncan (2014) J. Vis. Exp. 18:e51383. The specific and relative binding affinities described in this disclosure between an immunomodulatory polypeptide and its cognate co-immunomodulatory polypeptide, or between a synTac and its cognate co-immunomodulatory polypeptide, can be determined using the following procedures.

To determine binding affinity between a TMAPP and its cognate co-immunomodulatory polypeptide, a BLI assay can be carried out using an Octet RED 96 (Pal FortéBio) instrument, or a similar instrument, as follows. A TMAPP (e.g., a TMAPP of the present disclosure; a control TMAPP (where a control TMAPP comprises a wild-type immunomodulatory polypeptide)) is immobilized onto an insoluble support (a “biosensor”). The immobilized TMAPP is the “target.” Immobilization can be effected by immobilizing a capture antibody onto the insoluble support, where the capture antibody immobilizes the TMAPP. For example, immobilization can be effected by immobilizing anti-Fc (e.g., anti-human IgG Fc) antibodies onto the insoluble support, where the immobilized anti-Fc antibodies bind to and immobilize the TMAPP (where the TMAPP comprises an IgFc polypeptide). A co-immunomodulatory polypeptide is applied, at several different concentrations, to the immobilized TMAPP, and the instrument's response recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the co-immunomodulatory polypeptide to the immobilized TMAPP is conducted at 30° C. As a positive control for binding affinity, an anti-MHC Class II monoclonal antibody can be used. For example, an anti-HLD-DR3 monoclonal antibody such as the 16-23 antibody (Sigma; also referred to as “16.23”; see, e.g., Pious et al. (1985) J. Exp. Med. 162:1193; Mellins et al. (1991) J. Exp. Med. 174:1607; ECACC hybridoma collection 16-23, ECACC 99043001) can be used as a positive control for binding affinity. As another example, a pan-HLA Class II antibody, such as the HKB1 antibody (Immunotools; Holte et al. (1989) Eur. J. Immunol. 19:1221) can be used as a positive control for binding affinity. A standard curve can be generated using serial dilutions of the anti-MHC Class II monoclonal antibody. The co-immunomodulatory polypeptide, or the anti-MHC Class II mAb, is the “analyte.” BLI analyzes the interference pattern of white light reflected from two surfaces: i) from the immobilized polypeptide (“target”); and ii) an internal reference layer. A change in the number of molecules (“analyte”; e.g., co-immunomodulatory polypeptide; anti-HLA antibody) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d}/ₐ) gives rise to the affinity constant K_{D}.

As noted above, determining binding affinity between an immunomodulatory polypeptide (e.g., IL-2 or an IL-2 variant) and its cognate co-immunomodulatory polypeptide (e.g., IL-2R) also can be determined by BLI. The assay is similar to that described above for the TMAPP. A BLI assay can be carried out using an Octet RED 96 (Pal FortéBio) instrument, or a similar instrument, as follows. A component immunomodulatory polypeptide of a TMAPP of the present disclosure (e.g., a variant IL-2 polypeptide of the present disclosure); and a control immunomodulatory polypeptide (where a control immunomodulatory polypeptide comprises a wild-type immunomodulatory polypeptide, e.g. wild-type IL-2)) are immobilized onto an insoluble support (a “biosensor”). The immunomodulatory polypeptide is the “target.” Immobilization can be effected by immobilizing a capture antibody onto the insoluble support, where the capture antibody immobilizes the immunomodulatory polypeptide. For example, if the target is fused to an immuno-affinity tag (e.g. FLAG, human IgG Fc) immobilization can be effected by immobilizing with the appropriate antibody to the immuno-affinity tag (e.g. anti-human IgG Fc) onto the insoluble support, where the immobilized antibodies bind to and immobilize the immunomodulatory polypeptide (where the immunomodulatory polypeptide comprises an IgFc polypeptide). A co-immunomodulatory polypeptide (or polypeptides) is applied, at several different concentrations, to the immobilized immunomodulatory polypeptide, and the instrument's response recorded. Alternatively, a co-immunomodulatory polypeptide (or polypeptides) is immobilized to the biosensor (e.g., for the IL-2 receptor heterotrimer, as a monomeric subunit, heterodimeric subcomplex, or the complete heterotrimer) and the immunomodulatory polypeptide is applied, at several different concentrations, to the immobilized coimmunomodulatory polypeptide(s), and the instrument's response is recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the co-immunomodulatory polypeptide to the immobilized immunomodulatory polypeptide is conducted at 30° C. BLI analyzes the interference pattern of white light reflected from two surfaces: i) from the immobilized polypeptide (“target”); and ii) an internal reference layer. A change in the number of molecules (“analyte”; e.g., co-immunomodulatory polypeptide) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d}/ₐ) gives rise to the affinity constant K_{D}. Determining the binding affinity of both a wild-type immunomodulatory polypeptide (e.g., IL-2) for its receptor (e.g., IL-2R) and a variant immunomodulatory polypeptide (e.g., an IL-2 variant as disclosed herein) for its cognate co-immunomodulatory polypeptide (e.g., its receptor) (e.g., IL-2R) thus allows one to determine the relative binding affinity of the variant co-immunomodulatory polypeptide, as compared to the wild-type co-immunomodulatory polypeptide, for the cognate co-immunomodulatory polypeptide. That is, one can determine whether the binding affinity of a variant immunomodulatory polypeptide for its receptor (its cognate co-immunomodulatory polypeptide) is reduced as compared to the binding affinity of the wild-type immunomodulatory polypeptide for the same cognate co-immunomodulatory polypeptide, and, if so, what is the percentage reduction from the binding affinity of the wild-type co-immunomodulatory polypeptide.

The BLI assay is carried out in a multi-well plate. To run the assay, the plate layout is defined, the assay steps are defined, and biosensors are assigned in Octet Data Acquisition software. The biosensor assembly is hydrated. The hydrated biosensor assembly and the assay plate are equilibrated for 10 minutes on the Octet instrument. Once the data are acquired, the acquired data are loaded into the Octet Data Analysis software. The data are processed in the Processing window by specifying method for reference subtraction, y-axis alignment, inter-step correction, and Savitzky-Golay filtering. Data are analyzed in the Analysis window by specifying steps to analyze (Association and Dissociation), selecting curve fit model (1:1), fitting method (global), and window of interest (in seconds). The quality of fit is evaluated. K_{D} values for each data trace (analyte concentration) can be averaged if within a 3-fold range. K_{D} error values should be within one order of magnitude of the affinity constant values; R² values should be above 0.95. See, e.g., Abdiche et al. (2008) J. Anal. Biochem. 377:209.

In some cases, the ratio of: i) the binding affinity of a control TMAPP (where the control TMAPP comprises a wild-type immunomodulatory polypeptide) to a cognate co-immunomodulatory polypeptide to ii) the binding affinity of a TMAPP of the present disclosure comprising a variant of the wild-type immunomodulatory polypeptide to the cognate co-immunomodulatory polypeptide, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, the ratio of: i) the binding affinity of a control TMAPP (where the control TMAPP comprises a wild-type immunomodulatory polypeptide) to a cognate co-immunomodulatory polypeptide to ii) the binding affinity of a TMAPP of the present disclosure comprising a variant of the wild-type immunomodulatory polypeptide to the cognate co-immunomodulatory polypeptide, when measured by BLI, is in a range of from 1.5:1 to 10⁶:1, e.g., from 1.5:1 to 10:1, from 10:1 to 50:1, from 50:1 to 10²:1, from 102:1 to 10³:1, from 10³:1 to 10⁴:1, from 10⁴:1 to 10⁵:1, or from 10⁵:1 to 10⁶:1.

The epitope present in a TMAPP of the present disclosure binds to a T-cell receptor (TCR) on a T cell with an affinity of at least 100 μM (e.g., at least 10 μM, at least 1 μM, at least 100 nM, at least 10 nM, or at least 1 nM). In some cases, the epitope present in a TMAPP of the present disclosure binds to a TCR on a T cell with an affinity of from about 10⁻⁴ M to about 5×10⁻⁴ M, from about 5×10⁻⁴ M to about 10 M, from about 10 M to 5×10 M, from about 5×10⁻⁵ M to 10⁻⁶ M, from about 10⁻⁶ M to about 5×10⁻⁶ M, from about 5×10⁻⁶ M to about 10⁻⁷ M, from about 10⁻⁷ M to about 5×10⁻⁷ M, from about 5×10⁻⁷ M to about 10⁻⁸ M, or from about 10⁻⁸ M to about 10⁻⁹ M. Expressed another way, in some cases, the epitope present in a TMAPP of the present disclosure binds to a TCR on a T cell with an affinity of from about 1 nM to about 5 nM, from about 5 nM to about 10 nM, from about 10 nM to about 50 nM, from about 50 nM to about 100 nM, from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, from about 75 μM to about 100 μM.

In some cases, a variant immunomodulatory polypeptide present in a TMAPP of the present disclosure has a binding affinity for a cognate co-immunomodulatory polypeptide that is from 1 nM to 100 nM, or from 100 nM to 100 μM. For example, in some cases, a variant immunomodulatory polypeptide present in a TMAPP of the present disclosure has a binding affinity for a cognate co-immunomodulatory polypeptide that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM. In some cases, a variant immunomodulatory polypeptide present in a TMAPP of the present disclosure has a binding affinity for a cognate co-immunomodulatory polypeptide that is from about 1 nM to about 5 nM, from about 5 nM to about 10 nM, from about 10 nM to about 50 nM, from about 50 nM to about 100 nM.

### PD-L1 Variants

As one non-limiting example, in some cases, a variant immunomodulatory polypeptide present in a TMAPP of the present disclosure is a variant PD-L1 polypeptide. Wild-type PD-L1 binds to PD1.

A wild-type human PD-L1 polypeptide can comprise the following amino acid sequence: MRIFAVFIFM TYWHLLNAFT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKICLT LSPST (SEQ ID NO: 1).

A wild-type human PD-L1 ectodomain can comprise the following amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:2).

A wild-type PD-1 polypeptide can comprise the following amino acid sequence:

(SEQ ID NO: 3)
PGWFLDSPDR PWNPPTFSPA LLVVTEGDNA TFTCSFSNTS
ESFVLNWYRM SPSNQTDKLA AFPEDRSQPG QDCRFRVTQL
PNGRDFHMSV VRARRNDSGT YLCGAISLAP KAQIKESLRA
ELRVTERRAE VPTAHPSPSP RPAGQFQTLV VGVVGGLLGS
LVLLVWVLAV ICSRAARGTI GARRTGQPLK EDPSAVPVFS
VDYGELDFQW REKTPEPPVP CVPEQTEYAT IVFPSGMGTS
SPARRGSADG PRSAQPLRPE DGHCSWPL.

In some cases, a variant PD-L1 polypeptide exhibits reduced binding affinity to PD-1 (e.g., a PD-1 polypeptide comprising the amino acid sequence set forth in SEQ ID NO:3), compared to the binding affinity of a PD-L1 polypeptide comprising the amino acid sequence set forth in SEQ ID NO:1 or SEQ ID NO:2. For example, in some cases, a variant PD-L1 polypeptide of the present disclosure binds PD-1 (e.g., a PD-1 polypeptide comprising the amino acid sequence set forth in SEQ ID NO:3) with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a PD-L1 polypeptide comprising the amino acid sequence set forth in SEQ ID NO:1 or SEQ ID NO:2.

In some cases, a variant PD-L1 polypeptide has a binding affinity to PD-1 that is from 1 nM to 1 mM. In some cases, a variant PD-L1 polypeptide of the present disclosure has a binding affinity to PD-1 that is from 100 nM to 100 μM. As another example, in some cases, a variant PD-L1 polypeptide has a binding affinity for PD1 (e.g., a PD1 polypeptide comprising the amino acid sequence set forth in SEQ ID NO:3) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant PD-L1 polypeptide has a single amino acid substitution compared to the PD-L1 amino acid sequence set forth in SEQ ID NO:1 or SEQ ID NO:2. In some cases, a variant PD-L1 polypeptide has from 2 to 10 amino acid substitutions compared to the PD-L1 amino acid sequence set forth in SEQ ID NO:1 or SEQ ID NO:2. In some cases, a variant PD-L1 polypeptide has 2 amino acid substitutions compared to the PD-L1 amino acid sequence set forth in SEQ ID NO:1 or SEQ ID NO:2. In some cases, a variant PD-L1 polypeptide has 3 amino acid substitutions compared to the PD-L1 amino acid sequence set forth in SEQ ID NO:1 or SEQ ID NO:2. In some cases, a variant PD-L1 polypeptide has 4 amino acid substitutions compared to the PD-L1 amino acid sequence set forth in SEQ ID NO:1 or SEQ ID NO:2. In some cases, a variant PD-L1 polypeptide has 5 amino acid substitutions compared to the PD-L1 amino acid sequence set forth in SEQ ID NO:1 or SEQ ID NO:2. In some cases, a variant PD-L1 polypeptide has 6 amino acid substitutions compared to the PD-L1 amino acid sequence set forth in SEQ ID NO:1 or SEQ ID NO:2. In some cases, a variant PD-L1 polypeptide has 7 amino acid substitutions compared to the PD-L1 amino acid sequence set forth in SEQ ID NO:1 or SEQ ID NO:2. In some cases, a variant PD-L1 polypeptide has 8 amino acid substitutions compared to the PD-L1 amino acid sequence set forth in SEQ ID NO:1 or SEQ ID NO:2. In some cases, a variant PD-L1 polypeptide has 9 amino acid substitutions compared to the PD-L1 amino acid sequence set forth in SEQ ID NO:1 or SEQ ID NO:2. In some cases, a variant PD-L1 polypeptide has 10 amino acid substitutions compared to the PD-L1 amino acid sequence set forth in SEQ ID NO:1 or SEQ ID NO:2.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKXLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:108), where X is any amino acid other than Asp. In some cases, X is Ala. In some cases, X is Arg.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKDLYVV EYGSNMTIEC KFPVEKQLDL AALXVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:109), where X is any amino acid other than Ile. In some cases, X is Asp.

A suitable PD-L1 variant includes a polypeptide that comprises an amino acid sequence having at least 90%, at least 95%, at least 98%, at least 99%, or 100%, amino acid sequence identity to the following amino acid sequence:

FT VTVPKDLYVV EYGSNMTIEC KFPVEKQLDL AALIVYWEME DKNIIQFVHG EXDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:110), where X is any amino acid other than Glu. In some cases, X is Arg.

### CD80 Variants

In some cases, a variant immunomodulatory polypeptide present in a TMAPP of the present disclosure is a variant CD80 polypeptide. Wild-type CD80 binds to CD28.

A wild-type amino acid sequence of the ectodomain of human CD80 can be as follows:

(SEQ ID NO: 4)
VIHVTK EVKEVATLSC GHNVSVEELA QTRIYWQKEK KMVLTMMSGD
MNIWPEYKNR TIFDITNNLS IVILALRPSD EGTYECVVLK
YEKDAFKREH LAEVTLSVKA DFPTPSISDF EIPTSNIRRI
ICSTSGGFPE PHLSWLENGE ELNAINTTVS QDPETELYAV
SSKLDFNMTT NHSFMCLIKY GHLRVNQTFN WNTTKQEHFP DN.

A wild-type CD28 amino acid sequence can be as follows: MLRLLLALNL FPSIQVTGNK ILVKQSPMLV AYDNAVNLSC KYSYNLFSRE FRASLHKGLD SAVEVCVVYG NYSQQLQVYS KTGFNCDGKL GNESVTFYLQ NLYVNQTDIY FCKIEVMYPP PYLDNEKSNG TIIHVKGKHL CPSPLFPGPS KPFWVLVVVG GVLACYSLLV TVAFIIFWVR SKRSRLLHSD YMNMTPRRPG PTRKHYQPYA PPRDFAAYRS(SEQID NO:5).

A wild-type CD28 amino acid sequence can be as follows: MLRLLLALNL FPSIQVTGNK ILVKQSPMLV AYDNAVNLSW KHLCPSPLFP GPSKPFWVLV VVGGVLACYS LLVTVAFIIF WVRSKRSRLL HSDYMNMTPR RPGPTRKHYQ PYAPPRDFAA YRS (SEQ ID NO:6)

A wild-type CD28 amino acid sequence can be as follows: MLRLLLALNL FPSIQVTGKH LCPSPLFPGP SKPFWVLVVV GGVLACYSLL VTVAFIIFWV RSKRSRLLHS DYMNMTPRRP GPTRKHYQPY APPRDFAAYR S (SEQ ID NO:7).

In some cases, a variant CD80 polypeptide exhibits reduced binding affinity to CD28, compared to the binding affinity of a CD80 polypeptide comprising the amino acid sequence set forth in SEQ ID NO:4 for CD28. For example, in some cases, a variant CD80 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD80 polypeptide comprising the amino acid sequence set forth in SEQ ID NO:4 for CD28 (e.g., a CD28 polypeptide comprising the amino acid sequence set forth in one of SEQ ID NO:5, 6, or 7).

In some cases, a variant CD80 polypeptide has a binding affinity to CD28 that is from 100 nM to 100 μM. As another example, in some cases, a variant CD80 polypeptide of the present disclosure has a binding affinity for CD28 (e.g., a CD28 polypeptide comprising the amino acid sequence set forth in SEQ ID NO:5, SEQ ID NO:6, or SEQ ID NO:7) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD80 polypeptide has a single amino acid substitution compared to the CD80 amino acid sequence set forth in SEQ ID NO:4. In some cases, a variant CD80 polypeptide has from 2 to 10 amino acid substitutions compared to the CD80 amino acid sequence set forth in SEQ ID NO:4. In some cases, a variant CD80 polypeptide has 2 amino acid substitutions compared to the CD80 amino acid sequence set forth in SEQ ID NO:4. In some cases, a variant CD80 polypeptide has 3 amino acid substitutions compared to the CD80 amino acid sequence set forth in SEQ ID NO:4. In some cases, a variant CD80 polypeptide has 4 amino acid substitutions compared to the CD80 amino acid sequence set forth in SEQ ID NO:4. In some cases, a variant CD80 polypeptide has 5 amino acid substitutions compared to the CD80 amino acid sequence set forth in SEQ ID NO:4. In some cases, a variant CD80 polypeptide has 6 amino acid substitutions compared to the CD80 amino acid sequence set forth in SEQ ID NO:4. In some cases, a variant CD80 polypeptide has 7 amino acid substitutions compared to the CD80 amino acid sequence set forth in SEQ ID NO:4. In some cases, a variant CD80 polypeptide has 8 amino acid substitutions compared to the CD80 amino acid sequence set forth in SEQ ID NO:4. In some cases, a variant CD80 polypeptide has 9 amino acid substitutions compared to the CD80 amino acid sequence set forth in SEQ ID NO:4. In some cases, a variant CD80 polypeptide has 10 amino acid substitutions compared to the CD80 amino acid sequence set forth in SEQ ID NO:4.

Suitable CD80 variants include a polypeptide that comprises an amino acid sequence having at least 90%, at least 95%, at least 98%, at least 99%, or 100%, amino acid sequence identity to any one of the following amino acid sequences:

VIHVTK EVKEVATLSC GHXVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:111), where X is any amino acid other than Asn. In some cases, X is Ala;

VIHVTK EVKEVATLSC GHNVSVEELA QTRIYWQKEK KMVLTMMSGD MNIWPEYKNR TIFDITXNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 112), where X is any amino acid other than Asn. In some cases, X is Ala;

VIHVTK EVKEVATLSC GHNVSVEELA QTRIYWQKEK KMVLTMMSGD MNIWPEYKNR TIFDITNNLS XVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 113), where X is any amino acid other than Ile. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLX YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 114), where X is any amino acid other than Lys. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS XDPETELYAV SSKLDFNMTT NHSFMCLIKY GHLRVNQTFN WNTTKQEHFP DN (SEQ ID NO:115), where X is any amino acid other than Gln. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QXPETELYAV SSKLDFNMTT NHSFMCLIKY GHLRVNQTFN WNTTKQEHFP DN (SEQ ID NO: 116), where X is any amino acid other than Asp. In some cases, X is Ala;

VIHVTK EVKEVATLSC GHNVSVEEXA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 117), where X is any amino acid other than Leu. In some cases, X is Ala;

VIHVTK EVKEVATLSC GHNVSVEELA QTRIXWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 118), where X is any amino acid other than Tyr. In some cases, X is Ala;

VIHVTK EVKEVATLSC GHNVSVEELA QTRIYWXKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 119), where X is any amino acid other than Gln. In some cases, X is Ala;

VIHVTK EVKEVATLSC GHNVSVEELA QTRIYWQKEK KXVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 120), where X is any amino acid other than Met. In some cases, X is Ala;

VIHVTK EVKEVATLSC GHNVSVEELA QTRIYWQKEK KMXLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:121), where X is any amino acid other than Val. In some cases, X is Ala;

VIHVTK EVKEVATLSC GHNVSVEELA QTRIYWQKEK KMVLTMMSGD MNXWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 122), where X is any amino acid other than Ile. In some cases, X is Ala;

VIHVTK EVKEVATLSC GHNVSVEELA QTRIYWQKEK KMVLTMMSGD MNIWPEXKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 123), where X is any amino acid other than Tyr. In some cases, X is Ala;

VIHVTK EVKEVATLSC GHNVSVEELA QTRIYWQKEK KMVLTMMSGD MNIWPEYKNR TIFXITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 124), where X is any amino acid other than Asp. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DXPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:125), where X is any amino acid other than Phe. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVX QDPETELYAV SSKLDFNMTT NHSFMCLIKY GHLRVNQTFN WNTTKQEHFP DN (SEQ ID NO: 126), where X is any amino acid other than Ser. In some cases, X is Ala; and

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTXSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 127), where X is any amino acid other than Pro. In some cases, X is Ala.

### CD86 Variants

In some cases, a variant immunomodulatory polypeptide present in a T TMAPP of the present disclosure is a variant CD86 polypeptide. Wild-type CD86 binds to CD28.

The amino acid sequence of the full ectodomain of a wild-type human CD86 can be as follows:

(SEQ ID NO: 8)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGIMQKSQDNVTELYDVSISLSVSFPDVTSNMTIFCILETD
KTRLLSSPFSIELEDPQPPPDHIP.

The amino acid sequence of the IgV domain of a wild-type human CD86 can be as follows:

(SEQ ID NO: 9)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVL.

In some cases, a variant CD86 polypeptide exhibits reduced binding affinity to CD28, compared to the binding affinity of a CD86 polypeptide comprising the amino acid sequence set forth in SEQ ID NO:8 or SEQ ID NO:9 for CD28. For example, in some cases, a variant CD86 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD86 polypeptide comprising the amino acid sequence set forth in SEQ ID NO:8 or SEQ ID NO:9 for CD28 (e.g., a CD28 polypeptide comprising the amino acid sequence set forth in one of SEQ ID NO:5, 6, or 7).

In some cases, a variant CD86 polypeptide has a binding affinity to CD28 that is from 100 nM to 100 μM. As another example, in some cases, a variant CD86 polypeptide of the present disclosure has a binding affinity for CD28 (e.g., a CD28 polypeptide comprising the amino acid sequence set forth in one of SEQ ID NOs:5, 6, or 7) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD86 polypeptide has a single amino acid substitution compared to the CD86 amino acid sequence set forth in SEQ ID NO:8. In some cases, a variant CD86 polypeptide has from 2 to 10 amino acid substitutions compared to the CD86 amino acid sequence set forth in SEQ ID NO:8. In some cases, a variant CD86 polypeptide has 2 amino acid substitutions compared to the CD86 amino acid sequence set forth in SEQ ID NO:8. In some cases, a variant CD86 polypeptide has 3 amino acid substitutions compared to the CD86 amino acid sequence set forth in SEQ ID NO:8. In some cases, a variant CD86 polypeptide has 4 amino acid substitutions compared to the CD86 amino acid sequence set forth in SEQ ID NO:8. In some cases, a variant CD86 polypeptide has 5 amino acid substitutions compared to the CD86 amino acid sequence set forth in SEQ ID NO:8. In some cases, a variant CD86 polypeptide has 6 amino acid substitutions compared to the CD86 amino acid sequence set forth in SEQ ID NO:8. In some cases, a variant CD86 polypeptide has 7 amino acid substitutions compared to the CD86 amino acid sequence set forth in SEQ ID NO:8. In some cases, a variant CD86 polypeptide has 8 amino acid substitutions compared to the CD86 amino acid sequence set forth in SEQ ID NO:8. In some cases, a variant CD86 polypeptide has 9 amino acid substitutions compared to the CD86 amino acid sequence set forth in SEQ ID NO:8. In some cases, a variant CD86 polypeptide has 10 amino acid substitutions compared to the CD86 amino acid sequence set forth in SEQ ID NO:8.

In some cases, a variant CD86 polypeptide has a single amino acid substitution compared to the CD86 amino acid sequence set forth in SEQ ID NO:9. In some cases, a variant CD86 polypeptide has from 2 to 10 amino acid substitutions compared to the CD86 amino acid sequence set forth in SEQ ID NO:9. In some cases, a variant CD86 polypeptide has 2 amino acid substitutions compared to the CD86 amino acid sequence set forth in SEQ ID NO:9. In some cases, a variant CD86 polypeptide has 3 amino acid substitutions compared to the CD86 amino acid sequence set forth in SEQ ID NO:9. In some cases, a variant CD86 polypeptide has 4 amino acid substitutions compared to the CD86 amino acid sequence set forth in SEQ ID NO:9. In some cases, a variant CD86 polypeptide has 5 amino acid substitutions compared to the CD86 amino acid sequence set forth in SEQ ID NO:9. In some cases, a variant CD86 polypeptide has 6 amino acid substitutions compared to the CD86 amino acid sequence set forth in SEQ ID NO:9. In some cases, a variant CD86 polypeptide has 7 amino acid substitutions compared to the CD86 amino acid sequence set forth in SEQ ID NO:9. In some cases, a variant CD86 polypeptide has 8 amino acid substitutions compared to the CD86 amino acid sequence set forth in SEQ ID NO:9. In some cases, a variant CD86 polypeptide has 9 amino acid substitutions compared to the CD86 amino acid sequence set forth in SEQ ID NO:9. In some cases, a variant CD86 polypeptide has 10 amino acid substitutions compared to the CD86 amino acid sequence set forth in SEQ ID NO:9.

Suitable CD86 variants include a polypeptide that comprises an amino acid sequence having at least 90%, at least 95%, at least 98%, at least 99%, or 100%, amino acid sequence identity to any one of the following amino acid sequences:

APLKIQAYFNETADLPCQFANSQNQSLSELVVFWQDQENLVLNEVYLGKEKFDSVHSKY MXRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:128), where X is any amino acid other than Asn. In some cases, X is Ala;

APLKIQAYFNETADLPCQFANSQNQSLSELVVFWQDQENLVLNEVYLGKEKFDSVHSKY MNRTSFXSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:129), where X is any amino acid other than Asp. In some cases, X is Ala;

APLKIQAYFNETADLPCQFANSQNQSLSELVVFWQDQENLVLNEVYLGKEKFDSVHSKY MNRTSFDSDSXTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:130), where X is any amino acid other than Trp. In some cases, X is Ala;

APLKIQAYFNETADLPCQFANSQNQSLSELVVFWQDQENLVLNEVYLGKEKFDSVHSKY MNRTSFDSDSWTLRLHNLQIKDKGLYQCIIHXKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:131), where X is any amino acid other than His. In some cases, X is Ala;

APLKIQAYFNETADLPCQFANSQNQSLSELVVFWQDQENLVLNEVYLGKEKFDSVHSKY MXRTSFDSDSWTLRLHNLQIKDKGLYQCIIHHKKPTGMIRIHQMNSELSVL (SEQ ID NO:132), where X is any amino acid other than Asn. In some cases, X is Ala;

APLKIQAYFNETADLPCQFANSQNQSLSELVVFWQDQENLVLNEVYLGKEKFDSVHSKY MNRTSFXSDSWTLRLHNLQIKDKGLYQCIIHHKKPTGMIRIHQMNSELSVL (SEQ ID NO:133), where X is any amino acid other than Asp. In some cases, X is Ala;

APLKIQAYFNETADLPCQFANSQNQSLSELVVFWQDQENLVLNEVYLGKEKFDSVHSKY MNRTSFDSDSXTLRLHNLQIKDKGLYQCIIHHKKPTGMIRIHQMNSELSVL (SEQ ID NO:134), where X is any amino acid other than Trp. In some cases, X is Ala;

APLKIQAYFNETADLPCQFANSQNQSLSELVVFWQDQENLVLNEVYLGKEKFDSVHSKY MNRTSFDSDSWTLRLHNLQIKDKGLYQCIIHXKKPTGMIRIHQMNSELSVL (SEQ ID NO:135), where X is any amino acid other than His. In some cases, X is Ala;

APLKIQAYFNETADLPCQFANSQNQSLSELVVFWQDQENLXLNEVYLGKEKFDSVHSKY MNRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:136), where X is any amino acid other than Val. In some cases, X is Ala;

APLKIQAYFNETADLPCQFANSQNQSLSELVVFWQDQENLXLNEVYLGKEKFDSVHSKY MNRTSFDSDSWTLRLHNLQIKDKGLYQCIIHHKKPTGMIRIHQMNSELSVL (SEQ ID NO:137), where X is any amino acid other than Val. In some cases, X is Ala;

APLKIQAYFNETADLPCQFANSQNQSLSELVVFWXDQENLVLNEVYLGKEKFDSVHSKY MNRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:138), where X is any amino acid other than Gln. In some cases, X is Ala;

APLKIQAYFNETADLPCQFANSQNQSLSELVVFWXDQENLVLNEVYLGKEKFDSVHSKY MNRTSFDSDSWTLRLHNLQIKDKGLYQCIIHHKKPTGMIRIHQMNSELSVL (SEQ ID NO:139), where X is any amino acid other than Gln. In some cases, X is Ala;

APLKIQAYFNETADLPCQFANSQNQSLSELVVXWQDQENLVLNEVYLGKEKFDSVHSKY MNRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:140), where X is any amino acid other than Phe. In some cases, X is Ala;

APLKIQAYFNETADLPCQFANSQNQSLSELVVXWQDQENLVLNEVYLGKEKFDSVHSKY MNRTSFDSDSWTLRLHNLQIKDKGLYQCIIHHKKPTGMIRIHQMNSELSVL (SEQ ID NO:141), where X is any amino acid other than Phe. In some cases, X is Ala;

APLKIQAYFNETADLPCQFANSQNQSLSELVVFWQDQENLVLNEVYLGKEKFDSVHSKY MNRTSFDSDSWTXRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:142), where X is any amino acid other than Leu. In some cases, X is Ala;

APLKIQAYFNETADLPCQFANSQNQSLSELVVFWQDQENLVLNEVYLGKEKFDSVHSKY MNRTSFDSDSWTXRLHNLQIKDKGLYQCIIHHKKPTGMIRIHQMNSELSVL (SEQ ID NO:143), where X is any amino acid other than Leu. In some cases, X is Ala;

APLKIQAYFNETADLPCQFANSQNQSLSELVVFWQDQENLVLNEVYLGKEKFDSVHSKX MNRTSFDSDSWTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:144), where X is any amino acid other than Tyr. In some cases, X is Ala;

APLKIQAYFNETADLPCQFANSQNQSLSELVVFWQDQENLVLNEVYLGKEKFDSVHSKX MNRTSFDSDSWTLRLHNLQIKDKGLYQCIIHHKKPTGMIRIHQMNSELSVL (SEQ ID NO:145), where X is any amino acid other than Tyr. In some cases, X is Ala;

APLKIQAYFNETADLPCQFANSQNQSLSELVVFWQDQENLVLNEVYLGKEKFDSVHSKY MXRTSFDSDSWTLRLHNLQIKDKGLYQCIIHXKKPTGMIRIHQMNSELSVLANFSQPEIVPISN ITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:146), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSVHSKY MXRTSFDSDSWTLRLHNLQIKDKGLYQCIIHXKKPTGMIRIHQMNSELSVL (SEQ ID NO:147), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSVHSKY MNRTSF [symbol] SDSWTLRLHNLQIKDKGLYQCIIH [symbol] KKPTGMIRIHQMNSELSVLANFSQPEIVPIS NITENVYINLTCSSIHGYPEPKKMSVLLRTKNSTIEYDGIMQKSQDNVTELYDVSISLSVSFPD VTSNMTIFCILETDKTRLLSSPFSIELEDPQPPPDHIP (SEQ ID NO:148), where X₁ is any amino acid other than Asp, and X₂ is any amino acid other than His. In some cases, X₁ is Ala and X₂ is Ala;

APLKIQAYFNETADLPCQFANSQNQSLSELVVFWQDQENLVLNEVYLGKEKFDSVHSKY MNRTSF [symbol] SDSWTLRLHNLQIKDKGLYQCIIH [symbol] KKPTGMIRIHQMNSELSVL (SEQ ID NO: 149), where the first X is any amino acid other than Asn and the second X is any amino acid other than His. In some cases, the first and the second X are both Ala;

APLKIQAYFNETADLPCQFANSQNQSLSELVVFWQDOENLVLNEVYLGKEKFDSVHSKY M [symbol] RTSF [symbol] SDSWTLRLHNLQIKDKGLYQCIIH [symbol] KKPTGMIRIHQMNSELSVLANFSQPEIVPI SNITENVYINLTCSSIHGYPEPKKMSVLLRTKNSTIEYDGIMQKSQDNVTELYDVSISLSVSFP DVTSNMTIFCILETDKTRLLSSPFSIELEDPQPPPDHIP (SEQ ID NO:150), where X₁ is any amino acid other than Asn, X₂ is any amino acid other than Asp, and X₃ is any amino acid other than His. In some cases, X₁ is Ala, X₂ is Ala, and X₃ is Ala; and

APLKIQAYFNETADLPCQFANSQNQSLSELVVFWQDQENLVLNEVYLGKEKFDSVHSKY M [symbol] RTSF [symbol] SDSWTLRLHNLQIKDKGLYQCIIH [symbol] KKPTGMIRIHQMNSELSVL (SEQ ID NO:151), where X₁ is any amino acid other than Asn, X₂ is any amino acid other than Asp, and X₃ is any amino acid other than His. In some cases, X₁ is Ala, X₂ is Ala, and X₃ is Ala.

### 4-1BBL Variants

In some cases, a variant immunomodulatory polypeptide present in a TMAPP of the present disclosure is a variant 4-1BBL polypeptide. Wild-type 4-1BBL binds to 4-1BB (CD137).

A wild-type 4-1BBL amino acid sequence can be as follows: MEYASDASLD PEAPWPPAPR ARACRVLPWA LVAGLLLLLL LAAACAVFLA CPWAVSGARA SPGSAASPRL REGPELSPDD PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:10).

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOs:1-13, as follows:

(SEQ ID NO: 11)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 12)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.
(SEQ ID NO: 13)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wild-type 4-1BB amino acid sequence can be as follows: MGNSCYNIVA TLLLVLNFER TRSLQDPCSN CPAGTFCDNN RNQICSPCPP NSFSSAGGQR TCDICRQCKG VFRTRKECSS TSNAECDCTP GFHCLGAGCS MCEQDCKQGQ ELTKKGCKDC CFGTFNDQKR GICRPWTNCS LDGKSVLVNG TKERDVVCGP SPADLSPGAS SVTPPAPARE PGHSPQIISF FLALTSTALL FLLFFLTLRF SVVKRGRKKL LYIFKQPFMR PVQTTQEEDG CSCRFPEEEE GGCEL (SEQ ID NO:14).

In some cases, a variant 4-1BBL polypeptide exhibits reduced binding affinity to 4-1BB, compared to the binding affinity of a 4-1BBL polypeptide comprising the amino acid sequence set forth in one of SEQ ID NOs:10⁻¹³. For example, in some cases, a variant 4-1BBL polypeptide of the present disclosure binds 4-1BB with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence set forth in one of SEQ ID NOs:10⁻¹³ for a 4-1BB polypeptide (e.g., a 4-1BB polypeptide comprising the amino acid sequence set forth in SEQ ID NO: 14), when assayed under the same conditions.

In some cases, a variant 4-1BBL polypeptide has a binding affinity to 4-1BB that is from 100 nM to 100 μM. As another example, in some cases, a variant 4-1BBL polypeptide has a binding affinity for 4-1BB (e.g., a 4-1BB polypeptide comprising the amino acid sequence set forth in SEQ ID NO:14) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide has a single amino acid substitution compared to the 4-1BBL amino acid sequence set forth in one of SEQ ID NOs:10⁻¹³. In some cases, a variant 4-1BBL polypeptide has from 2 to 10 amino acid substitutions compared to the 4-1BBL amino acid sequence set forth in one of SEQ ID NOs:10⁻¹³. In some cases, a variant 4-1BBL polypeptide has 2 amino acid substitutions compared to the 4-1BBL amino acid sequence set forth in one of SEQ ID NOs:10⁻¹³. In some cases, a variant 4-1BBL polypeptide has 3 amino acid substitutions compared to the 4-1BBL amino acid sequence set forth in one of SEQ ID NOs:10⁻¹³. In some cases, a variant 4-1BBL polypeptide has 4 amino acid substitutions compared to the 4-1BBL amino acid sequence set forth in one of SEQ ID NOs:10⁻¹³. In some cases, a variant 4-1BBL polypeptide has 5 amino acid substitutions compared to the 4-1BBL amino acid sequence set forth in one of SEQ ID NOs:10⁻¹³. In some cases, a variant 4-1BBL polypeptide has 6 amino acid substitutions compared to the 4-1BBL amino acid sequence set forth in one of SEQ ID NOs:10⁻¹³. In some cases, a variant 4-1BBL polypeptide has 7 amino acid substitutions compared to the 4-1BBL amino acid sequence set forth in one of SEQ ID NOs:10⁻¹³. In some cases, a variant 4-1BBL polypeptide has 8 amino acid substitutions compared to the 4-1BBL amino acid sequence set forth in one of SEQ ID NOs:10⁻¹³. In some cases, a variant 4-1BBL polypeptide has 9 amino acid substitutions compared to the 4-1BBL amino acid sequence set forth in one of SEQ ID NOs:10⁻¹³. In some cases, a variant 4-1BBL polypeptide has 10 amino acid substitutions compared to the 4-1BBL amino acid sequence set forth in one of SEQ ID NOs:10⁻¹³.

Suitable 4-1BBL variants include a polypeptide that comprises an amino acid sequence having at least 90%, at least 95%, at least 98%, at least 99%, or 100%, amino acid sequence identity to any one of the following amino acid sequences:

PAGLLDLRQG MFAQLVAQNV LLIDGPLSWY SDPGLAGVSL TGGLSYXEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:152), where X is any amino acid other than Lys. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWXLTQ GATVLGLFRV TPEIPAGLPS PRSE (SEQ ID NO:153), where X is any amino acid other than Gln. In some cases, X is Ala;

PAGLLDLRQG XFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:154), where X is any amino acid other than Met. In some cases, X is Ala;

PAGLLDLRQG MXAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:155), where X is any amino acid other than Phe. In some cases, X is Ala;

PAGLLDLRQG MFAXLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:156), where X is any amino acid other than Gln. In some cases, X is Ala;

PAGLLDLRQG MFAQXVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:157), where X is any amino acid other than Leu. In some cases, X is Ala;

PAGLLDLRQG MFAQLXAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:158), where X is any amino acid other than Val. In some cases, X is Ala;

PAGLLDLRQG MFAQLVAXNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:159), where X is any amino acid other than Gln. In some cases, X is Ala;

PAGLLDLRQG MFAQLVAQXV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:160), where X is any amino acid other than Asn. In some cases, X is Ala;

PAGLLDLRQG MFAQLVAQNX LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:161), where X is any amino acid other than Val. In some cases, X is Ala;

PAGLLDLRQG MFAQLVAQNV XLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:162), where X is any amino acid other than Leu. In some cases, X is Ala;

PAGLLDLRQG MFAQLVAQNV LXIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:163), where X is any amino acid other than Leu. In some cases, X is Ala;

PAGLLDLRQG MFAQLVAQNV LLXDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:164), where X is any amino acid other than Ile. In some cases, X is Ala;

PAGLLDLRQG MFAQLVAQNV LLIXGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:165), where X is any amino acid other than Asp. In some cases, X is Ala;

PAGLLDLRQG MFAQLVAQNV LLIDXPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:166), where X is any amino acid other than Gly. In some cases, X is Ala;

PAGLLDLRQG MFAQLVAQNV LLIGGXLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:167), where X is any amino acid other than Pro. In some cases, X is Ala;

PAGLLDLRQG MFAQLVAQNV LLIGGPXSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:168), where X is any amino acid other than Leu. In some cases, X is Ala;

PAGLLDLRQG MFAQLVAQNV LLIGGPLXWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:169), where X is any amino acid other than Ser. In some cases, X is Ala;

PAGLLDLRQG MFAQLVAQNV LLIGGPLSXY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 170), where X is any amino acid other than Trp. In some cases, X is Ala;

PAGLLDLRQG MFAQLVAQNV LLIGGPLSWX SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:171), where X is any amino acid other than Tyr. In some cases, X is Ala;

PAGLLDLRQG MFAQLVAQNV LLIGGPLSWY XDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 172), where X is any amino acid other than Ser. In some cases, X is Ala;

PAGLLDLRQG MFAQLVAQNV LLIGGPLSWY SXPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 173), where X is any amino acid other than Asp. In some cases, X is Ala;

PAGLLDLRQG MFAQLVAQNV LLIGGPLSWY SDXGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 174), where X is any amino acid other than Pro. In some cases, X is Ala;

PAGLLDLRQG MFAQLVAQNV LLIGGPLSWY SDPXLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 175), where X is any amino acid other than Gly. In some cases, X is Ala;

PAGLLDLRQG MFAQLVAQNV LLIGGPLSWY SDPGXAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 176), where X is any amino acid other than Leu. In some cases, X is Ala;

PAGLLDLRQG MFAQLVAQNV LLIGGPLSWY SDPGLAXVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 177), where X is any amino acid other than Gly. In some cases, X is Ala;

PAGLLDLRQG MFAQLVAQNV LLIGGPLSWY SDPGLAGXSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 178), where X is any amino acid other than Val. In some cases, X is Ala;

PAGLLDLRQG MFAQLVAQNV LLIGGPLSWY SDPGLAGVXL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 179), where X is any amino acid other than Ser. In some cases, X is Ala;

PAGLLDLRQG MFAQLVAQNV LLIGGPLSWY SDPGLAGVSX TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 180), where X is any amino acid other than Leu. In some cases, X is Ala;

PAGLLDLRQG MFAQLVAQNV LLIGGPLSWY SDPGLAGVSL XGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:181), where X is any amino acid other than Thr. In some cases, X is Ala;

PAGLLDLRQG MFAQLVAQNV LLIGGPLSWY SDPGLAGVSL TXGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 182), where X is any amino acid other than Gly. In some cases, X is Ala;

PAGLLDLRQG MFAQLVAQNV LLIGGPLSWY SDPGLAGVSL TGXLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 183), where X is any amino acid other than Gly. In some cases, X is Ala;

PAGLLDLRQG MFAQLVAQNV LLIGGPLSWY SDPGLAGVSL TGGXSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 184), where X is any amino acid other than Leu. In some cases, X is Ala;

PAGLLDLRQG MFAQLVAQNV LLIGGPLSWY SDPGLAGVSL TGGLXYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 185), where X is any amino acid other than Ser. In some cases, X is Ala;

PAGLLDLRQG MFAQLVAQNV LLIGGPLSWY SDPGLAGVSL TGGLSXKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 186), where X is any amino acid other than Tyr. In some cases, X is Ala;

PAGLLDLRQG MFAQLVAQNV LLIGGPLSWY SDPGLAGVSL TGGLSYKXDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 187), where X is any amino acid other than Glu. In some cases, X is Ala;

PAGLLDLRQG MFAQLVAQNV LLIGGPLSWY SDPGLAGVSL TGGLSYKEXT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:188), where X is any amino acid other than Asp. In some cases, X is Ala;

PAGLLDLRQG MFAQLVAQNV LLIGGPLSWY SDPGLAGVSL TGGLSYKEDX KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 189), where X is any amino acid other than Thr. In some cases, X is Ala;

PAGLLDLRQG MFAQLVAQNV LLIGGPLSWY SDPGLAGVSL TGGLSYKEDT XELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 190), where X is any amino acid other than Lys. In some cases, X is Ala;

PAGLLDLRQG MFAQLVAQNV LLIGGPLSWY SDPGLAGVSL TGGLSYKEDT KXLVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:191), where X is any amino acid other than Glu. In some cases, X is Ala;

PAGLLDLRQG MFAQLVAQNV LLIGGPLSWY SDPGLAGVSL TGGLSYKEDT KELVVAKAGV YYVXFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 192), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFXQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:913), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFFXLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 194), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQXELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 195), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLXLR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 196), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLEXR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 197), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELX RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 198), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR XVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO: 199), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RXVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:200), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVXAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:201), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVVAXEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:202), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGXGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:203), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEXSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:204), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGXGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:205), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVXLPPASS EARNSAFGFQ GRLLHLSAGQ RLGVHLHTEA RARHAWQLTQ GATVLGLFRV TPEIPAGLPS PRSE (SEQ ID NO:206), where X is any amino acid other than Asp. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDXPPASS EARNSAFGFQ GRLLHLSAGQ RLGVHLHTEA RARHAWQLTQ GATVLGLFRV TPEIPAGLPS PRSE (SEQ ID NO:207), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLXPASS EARNSAFGFQ GRLLHLSAGQ RLGVHLHTEA RARHAWQLTQ GATVLGLFRV TPEIPAGLPS PRSE (SEQ ID NO:208), where X is any amino acid other than Pro. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPAXS EARNSAFGFQ GRLLHLSAGQ RLGVHLHTEA RARHAWQLTQ GATVLGLFRV TPEIPAGLPS PRSE (SEQ ID NO:209), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASX EARNSAFGFQ GRLLHLSAGQ RLGVHLHTEA RARHAWQLTQ GATVLGLFRV TPEIPAGLPS PRSE (SEQ ID NO:210), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS XARNSAFGFQ GRLLHLSAGQ RLGVHLHTEA RARHAWQLTQ GATVLGLFRV TPEIPAGLPS PRSE (SEQ ID NO:211), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EAXNSAFGFQ GRLLHLSAGQ RLGVHLHTEA RARHAWQLTQ GATVLGLFRV TPEIPAGLPS PRSE (SEQ ID NO:212), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARXSAFGFQ GRLLHLSAGQ RLGVHLHTEA RARHAWQLTQ GATVLGLFRV TPEIPAGLPS PRSE (SEQ ID NO:213), where X is any amino acid other than Asn. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNXAFGFQ GRLLHLSAGQ RLGVHLHTEA RARHAWQLTQ GATVLGLFRV TPEIPAGLPS PRSE (SEQ ID NO:214), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAXGFQ GRLLHLSAGQ RLGVHLHTEA RARHAWQLTQ GATVLGLFRV TPEIPAGLPS PRSE (SEQ ID NO:215), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGX RLGVHLHTEA RARHAWQLTQ GATVLGLFRV TPEIPAGLPS PRSE (SEQ ID NO:216), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ XLGVHLHTEA RARHAWQLTQ GATVLGLFRV TPEIPAGLPS PRSE (SEQ ID NO:217), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RXGVHLHTEA RARHAWQLTQ GATVLGLFRV TPEIPAGLPS PRSE (SEQ ID NO:218), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLXVHLHTEA RARHAWQLTQ GATVLGLFRV TPEIPAGLPS PRSE (SEQ ID NO:219), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGXHLHTEA RARHAWQLTQ GATVLGLFRV TPEIPAGLPS PRSE (SEQ ID NO:220), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVXLHTEA RARHAWQLTQ GATVLGLFRV TPEIPAGLPS PRSE (SEQ ID NO:221), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHXHTEA RARHAWQLTQ GATVLGLFRV TPEIPAGLPS PRSE (SEQ ID NO:222), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLXTEA RARHAWQLTQ GATVLGLFRV TPEIPAGLPS PRSE (SEQ ID NO:223), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHXEA RARHAWQLTQ GATVLGLFRV TPEIPAGLPS PRSE (SEQ ID NO:224), where X is any amino acid other than Thr. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTXA RARHAWQLTQ GATVLGLFRV TPEIPAGLPS PRSE (SEQ ID NO:225), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA XARHAWQLTQ GATVLGLFRV TPEIPAGLPS PRSE (SEQ ID NO:226), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RAXHAWQLTQ GATVLGLFRV TPEIPAGLPS PRSE (SEQ ID NO:227), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARXAWQLTQ GATVLGLFRV TPEIPAGLPS PRSE (SEQ ID NO:228), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAXQLTQ GATVLGLFRV TPEIPAGLPS PRSE (SEQ ID NO:229), where X is any amino acid other than Trp. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQXTQ GATVLGLFRV TPEIPAGLPS PRSE (SEQ ID NO:230), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLXQ GATVLGLFRV TPEIPAGLPS PRSE (SEQ ID NO:231), where X is any amino acid other than Thr. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTX GATVLGLFRV TPEIPAGLPS PRSE (SEQ ID NO:232), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ XATVLGLFRV TPEIPAGLPS PRSE (SEQ ID NO:233), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GAXVLGLFRV TPEIPAGLPS PRSE (SEQ ID NO:234), where X is any amino acid other than Thr. In some cases, X is Ala; and

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATXLGLFRV TPEIPAGLPS PRSE (SEQ ID NO:235), where X is any amino acid other than Val. In some cases, X is Ala.

IL-2 variants

In some cases, a variant immunomodulatory polypeptide present in a TMAPP of the present disclosure is a variant IL-2 polypeptide. Wild-type IL-2 binds to IL-2 receptor (IL-2R).

A wild-type IL-2 amino acid sequence can be as follows: APTSSSTKKT QLQLEHLLLD LQMILNGINN YKNPKLTRML TFKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:15).

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122: and a gamma chain (IL-2Rγ; also referred to as CD132). Amino acid sequences of human IL-2Rα, IL2Rβ, and IL-2Rγ can be as follows.

Human IL-2Rα:
(SEQ ID NO: 16)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:
(SEQ ID NO: 17)
VNG TSQFTCFYNS RANISCVWSQ DGALQDTSCQ VHAWPDRRRW
NQTCELLPVS QASWACNLIL GAPDSQKLTT VDIVTLRVLC
REGVRWRVMA IQDFKPFENL RLMAPISLQV VHVETHRCNI
SWEISQASHY FERHLEFEAR TLSPGHTWEE APLLTLKQKQ
EWICLETLTP DTQYEFQVRV KPLQGEFTTW SPWSQPLAFR
TKPAALGKDT IPWLGHLLVG LSGAFGFIIL VYLLINCRNT
GPWLKKVLKC NTPDPSKFFS QLSSEHGGDV QKWLSSPFPS
SSFSPGGLAP EISPLEVLER DKVTQLLLQQ DKVPEPASLS
SNHSLTSCFT NQGYFFFHLP DALEIEACQV YFTYDPYSEE
DPDEGVAGAP TGSSPQPLQP LSGEDDAYCT FPSRDDLLLF
SPSLLGGPSP PSTAPGGSGA GEERMPPSLQ ERVPRDWDPQ
PLGPPTPGVP DLVDFQPPPE LVLREAGEEV PDAGPREGVS
FPWSRPPGQG EFRALNARLP LNTDAYLSLQ ELQGQDPTHL V.
Human IL-2Rγ:
(SEQ ID NO: 18)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

In some cases, where a TMAPP of the present disclosure comprises a variant IL-2 polypeptide, a “cognate co-immunomodulatory polypeptide” is an IL-2R comprising polypeptides comprising the amino acid sequences of SEQ ID NO:16, 17, and 18.

In some cases, a variant IL-2 polypeptide exhibits reduced binding affinity to IL-2R, compared to the binding affinity of a IL-2 polypeptide comprising the amino acid sequence set forth in SEQ ID NO:15. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the amino acid sequence set forth in SEQ ID NO:15 for an IL-2R (e.g., an IL-2R comprising polypeptides comprising the amino acid sequence set forth in SEQ ID NOs:16-18), when assayed under the same conditions.

In some cases, a variant IL-2 polypeptide has a binding affinity to IL-2R that is from 100 nM to 100 μM. As another example, in some cases, a variant IL-2 polypeptide has a binding affinity for IL-2R (e.g., an IL-2R comprising polypeptides comprising the amino acid sequence set forth in SEQ ID NOs:16-18) that is from about 100 nM to 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, to about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant IL-2 polypeptide has a single amino acid substitution compared to the IL-2 amino acid sequence set forth in SEQ ID NO:15. In some cases, a variant IL-2 polypeptide has from 2 to 10 amino acid substitutions compared to the IL-2 amino acid sequence set forth in SEQ ID NO:15. In some cases, a variant IL-2 polypeptide has 2 amino acid substitutions compared to the IL-2 amino acid sequence set forth in SEQ ID NO:15. In some cases, a variant IL-2 polypeptide has 3 amino acid substitutions compared to the IL-2 amino acid sequence set forth in SEQ ID NO:15. In some cases, a variant IL-2 polypeptide has 4 amino acid substitutions compared to the IL-2 amino acid sequence set forth in SEQ ID NO:15. In some cases, a variant IL-2 polypeptide has 5 amino acid substitutions compared to the IL-2 amino acid sequence set forth in SEQ ID NO:15. In some cases, a variant IL-2 polypeptide has 6 amino acid substitutions compared to the IL-2 amino acid sequence set forth in SEQ ID NO:15. In some cases, a variant IL-2 polypeptide has 7 amino acid substitutions compared to the IL-2 amino acid sequence set forth in SEQ ID NO:15. In some cases, a variant IL-2 polypeptide has 8 amino acid substitutions compared to the IL-2 amino acid sequence set forth in SEQ ID NO:15. In some cases, a variant IL-2 polypeptide has 9 amino acid substitutions compared to the IL-2 amino acid sequence set forth in SEQ ID NO:15. In some cases, a variant IL-2 polypeptide has 10 amino acid substitutions compared to the IL-2 amino acid sequence set forth in SEQ ID NO:15.

Suitable IL-2 variants include a polypeptide that comprises an amino acid sequence having at least 90%, at least 95%, at least 98%, at least 99%, or 100%, amino acid sequence identity to any one of the following amino acid sequences:

APTSSSTKKT QLQLEHLLLD LQMILNGINN YKNPKLTRML TXKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:236), where X is any amino acid other than Phe. In some cases, X is Ala;

APTSSSTKKT QLQLEHLLLX LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:237), where X is any amino acid other than Asp. In some cases, X is Ala;

APTSSSTKKT QLQLXHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:238), where X is any amino acid other than Glu. In some cases, X is Ala;

APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:239), where X is any amino acid other than His. In some cases, X is Ala. In some cases, X is Arg. In some cases, X is Asn. In some cases, X is Asp. In some cases, X is Cys. In some cases, X is Glu. In some cases, X is Gln. In some cases, X is Gly. In some cases, X is Ile. In some cases, X is Lys. In some cases, X is Leu. In some cases, X is Met. In some cases, X is Phe. In some cases, X is Pro. In some cases, X is Ser. In some cases, X is Thr. In some cases, X is Tyr. In some cases, X is Trp. In some cases, X is Val;

APTSSSTKKT QLQLEHLLLD LQMILNGINN YKNPKLTRML TFKFXMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:240), where X is any amino acid other than Tyr. In some cases, X is Ala;

APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCXSIIS TLT (SEQ ID NO:241), where X is any amino acid other than Gln. In some cases, X is Ala;

APTSSSTKKT QLQLE [symbol] LLLD LQMILNGINN YKNPKLTRML T [symbol] KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:242), where X₁ is any amino acid other than His, and where X₂ is any amino acid other than Phe. In some cases, X₁ is Ala. In some cases, X₂ is Ala. In some cases, X₁ is Ala; and X₂ is Ala;

APTSSSTKKT QLQLEHLLL [symbol] LQMILNGINN YKNPKLTRML T [symbol] KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:243), where X₁ is any amino acid other than Asp; and where X₂ is any amino acid other than Phe. In some cases, X₁ is Ala. In some cases, X₂ is Ala. In some cases, X₁ is Ala; and X₂ is Ala;

APTSSSTKKT QLQL [symbol] HLLL [symbol] LQMILNGINN YKNPKLTRML T [symbol] KFYMPKKA TELKHLQCLE EELKPLEEVL NLAQSKNFHL RPRDLISNIN VIVLELKGSE TTFMCEYADE TATIVEFLNR WITFCQSIIS TLT(SEQ ID NO:244), where X₁ is any amino acid other than Glu; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLE [symbol] LLL [symbol] LQMILNGINN YKNPKLTRML T [symbol] KFYMPKKA TELKHLQCLE EELKPLEEVL NLAQSKNFHL RPRDLISNIN VIVLELKGSE TTFMCEYADE TATIVEFLNR WITFCQSIIS TLT(SEQ ID NO:245), where X₁ is any amino acid other than His; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEHLLL [symbol] LQMILNGINN YKNPKLTRML T [symbol] KFYMPKKA TELKHLQCLE EELKPLEEVL NLAQSKNFHL RPRDLISNIN VIVLELKGSE TTFMCEYADE TATIVEFLNR WITFC [symbol] SIIS TLT (SEQ ID NO:246), where X₁ is any amino acid other than Asp; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEHLLL [symbol] LQMILNGINN YKNPKLTRML T [symbol] KF [symbol] MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:247), where X₁ is any amino acid other than Asp; where X₂ is any amino acid other than Phe; and where X₃ is any amino acid other than Tyr. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLE [symbol] LLL [symbol] LQMILNGINN YKNPKLTRML T [symbol] KF [symbol] MPKKA TELKHLQCLE EELKPLEEVL NLAQSKNFHL RPRDLISNIN VIVLELKGSE [symbol] TTFMCEYADE TATIVEFLNR WITFCQSIIS TLT (SEQ ID NO:248), where X₁ is any amino acid other than His; where X₂ is any amino acid other than Asp; where X₃ is any amino acid other than Phe; and where X₄ is any amino acid other than Tyr. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;

APTSSSTKKT QLQLEHLLL [symbol] LQMILNGINN YKNPKLTRML T [symbol] KF [symbol] MPKKA TELKHLQCLE EELKPLEEVL NLAQSKNFHL RPRDLISNIN VIVLELKGSE TTFMCEYADE TATIVEFLNR WITFC [symbol] SLLS TLT (SEQ ID NO:249), where X₁ is any amino acid other than Asp; where X₂ is any amino acid other than Phe; where X₃ is any amino acid other than Tyr; and where X₄ is any amino acid other than Gln. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;

APTSSSTKKT QLQLE [symbol] LLL [symbol] LQMILNGINN YKNPKLTRML T [symbol] KF [symbol] MPKKA TELKHLQCLE EELKPLEEVL NLAQSKNFHL RPRDLISNIN VIVLELKGSE TTFMCEYADE TATIVEFLNR WITFC [symbol] SIIS TLT (SEQ ID NO:250), where X₁ is any amino acid other than His; where X₂ is any amino acid other than Asp; where X₃ is any amino acid other than Phe; where X₄ is any amino acid other than Tyr; and where X₅ is any amino acid other than Gln. In some cases, X₁ is Ala. In some cases, X₂ is Ala. In some cases, X₃ is Ala. In some cases, X₄ is Ala. In some cases, X₅ is Ala. In some cases, X₁ is Ala; X₂ is Ala; X₃ is Ala; X₄ is Ala; X₅ is Ala; and

[symbol]

APTSSSTKKT QLQLE [symbol] LLLD LQMILNGINN YKNPKLTRML T [symbol] KFYMPKKA TELKHLQCLE EELKPLEEVL NLAQSKNFHL RPRDLISNIN VIVLELKGSE TTFMCEYADE TATIVEFLNR WITFC [symbol] SIIS TLT (SEQ ID NO:251), where X₁ is any amino acid other than His; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

### Dimerizer Pairs

As noted above, in some cases, an antigen-presenting polypeptide of the present disclosure (including a TMAPP of the present disclosure) comprises a dimerizer pair of polypeptides. For example, where an antigen-presenting polypeptide of the present disclosure (including a TMAPP of the present disclosure) is a multimeric polypeptide comprising at least a first and a second polypeptide, in some cases, the first polypeptide comprises a first member of a dimerization pair, and the second polypeptide comprising a second member of the dimerization pair.

Dimerization peptides are known in the art; and any known dimerization peptide is suitable for use. Dimerization peptides include polypeptides of the collectin family (e.g., ACRP30 or ACRP30-like proteins) which contain collagen domains consisting of collagen repeats Gly-Xaa-Xaa. Other dimerization peptides include coiled-coil domains and leucine-zipper domains. A collagen domain can comprise (Gly-Xaa-Xaa)ₙ, where Xaa is any amino acid, and where n is an integer from 10 to 40. In some cases, a collagen domain comprises (Gly-Xaa-Pro)ₙ, where Xaa is any amino acid and n is an integer from 10 to 40. Dimerization peptides are well known in the art; see, e.g., U.S. Patent Publication No. 2003/0138440.

In some cases, a dimerization pair includes two leucine zipper polypeptides that bind to one another. Non-limiting examples of leucine-zipper polypeptides include, e.g., a peptide of any one of the following amino acid sequences: RMKQIEDKIEEILSKIYHIENEIARIKKLIGER (SEQ ID NO:252); LSSIEKKQEEQTSWLIWISNELTLIRNELAQS (SEQ ID NO:253); LSSIEKKLEEITSQLIQISNELTLIRNELAQ (SEQ ID NO:254); LSSIEKKLEEITSQLIQIRNELTLIRNELAQ (SEQ ID NO:255); LSSIEKKLEEITSQLQQIRNELTLIRNELAQ (SEQ ID NO:256); LSSLEKKLEELTSQLIQLRNELTLLRNELAQ (SEQ ID NO:257); ISSLEKKIEELTSQIQQLRNEITLLRNEIAQ (SEQ ID NO:258).

In some cases, a leucine zipper polypeptide comprises the following amino acid sequence: LEIEAAFLERENTALETRVAELRQRVQRLRNRVSQYRTRYGPLGGGK (SEQ ID NO:259).

Additional leucine-zipper polypeptides are known in the art, any of which is suitable for use in an antigen-presenting polypeptide of the present disclosure.

A collagen oligomerization peptide can comprise the following amino acid sequence:

(SEQ ID NO: 260)
VTAFSNMDDMLQKAHLVIEGTFIYLRDSTEFFIRVRDGWKKLQLGELIP
IPADSPPPPALSSNP.

Coiled-coil dimerization peptides are known in the art. For example, a coiled-coil dimerization peptide can be a peptide of any one of the following amino acid sequences:

(SEQ ID NO: 261)
LKSVENRLAVVENQLKTVIEELKTVKDLLSN;
(SEQ ID NO: 262)
LARIEEKLKTIKAQLSEIASTLNMIREQLAQ;
(SEQ ID NO: 263)
VSRLEEKVKTLKSQVTELASTVSLLREQVAQ;
(SEQ ID NO: 264)
IQSEKKIEDISSLIGQIQSEITLIRNEIAQ;
(SEQ ID NO: 265)
LMSLEKKLEELTQTLMQLQNELSMLKNELAQ.

In some cases, a dimerization peptide comprises at least one cysteine residue. Examples include, e.g.: VDLEGSTSNGRQCAGIRL (SEQ ID NO:266); EDDVTTTEELAPALVPPPKGTCAGWMA (SEQ ID NO:267); and GHDQETTTQGPGVLLPLPKGACTGQMA (SEQ ID NO:268).

### Additional Polypeptides

A polypeptide chain of an APP of the present disclosure (including a TMAPP of the present disclosure) can include one or more polypeptides in addition to those described above. Suitable additional polypeptides include epitope tags and affinity domains. The one or more additional polypeptide can be included at the N-terminus of a polypeptide chain of an APP of the present disclosure, at the C-terminus of a polypeptide chain of an APP of the present disclosure, or internally within a polypeptide chain of an APP of the present disclosure.

### Epitope Tag

Suitable epitope tags include, but are not limited to, hemagglutinin (HA; e.g., YPYDVPDYA (SEQ ID NO:269); FLAG (e.g., DYKDDDDK (SEQ ID NO:270); c-myc (e.g., EQKLISEEDL; SEQ ID NO:271), and the like.

### Affinity Domain

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH) (SEQ ID NO:272), HisX6 (HHHHHH) (SEQ ID NO:273), C-myc (EQKLISEEDL) (SEQ ID NO:271), Flag (DYKDDDDK) (SEQ ID NO:270), StrepTag (WSHPQFEK) (SEQ ID NO:274), hemagglutinin, e.g., HA Tag (YPYDVPDYA) (SEQ ID NO:269), glutathione-S-transferase (GST), thioredoxin, cellulose binding domain, RYIRS (SEQ ID NO:275), Phe-His-His-Thr (SEQ ID NO:276), chitin binding domain, S-peptide, T7 peptide, SH2 domain, C-end RNA tag, WEAAAREACCRECCARA (SEQ ID NO:277), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

### Drug Conjugates

A polypeptide chain of an APP of the present disclosure can comprise a small molecule drug linked (e.g., covalently attached) to the polypeptide chain. For example, where an APP of the present disclosure comprises an Fc polypeptide, the Fc polypeptide can comprise a covalently linked small molecule drug. In some cases, the small molecule drug is a cancer chemotherapeutic agent, e.g., a cytotoxic agent. A polypeptide chain of an APP of the present disclosure can comprise a cytotoxic agent linked (e.g., covalently attached) to the polypeptide chain. For example, where an APP of the present disclosure comprises an Fc polypeptide, the Fc polypeptide can comprise a covalently linked cytotoxic agent. Cytotoxic agents include prodrugs.

A drug (e.g., a cancer chemotherapeutic agent) can be linked directly or indirectly to a polypeptide chain of an APP of the present disclosure. For example, where an APP of the present disclosure comprises an Fc polypeptide, a drug (e.g., a cancer chemotherapeutic agent) can be linked directly or indirectly to the Fc polypeptide. Direct linkage can involve linkage directly to an amino acid side chain. Indirect linkage can be linkage via a linker. A drug (e.g., a cancer chemotherapeutic agent) can be linked to a polypeptide chain (e.g., an Fc polypeptide) of an APP of the present disclosure via a thioether bond, an amide bond, a carbamate bond, a disulfide bond, or an ether bond.

Linkers include cleavable linkers and non-cleavable linkers. In some cases, the linker is a protease-cleavable linker. Suitable linkers include, e.g., peptides (e.g., from 2 to 10 amino acids in length; e.g., 2, 3, 4, 5, 6, 7, 8, 9, or 10 amino acids in length), alkyl chains, poly(ethylene glycol), disulfide groups, thioether groups, acid labile groups, photolabile groups, peptidase labile groups, and esterase labile groups. Non-limiting example of suitable linkers are: i) N-succinimidyl-[(N-maleimidopropionamido)-tetraethyleneglycol]ester (NHS-PEG4-maleimide); ii) N-succinimidyl 4-(2-pyridyldithio)butanoate (SPDB); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); K-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

A polypeptide (e.g., an Fc polypeptide) can be modified with crosslinking reagents such as succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS or succinimidyl-iodoacetate, as described in the literature, to introduce 1-10 reactive groups. The modified Fc polypeptide is then reacted with a thiol-containing cytotoxic agent to produce a conjugate.

For example, where an APP of the present disclosure comprises an Fc polypeptide, the polypeptide chain comprising the Fc polypeptide can be of the formula (A)-(L)-(C), where (A) is the polypeptide chain comprising the Fc polypeptide; where (L), if present, is a linker; and where (C) is a cytotoxic agent. (L), if present, links (A) to (C). In some cases, the polypeptide chain comprising the Fc polypeptide can comprise more than one cytotoxic agent (e.g., 2, 3, 4, or 5, or more than 5, cytotoxic agents).

Suitable drugs include, e.g., rapamycin. Suitable drugs include, e.g., retinoids, such as all-trans retinoic acid (ATRA); vitamin D3; a vitamin D3 analog; and the like. As noted above, in some cases, a drug is a cytotoxic agent. Cytotoxic agents are known in the art. A suitable cytotoxic agent can be any compound that results in the death of a cell, or induces cell death, or in some manner decreases cell viability, and includes, for example, maytansinoids and maytansinoid analogs, benzodiazepines, taxoids, CC-1065 and CC-1065 analogs, duocarmycins and duocarmycin analogs, enediynes, such as calicheamicins, dolastatin and dolastatin analogs including auristatins, tomaymycin derivatives, leptomycin derivatives, methotrexate, cisplatin, carboplatin, daunorubicin, doxorubicin, vincristine, vinblastine, melphalan, mitomycin C, chlorambucil and morpholino doxorubicin.

For example, in some cases, the cytotoxic agent is a compound that inhibits microtubule formation in eukaryotic cells. Such agents include, e.g., maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. Maytansinoid compounds include, e.g., N(2′)-deacetyl-N(2′)-(3-mercapto-1-oxopropyl)-maytansine (DM1); N(2′)-deacetyl-N(2′)-(4-mercapto-1-oxopentyl)-maytansine (DM3); and N(2′)-deacetyl-N2-(4-mercapto-4-methyl-1-oxopentyl)-maytansine (DM4). Benzodiazepines include, e.g., indolinobenzodiazepines and oxazolidinobenzodiazepines.

Cytotoxic agents are known in the art. A suitable cytotoxic agent can be any compound that results in the death of a cell, or induces cell death, or in some manner decreases cell viability, and includes, for example, maytansinoids and maytansinoid analogs, benzodiazepines, taxoids, CC-1065 and CC-1065 analogs, duocarmycins and duocarmycin analogs, enediynes, such as calicheamicins, dolastatin and dolastatin analogs including auristatins, tomaymycin derivatives, leptomycin derivatives, methotrexate, cisplatin, carboplatin, daunorubicin, doxorubicin, vincristine, vinblastine, melphalan, mitomycin C, chlorambucil and morpholino doxorubicin.

Cytotoxic agents include taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; maytansine or an analog or derivative thereof; an auristatin or a functional peptide analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite; 6 mercaptopurine; 6 thioguanine; cytarabine; fludarabin; 5 fluorouracil; decarbazine; hydroxyurea; asparaginase; gemcitabine; cladribine; an alkylating agent; a platinum derivative; duocarmycin A; duocarmycin SA; rachelmycin (CC-1065) or an analog or derivative thereof; an antibiotic; pyrrolo[2,1-c][1,4]-benzodiazepines (PDB); diphtheria toxin; ricin toxin; cholera toxin; a Shiga-like toxin; LT toxin; C3 toxin; Shiga toxin; pertussis toxin; tetanus toxin; soybean Bowman-Birk protease inhibitor; Pseudomonas exotoxin; alorin; saporin; modeccin; gelanin; abrin A chain; modeccin A chain; alpha-sarcin; Aleurites fordii proteins; dianthin proteins; Phytolacca americana proteins; Momordica charantia inhibitor; curcin; crotin; Sapaonaria officinalis inhibitor; gelonin; mitogellin; restrictocin; phenomycin; enomycin toxins; ribonuclease (RNase); DNase I; Staphylococcal enterotoxin A; pokeweed antiviral protein; diphtherin toxin; and Pseudomonas endotoxin.

In some cases, the cytotoxic agent is a compound that inhibits microtubule formation in eukaryotic cells. Such agents include, e.g., maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. Maytansinoid compounds include, e.g., N(2′)-deacetyl-N(2′)-(3-mercapto-1-oxopropyl)-maytansine (DM1); N(2′)-deacetyl-N(2′)-(4-mercapto-1-oxopentyl)-maytansine (DM3); and N(2′)-deacetyl-N2-(4-mercapto-4-methyl-1-oxopentyl)-maytansine (DM4). Benzodiazepines include, e.g., indolinobenzodiazepines and oxazolidinobenzodiazepines.

### Nucleic Acids

The present disclosure provides a nucleic acid comprising a nucleotide sequence encoding an APP of the present disclosure. The present disclosure provides a nucleic acid comprising a nucleotide sequence encoding a TMAPP of the present disclosure.

### Nucleic Acids Encoding Single-Chain Antigen-Presenting Polypeptides of the Present Disclosure

As described above, in some cases, an APP of the present disclosure comprises a single polypeptide chain. As described above, in some cases, a TMAPP of the present disclosure comprises a single polypeptide chain. The present disclosure provides a nucleic acid comprising a nucleotide sequence encoding a single-chain APP of the present disclosure (including a single-chain TMAPP of the present disclosure).

### Nucleic Acid(s) Encoding Multimeric Polypeptides of the Present Disclosure

As noted above, in some cases, an APP of the present disclosure (including a TMAPP of the present disclosure) comprises at least 2 separate polypeptide chains. The present disclosure provides nucleic acids comprising nucleotide sequences encoding a multimeric APP (e.g., a multimeric TMAPP) of the present disclosure. In some cases, the individual polypeptide chains of a multimeric polypeptide (a multimeric APP of the present disclosure; a multimeric TMAPP of the present disclosure) of the present disclosure are encoded in separate nucleic acids. In some cases, all polypeptide chains of a multimeric polypeptide of the present disclosure are encoded in a single nucleic acid. In some cases, a first nucleic acid comprises a nucleotide sequence encoding a first polypeptide of a multimeric polypeptide of the present disclosure; and a second nucleic acid comprises a nucleotide sequence encoding a second polypeptide of a multimeric polypeptide of the present disclosure. In some cases, single nucleic acid comprises a nucleotide sequence encoding a first polypeptide of a multimeric polypeptide of the present disclosure and a second polypeptide of a multimeric polypeptide of the present disclosure.

### Separate Nucleic Acids Encoding Individual Polypeptide Chains of a Multimeric Polypeptide

The present disclosure provides nucleic acids comprising nucleotide sequences encoding a multimeric polypeptide of the present disclosure. As noted above, in some cases, the individual polypeptide chains of a multimeric polypeptide of the present disclosure are encoded in separate nucleic acids. In some cases, nucleotide sequences encoding the separate polypeptide chains of a multimeric polypeptide of the present disclosure are operably linked to transcriptional control elements, e.g., promoters, such as promoters that are functional in a eukaryotic cell, where the promoter can be a constitutive promoter or an inducible promoter.

For example, the present disclosure provides a first nucleic acid and a second nucleic acid, where the first nucleic acid comprises a nucleotide sequence encoding the first polypeptide of a multimeric polypeptide of the present disclosure, and where the second nucleic acid comprises a nucleotide sequence encoding the second polypeptide of the multimeric polypeptide. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

As one non-limiting example, the present disclosure provides a first nucleic acid and a second nucleic acid, where the first nucleic acid comprises a nucleotide sequence encoding a first polypeptide of a multimeric polypeptide of the present disclosure, where the first polypeptide comprises, in order from N-terminus to C-terminus: a) an epitope (e.g., a T-cell epitope); b) a first MHC Class II polypeptide; and c) an immunomodulatory polypeptide (e.g., a reduced-affinity variant, as described above); and where the second nucleic acid comprises a nucleotide sequence encoding a second polypeptide of a multimeric polypeptide of the present disclosure, where the second polypeptide comprises, in order from N-terminus to C-terminus: a) a second MHC Class II polypeptide; and b) an Ig Fc polypeptide. Suitable T-cell epitopes, MHC polypeptides, immunomodulatory polypeptides, and Ig Fc polypeptides, are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Nucleic Acid Encoding Two or More Polypeptides Present in a Multimeric Polypeptide

The present disclosure provides a nucleic acid comprising nucleotide sequences encoding at least the first polypeptide and the second polypeptide of a multimeric polypeptide of the present disclosure. In some cases, where a multimeric polypeptide of the present disclosure includes a first, second, and third polypeptide, the nucleic acid includes a nucleotide sequence encoding the first, second, and third polypeptides. In some cases, the nucleotide sequences encoding the first polypeptide and the second polypeptide of a multimeric polypeptide of the present disclosure includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a multimeric polypeptide of the present disclosure includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a multimeric polypeptide of the present disclosure includes a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a multimeric polypeptide of the present disclosure; in any of these embodiments, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a multimeric polypeptide of the present disclosure; and a second nucleic acid (e.g., a recombinant expression vector, an mRNA, a viral RNA, etc.) comprises a nucleotide sequence encoding a second polypeptide chain of a multimeric polypeptide of the present disclosure. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

### Recombinant Expression Vectors

The present disclosure provides recombinant expression vectors comprising nucleic acids of the present disclosure. In some cases, the recombinant expression vector is a non-viral vector. In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some cases, a nucleotide sequence encoding an APP of the present disclosure is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

### Genetically Modified Host Cells

The present disclosure provides a genetically modified host cell, where the host cell is genetically modified with a nucleic acid(s) of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

Genetically modified host cells can be used to produce an APP of the present disclosure. For example, a genetically modified host cell can be used to produce a multimeric TMAPP of the present disclosure, or a single-chain TMAPP of the present disclosure. An expression vector(s) comprising nucleotide sequences encoding the polypeptide(s) is/are introduced into a host cell, generating a genetically modified host cell, which genetically modified host cell produces the polypeptide(s).

### Compositions

The present disclosure provides compositions, including pharmaceutical compositions, comprising an APP of the present disclosure. The present disclosure provides compositions, including pharmaceutical compositions, comprising a TMAPP of the present disclosure. The present disclosure provides compositions, including pharmaceutical compositions, comprising a nucleic acid or a recombinant expression vector of the present disclosure.

### Compositions Comprising an Antigen-Presenting Polypeptide

A composition of the present disclosure can comprise, in addition to an APP of the present disclosure or a TMAPP of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise: i) an APP of the present disclosure or a TMAPP of the present disclosure; and ii) a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include aqueous solution, powder form, granules, tablets, pills, suppositories, capsules, suspensions, sprays, and the like. The composition may be formulated according to the various routes of administration described below.

Where an APP of the present disclosure or a TMAPP of the present disclosure is administered as an injectable (e.g. subcutaneously, intraperitoneally, intramuscularly, intralymphatically, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the subject protein following administration. For example, the protein may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a composition of the present disclosure comprises: a) an APP of the present disclosure; and b) saline (e.g., 0.9% NaCl). In some cases, a composition of the present disclosure comprises: a) a TMAPP of the present disclosure; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) a TMAPP of the present disclosure; and b) saline (e.g., 0.9% NaCl), where the composition is sterile and is free of detectable pyrogens and/or other toxins.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be present in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of an APP of the present disclosure or a TMAPP of the present disclosure in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

### Compositions Comprising a Nucleic Acid or a Recombinant Expression Vector

The present disclosure provides compositions, e.g., pharmaceutical compositions, comprising a nucleic acid or a recombinant expression vector of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) one or more nucleic acids or one or more recombinant expression vectors comprising nucleotide sequences encoding an APP of the present disclosure or a TMAPP of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, in some embodiments, a subject formulation comprises a nucleic acid or recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A formulation comprising a subject nucleic acid or recombinant expression vector can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a subject nucleic acid or recombinant expression vector.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and in emulsions is well known in the art. Surfactants and their uses are further described in U.S. Pat. No. 6,287,860.

In one embodiment, various penetration enhancers are included, to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanoiamine, N,N-dimethylacetamide, N,N-dimethylformamide, 2-pyrrolidone and derivatives thereof, tetrahydrofurfuryl alcohol, and AZONE™.

### Methods

An APP of the present disclosure is useful for various research and diagnostic purposes. For example, an APP of the present disclosure can be used to label, directly or indirectly, an antigen-specific T cell.

A TMAPP of the present disclosure is useful for modulating an activity of a T cell. Thus, the present disclosure provides methods of modulating an activity of a T cell, the methods generally involving contacting a target T cell with a TMAPP of the present disclosure.

### Methods of Detecting an Antigen-Specific T Cell

The present disclosure provides a method of detecting an antigen-specific T-cell. The methods comprise contacting a T cell with an APP of the present disclosure; and detecting binding of the APP to the T cell.

The present disclosure provides a method of detecting an antigen-specific T cell, the method comprising contacting a T cell with an APP of the present disclosure, wherein binding of the APP to the T cell indicates that the T cell is specific for the epitope present in the APP.

In some cases, the APP comprises a detectable label. Suitable detectable labels include, but are not limited to, a radioisotope, a fluorescent polypeptide, or an enzyme that generates a fluorescent product, and an enzyme that generates a colored product. Where the APP comprises a detectable label, binding of the APP to the T cell is detected by detecting the detectable label.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of the APP to the T cell is detected using a detectably labeled antibody specific for the APP. An antibody specific for the APP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

### Methods of Modulating T Cell Activity

The present disclosure provides a method of selectively modulating the activity of an epitope-specific T cell, the method comprising contacting the T cell with a TMAPP of the present disclosure, where contacting the T cell with a TMAPP of the present disclosure selectively modulates the activity of the epitope-specific T cell. In some cases, the contacting occurs in vitro. In some cases, the contacting occurs in vivo. In some cases, the contacting occurs ex vivo.

In some cases, the T cell being contacted with a TMAPP of the present disclosure is a regulatory T cell (Treg). Tregs are CD4⁺, FOXP3⁺, and CD25⁺. Tregs can suppress autoreactive T cells. In some cases, a method of the present disclosure activates Tregs, thereby reducing autoreactive T cell activity.

The present disclosure provides a method of increasing proliferation of Tregs, the method comprising contacting Tregs with a TMAPP of the present disclosure, where the contacting increases proliferation of Tregs. The present disclosure provides a method of increasing the number of Tregs in an individual, the method comprising administering to the individual a TMAPP of the present disclosure, where the administering results in an increase in the number of Tregs in the individual. For example, the number of Tregs can be increased by at least 5%, at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, or more than 10-fold.

In some cases, the cell being contacted is a helper T cell, where contacting the helper T cell with a TMAPP of the present disclosure results in activation of the helper T cell. In some cases, activation of the helper T cell results in an increase in the activity and/or number of CD8⁺ cytotoxic T cells, e.g., CD8⁺ cytotoxic T cells that target and kill a cancer cell.

### Treatment Methods

The present disclosure provides treatment methods, the methods comprising administering to the individual an amount of a TMAPP of the present disclosure, or one or more nucleic acids encoding the TMAPP, effective to selectively modulate the activity of an epitope-specific T cell in an individual and to treat the individual. In some cases, a treatment method of the present disclosure comprises administering to an individual in need thereof one or more recombinant expression vectors comprising nucleotide sequences encoding a TMAPP of the present disclosure. In some cases, a treatment method of the present disclosure comprises administering to an individual in need thereof one or more mRNA molecules comprising nucleotide sequences encoding a TMAPP of the present disclosure. In some cases, a treatment method of the present disclosure comprises administering to an individual in need thereof a TMAPP of the present disclosure. Conditions that can be treated include cancer and autoimmune disorders.

The present disclosure provides a method of selectively modulating the activity of an epitope-specific T cell in an individual, the method comprising administering to the individual an effective amount of a TMAPP of the present disclosure, or one or more nucleic acids (e.g., expression vectors; mRNA; etc.) comprising nucleotide sequences encoding the TMAPP, where the TMAPP selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a TMAPP of the present disclosure (e.g., a multimeric TMAPP of the present disclosure; or a single-chain TMAPP of the present disclosure). In some cases, the disease or disorder is an autoimmune disease or disorder. In some cases, the disease or disorder is cancer.

In some cases, the immunomodulatory polypeptide is an activating polypeptide, and the TMAPP activates the epitope-specific T cell. In some cases, the epitope is a cancer-associated epitope, and the TMAPP (e.g., a multimeric TMAPP of the present disclosure; or a single-chain TMAPP of the present disclosure) increases the activity of a T cell specific for the cancer-associate epitope.

The present disclosure provides a method of treating cancer in an individual, the method comprising administering to the individual an effective amount of a TMAPP of the present disclosure, or one or more nucleic acids (e.g., expression vectors; mRNA; etc.) comprising nucleotide sequences encoding the TMAPP, where the TMAPP comprises a T-cell epitope that is a cancer epitope, and where the multimeric polypeptide comprises a stimulatory immunomodulatory polypeptide. In some cases, an “effective amount” of a TMAPP of the present disclosure is an amount that, when administered in one or more doses to an individual in need thereof, reduces the number of cancer cells in the individual. For example, in some cases, an “effective amount” of a TMAPP of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the TMAPP, or in the absence of administration with the TMAPP. In some cases, an “effective amount” of a TMAPP of the present disclosure is an amount that, when administered in one or more doses to an individual in need thereof, reduces the number of cancer cells in the individual to undetectable levels. In some cases, an “effective amount” of a TMAPP of the present disclosure is an amount that, when administered in one or more doses to an individual in need thereof, reduces the tumor mass in the individual. For example, in some cases, an “effective amount” of a TMAPP of the present disclosure is an amount that, when administered in one or more doses to an individual in need thereof, reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the TMAPP, or in the absence of administration with the TMAPP. In some cases, an “effective amount” of a TMAPP of the present disclosure is an amount that, when administered in one or more doses to an individual in need thereof (an individual having a tumor), reduces the tumor volume in the individual. For example, in some cases, an “effective amount” of a TMAPP of the present disclosure is an amount that, when administered in one or more doses to an individual in need thereof (an individual having a tumor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the TMAPP, or in the absence of administration with the TMAPP. In some cases, an “effective amount” of a TMAPP of the present disclosure is an amount that, when administered in one or more doses to an individual in need thereof, increases survival time of the individual. For example, in some cases, an “effective amount” of a TMAPP of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the TMAPP.

In some cases, the immunomodulatory polypeptide is an inhibitory polypeptide, and a T TMAPP of the present disclosure inhibits activity of the epitope-specific T cell. In some cases, the epitope is a self-epitope, and a TMAPP of the present disclosure selectively inhibits the activity of a T cell specific for the self-epitope.

The present disclosure provides a method of treating an autoimmune disorder in an individual, the method comprising administering to the individual an effective amount of a TMAPP of the present disclosure, or one or more nucleic acids comprising nucleotide sequences encoding the TMAPP, where the TMAPP (e.g., a multimeric TMAPP of the present disclosure; or a single-chain TMAPP of the present disclosure) comprises a T-cell epitope that is a self epitope, and where the TMAPP comprises an inhibitory immunomodulatory polypeptide. In some cases, an “effective amount” of a TMAPP of the present disclosure is an amount that, when administered in one or more doses to an individual in need thereof, reduces the number self-reactive T cells by at least 10%, at least 15%, at least 20%, at least 25%, at least 30%, at least 40%, at least 50%, at least 60%, at least 70%, at least 80%, at least 90%, or at least 95%, compared to number of self-reactive T cells in the individual before administration of the TMAPP, or in the absence of administration with the TMAPP. In some cases, an “effective amount” of a TMAPP of the present disclosure is an amount that, when administered in one or more doses to an individual in need thereof, reduces production of Th2 cytokines in the individual. In some cases, an “effective amount” of a TMAPP of the present disclosure is an amount that, when administered in one or more doses to an individual in need thereof, ameliorates one or more symptoms associated with an autoimmune disease in the individual.

As noted above, in some cases, in carrying out a subject treatment method, a TMAPP of the present disclosure is administered to an individual in need thereof, as the polypeptide per se. In other instances, in carrying out a subject treatment method, one or more nucleic acids comprising nucleotide sequences encoding a TMAPP is/are administering to an individual in need thereof. Thus, in other instances, one or more nucleic acids of the present disclosure, e.g., one or more recombinant expression vectors of the present disclosure, is/are administered to an individual in need thereof.

### Formulations

Suitable formulations are described above, where suitable formulations include a pharmaceutically acceptable excipient. In some cases, a suitable formulation comprises: a) a TMAPP of the present disclosure; and b) a pharmaceutically acceptable excipient. In some cases, a suitable formulation comprises: a) a nucleic acid comprising a nucleotide sequence encoding a TMAPP of the present disclosure; and b) a pharmaceutically acceptable excipient; in some instances, the nucleic acid is an mRNA. In some cases, a suitable formulation comprises: a) a first nucleic acid comprising a nucleotide sequence encoding the first polypeptide of a TMAPP of the present disclosure; b) a second nucleic acid comprising a nucleotide sequence encoding the second polypeptide of a TMAPP of the present disclosure; and c) a pharmaceutically acceptable excipient. In some cases, a suitable formulation comprises: a) a recombinant expression vector comprising a nucleotide sequence encoding a TMAPP of the present disclosure; and b) a pharmaceutically acceptable excipient. In some cases, a suitable formulation comprises: a) a first recombinant expression vector comprising a nucleotide sequence encoding the first polypeptide of a TMAPP of the present disclosure; b) a second recombinant expression vector comprising a nucleotide sequence encoding the second polypeptide of a TMAPP of the present disclosure; and c) a pharmaceutically acceptable excipient.

Suitable pharmaceutically acceptable excipients are described above.

### Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A multimeric polypeptide or a single-chain polypeptide of the present disclosure (e.g., a multimeric TMAPP or a single-chain TMAPP) may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute. A TMAPP of the present disclosure can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a TMAPP of the present disclosure is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a multimeric polypeptide or a single-chain polypeptide of the present disclosure (e.g., a multimeric TMAPP or a single-chain TMAPP) is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

Those of skill will readily appreciate that dose levels can vary as a function of the specific multimeric polypeptide or single-chain polypeptide (multimeric TMAPP or single-chain TMAPP), the severity of the symptoms and the susceptibility of the subject to side effects. Preferred dosages for a given compound are readily determinable by those of skill in the art by a variety of means.

In some cases, multiple doses of a TMAPP of the present disclosure, a nucleic acid of the present disclosure, or a recombinant expression vector of the present disclosure are administered. The frequency of administration of a TMAPP of the present disclosure, a nucleic acid of the present disclosure, or a recombinant expression vector of the present disclosure can vary depending on any of a variety of factors, e.g., severity of the symptoms, etc. For example, in some cases, a TMAPP of the present disclosure, a nucleic acid of the present disclosure, or a recombinant expression vector of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a TMAPP of the present disclosure, a nucleic acid of the present disclosure, or a recombinant expression vector of the present disclosure, e.g., the period of time over which a TMAPP of the present disclosure, a nucleic acid of the present disclosure, or a recombinant expression vector of the present disclosure is administered, can vary, depending on any of a variety of factors, e.g., patient response, etc. For example, a TMAPP of the present disclosure, a nucleic acid of the present disclosure, or a recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

An active agent (a TMAPP of the present disclosure, a nucleic acid of the present disclosure, or a recombinant expression vector of the present disclosure) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intralymphatic, intracranial, subcutaneous, intradermal, topical application, intravenous, intraarterial, rectal, nasal, oral, and other enteral and parenteral routes of administration. Routes of administration may be combined, if desired, or adjusted depending upon the TMAPP and/or the desired effect. A TMAPP of the present disclosure, or a nucleic acid or recombinant expression vector of the present disclosure, can be administered in a single dose or in multiple doses.

In some cases, a TMAPP of the present disclosure, a nucleic acid of the present disclosure, or a recombinant expression vector of the present disclosure is administered intravenously. In some cases, a TMAPP of the present disclosure, a nucleic acid of the present disclosure, or a recombinant expression vector of the present disclosure is administered intramuscularly. In some cases, a TMAPP of the present disclosure, a nucleic acid of the present disclosure, or a recombinant expression vector of the present disclosure is administered intralymphatically. In some cases, a TMAPP of the present disclosure, a nucleic acid of the present disclosure, or a recombinant expression vector of the present disclosure is administered locally. In some cases, a TMAPP of the present disclosure, a nucleic acid of the present disclosure, or a recombinant expression vector of the present disclosure is administered intratumorally. In some cases, a TMAPP of the present disclosure, a nucleic acid of the present disclosure, or a recombinant expression vector of the present disclosure is administered peritumorally. In some cases, a TMAPP of the present disclosure, a nucleic acid of the present disclosure, or a recombinant expression vector of the present disclosure is administered intracranially. In some cases, a TMAPP of the present disclosure, a nucleic acid of the present disclosure, or a recombinant expression vector of the present disclosure is administered subcutaneously.

In some cases, a TMAPP of the present disclosure is administered intravenously. In some cases, a TMAPP of the present disclosure is administered intramuscularly. In some cases, a TMAPP of the present disclosure is administered locally. In some cases, a TMAPP of the present disclosure is administered intratumorally. In some cases, a TMAPP of the present disclosure is administered peritumorally. In some cases, a TMAPP of the present disclosure is administered intracranially. In some cases, a TMAPP of the present disclosure is administered subcutaneously. In some cases, a TMAPP of the present disclosure is administered intralymphatically.

A TMAPP of the present disclosure, a nucleic acid of the present disclosure, or a recombinant expression vector of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, intralymphatic, peritumoral, and intravenous routes, i.e., any route of administration other than through the alimentary canal. Parenteral administration can be carried to effect systemic or local delivery of a TMAPP of the present disclosure, a nucleic acid of the present disclosure, or a recombinant expression vector of the present disclosure. Where systemic delivery is desired, administration typically involves invasive or systemically absorbed topical or mucosal administration of pharmaceutical preparations.

### Subjects Suitable for Treatment

Subjects suitable for treatment with a method of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment.

Subjects suitable for treatment with a method of the present disclosure include individuals who have an autoimmune disease, including individuals who have been diagnosed as having an autoimmune disease, and individuals who have been treated for a autoimmune disease but who failed to respond to the treatment. Autoimmune diseases that can be treated with a method of the present disclosure include, but are not limited to, celiac disease, multiple sclerosis, rheumatoid arthritis, type I autoimmune diabetes (IDDM), Crohn's disease, systemic lupus erythematosus (SLE), autoimmuneencephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, and Sjogren's syndrome.

### Methods of Selectively Delivering a Costimulatory Polypeptide

The present disclosure provides a method of delivering a costimulatory polypeptide such as IL-2, or a reduced-affinity variant of a naturally occurring costimulatory polypeptide such as an IL-2 variant disclosed herein, to a selected T cell or a selected T cell population, e.g., in a manner such that a TCR specific for a given epitope is targeted. The present disclosure provides a method of delivering a costimulatory polypeptide such as IL-2, or a reduced-affinity variant of a naturally occurring costimulatory polypeptide such as an IL-2 variant disclosed herein, selectively to a target T cell bearing a TCR specific for the epitope present in a TMAPP of the present disclosure. The method comprises contacting a population of T cells with a TMAPP of the present disclosure. The population of T cells can be a mixed population that comprises: i) the target T cell; and ii) non-target T cells that are not specific for the epitope (e.g., T cells that are specific for an epitope(s) other than the epitope to which the epitope-specific T cell binds). The epitope-specific T cell is specific for the epitope-presenting peptide present in the TMAPP, and binds to the peptide HLA complex or peptide MHC complex provided by the TMAPP. Contacting the population of T cells with the TMAPP delivers the costimulatory polypeptide (e.g., IL-2 or a reduced-affinity variant of IL-2) present in the TMAPP selectively to the T cell(s) that are specific for the epitope present in the TMAPP.

Thus, the present disclosure provides a method of delivering a costimulatory polypeptide such as IL-2, or a reduced-affinity variant of a naturally occurring costimulatory polypeptide such as an IL-2 variant disclosed herein, or a combination of both, selectively to a target T cell, the method comprising contacting a mixed population of T cells with a TMAPP of the present disclosure. The mixed population of T cells comprises the target T cell and non-target T cells. The target T cell is specific for the epitope present within the TMAPP. Contacting the mixed population of T cells with a TMAPP of the present disclosure delivers the costimulatory polypeptide(s) present within the TMAPP to the target T cell.

For example, a TMAPP of the present disclosure is contacted with a population of T cells comprising: i) a target T cell(s) that is specific for the epitope present in the TMAPP; and ii) a non-target T cell(s), e.g., a T cell(s) that is specific for a second epitope(s) that is not the epitope present in the TMAPP. Contacting the population results in selective delivery of the costimulatory polypeptide(s) (e.g., naturally-occurring costimulatory polypeptide (e.g., naturally occurring IL-2) or reduced-affinity variant of a naturally occurring costimulatory polypeptide (e.g., an IL-2 variant disclosed herein)), which is present in the TMAPP, to the target T cell. Thus, e.g., less than 50%, less than 40%, less than 30%, less than 25%, less than 20%, less than 15%, less than 10%, less than 5%, or less than 4%, 3%, 2% or 1%, of the non-target T cells bind the TMAPP and, as a result, the costimulatory polypeptide (e.g., IL-2 or IL-2 variant) is not delivered to the non-target T cells.

In some cases, the population of T cells is in vitro. In some cases, the population of T cells is in vitro, and a biological response (e.g., T cell activation and/or expansion and/or phenotypic differentiation) of the target T cell population to the TMAPP of the present disclosure is elicited in the context of an in vitro culture. For example, a mixed population of T cells can be obtained from an individual, and can be contacted with the TMAPP in vitro. Such contacting can comprise single or multiple exposures of the population of T cells to a defined dose(s) and/or exposure schedule(s). In some cases, said contacting results in selectively binding/activating and/or expanding target T cells within the population of T cells, and results in generation of a population of activated and/or expanded target T cells. As an example, a mixed population of T cells can be peripheral blood mononuclear cells (PBMC). For example, PBMC from a patient can be obtained by standard blood drawing and PBMC enrichment techniques before being exposed to 0.1-1000 nM of a TMAPP of the present disclosure under standard lymphocyte culture conditions. At time points before, during, and after exposure of the mixed T cell population at a defined dose and schedule, the abundance of target T cells in the in vitro culture can be monitored by specific peptide-MHC multimers and/or phenotypic markers and/or functional activity (e.g. cytokine ELISpot assays). In some cases, upon achieving an optimal abundance and/or phenotype of antigen specific cells in vitro, all or a portion of the population of activated and/or expanded target T cells is administered to the individual (the individual from whom the mixed population of T cells was obtained).

In some cases, the population of T cells is in vitro. For example, a mixed population of T cells is obtained from an individual, and is contacted with a TMAPP of the present disclosure in vitro. Such contacting, which can comprise single or multiple exposures of the T cells to a defined dose(s) and/or exposure schedule(s) in the context of in vitro cell culture, can be used to determine whether the mixed population of T cells includes T cells that are specific for the epitope presented by the TMAPP. The presence of T cells that are specific for the epitope of the TMAPP can be determined by assaying a sample comprising a mixed population of T cells, which population of T cells comprises T cells that are not specific for the epitope (non-target T cells) and may comprise T cells that are specific for the epitope (target T cells). Known assays can be used to detect activation and/or proliferation of the target T cells, thereby providing an ex vivo assay that can determine whether a particular TMAPP possesses an epitope that binds to T cells present in the individual and thus whether the TMAPP has potential use as a therapeutic composition for that individual. Suitable known assays for detection of activation and/or proliferation of target T cells include, e.g., flow cytometric characterization of T cell phenotype and/or antigen specificity and/or proliferation. Such an assay to detect the presence of epitope-specific T cells, e.g., a companion diagnostic, can further include additional assays (e.g. effector cytokine ELISpot assays) and/or appropriate controls (e.g. antigen-specific and antigen-nonspecific multimeric peptide-HLA staining reagents) to determine whether the TMAPP is selectively binding/activating and/or expanding the target T cell. Thus, for example, the present disclosure provides a method of detecting, in a mixed population of T cells obtained from an individual, the presence of a target T cell that binds an epitope of interest, the method comprising: a) contacting in vitro the mixed population of T cells with a TMAPP of the present disclosure, wherein the multimeric polypeptide comprises the epitope of interest; and b) detecting activation and/or proliferation of T cells in response to said contacting, wherein activated and/or proliferated T cells indicates the presence of the target T cell. Alternatively, and/or in addition, if activation and/or expansion (proliferation) of the desired T cell population is obtained using the TMAPP, then all or a portion of the population of T cells comprising the activated/expanded T cells can be administered back to the individual as a therapy.

In some instances, the population of T cells is in vivo in an individual. In such instances, a method of the present disclosure for selectively delivering a costimulatory polypeptide (e.g., IL-2 or a reduced-affinity IL-2) to an epitope-specific T cell comprises administering the TMAPP to the individual.

The epitope-specific T cell to which a costimulatory polypeptide (e.g., IL-2 or a reduced-affinity IL-2) is being selectively delivered is also referred to herein as a “target T cell.” In some cases, the target T cell is a regulatory T cell (Treg). In some cases, the Treg inhibits or suppresses activity of an autoreactive T cell. In some cases, the target T cell is a cytotoxic T cell. For example, the target T cell can be a cytotoxic T cell specific for a cancer epitope (e.g., an epitope presented by a cancer cell).

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-135 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A multimeric antigen-presenting polypeptide comprising: a) a first polypeptide comprising: i) a first major histocompatibility complex (MHC) Class II polypeptide; and b) a second polypeptide comprising: i) a second MHC Class II polypeptide; and ii) optionally an immunoglobulin (Ig) Fc polypeptide or a non-Ig scaffold, wherein the multimeric polypeptide comprises an epitope capable of being bound by a T-cell receptor (TCR), wherein the epitope is: A) at the N-terminus of the first polypeptide; or B) at the N-terminus of the second polypeptide.

Aspect 2. The multimeric antigen-presenting polypeptide of aspect 1, wherein: a) the first polypeptide comprises, in order from N-terminus to C-terminus: i) the epitope; ii) an MHC Class II α1 polypeptide; and iii) an MHC Class II α2 polypeptide; and b) the second polypeptide comprises, in order from N-terminus to C-terminus: i) an MHC Class II β1 polypeptide; and ii) an MHC Class II β2 polypeptide.

Aspect 3. The multimeric antigen-presenting polypeptide of aspect 1, wherein: a) the first polypeptide comprises, in order from N-terminus to C-terminus: i) the epitope; ii) an MHC Class II β1 polypeptide; and iii) an MHC Class II β2 polypeptide; and b) the second polypeptide comprises, in order from N-terminus to C-terminus: i) an MHC Class II α1 polypeptide; and ii) an MHC Class II α2 polypeptide.

Aspect 4. The multimeric antigen-presenting polypeptide of aspect 1, wherein: a) the first polypeptide comprises, in order from N-terminus to C-terminus: i) the epitope; ii) an MHC Class II β1 polypeptide; iii) an MHC Class II α1 polypeptide; and iv) an MHC Class II α2 polypeptide; and b) the second polypeptide comprises an MHC Class II β2 polypeptide.

Aspect 5. The multimeric antigen-presenting polypeptide of aspect 1, wherein: a) the first polypeptide comprises, in order from N-terminus to C-terminus: i) the epitope; and ii) an MHC Class II β2 polypeptide; and b) the second polypeptide comprises, in order from N-terminus to C-terminus: i) an MHC Class II β1 polypeptide; ii) an MHC Class II α1 polypeptide; and iii) an MHC Class II α2 polypeptide.

Aspect 6. The multimeric antigen-presenting polypeptide of any one of aspects 1-5, wherein the first polypeptide comprises an Ig Fc polypeptide at the C-terminus.

Aspect 7. The multimeric antigen-presenting polypeptide of any one of aspects 1-5, wherein the second polypeptide comprises an Ig Fc polypeptide at the C-terminus.

Aspect 8. The multimeric antigen-presenting polypeptide of any one of aspects 1-7, wherein: a) the first polypeptide comprises a first dimerization polypeptide; and b) the second polypeptide comprises a second dimerization polypeptide.

Aspect 9. The multimeric antigen-presenting polypeptide of aspect 8, wherein the first and the second dimerization polypeptides are leucine-zipper polypeptides, collagen dimerization polypeptides, or coiled-coil polypeptides.

Aspect 10. The multimeric antigen-presenting polypeptide of any one of aspects 2-8, wherein the MHC Class II α1 polypeptide comprises an amino acid sequence having at least 95% amino acid sequence identity to an MHC Class II α1 polypeptide depicted in any one of FIGS. 6, 11, 13, 15, 17, and 18.

Aspect 11. The multimeric antigen-presenting polypeptide of any one of aspects 2-8, wherein the MHC Class II α2 polypeptide comprises an amino acid sequence having at least 95% amino acid sequence identity to an MHC Class II α2 polypeptide depicted in any one of FIGS. 6, 11, 13, 15, 17, and 18.

Aspect 12. The multimeric antigen-presenting polypeptide of any one of aspects 2-8, wherein the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 13. The multimeric antigen-presenting polypeptide of any one of aspects 2-8, wherein the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 14. A single-chain antigen-presenting polypeptide comprising: i) a major histocompatibility complex (MHC) Class II α1 polypeptide; ii) a Class II MHC α2 polypeptide; iii) a Class II MHC β1 polypeptide; iv) a Class II MHC β2 polypeptide; v) an epitope capable of being bound by a T-cell receptor (TCR); and vi) optionally an immunoglobulin (Ig) Fc polypeptide or a non-Ig scaffold.

Aspect 15. The single-chain antigen-presenting polypeptide of aspect 14, wherein the polypeptide comprises, in order from N-terminus to C-terminus: i) the epitope; ii) the Class II MHC β1 polypeptide; iii) the Class II MHC α1 polypeptide; iv) the Class II MHC α2 polypeptide; and v) the Class II MHC β2 polypeptide.

Aspect 16. The single-chain antigen-presenting polypeptide of aspect 14, wherein the polypeptide comprises, in order from N-terminus to C-terminus: i) the epitope; ii) the Class II MHC β1 polypeptide; iii) the Class II MHC β2 polypeptide; iv) the Class II MHC α1 polypeptide; and v) the Class II MHC α2 polypeptide.

Aspect 17. The single-chain antigen-presenting polypeptide of 15 or aspect 16, comprising an immunoglobulin Fc polypeptide at the C-terminus.

Aspect 18. The single-chain antigen-presenting polypeptide of aspect 15, comprising a linker.

Aspect 19. The single-chain antigen-presenting polypeptide of aspect 18, wherein the linker is between the epitope and the Class II MHC β1 polypeptide.

Aspect 20. The single-chain antigen-presenting polypeptide of aspect 16, comprising a linker.

Aspect 21. The single-chain antigen-presenting polypeptide of aspect 20, wherein the linker is between the epitope and the Class II MHC β1 polypeptide.

Aspect 22. The single-chain antigen-presenting polypeptide of any one of aspects 14-21, wherein the MHC Class II α1 polypeptide comprises an amino acid sequence having at least 95% amino acid sequence identity to an MHC Class II α1 polypeptide depicted in any one of FIGS. 6, 11, 13, 15, 17, and 18.

Aspect 23. The single-chain antigen-presenting polypeptide of any one of aspects 14-21, wherein the MHC Class II α2 polypeptide comprises an amino acid sequence having at least 95% amino acid sequence identity to an MHC Class II α2 polypeptide depicted in any one of FIGS. 6, 11, 13, 15, 17, and 18.

Aspect 24. The single-chain antigen-presenting polypeptide of any one of aspects 14-21, wherein the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 25. The single-chain antigen-presenting polypeptide of any one of aspects 14-21, wherein the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 26. A multimeric T-cell modulatory antigen-presenting polypeptide comprising: a) a first polypeptide comprising: i) an epitope capable of being bound by a T-cell receptor (TCR); ii) a first major histocompatibility complex (MHC) Class II polypeptide; and b) a second polypeptide comprising: i) a second MHC Class II polypeptide; and wherein one or both polypeptides of the multimeric polypeptide comprises one or more immunomodulatory domains, and wherein one or both polypeptides of the multimeric polypeptide optionally comprise an immunoglobulin (Ig) Fc polypeptide or a non-Ig scaffold.

Aspect 27. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II β2 polypeptide; and iv) an immunomodulatory domain; and b) the second polypeptide comprises, in order from N-terminus to C-terminus: i) an MHC Class II α1 polypeptide; and ii) an MHC Class II α2 polypeptide.

Aspect 28. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II β2 polypeptide; and iv) an immunomodulatory domain; and b) the second polypeptide comprises, in order from N-terminus to C-terminus: i) an MHC Class II α1 polypeptide; ii) an MHC Class II α2 polypeptide; and iii) an Ig Fc polypeptide.

Aspect 29. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II β2 polypeptide; iv) an immunomodulatory domain; and v) a first dimerization polypeptide; and b) the second polypeptide comprises, in order from N-terminus to C-terminus: i) an MHC Class II α1 polypeptide; ii) an MHC Class II α2 polypeptide; and iii) a second dimerization polypeptide.

Aspect 30. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II β2 polypeptide; and b) the second polypeptide comprises, in order from N-terminus to C-terminus: i) an immunomodulatory domain; ii) an MHC Class II α1 polypeptide; and iii) an MHC Class II α2 polypeptide.

Aspect 31. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II β2 polypeptide; and b) the second polypeptide comprises, in order from N-terminus to C-terminus: i) an immunomodulatory domain; ii) an MHC Class II α1 polypeptide; iii) an MHC Class II α2 polypeptide; and iv) an Ig Fc polypeptide.

Aspect 32. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II β2 polypeptide; and iv) a first dimerization polypeptide; and b) the second polypeptide comprises, in order from N-terminus to C-terminus: i) an immunomodulatory domain; ii) an MHC Class II α1 polypeptide; iii) an MHC Class II α2 polypeptide; and iv) a second dimerization polypeptide.

Aspect 33. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II α1 polypeptide; iv) an MHC Class II α2 polypeptide; and b) the second polypeptide comprises, in order from N-terminus to C-terminus: i) an immunomodulatory domain; and ii) an MHC Class II β2 polypeptide.

Aspect 34. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II α1 polypeptide; iv) an MHC Class II α2 polypeptide; and b) the second polypeptide comprises, in order from N-terminus to C-terminus: i) an immunomodulatory domain; ii) an MHC Class II β2 polypeptide; and iii) an Ig Fc polypeptide.

Aspect The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II α1 polypeptide; iv) an MHC Class II α2 polypeptide; and v) a first dimerization polypeptide; and b) the second polypeptide comprises, in order from N-terminus to C-terminus: i) an immunomodulatory domain; ii) an MHC Class II β2 polypeptide; and iii) a second dimerization polypeptide.

Aspect 36. The multimeric T-cell modulatory antigen-presenting polypeptide of aspect 26, wherein: a) the first polypeptide comprises, in order from N-terminus to C-terminus: i) the epitope; ii) an MHC Class II β2 polypeptide; iii) an immunomodulatory domain; and iv) an Ig Fc polypeptide; and b) the second polypeptide comprises, in order from N-terminus to C-terminus: i) an MHC Class II β1 polypeptide; ii) an MHC Class II α1 polypeptide; and iii) an MHC Class II α2 polypeptide.

Aspect 37. The multimeric T-cell modulatory antigen-presenting polypeptide of aspect 26, wherein: a) the first polypeptide comprises, in order from N-terminus to C-terminus: i) the epitope; ii) an MHC Class II β1 polypeptide; iii) an MHC Class II α1 polypeptide; iv) an MHC Class II α2 polypeptide; v) a first dimerization polypeptide; and vi) an Ig Fc polypeptide; and b) the second polypeptide comprises, in order from N-terminus to C-terminus: i) an immunomodulatory domain; ii) an MHC Class II β2 polypeptide; and iii) a second dimerization polypeptide.

Aspect 38. The multimeric T-cell modulatory antigen-presenting polypeptide of aspect 37, wherein the second polypeptide comprises 2 copies of the immunomodulatory domain.

Aspect 39. The multimeric T-cell modulatory antigen-presenting polypeptide of any one of aspects 26-38, comprising a linker.

Aspect 40. The multimeric T-cell modulatory antigen-presenting polypeptide of any one of aspects 26-38, wherein the MHC Class II α1 polypeptide comprises an amino acid sequence having at least 95% amino acid sequence identity to an MHC Class II α1 polypeptide depicted in any one of FIGS. 6, 11, 13, 15, 17, and 18.

Aspect 41. The multimeric T-cell modulatory antigen-presenting polypeptide of any one of aspects 26-38, wherein the MHC Class II α2 polypeptide comprises an amino acid sequence having at least 95% amino acid sequence identity to an MHC Class II α2 polypeptide depicted in any one of FIGS. 6, 11, 13, 15, 17, and 18.

Aspect 42. The multimeric T-cell modulatory antigen-presenting polypeptide of any one of aspects 26-38, wherein the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 43. The multimeric T-cell modulatory antigen-presenting polypeptide of any one of aspects 26-38, wherein the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 44. The multimeric T-cell modulatory antigen-presenting polypeptide of any one of aspects 26-38, wherein the immunomodulatory polypeptide comprises the amino acid sequence of a naturally-occurring immunomodulatory polypeptide.

Aspect 45. The multimeric T-cell modulatory antigen-presenting polypeptide of aspect 44, wherein the immunomodulatory polypeptide is selected from the group consisting of IL-2, 4-1BBL, PD-L1, CD80, CD86, B7-1, ICOS-L, OX-40L, FasL, TGFβ, JAG1, CD70, ICAM, and PD-L2.

Aspect 46. The multimeric T-cell modulatory antigen-presenting polypeptide of any one of aspects 26-45, wherein the immunomodulatory polypeptide is a variant immunomodulatory polypeptide that comprises an amino acid sequence having from 1 to 10 amino acid substitutions compared to the amino acid sequence of a naturally-occurring immunomodulatory polypeptide, wherein the variant immunomodulatory polypeptide has reduced affinity for a co-immunomodulatory polypeptide, compared to the affinity of the naturally-occurring immunomodulatory polypeptide for the co-immunomodulatory polypeptide.

Aspect 47. The multimeric T-cell modulatory antigen-presenting polypeptide of aspect 46, wherein the variant immunomodulatory polypeptide is a variant 4-1BBL polypeptide.

Aspect 48. The multimeric T-cell modulatory antigen-presenting polypeptide of aspect 46, wherein the variant immunomodulatory polypeptide is a variant CD80 polypeptide.

Aspect 49. The multimeric T-cell modulatory antigen-presenting polypeptide of aspect 46, wherein the variant immunomodulatory polypeptide is a variant IL-2 polypeptide.

Aspect 50. The multimeric T-cell modulatory antigen-presenting polypeptide of aspect 46, wherein the variant immunomodulatory polypeptide is a variant CD86 polypeptide.

Aspect 51. The multimeric T-cell modulatory antigen-presenting polypeptide of aspect 46, wherein the variant immunomodulatory polypeptide is a variant PD-L1 polypeptide.

Aspect 52. The multimeric T-cell modulatory antigen-presenting polypeptide of any one of aspects 26-51, wherein the multimeric polypeptide comprises two immunomodulatory polypeptides.

Aspect 53. The multimeric T-cell modulatory antigen-presenting polypeptide of aspect 52, wherein the two immunomodulatory polypeptides are on the same polypeptide chain.

Aspect 54. The multimeric T-cell modulatory antigen-presenting polypeptide of aspect 52, wherein the two immunomodulatory polypeptides are on separate polypeptide chains.

Aspect 55. The multimeric T-cell modulatory antigen-presenting polypeptide of any one of aspects 52-54, wherein the two immunomodulatory polypeptides comprise the same amino acid sequence.

Aspect 56. The multimeric T-cell modulatory antigen-presenting polypeptide of any one of aspects 26-55, wherein the multimeric polypeptide comprises a peptide linker between one or more of: a) the epitope and the MHC polypeptide; b) any two adjacent MHC polypeptides; c) the MHC polypeptide and the Fc polypeptide; and d) two adjacent immunomodulatory polypeptides.

Aspect 57. The multimeric T-cell modulatory antigen-presenting polypeptide of aspect 56, wherein the linker has a length of from 20 amino acids to 40 amino acids.

Aspect 58. The multimeric T-cell modulatory antigen-presenting polypeptide of 56 or 57, wherein the linker is a peptide of the formula (GGGGS)n (SEQ ID NO:75), where n is 1, 2, 3, 4, 5, 6, 7, or 8.

Aspect 59. A single-chain T-cell modulatory antigen-presenting polypeptide comprising: i) an epitope capable of being bound by a T-cell receptor (TCR); ii) an major histocompatibility complex (MHC) Class II α1 polypeptide; iii) an MHC Class II α2 polypeptide; iv) an MHC Class II β1 polypeptide; v) an MHC Class II β2 polypeptide; vi) an immunomodulatory polypeptide; and vii) optionally an immunoglobulin (Ig) Fc polypeptide or a non-Ig scaffold.

Aspect 60. The single-chain T-cell modulatory antigen-presenting polypeptide of aspect 59 comprising, in order from N-terminus to C-terminus: i) the epitope; ii) the MHC Class II β1 polypeptide; iii) the MHC Class II α1 polypeptide; iv) the MHC Class II α2 polypeptide; v) the MHC Class II β2 polypeptide; and vi) the immunomodulatory polypeptide.

Aspect 61. The single-chain T-cell modulatory antigen-presenting polypeptide of aspect 59 comprising, in order from N-terminus to C-terminus: i) the epitope; ii) a first immunomodulatory polypeptide; iii) the MHC Class II β1 polypeptide; iv) the MHC Class II α1 polypeptide; v) the MHC Class II α2 polypeptide; vi) the MHC Class II β2 polypeptide; and vii) a second immunomodulatory polypeptide, wherein the first and the second immunomodulatory polypeptides comprise the same amino acid sequence.

Aspect 62. The single-chain T-cell modulatory antigen-presenting polypeptide of aspect 59 comprising, in order from N-terminus to C-terminus: i) the immunomodulatory polypeptide; ii) the epitope; iii) the MHC Class II β1 polypeptide; iv) the MHC Class II α1 polypeptide; v) the MHC Class II α2 polypeptide; and vi) the MHC Class II β2 polypeptide.

Aspect 63. The single-chain T-cell modulatory antigen-presenting polypeptide of aspect 59 comprising, in order from N-terminus to C-terminus: i) the epitope; ii) the MHC Class II β1 polypeptide; iii) the MHC Class II β2 polypeptide; iv) the MHC Class II α1 polypeptide; v) the MHC Class II α2 polypeptide; and vi) the immunomodulatory polypeptide.

Aspect 64. The single-chain T-cell modulatory antigen-presenting polypeptide of aspect 59 comprising, in order from N-terminus to C-terminus: i) the epitope; ii) the immunomodulatory polypeptide; iii) the MHC Class II β1 polypeptide; iv) the MHC Class II β2 polypeptide; v) the MHC Class II α1 polypeptide; and vi) the MHC Class II α2 polypeptide.

Aspect 65/The single-chain T-cell modulatory antigen-presenting polypeptide of aspect 59 comprising, in order from N-terminus to C-terminus: i) the immunomodulatory polypeptide; ii) the epitope; iii) the MHC Class II β1 polypeptide; iv) the MHC Class II β2 polypeptide; v) the MHC Class II α1 polypeptide; and vi) the MHC Class II α2 polypeptide.

Aspect 66. The single-chain T-cell modulatory antigen-presenting polypeptide of any one of aspects 59-65, wherein the MHC Class II α1 polypeptide comprises an amino acid sequence having at least 95% amino acid sequence identity to an MHC Class II α1 polypeptide depicted in any one of FIGS. 6, 11, 13, 15, 17, and 18.

Aspect 67. The single-chain T-cell modulatory antigen-presenting polypeptide of any one of aspects 59-65, wherein the MHC Class II α2 polypeptide comprises an amino acid sequence having at least 95% amino acid sequence identity to an MHC Class II α2 polypeptide depicted in any one of FIGS. 6, 11, 13, 15, 17, and 18.

Aspect 68. The single-chain T-cell modulatory antigen-presenting polypeptide of any one of aspects 59-65, wherein the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 69. The single-chain T-cell modulatory antigen-presenting polypeptide of any one of aspects 59-65, wherein the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

Aspect 70. The single-chain T-cell modulatory antigen-presenting polypeptide of any one of aspects 59-69, wherein the immunomodulatory polypeptide comprises the amino acid sequence of a naturally-occurring immunomodulatory polypeptide.

Aspect 71. The single-chain T-cell modulatory antigen-presenting polypeptide of aspect 70, wherein the immunomodulatory polypeptide is selected from the group consisting of IL-2, 4-1BBL, PD-L1, CD80, CD86, B7-1, ICOS-L, OX-40L, FasL, JAG1, TGFβ, CD70, ICAM, and PD-L2.

Aspect 72. The single-chain T-cell modulatory antigen-presenting polypeptide of any one of aspects 59-69, wherein the immunomodulatory polypeptide is a variant immunomodulatory polypeptide that comprises an amino acid sequence having from 1 to 10 amino acid substitutions compared to the amino acid sequence of a naturally-occurring immunomodulatory polypeptide, wherein the variant immunomodulatory polypeptide has reduced affinity for a co-immunomodulatory polypeptide, compared to the affinity of the naturally-occurring immunomodulatory polypeptide for the co-immunomodulatory polypeptide.

Aspect 73. The single-chain T-cell modulatory antigen-presenting polypeptide of aspect 72, wherein the variant immunomodulatory polypeptide is a variant 4-1BBL polypeptide.

Aspect 74. The single-chain T-cell modulatory antigen-presenting polypeptide of aspect 72, wherein the variant immunomodulatory polypeptide is a variant CD80 polypeptide.

Aspect 75. The single-chain T-cell modulatory antigen-presenting polypeptide of aspect 72, wherein the variant immunomodulatory polypeptide is a variant IL-2 polypeptide.

Aspect 76. The single-chain T-cell modulatory antigen-presenting polypeptide of aspect 72, wherein the variant immunomodulatory polypeptide is a variant CD86 polypeptide.

Aspect 77. The single-chain T-cell modulatory antigen-presenting polypeptide of aspect 72, wherein the variant immunomodulatory polypeptide is a variant PD-L1 polypeptide.

Aspect 78. The single-chain T-cell modulatory antigen-presenting polypeptide of any one of aspects 59-77, wherein the polypeptide comprises two immunomodulatory polypeptides.

Aspect 79. The single-chain T-cell modulatory antigen-presenting polypeptide of aspect 78, wherein the two immunomodulatory polypeptides comprise the same amino acid sequence.

Aspect 80. The single-chain T-cell modulatory antigen-presenting polypeptide of any one of aspects 59-79, wherein the multimeric polypeptide comprises a peptide linker between one or more of: a) the epitope and the MHC polypeptide; b) any two adjacent MHC polypeptides; c) the MHC polypeptide and the Fc polypeptide; and d) two adjacent immunomodulatory polypeptides.

Aspect 81. The single-chain T-cell modulatory antigen-presenting polypeptide of aspect 80, wherein the linker has a length of from 20 amino acids to 40 amino acids.

Aspect 82. The single-chain T-cell modulatory antigen-presenting polypeptide of aspect 80 or aspect 81, wherein the linker is a peptide of the formula (GGGGS)n, where n is 1, 2, 3, 4, 5, 6, 7, or 8.

Aspect 83. The multimeric T-cell modulatory antigen-presenting polypeptide of any one of aspects 26-58, or the single-chain T-cell modulatory antigen-presenting polypeptide of any one of aspects 59-82, comprising an Ig Fc polypeptide, and wherein the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide.

Aspect 84. The multimeric T-cell modulatory antigen-presenting polypeptide or single-chain T-cell modulatory antigen-presenting polypeptide of aspect 83, wherein a drug is conjugated to the Ig Fc polypeptide.

Aspect 85. The multimeric T-cell modulatory antigen-presenting polypeptide of any one of aspects 26-58, or the single-chain T-cell modulatory antigen-presenting polypeptide of any one of aspects 59-82, wherein the epitope is a cancer epitope.

Aspect 86. The multimeric T-cell modulatory antigen-presenting polypeptide of any one of aspects 26-58, or the single-chain T-cell modulatory antigen-presenting polypeptide of any one of aspects 59-82, wherein the epitope is an auto-epitope.

Aspect 87. A composition comprising: a) an antigen-presenting polypeptide of any one of aspects 1-86; and b) a buffer.

Aspect 88. A composition comprising: a) the T-cell modulatory antigen-presenting polypeptide of any one of aspects 26-82; and b) a pharmaceutically acceptable excipient.

Aspect 89. A composition comprising: a) the T-cell modulatory antigen-presenting polypeptide of any one of aspects 26-82; and b) saline.

Aspect 90. The composition of aspect 89, wherein the saline is 0.9% NaCl.

Aspect 91. The composition of aspect 89 or aspect 90, wherein the composition is sterile.

Aspect 92. One or more nucleic acids comprising nucleotide sequences encoding the antigen-presenting polypeptide of any one of aspects 1-25.

Aspect 93. One or more recombinant expression vectors comprising the one or more nucleic acids of aspect 92.

Aspect 94. A host cell genetically modified with the one or more nucleic acids of aspect 92 or the one or more recombinant expression vectors of aspect 93.

Aspect 95. The host cell of aspect 94, wherein the host cell is a eukaryotic cell.

Aspect 96. One or more nucleic acids comprising nucleotide sequences encoding the T-cell modulatory antigen-presenting polypeptide of any one of aspects 26-82.

Aspect 97. One or more recombinant expression vectors comprising the one or more nucleic acids of aspect 96.

Aspect 98. A host cell genetically modified with the one or more nucleic acids of aspect 91 or the one or more recombinant expression vectors of aspect 97.

Aspect 99. The host cell of aspect 98, wherein the host cell is a eukaryotic cell.

Aspect 100. A method of detecting an antigen-specific T cell, the method comprising contacting a T cell with the antigen-presenting polypeptide of any one of aspects 1-25, wherein binding of the antigen-presenting polypeptide to the T cell indicates that the T cell is specific for the epitope present in the antigen-presenting polypeptide.

Aspect 101. The method of aspect 100, wherein the antigen-presenting polypeptide comprises a detectable label.

Aspect 102. The method of aspect 101, wherein the detectable label is a radioisotope, a fluorescent polypeptide, or an enzyme that generates a fluorescent product, an enzyme that generates a colored product.

Aspect 103. The method of aspect 100, wherein binding of the antigen-presenting polypeptide to the T cell is detected using a detectably labeled antibody specific for the antigen-presenting polypeptide.

Aspect 104. The method of any one of aspects 100-102, wherein the T cell is present in a sample comprising a plurality of T cells.

Aspect 105. A method of selectively modulating the activity of an epitope-specific T cell, the method comprising contacting the T cell with the T-cell modulatory antigen-presenting polypeptide of any one of aspects 26-82, wherein said contacting selectively modulates the activity of the epitope-specific T cell.

Aspect 106. The method of aspect 105, wherein said contacting is in vitro.

Aspect 107. The method of aspect 105, wherein said contacting is in vivo.

Aspect 108. The method of any one of aspects 105-107, wherein the T-cell is a regulatory T cell (Treg).

Aspect 109. The method of aspect 108, wherein said contacting activates the Treg and reduces activity of an autoreactive T cell.

Aspect 110. The method of any one of aspects 105-109, wherein the T-cell is a CD4⁺ T helper cell, and wherein said contacting activates the CD4⁺ T cell.

Aspect 111. The method of aspect 109, wherein said activated CD4⁺ T cell activates a CD8⁺ T cell.

Aspect 112. The method of aspect 106, wherein the CD8⁺ T cell is specific for a cancer epitope presented by the T-cell modulatory antigen-presenting polypeptide.

Aspect 113. The method of any one of aspects 106 and 107-112, comprising administering the T-cell modulatory antigen-presenting polypeptide to an individual in need thereof.

Aspect 114. The method of aspect 113, wherein said administering is systemic.

Aspect 115. The method of aspect 113, wherein said administering is local.

Aspect 116. The method of aspect 113, wherein said administering is peritumoral.

Aspect 117. The method of aspect 113, wherein said administering is via intravenous administration.

Aspect 118. The method of any one of aspects 105-117, wherein the individual is a human.

Aspect 119. The method of aspect 118, wherein the individual has an autoimmune disease.

Aspect 120. The method of aspect 118, wherein the individual has a cancer.

Aspect 121. A treatment method, the method comprising administering to an individual in need thereof an effective amount of the T-cell modulatory antigen-presenting polypeptide of any one of aspects 26-82, wherein said administering treats the individual.

Aspect 122. The method of aspect 121, wherein the individual has cancer, and wherein said administering treats the cancer.

Aspect 123. The method of aspect 121, wherein the individual has an autoimmune disorder, and wherein said administering treats the autoimmune disorder.

Aspect 124. The method of any one of aspects 121-123, wherein said administering is via intravenous administration.

Aspect 125. The method of any one of aspects 121-123, wherein said administering is via local administration.

Aspect 126. The method of any one of aspects 121-123, wherein said administering is via systemic administration.

Aspect 127. A method of delivering a costimulatory polypeptide selectively to target a T cell, the method comprising contacting a mixed population of T cells with a T-cell modulatory antigen-presenting polypeptide of any one of aspects 26-86, wherein the mixed population of T cells comprises the target T cell and non-target T cells, wherein the target T cell is specific for the epitope present within the T-cell modulatory antigen-presenting polypeptide, and wherein said contacting delivers the costimulatory polypeptide present within the T-cell modulatory antigen-presenting polypeptide to the target T cell.

Aspect 128. The method of aspect 127, wherein the population of T cells is in vitro.

Aspect 129. The method of aspect 127, wherein the population of T cells is in vivo in an individual.

Aspect 130. The method of aspect 129, comprising administering the T-cell modulatory antigen-presenting polypeptide to the individual.

Aspect 131. The method of any one of aspects 127-130, wherein the target T cell is a regulatory T cell.

Aspect 132. The method of any one of aspects 127-130, wherein the target T cell is a cytotoxic T cell.

Aspect 133. The method of aspect 127 or 128, wherein the mixed population of T cells is an in vitro population of mixed T cells obtained from an individual, and wherein said contacting results in activation and/or proliferation of the target T cell, generating a population of activated and/or proliferated target T cells.

Aspect 134. The method of aspect 133, further comprising administering the population of activated and/or proliferated target T cells to the individual.

Aspect 135. A method of detecting, in a mixed population of T cells obtained from an individual, the presence of a target T cell that binds an epitope of interest, the method comprising: a) contacting in vitro the mixed population of T cells with the T-cell modulatory antigen-presenting polypeptide of any one of aspects 26-86, wherein the T-cell modulatory antigen-presenting polypeptide comprises the epitope of interest; and b) detecting activation and/or proliferation of T cells in response to said contacting, wherein activated and/or proliferated T cells indicates the presence of the target T cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Production of Antigen-Presenting Polypeptides

To optimize production of intact and stable MHC Class II antigen-presenting polypeptides, various structural arrangements of antigen-presenting polypeptides comprising MHC Class II polypeptides were synthesized, expressed, and purified using Protein A affinity chromatography.

The expression vector used was pD2610-v10: CMV(v10)-ORF, Mamm-ElecD (from ATUM). A nucleic acid comprising a nucleotide sequence encoding an antigen-presenting polypeptide(s) (e.g., an MHC Class II synTac) were inserted into the expression vector, to generate a recombinant expression vector encoding the antigen-presenting polypeptide(s) (e.g., an MHC Class II synTac). The recombinant expression vectors were introduced into ExpiCHO cells (Thermo; modified Chinese Hamster Ovary (CHO) cells; see, e.g., Jain et al. (2017) Protein Expr. Purif 134:38) using standard methods, generating genetically modified ExpiCHO cells. The genetically modified ExpiCHO cells were cultured in vitro in standard culture medium. The antigen-presenting polypeptide(s) (e.g., an MHC Class II synTac) were produced by the genetically modified ExpiCHO cells, and secreted into the culture medium. The antigen-presenting polypeptide(s) (e.g., an MHC Class II synTac) were purified from the culture medium using protein A affinity chromatography.

Briefly, a column pre-packed with κ mL of mAb Select SuRe (GE Cat. #11003495) (protein A coupled to beads) was used. The flow rate used was 1.0 mL/minute. The culture medium was loaded onto the column at 1.0 mL/min. Before loading the culture medium, the column was equilibrated with 5 column volumes (CV) of equilibration buffer (lx phosphate-buffered saline (PBS), 20 mM EDTA). After the culture medium was loaded onto the column, the column was washed with 5 CV equilibration buffer, then washed with 10 CV wash buffer (1×PBS+863 mM NaCl (1 M total NaCl), 5 mM EDTA). Next, the column was washed with 5 CV equilibration buffer. Finally, the antigen-presenting polypeptide(s) (e.g., an MHC Class II synTac) bound to the column was eluted with elution buffer (50 mM glycine, pH 2.8, 500 mM NaCl); and 25-mL fractions were collected. Neutralization buffer (Tris-HCl, pH 9.0) was added to the collected fractions. Peak fractions were pooled, dialyzed against dialysis buffer (PBS+363 mM NaCl), then concentrated. The concentrated product was then subjected to size exclusion chromatography.

For the design of the MHC Class II synTacs, parameters varied included orientation of the MHC Class II alpha and beta chains, Fc placement, IL2 (MOD) placement, and length and content of the various linkers. The variants presented include single-chain as well as two-chain versions, each with the MHC Class II β-1 domain linked N-terminal to the α-1 domain, and with β-2 either C-terminal to α-2 or on a separate chain, as shown schematically in FIG. 24. Single-chain variants with and without the β-2 domain or the IL2 fusion are shown, as well as two-chain versions with and without the bZIP dimerization domain. A two-chain version with the CMV peptide epitope instead of the hemagglutinin (HA) peptide epitope is shown, as well one version with the MHC Class II DR4 instead of the DR1 allele, with a proinsulin peptide.

Antigen-presenting polypeptides, with or without immunomodulatory polypeptides, were generated. Amino acid sequences of the antigen-presenting polypeptides, and nucleotide sequences encoding the polypeptides, are provided in FIG. 25-35. The polypeptides included single-chain polypeptides and multimeric polypeptides. The antigen-presenting polypeptides are as follows:

1) 1599—This is a single-chain polypeptide comprising a variant IL-2 immunomodulatory polypeptide. The 1599 polypeptide also includes an HLA β2 polypeptide. The 1599 polypeptide includes: i) an epitope (a hemagglutinin epitope); ii) HLA DRB1 β1; iii) HLA DRA α1 and α2; iv) HLA DRB1 β2; v) a variant IL-2 immunomodulatory polypeptide; and v) an IgG1 Fc.

2) 1559—This is a single-chain polypeptide comprising an HLA β2 polypeptide. The 1559 polypeptide lacks an immunomodulatory polypeptide. The 1559 polypeptide includes: i) an epitope (a hemagglutinin (HA) epitope); ii) HLA DRB1 β1; iii) HLA DRA α1 and α2; iv) HLA DRB1 β2; and v) an IgG1 Fc.

3) 1601—This is a single-chain polypeptide a variant IL-2 immunomodulatory polypeptide. The 1601 polypeptide lacks an HLA β2 polypeptide. The 1601 polypeptide includes: i) an epitope (a hemagglutinin epitope); ii) HLA DRB1 β1; iii) HLA DRA α1 and α2; iv) 2 copies of a variant IL-2 immunomodulatory polypeptide; and v) an IgG1 Fc polypeptide.

4) 1452+1661—This is a multimeric antigen-presenting polypeptide. The epitope is a hemagglutinin epitope. It includes HLA DRB1 and DRA MHC Class II polypeptides. Both polypeptide chains include leucine zipper dimerizer peptides. The 1452 polypeptide includes an IgG1 Fc polypeptide.

5) 1659+1664—This is a multimeric antigen-presenting polypeptide. It includes HLA DRB1 and DRA MHC Class II polypeptides. The epitope is a hemagglutinin epitope. The 1664 polypeptide includes 2 copies of a variant IL-2 immunomodulatory polypeptide. Both polypeptide chains lack leucine zipper dimerizer peptides. The 1659 polypeptide includes an IgG1 Fc polypeptide.

6) 1637+1408—This is a multimeric antigen-presenting polypeptide. It includes HLA DRB1 and DRA MHC Class II polypeptides. The epitope is a CMV epitope. The 1408 polypeptide includes 2 copies of a variant IL-2 immunomodulatory polypeptide. Both chains include a leucine zipper (bZIP). The 1637 polypeptide includes an IgG1 Fc polypeptide.

7) 1639+1640—This is a multimeric antigen-presenting polypeptide. It includes HLA DRB1-4 and DRA MHC Class II polypeptides. The epitope is a proinsulin epitope. Both chains include a leucine zipper (bZIP). The 1640 chain includes 2 copies of a variant IL-2 immunomodulatory polypeptide. The 1639 polypeptide includes an IgG1 Fc polypeptide.

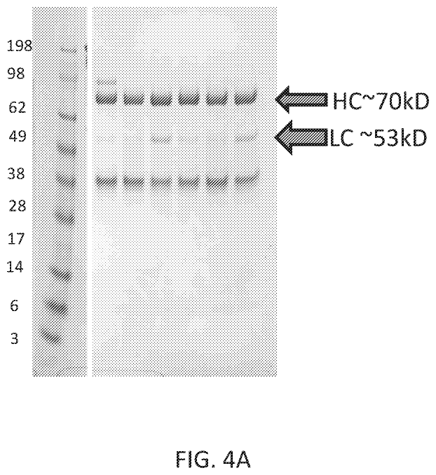

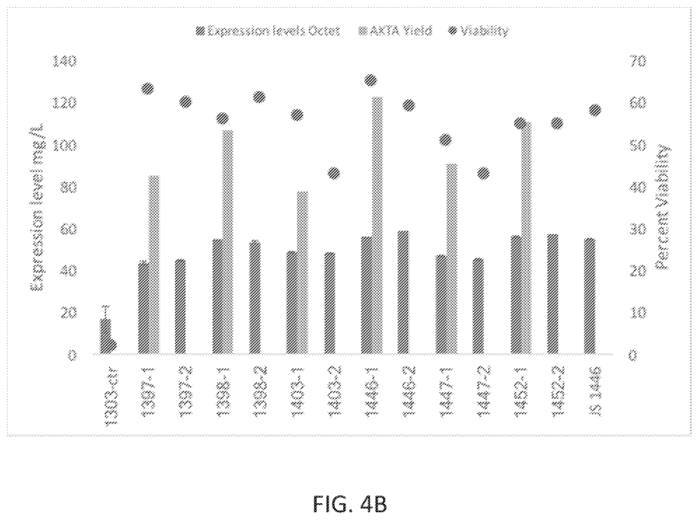

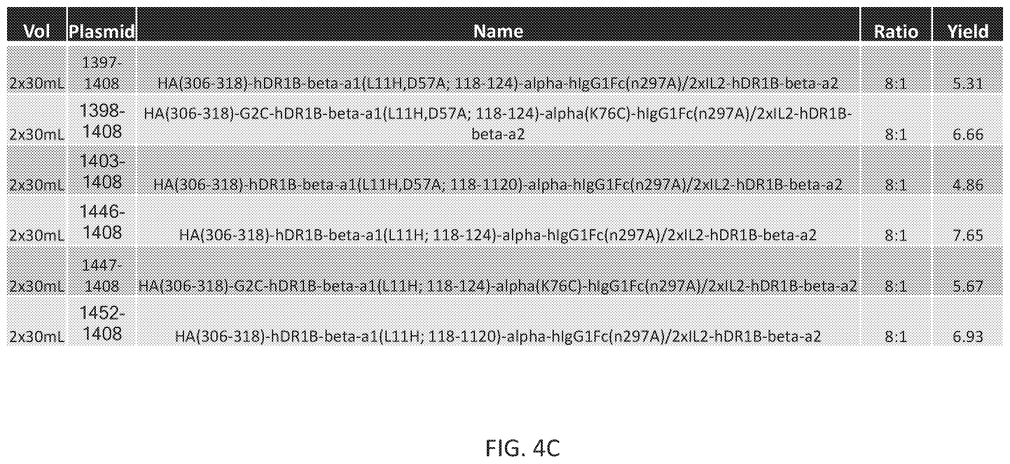

Expression constructs comprising nucleotide sequences encoding the above-described polypeptides were introduced into a mammalian cell line. The produced polypeptides were loaded onto a reducing polyacrylamide gel. FIG. 4A-4B depict gel analysis (FIG. 4A) and expression levels (FIG. 4B) of various multimeric polypeptides described in FIG. 4C. As shown in FIG. 24, left panel, all polypeptides were produced in detectable amounts. The various constructs are depicted schematically in FIG. 24, right panel.

The single-chain version without the β-2 domain (and with IL2) demonstrated a robust expression level and an intact, homogeneous product upon Protein A purification (lane 3). The addition of the β-2 domain resulted in lower expression as well as de-stabilization of the molecule as indicated by a prominent breakdown product observed on the analytical gel (lane 1). Removal of the IL2 (while retaining the (3-2) resulted in even lower expression, but with minimal breakdown product (lane 2).

With the β-2 domain on a separate chain, robust assembly was observed with incorporation of the bZIP leucine zipper dimerization domain (lane 5). Without the bZIP domain, modest expression and production of intact Fc-containing chain was observed; β-2 chain was incorporated (lane 4). Switching from the HA peptide to the CMV peptide in the two-chain bZIP model resulted in very robust expression of intact product (lane 6). Changing the β-2 allele from DR1 to DR4 along with the proinsulin peptide also resulted in robust expression of intact product (lane 7).

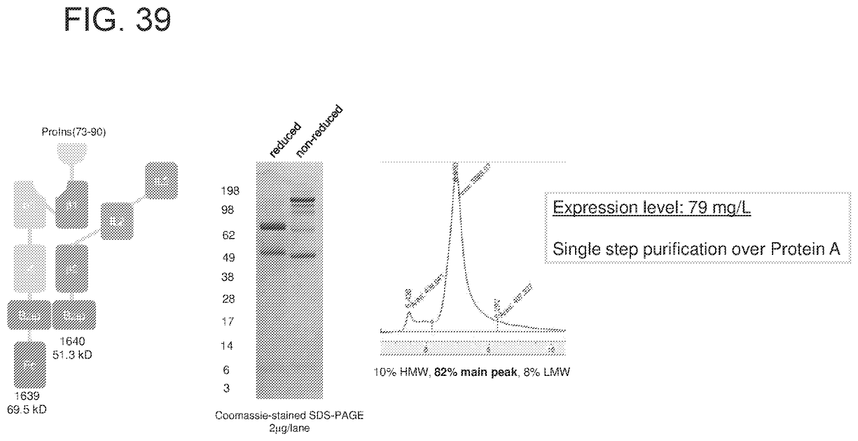

Production of the 1639+1640 multimeric antigen-presenting polypeptide is depicted in FIG. 39. A Coomassie-stained SDS-PAGE gel, under reducing and non-reducing conditions, following a single-step purification over a Protein A column, is shown. The gel shows the 69.5 kD 1639 chain and the 51.3 kD 1640 chain. Density scan of the gel indicated that the 1639+1630 multimeric polypeptide was produced at about 79 mg/L.

Therefore, intact, stable MHC Class II antigen-presenting polypeptides were synthesized, expressed and purified. These represented design models that incorporated either a single-chain or a two-chain system. Incorporation of both the β-2 domain and a dimerization domain resulted in robust expression using the two-chain system. The peptide chosen for binding to the MHC can provide stabilization. Further, intact, stable MHC Class II antigen-presenting polypeptides were generated with MHC Class II polypeptides of two different MHC alleles.

### Example 2: Further Antigen-Presenting Polypeptides

Constructs encoding a T-cell modulatory antigen-presenting multimeric polypeptide were generated, in which the first polypeptide included: i) an epitope; ii) an HLA P31 polypeptide; iii) an HLA α1 polypeptide; and iv) an HLA α2 polypeptide; and in which the second polypeptide included: i) two copies of a variant IL-2 immunomodulatory polypeptide; ii) an HLA β2 polypeptide; and iii) an Ig Fc polypeptide.

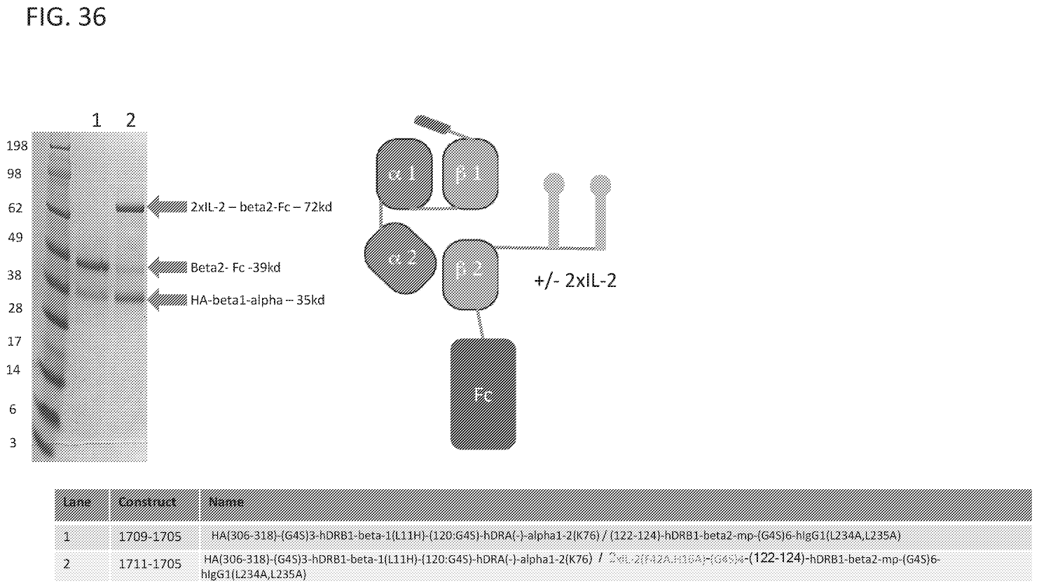

The multimeric polypeptide encoded by the constructs was produced, as described in Example 1; and the multimeric polypeptide so produced was analyzed. FIG. 36 schematically depicts the multimeric polypeptide.

1) 1711+1705—This is a multimeric antigen-presenting polypeptide. It includes HLA DRB1 Class II polypeptides. The epitope is a hemagglutinin epitope. The 1711 polypeptide includes 2 copies of a variant IL-2 immunomodulatory polypeptide; an HLA DRB1 β2 polypeptide; and an IgG1 Fc polypeptide. The 1705 polypeptide includes the epitope-presenting peptide; an HLA DRB1 β1 polypeptide; an HLA DRA α1 polypeptide; and an HLA DRA α2 polypeptide.

2) 1709+1705. This multimeric polypeptide is like 1711+1705, except that the 1709 polypeptide does not include any immunomodulatory polypeptides. Thus, the 1709 polypeptide includes only the HLA DRB1 β2 polypeptide and the IgG1 Fc polypeptide present in the 1711 polypeptide.

The expression results are provided in FIG. 36. The gel analysis indicates that intact polypeptides were generated. The expression levels were 10⁻¹⁵ mg/L. While the 35 kD band includes Fc breakdown product, Western blot analysis indicated that the 35 kD band also includes the peptide-β1-α1-α2 chain.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A multimeric T-cell modulatory antigen-presenting polypeptide comprising:
a) a first polypeptide comprising:
i) an epitope capable of being bound by a T-cell receptor (TCR);
ii) a first major histocompatibility complex (MHC) Class II polypeptide; and
b) a second polypeptide comprising:
i) a second MHC Class II polypeptide; and
wherein one or both polypeptides of the multimeric polypeptide comprises one or more immunomodulatory domains, and
wherein one or both polypeptides of the multimeric polypeptide optionally comprise an immunoglobulin (Ig) Fc polypeptide or a non-Ig scaffold.

2. The multimeric T-cell modulatory antigen-presenting polypeptide of claim 1, wherein:
a1) the first polypeptide comprises, in order from N-terminus to C-terminus:
i) the epitope;
ii) an MHC Class II β1 polypeptide;
iii) an MHC Class II β2 polypeptide; and
iv) an immunomodulatory domain; and
b1) the second polypeptide comprises, in order from N-terminus to C-terminus:
i) an MHC Class II α1 polypeptide;
ii) an MHC Class II α2 polypeptide; or
a2) the first polypeptide comprises, in order from N-terminus to C-terminus:
i) the epitope;
ii) an MHC Class II β1 polypeptide;
iii) an MHC Class II β2 polypeptide; and
iv) an immunomodulatory domain; and
b2) the second polypeptide comprises, in order from N-terminus to C-terminus:
i) an MHC Class II α1 polypeptide;
ii) an MHC Class II α2 polypeptide; and
iii) an Ig Fc polypeptide; or
a3) the first polypeptide comprises, in order from N-terminus to C-terminus:
i) the epitope;
ii) an MHC Class II β1 polypeptide;
iii) an MHC Class II β2 polypeptide;
iv) an immunomodulatory domain; and
v) a first dimerization polypeptide; and
b3) the second polypeptide comprises, in order from N-terminus to C-terminus:
i) an MHC Class II α1 polypeptide;
ii) an MHC Class II α2 polypeptide; and
iii) a second dimerization polypeptide; or
a4) the first polypeptide comprises, in order from N-terminus to C-terminus:
i) the epitope;
ii) an MHC Class II β1 polypeptide;
iii) an MHC Class II β2 polypeptide; and
b4) the second polypeptide comprises, in order from N-terminus to C-terminus:
i) an immunomodulatory domain;
ii) an MHC Class II α1 polypeptide; and
iii) an MHC Class II α2 polypeptide; or
a5) the first polypeptide comprises, in order from N-terminus to C-terminus:
i) the epitope;
ii) an MHC Class II β1 polypeptide;
iii) an MHC Class II β2 polypeptide; and
b5) the second polypeptide comprises, in order from N-terminus to C-terminus:
i) an immunomodulatory domain;
ii) an MHC Class II α1 polypeptide;
iii) an MHC Class II α2 polypeptide; and
iv) an Ig Fc polypeptide; or
a6) the first polypeptide comprises, in order from N-terminus to C-terminus:
i) the epitope;
ii) an MHC Class II β1 polypeptide;
iii) an MHC Class II β2 polypeptide; and
iv) a first dimerization polypeptide; and
b6) the second polypeptide comprises, in order from N-terminus to C-terminus:
i) an immunomodulatory domain;
ii) an MHC Class II α1 polypeptide;
iii) an MHC Class II α2 polypeptide; and
iv) a second dimerization polypeptide; or
a7) the first polypeptide comprises, in order from N-terminus to C-terminus:
i) the epitope;
ii) an MHC Class II β1 polypeptide;
iii) an MHC Class II α1 polypeptide;
iv) an MHC Class II α2 polypeptide; and
b7) the second polypeptide comprises, in order from N-terminus to C-terminus:
i) an immunomodulatory domain; and
ii) an MHC Class II β2 polypeptide; or
a8) the first polypeptide comprises, in order from N-terminus to C-terminus:
i) the epitope;
ii) an MHC Class II β1 polypeptide;
iii) an MHC Class II α1 polypeptide;
iv) an MHC Class II α2 polypeptide; and
b8) the second polypeptide comprises, in order from N-terminus to C-terminus:
i) an immunomodulatory domain;
ii) an MHC Class II β2 polypeptide; and
iii) an Ig Fc polypeptide; or
a9) the first polypeptide comprises, in order from N-terminus to C-terminus:
i) the epitope;
ii) an MHC Class II β1 polypeptide;
iii) an MHC Class II α1 polypeptide;
iv) an MHC Class II α2 polypeptide; and
v) a first dimerization polypeptide; and
b9) the second polypeptide comprises, in order from N-terminus to C-terminus:
i) an immunomodulatory domain;
ii) an MHC Class II β2 polypeptide; and
iii) a second dimerization polypeptide; or
a10) the first polypeptide comprises, in order from N-terminus to C-terminus:
i) the epitope;
ii) an MHC Class II β2 polypeptide;
iii) an immunomodulatory domain; and
iv) an Ig Fc polypeptide; and
b10) the second polypeptide comprises, in order from N-terminus to C-terminus:
i) an MHC Class II β1 polypeptide;
ii) an MHC Class II α1 polypeptide; and
iii) an MHC Class II α2 polypeptide; or
a11) the first polypeptide comprises, in order from N-terminus to C-terminus:
i) the epitope;
ii) an MHC Class II β1 polypeptide;
iii) an MHC Class II α1 polypeptide;
iv) an MHC Class II α2 polypeptide;
v) a first dimerization polypeptide; and
vi) an Ig Fc polypeptide; and
b11) the second polypeptide comprises, in order from N-terminus to C-terminus:
i) an immunomodulatory domain;
ii) an MHC Class II β2 polypeptide; and
iii) a second dimerization polypeptide.

3. A single-chain T-cell modulatory antigen-presenting polypeptide comprising:
i) an epitope capable of being bound by a T-cell receptor (TCR);
ii) an major histocompatibility complex (MHC) Class II α1 polypeptide;
iii) an MHC Class II α2 polypeptide;
iv) an MHC Class II β1 polypeptide;
v) an MHC Class II β2 polypeptide;
vi) an immunomodulatory polypeptide; and
vii) optionally an immunoglobulin (Ig) Fc polypeptide or a non-Ig scaffold.

4. The single-chain T-cell modulatory antigen-presenting polypeptide of claim 3, wherein the single-chain T-cell modulatory antigen-presenting polypeptide:
a) comprises, in order from N-terminus to C-terminus:
i) the epitope;
ii) the MHC Class II β1 polypeptide;
iii) the MHC Class II α1 polypeptide;
iv) the MHC Class II α2 polypeptide;
v) the MHC Class II β2 polypeptide; and
vi) the immunomodulatory polypeptide; or
b) comprises, in order from N-terminus to C-terminus:
i) the epitope;
ii) a first immunomodulatory polypeptide;
iii) the MHC Class II β1 polypeptide;
iv) the MHC Class II α1 polypeptide;
v) the MHC Class II α2 polypeptide;
vi) the MHC Class II β2 polypeptide; and
vii) a second immunomodulatory polypeptide, wherein the first and the second immunomodulatory polypeptides comprise the same amino acid sequence; or
c) comprises, in order from N-terminus to C-terminus:
i) the immunomodulatory polypeptide;
ii) the epitope;
iii) the MHC Class II β1 polypeptide;
iv) the MHC Class II α1 polypeptide;
v) the MHC Class II α2 polypeptide; and
vi) the MHC Class II β2 polypeptide; or
d) comprises, in order from N-terminus to C-terminus:
i) the epitope;
ii) the MHC Class II β1 polypeptide;
iii) the MHC Class II β2 polypeptide;
iv) the MHC Class II α1 polypeptide;
v) the MHC Class II α2 polypeptide; and
vi) the immunomodulatory polypeptide; or
e) comprises, in order from N-terminus to C-terminus:
i) the epitope;
ii) the immunomodulatory polypeptide;
iii) the MHC Class II β1 polypeptide;
iv) the MHC Class II β2 polypeptide;
v) the MHC Class II α1 polypeptide; and
vi) the MHC Class II α2 polypeptide; or
f) comprises, in order from N-terminus to C-terminus:
i) the immunomodulatory polypeptide;
ii) the epitope;
iii) the MHC Class II β1 polypeptide;
iv) the MHC Class II β2 polypeptide;
v) the MHC Class II α1 polypeptide; and
vi) the MHC Class II α2 polypeptide.

5. The multimeric T-cell modulatory antigen-presenting polypeptide of claim 1 or claim 2, or the single-chain T-cell modulatory antigen-presenting polypeptide of claim 3 or claim 4, wherein:
a) the MHC Class II α1 polypeptide comprises an amino acid sequence having at least 95% amino acid sequence identity to an MHC Class II α1 polypeptide depicted in any one of FIGS. 6, 11, 13, 15, 17, and 18; and/or;
b) the MHC Class II α2 polypeptide comprises an amino acid sequence having at least 95% amino acid sequence identity to an MHC Class II α2 polypeptide depicted in any one of FIGS. 6, 11, 13, 15, 17, and 18; and/or
c) the MHC Class II β1 polypeptide comprises an amino acid sequence having at least 95% amino acid sequence identity to an MHC Class II β1 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B; and/or
d) the MHC Class II β2 polypeptide comprises an amino acid sequence having at least 95% amino acid sequence identity to an MHC Class II β2 polypeptide depicted in any one of FIG. 7A-7J, FIG. 8A-8B, FIG. 9, FIG. 10, FIG. 12, FIG. 14, FIG. 16, FIG. 19A-19B, and FIG. 20A-20B.

6. The multimeric T-cell modulatory antigen-presenting polypeptide of claim 1 or claim 2, or the single-chain T-cell modulatory antigen-presenting polypeptide of claim 3 or claim 4, wherein the immunomodulatory polypeptide:
a) comprises the amino acid sequence of a naturally-occurring immunomodulatory polypeptide; or
b) is a variant immunomodulatory polypeptide that comprises an amino acid sequence having from 1 to 10 amino acid substitutions compared to the amino acid sequence of a naturally-occurring immunomodulatory polypeptide, wherein the variant immunomodulatory polypeptide has reduced affinity for a co-immunomodulatory polypeptide, compared to the affinity of the naturally-occurring immunomodulatory polypeptide for the co-immunomodulatory polypeptide.

7. The multimeric T-cell modulatory antigen-presenting polypeptide of claim 1 or claim 2, or the single-chain T-cell modulatory antigen-presenting polypeptide of claim 3 or claim 4, comprising two or more immunomodulatory polypeptides.

8. The multimeric T-cell modulatory antigen-presenting polypeptide of claim 1 or claim 2, or the single-chain T-cell modulatory antigen-presenting polypeptide of claim 3 or claim 4, wherein:
a) the epitope is a cancer epitope; or
b) the epitope is an auto-epitope.

9. A multimeric antigen-presenting polypeptide comprising:
a) a first polypeptide comprising:
i) a first major histocompatibility complex (MHC) Class II polypeptide; and
b) a second polypeptide comprising:
i) a second MHC Class II polypeptide; and
ii) optionally an immunoglobulin (Ig) Fc polypeptide or a non-Ig scaffold,
wherein the multimeric polypeptide comprises an epitope capable of being bound by a T-cell receptor (TCR), wherein the epitope is:
A) at the N-terminus of the first polypeptide; or
B) at the N-terminus of the second polypeptide.

10. The multimeric antigen-presenting polypeptide of claim 9, wherein:
a1) the first polypeptide comprises, in order from N-terminus to C-terminus:
i) the epitope;
ii) an MHC Class II α1 polypeptide; and
iii) an MHC Class II α2 polypeptide; and
b1) the second polypeptide comprises, in order from N-terminus to C-terminus:
i) an MHC Class II β1 polypeptide; and
ii) an MHC Class II β2 polypeptide; or
a2) the first polypeptide comprises, in order from N-terminus to C-terminus:
i) the epitope;
ii) an MHC Class II β1 polypeptide; and
iii) an MHC Class II β2 polypeptide; and
b2) the second polypeptide comprises, in order from N-terminus to C-terminus:
i) an MHC Class II α1 polypeptide; and
ii) an MHC Class II α2 polypeptide; or
a3) the first polypeptide comprises, in order from N-terminus to C-terminus:
i) the epitope;
ii) an MHC Class II β1 polypeptide;
iii) an MHC Class II α1 polypeptide; and
iv) an MHC Class II α2 polypeptide; and
b3) the second polypeptide comprises an MHC Class II β2 polypeptide; or
a4) the first polypeptide comprises, in order from N-terminus to C-terminus:
i) the epitope; and
ii) an MHC Class II β2 polypeptide; and
b4) the second polypeptide comprises, in order from N-terminus to C-terminus:
i) an MHC Class II β1 polypeptide;
ii) an MHC Class II α1 polypeptide; and
iii) an MHC Class II α2 polypeptide.

11. A single-chain antigen-presenting polypeptide comprising:
i) a major histocompatibility complex (MHC) Class II α1 polypeptide;
ii) a Class II MHC α2 polypeptide;
iii) a Class II MHC pl polypeptide;
iv) a Class II MHC β2 polypeptide;
v) an epitope capable of being bound by a T-cell receptor (TCR); and
vi) optionally an immunoglobulin (Ig) Fc polypeptide or a non-Ig scaffold.

12. The single-chain antigen-presenting polypeptide of claim 11, wherein:
a) the polypeptide comprises, in order from N-terminus to C-terminus:
i) the epitope;
ii) the Class II MHC pl polypeptide;
iii) the Class II MHC α1 polypeptide;
iv) the Class II MHC α2 polypeptide; and
v) the Class II MHC β2 polypeptide; or
b) the polypeptide comprises, in order from N-terminus to C-terminus:
i) the epitope;
ii) the Class II MHC pl polypeptide;
iii) the Class II MHC β2 polypeptide;
iv) the Class II MHC α1 polypeptide; and
v) the Class II MHC α2 polypeptide.

13. A composition comprising:
a1) the T-cell modulatory antigen-presenting polypeptide of any one of claims 1-8; and
b1) a pharmaceutically acceptable excipient; or
a2) an antigen-presenting polypeptide of any one of claims 9-12; and
b2) a buffer.

14. One or more nucleic acids comprising nucleotide sequences encoding:
a) the T-cell modulatory antigen-presenting polypeptide of any one of claims 1-8; or
b) the antigen-presenting polypeptide of any one of claims 9-12.

15. One or more recombinant expression vectors comprising the one or more nucleic acids of claim 14.

16. A host cell genetically modified with the one or more nucleic acids of claim 14 or the one or more recombinant expression vectors of claim 15.

17. A method of detecting an antigen-specific T cell, the method comprising contacting a T cell with the antigen-presenting polypeptide of any one of claims 9-12, wherein binding of the antigen-presenting polypeptide to the T cell indicates that the T cell is specific for the epitope present in the antigen-presenting polypeptide.

18. A method of selectively modulating the activity of an epitope-specific T cell, the method comprising contacting the T cell with the T-cell modulatory antigen-presenting polypeptide of any one of claims 1-8, wherein said contacting selectively modulates the activity of the epitope-specific T cell.

19. A treatment method, the method comprising administering to an individual in need thereof an effective amount of the T-cell modulatory antigen-presenting polypeptide of any one of claims 1-8, wherein said administering treats the individual.

20. A method of delivering a costimulatory polypeptide selectively to target a T cell, the method comprising contacting a mixed population of T cells with a T-cell modulatory antigen-presenting polypeptide of any one of claims 1-8, wherein the mixed population of T cells comprises the target T cell and non-target T cells,
wherein the target T cell is specific for the epitope present within the T-cell modulatory antigen-presenting polypeptide, and
wherein said contacting delivers the costimulatory polypeptide present within the T-cell modulatory antigen-presenting polypeptide to the target T cell.

21. A method of detecting, in a mixed population of T cells obtained from an individual, the presence of a target T cell that binds an epitope of interest, the method comprising:
a) contacting in vitro the mixed population of T cells with the T-cell modulatory antigen-presenting polypeptide of any one of claims 1-8, wherein the T-cell modulatory antigen-presenting polypeptide comprises the epitope of interest; and
b) detecting activation and/or proliferation of T cells in response to said contacting, wherein activated and/or proliferated T cells indicates the presence of the target T cell.


SEQUENCE LISTING (357 sequences)

SEQ ID NO: 1 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 2 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 3 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - protein, 208 aa, Homo sapiens [sequence omitted]

SEQ ID NO: 5 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 101 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 9 - protein, 110 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 10 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 11 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 12 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 14 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 15 - protein, 133 aa, Homo sapiens [sequence omitted]

SEQ ID NO: 16 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 17 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 18 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 19 - protein, 13 aa, Artificial sequence
  Synthetic polypeptide
PKYVKQNTLKLAT

SEQ ID NO: 20 - protein, 173 aa, Homo sapiens
VIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 21 - protein, 79 aa, Homo sapiens
VIIQAEFYLNPDQSGEFMFDFDGDEIFHVDMAKKETVWRLEEFGRFASFEAQGALANIAVDKANLEIMTKRSNYTPITN

SEQ ID NO: 22 - protein, 94 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 23 - protein, 191 aa, Homo sapiens
VPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYW

SEQ ID NO: 24 - protein, 98 aa, Homo sapiens
VPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSR

SEQ ID NO: 25 - protein, 93 aa, Homo sapiens
GFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYW

SEQ ID NO: 26 - protein, 179 aa, Homo sapiens
TKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHW

SEQ ID NO: 27 - protein, 85 aa, Homo sapiens
TKADHMGSYGPAFYQSYGASGQFTHEFDEEQLFSVDLKKSEAVWRLPEFGDFARFDPQGGLAGIAAIKAHLDILVERSNRSRAIN

SEQ ID NO: 28 - protein, 94 aa, Homo sapiens
VPPRVTVLPKSRVELGQPNILICIVDNIFPPVINITWLRNGQTVTEGVAQTSFYSQPDHLFRKFHYLPFVPSAEDVYDCQVEHWGLDAPLLRHW

SEQ ID NO: 29 - protein, 181 aa, Homo sapiens
AGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 30 - protein, 85 aa, Homo sapiens
AIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 31 - protein, 94 aa, Homo sapiens
DPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 32 - protein, 181 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 33 - protein, 87 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATN

SEQ ID NO: 34 - protein, 94 aa, Homo sapiens
EVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 35 - protein, 181 aa, Homo sapiens
EDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 36 - protein, 87 aa, Homo sapiens
EDIVADHVASYGVNFYQSHGPSGQYTHEFDGDEEFYVDLETKETVWQLPMFSKFISFDPQSALRNMAVGKHTLEFMMRQSNSTAATN

SEQ ID NO: 37 - protein, 94 aa, Homo sapiens
EVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 38 - protein, 197 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 39 - protein, 94 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 40 - protein, 103 aa, Homo sapiens
YPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 41 - protein, 197 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 42 - protein, 94 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRV

SEQ ID NO: 43 - protein, 103 aa, Homo sapiens
HPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 44 - protein, 208 aa, Homo sapiens
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 45 - protein, 105 aa, Homo sapiens
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRV

SEQ ID NO: 46 - protein, 103 aa, Homo sapiens
QPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 47 - protein, 207 aa, Homo sapiens
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 48 - protein, 105 aa, Homo sapiens
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRV

SEQ ID NO: 49 - protein, 102 aa, Homo sapiens
EPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 50 - protein, 189 aa, Homo sapiens
GGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHTGAPEPILRDW

SEQ ID NO: 51 - protein, 94 aa, Homo sapiens
GGFVAHVESTCLLDDAGTPKDFTYCISFNKDLLTCWDPEENKMAPCEFGVLNSLANVLSQHLNQKDTLMQRLRNGLQNCATHTQPFWGSLTNRT

SEQ ID NO: 52 - protein, 95 aa, Homo sapiens
RPPSVQVAKTTPFNTREPVMLACYVWGFYPAEVTITWRKNGKLVMPHSSAHKTAQPNGDWTYQTLSHLALTPSYGDTYTCVVEHTGAPEPILRDW

SEQ ID NO: 53 - protein, 188 aa, Homo sapiens
TDSPEDFVIQAKADCYFTNGTEKVQFVVRFIFNLEEYVRFDSDVGMFVALTKLGQPDAEQWNSRLDLLERSRQAVDGVCRHNYRLGAPFTVGRKVQPEVTVYPERTPLLHQHNLLHCSVTGFYPGDIKIKWFLNGQEERAGVMSTGPIRNGDWTFQTVVMLEMTPELGHVYTCLVDHSSLLSPVSVEW

SEQ ID NO: 54 - protein, 94 aa, Homo sapiens
TDSPEDFVIQAKADCYFTNGTEKVQFVVRFIFNLEEYVRFDSDVGMFVALTKLGQPDAEQWNSRLDLLERSRQAVDGVCRHNYRLGAPFTVGRK

SEQ ID NO: 55 - protein, 94 aa, Homo sapiens
VQPEVTVYPERTPLLHQHNLLHCSVTGFYPGDIKIKWFLNGQEERAGVMSTGPIRNGDWTFQTVVMLEMTPELGHVYTCLVDHSSLLSPVSVEW

SEQ ID NO: 56 - protein, 186 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 57 - protein, 92 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRR

SEQ ID NO: 58 - protein, 94 aa, Homo sapiens
VQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 59 - protein, 188 aa, Homo sapiens
RDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEW

SEQ ID NO: 60 - protein, 94 aa, Homo sapiens
RDSPEDFVFQFKGMCYFTNGTERVRLVTRYIYNREEYARFDSDVGVYRAVTPQGRPDAEYWNSQKEVLEGTRAELDTVCRHNYEVAFRGILQRR

SEQ ID NO: 61 - protein, 94 aa, Homo sapiens
VEPTVTISPSRTEALNHHNLLVCSVTDFYPGQIKVRWFRNDQEETAGVVSTPLIRNGDWTFQILVMLEMTPQRGDVYTCHVEHPSLQSPITVEW

SEQ ID NO: 62 - protein, 182 aa, Homo sapiens
DFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEW

SEQ ID NO: 63 - protein, 93 aa, Homo sapiens
DFLVQFKGMCYFTNGTERVRGVARYIYNREEYGRFDSDVGEFQAVTELGRSIEDWNNYKDFLEQERAAVDKVCRHNYEAELRTTLQRQVEPTV

SEQ ID NO: 64 - protein, 89 aa, Homo sapiens
TISPSRTEALNHHNLLVCSVTDFYPAQIKVRWFRNDQEETAGVVSTSLIRNGDWTFQILVMLEITPQRGDIYTCQVEHPSLQSPITVEW

SEQ ID NO: 65 - protein, 5 aa, Artificial sequence
  Synthetic polypeptide
  Misc_feature (4)..(4): an amino acid other than proline
  Misc_feature (4)..(4): any amino acid other than proline
VPGXG

SEQ ID NO: 66 - protein, 5 aa, Artificial sequence
  Synthetic polypeptide
GSGGS

SEQ ID NO: 67 - protein, 4 aa, Artificial sequence
  Synthetic polypeptide
GGGS

SEQ ID NO: 68 - protein, 4 aa, Artificial sequence
  Synthetic polypeptide
GGSG

SEQ ID NO: 69 - protein, 5 aa, Artificial sequence
  Synthetic polypeptide
GGSGG

SEQ ID NO: 70 - protein, 5 aa, Artificial sequence
  Synthetic polypeptide
GSGSG

SEQ ID NO: 71 - protein, 5 aa, Artificial sequence
  Synthetic polypeptide
GSGGG

SEQ ID NO: 72 - protein, 5 aa, Artificial sequence
  Synthetic polypeptide
GGGSG

SEQ ID NO: 73 - protein, 5 aa, Artificial sequence
  Synthetic polypeptide
GSSSG

SEQ ID NO: 74 - protein, 5 aa, Artificial sequence
  Synthetic polypeptide
GSSSS

SEQ ID NO: 75 - protein, 5 aa, Artificial sequence
  Synthetic polypeptide
  Misc_feature (1)..(5): This stretch of amino acid residues may be repeated
GGGGS

SEQ ID NO: 76 - protein, 5 aa, Artificial sequence
  Synthetic polypeptide
AAAGG

SEQ ID NO: 77 - protein, 15 aa, Artificial sequence
  Synthetic polypeptide
GCGASGGGGSGGGGS

SEQ ID NO: 78 - protein, 9 aa, Artificial sequence
  Synthetic polypeptide
LLMGTLGIV

SEQ ID NO: 79 - protein, 8 aa, Artificial sequence
  Synthetic polypeptide
TLGIVCPI

SEQ ID NO: 80 - protein, 10 aa, Artificial sequence
  Synthetic polypeptide
YMLDLQPETT

SEQ ID NO: 81 - protein, 9 aa, Artificial sequence
  Synthetic polypeptide
YMLDLQPET

SEQ ID NO: 82 - protein, 17 aa, Artificial sequence
  Synthetic polypeptide
GAGSLQPLALEGSLQKR

SEQ ID NO: 83 - protein, 15 aa, Artificial sequence
  Synthetic polypeptide
GIVDQCCTSICSLYQ

SEQ ID NO: 84 - protein, 15 aa, Artificial sequence
  Synthetic polypeptide
GIVEQCCTSICSLYQ

SEQ ID NO: 85 - protein, 13 aa, Artificial sequence
  Synthetic polypeptide
NFFRMVISNPAAT

SEQ ID NO: 86 - protein, 13 aa, Artificial sequence
  Synthetic polypeptide
NFIRMVISNPAAT

SEQ ID NO: 87 - protein, 20 aa, Artificial sequence
  Synthetic polypeptide
SFYLKNVQTQETRTLTQFHF

SEQ ID NO: 88 - protein, 133 aa, Homo sapiens
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 92 aa, Homo sapiens
DTRPRFLWQHKFECHFFNGTERVRLLERCIYNQEESVRFDSDVGEYRAVTELGRPDAEYWNSQKDLLEQRRAAVDTYCRHNYGVGESFTVQR

SEQ ID NO: 90 - protein, 84 aa, Homo sapiens
IKEEHVIIQAEFYLNPDQSGEFMFDFDGDEIFHVDMAKKETVWRLEEFGRFASFEAQGALANIAVDKANLEIMTKRSNYTPITN

SEQ ID NO: 91 - protein, 105 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPET

SEQ ID NO: 92 - protein, 47 aa, Artificial sequence
  Synthetic polypeptide
LEIRAAFLRQRNTALRTEVAELEQEVQRLENEVSQYETRYGPLGGGK

SEQ ID NO: 93 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 98 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

SEQ ID NO: 95 - protein, 103 aa, Homo sapiens
PKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKM

SEQ ID NO: 96 - protein, 14 aa, Artificial sequence
  Synthetic polypeptide
LPLKMLNIPSINVH

SEQ ID NO: 97 - protein, 92 aa, Homo sapiens
DTRPRFLWQHKFECHFFNGTERVRLLERCIYNQEESVRFDSDVGEYRAVTELGRPAAEYWNSQKDLLEQRRAAVDTYCRHNYGVGESFTVQR

SEQ ID NO: 98 - protein, 105 aa, Homo sapiens
VEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKM

SEQ ID NO: 99 - protein, 47 aa, Artificial sequence
  Synthetic polypeptide
LEIEAAFLERENTALETRVAELRQRVQRLRNRVSQYRTRYGPLGGGK

SEQ ID NO: 100 - protein, 92 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQR

SEQ ID NO: 101 - protein, 105 aa, Homo sapiens
VYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 102 - protein, 93 aa, homo sapiens
GDTRPRFLWQHKFECHFFNGTERVRLLERCIYNQEESVRFDSDVGEYRAVTELGRPDAEYWNSQKDLLEQRRAAVDTYCRHNYGVGESFTVQR

SEQ ID NO: 103 - protein, 79 aa, Homo sapiens
IKEEHVIIQAEFYLNPDQSGEFMFDFDGDEIFHVDMAKKETVWRLEEFGRFASFEAQGALANIAVDKANLEIMTKRSNY

SEQ ID NO: 104 - protein, 95 aa, Homo sapiens
EVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

SEQ ID NO: 105 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 96 aa, Homo sapiens
DTRPRFLWQHKFECHFFNGTERVRLLERCIYNQEESVRFDSDVGEYRAVTELGRPDAEYWNSQKDLLEQRRAAVDTYCRHNYGVGESFTVQRRVEP

SEQ ID NO: 107 - protein, 95 aa, Homo sapiens
KVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARS

SEQ ID NO: 108 - protein, 219 aa, Artificial sequence
  Synthetic polypeptide
  Misc_feature (8)..(8): any amino acid other than aspartic acid
FTVTVPKXLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 109 - protein, 219 aa, Artificial sequence
  Synthetic polypeptide
  Misc_feature (36)..(36): any amino acid other than isoleucine
FTVTVPKDLYVVEYGSNMTIECKFPVEKQLDLAALXVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 110 - protein, 219 aa, Artificial sequence
  Synthetic polypeptide
  Misc_feature (54)..(54): any amino acid other than glutamine
FTVTVPKDLYVVEYGSNMTIECKFPVEKQLDLAALIVYWEMEDKNIIQFVHGEXDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 111 - protein, 208 aa, Artificial sequence
  Synthetic polypeptide
  Misc_feature (19)..(19): any amino acid other than asparagine
VIHVTKEVKEVATLSCGHXVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 112 - protein, 208 aa, Artificial sequence
  Synthetic polypeptide
  Misc_feature (63)..(63): any amino acid other than asparagine
VIHVTKEVKEVATLSCGHNVSVEELAQTRIYWQKEKKMVLTMMSGDMNIWPEYKNRTIFDITXNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 113 - protein, 208 aa, Artificial sequence
  Synthetic polypeptide
  Misc_feature (67)..(67): any amino acid other than isoleucine
VIHVTKEVKEVATLSCGHNVSVEELAQTRIYWQKEKKMVLTMMSGDMNIWPEYKNRTIFDITNNLSXVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 114 - protein, 208 aa, Artificial sequence
  Synthetic polypeptide
  Misc_feature (86)..(86): Xaa is a lysine
VIHVTKEVKEVATLSCGHNVSVEELAQTRIYWQKEKKMVLTMMSGDMNIWPEYKNRTIFDITNNLSIVILALRPSDEGTYECVVLXYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 115 - protein, 208 aa, Artificial sequence
  Synthetic polypeptide
  Misc_feature (157)..(157): any amino acid other than glutamine
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSXDPETELYAVSSKLDFNMTTNHSFMCLIKYGHLRVNQTFNWNTTKQEHFPDN

SEQ ID NO: 116 - protein, 208 aa, Artificial sequence
  Synthetic polypeptide
  Misc_feature (158)..(158): any amino acid other than aspartic acid
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQXPETELYAVSSKLDFNMTTNHSFMCLIKYGHLRVNQTFNWNTTKQEHFPDN

SEQ ID NO: 117 - protein, 208 aa, Artificial sequence
  Synthetic polypeptide
  Misc_feature (25)..(25): any amino acid other than leucine
VIHVTKEVKEVATLSCGHNVSVEEXAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 118 - protein, 208 aa, Artificial sequence
  Synthetic polypeptide
  Misc_feature (31)..(31): any amino acid other than tyrosine
VIHVTKEVKEVATLSCGHNVSVEELAQTRIXWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 119 - protein, 208 aa, Artificial sequence
  Synthetic polypeptide
  Misc_feature (33)..(33): any amino acid other than glutamine
VIHVTKEVKEVATLSCGHNVSVEELAQTRIYWXKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 120 - protein, 208 aa, Artificial sequence
  Synthetic polypeptide
  Misc_feature (38)..(38): any amino acid other than methionine
VIHVTKEVKEVATLSCGHNVSVEELAQTRIYWQKEKKXVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 121 - protein, 208 aa, Artificial sequence
  Synthetic polypeptide
  Misc_feature (39)..(39): any amino acid other than valine
VIHVTKEVKEVATLSCGHNVSVEELAQTRIYWQKEKKMXLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 122 - protein, 208 aa, Artificial sequence
  Synthetic polypeptide
  Misc_feature (49)..(49): any amino acid other than isoleucine
VIHVTKEVKEVATLSCGHNVSVEELAQTRIYWQKEKKMVLTMMSGDMNXWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 123 - protein, 208 aa, Artificial sequence
  Synthetic polypeptide
  Misc_feature (53)..(53): any amino acid other than tyrosine
VIHVTKEVKEVATLSCGHNVSVEELAQTRIYWQKEKKMVLTMMSGDMNIWPEXKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 124 - protein, 208 aa, Artificial sequence
  Synthetic polypeptide
  Misc_feature (60)..(60): any amino acid other than aspartic acid
VIHVTKEVKEVATLSCGHNVSVEELAQTRIYWQKEKKMVLTMMSGDMNIWPEYKNRTIFXITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 125 - protein, 208 aa, Artificial sequence
  Synthetic polypeptide
  Misc_feature (108)..(108): any amino acid other than phenylalanine
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADXPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 126 - protein, 208 aa, Artificial sequence
  Synthetic polypeptide
  Misc_feature (156)..(156): amino acid other than serine
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVXQDPETELYAVSSKLDFNMTTNHSFMCLIKYGHLRVNQTFNWNTTKQEHFPDN

SEQ ID NO: 127 - protein, 208 aa, Artificial sequence
  Synthetic polypeptide
  Misc_feature (111)..(111): any amino acid other than alanine
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTXSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 128 - protein, 224 aa, Artificial sequence
  Synthetic polypeptide
  Misc_feature (61)..(61): any amino acid other than asparagine
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 129 - protein, 224 aa, Artificial sequence
  Synthetic polypeptide
  Misc_feature (66)..(66): any amino acid other than aspartic acid
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 130 - protein, 224 aa, Artificial sequence
  Synthetic polypeptide
  Misc_feature (70)..(70): Xaa is tryptophan
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 131 - protein, 224 aa, Artificial sequence
  Synthetic polypeptide
  Misc_feature (91)..(91): any amino acid other athan histidine
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 132 - protein, 110 aa, Artificial sequence
  Synthetic polypeptide
  Misc_feature (61)..(61): Xaa is a asparagine
APLKIQAYFNETADLPCQFANSQNQSLSELVVFWQDQENLVLNEVYLGKEKFDSVHSKYMXRTSFDSDSWTLRLHNLQIKDKGLYQCIIHHKKPTGMIRIHQMNSELSVL

SEQ ID NO: 133 - protein, 110 aa, Artificial sequence
  Synthetic polypeptide
  Misc_feature (66)..(66): any amino acid other than aspartic acid
APLKIQAYFNETADLPCQFANSQNQSLSELVVFWQDQENLVLNEVYLGKEKFDSVHSKYMNRTSFXSDSWTLRLHNLQIKDKGLYQCIIHHKKPTGMIRIHQMNSELSVL

SEQ ID NO: 134 - protein, 110 aa, Artificial sequence
  Synthetic polypeptide
  Misc_feature (70)..(70): any amino acid other than tryptophan
APLKIQAYFNETADLPCQFANSQNQSLSELVVFWQDQENLVLNEVYLGKEKFDSVHSKYMNRTSFDSDSXTLRLHNLQIKDKGLYQCIIHHKKPTGMIRIHQMNSELSVL

SEQ ID NO: 135 - protein, 110 aa, Artificial sequence
  Synthetic polypeptide
  Misc_feature (91)..(91): any amino acid other than histidine
APLKIQAYFNETADLPCQFANSQNQSLSELVVFWQDQENLVLNEVYLGKEKFDSVHSKYMNRTSFDSDSWTLRLHNLQIKDKGLYQCIIHXKKPTGMIRIHQMNSELSVL

SEQ ID NO: 136 - protein, 224 aa, Artificial sequence
  Synthetic polypeptide
  Misc_feature (41)..(41): any amino acid other than valine
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 137 - protein, 110 aa, Artificial sequence
  Synthetic polypeptide
  Misc_feature (41)..(41): any amino acid other than valine
APLKIQAYFNETADLPCQFANSQNQSLSELVVFWQDQENLXLNEVYLGKEKFDSVHSKYMNRTSFDSDSWTLRLHNLQIKDKGLYQCIIHHKKPTGMIRIHQMNSELSVL

SEQ ID NO: 138 - protein, 224 aa, Artificial sequence
  Synthetic polypeptide
  Misc_feature (35)..(35): any amino acid other than glutamine
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 139 - protein, 110 aa, Artificial sequence
  Synthetic polypeptide
  Misc_feature (35)..(35): any amino acid other than glutamine
APLKIQAYFNETADLPCQFANSQNQSLSELVVFWXDQENLVLNEVYLGKEKFDSVHSKYMNRTSFDSDSWTLRLHNLQIKDKGLYQCIIHHKKPTGMIRIHQMNSELSVL

SEQ ID NO: 140 - protein, 224 aa, Artificial sequence
  Synthetic polypeptide
  Misc_feature (33)..(33): any amino acid other than phenylalanine
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 141 - protein, 110 aa, Artificial sequence
  Synthetic polypeptide
  Misc_feature (33)..(33): any amino acid other than phenylalanine
APLKIQAYFNETADLPCQFANSQNQSLSELVVXWQDQENLVLNEVYLGKEKFDSVHSKYMNRTSFDSDSWTLRLHNLQIKDKGLYQCIIHHKKPTGMIRIHQMNSELSVL

SEQ ID NO: 142 - protein, 224 aa, Artificial sequence
  Synthetic polypeptide
  Misc_feature (72)..(72): any amino acid other than leucine
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 143 - protein, 110 aa, Artificial sequence
  Synthetic polypeptide
  Misc_feature (72)..(72): any amino acid other than leucine
APLKIQAYFNETADLPCQFANSQNQSLSELVVFWQDQENLVLNEVYLGKEKFDSVHSKYMNRTSFDSDSWTXRLHNLQIKDKGLYQCIIHHKKPTGMIRIHQMNSELSVL

SEQ ID NO: 144 - protein, 224 aa, Artificial sequence
  Synthetic polypeptide
  Misc_feature (59)..(59): any amino acid other than tyrosine
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 145 - protein, 110 aa, Artificial sequence
  Synthetic polypeptide
  Misc_feature (59)..(59): any amino acid other than tyrosine
APLKIQAYFNETADLPCQFANSQNQSLSELVVFWQDQENLVLNEVYLGKEKFDSVHSKXMNRTSFDSDSWTLRLHNLQIKDKGLYQCIIHHKKPTGMIRIHQMNSELSVL

SEQ ID NO: 146 - protein, 224 aa, Artificial sequence
  Synthetic polypeptide
  Misc_feature (61)..(61): any amino acid other than asparagine
  Misc_feature (91)..(91): any amino acid other than hisidine
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 147 - protein, 110 aa, Artificial sequence
  Synthetic polypeptide
  Misc_feature (61)..(61): any amino acid other than asparagine
  Misc_feature (91)..(91): any amino acid other than histidine
APLKIQAYFNETADLPCQFANSQNQSLSELVVFWQDQENLVLNEVYLGKEKFDSVHSKYMXRTSFDSDSWTLRLHNLQIKDKGLYQCIIHXKKPTGMIRIHQMNSELSVL

SEQ ID NO: 148 - protein, 224 aa, Artificial sequence
  Synthetic polypeptide
  Misc_feature (66)..(66): any amino acid other than asparagine
  Misc_feature (91)..(91): any amino acid other than histidine
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 149 - protein, 110 aa, Artificial sequence
  Synthetic polypeptide
  Misc_feature (66)..(66): any amino acid other than asparagine
  Misc_feature (91)..(91): any amino acid other than histidine
APLKIQAYFNETADLPCQFANSQNQSLSELVVFWQDQENLVLNEVYLGKEKFDSVHSKYMNRTSFXSDSWTLRLHNLQIKDKGLYQCIIHXKKPTGMIRIHQMNSELSVL

SEQ ID NO: 150 - protein, 224 aa, Artificial sequence
  Synthetic polypeptide
  misc_feature (61)..(61): any amino acid other than asparagine
  misc_feature (66)..(66): any amino acid other than aspartic acid
  misc_feature (91)..(91): any amino acid other than histidine
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 151 - protein, 110 aa, Artificial sequence
  Synthetic polypeptide
  Misc_feature (61)..(61): any amino acid other than asparagine
  Misc_feature (66)..(66): any amino acid other than aspartic acid
  Misc_feature (91)..(91): any amino acid other than histidine
APLKIQAYFNETADLPCQFANSQNQSLSELVVFWQDQENLVLNEVYLGKEKFDSVHSKYMXRTSFXSDSWTLRLHNLQIKDKGLYQCIIHXKKPTGMIRIHQMNSELSVL

SEQ ID NO: 152 - protein, 174 aa, Artificial sequence
  Synthetic polypeptide
  Misc_feature (47)..(47): any amino acid other than lysine
PAGLLDLRQGMFAQLVAQNVLLIDGPLSWYSDPGLAGVSLTGGLSYXEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 153 - protein, 174 aa, Artificial sequence
  Synthetic polypeptide
  Misc_feature (147)..(147): any amino acid other than glutamine
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWXLTQGATVLGLFRVTPEIPAGLPSPRSE

SEQ ID NO: 154 - protein, 174 aa, Artificial sequence
  Synthetic polypeptide
  Misc_feature (11)..(11): any amino acid other than methionine
PAGLLDLRQGXFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 155 - protein, 174 aa, Artificial sequence
  Synthetic polypeptide
  Misc_feature (12)..(12): any amino acid other than phenylalanine
PAGLLDLRQGMXAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 156 - protein, 174 aa, Artificial sequence
  Synthetic polypeptide
  Misc_feature (14)..(14): any amino acid other than glutamine
PAGLLDLRQGMFAXLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 157 - protein, 174 aa, Artificial sequence
  Synthetic polypeptide
  Misc_feature (15)..(15): any amino acid other than leucine
PAGLLDLRQGMFAQXVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 158 - protein, 174 aa, Artificial sequence
  Synthetic polypeptide
  Misc_feature (16)..(16): any amino acid other than valine
PAGLLDLRQGMFAQLXAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 159 - protein, 174 aa, Artificial sequence
  Synthetic polypeptide
  Misc_feature (18)..(18): any amino acid other than glutamine
PAGLLDLRQGMFAQLVAXNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 160 - protein, 174 aa, Artificial sequence
  Synthetic polypeptide
  Misc_feature (19)..(19): any amino acid other than asparagine
PAGLLDLRQGMFAQLVAQXVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 161 - protein, 174 aa, Artificial sequence
  Synthetic polypeptide
  Misc_feature (20)..(20): any amino acid other than valine
PAGLLDLRQGMFAQLVAQNXLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 162 - protein, 174 aa, Artificial sequence
  Synthetic polypeptide
  Misc_feature (21)..(21): any amino acid other than leucine
PAGLLDLRQGMFAQLVAQNVXLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 163 - protein, 174 aa, Artificial sequence
  Synthetic polypeptide
  Misc_feature (22)..(22): any amino acid other than leucine
PAGLLDLRQGMFAQLVAQNVLXIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 164 - protein, 174 aa, Artificial sequence
  Synthetic polypeptide
  Misc_feature (23)..(23): any amino acid other than isolucine
PAGLLDLRQGMFAQLVAQNVLLXDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 165 - protein, 174 aa, Artificial sequence
  Synthetic polypeptide
  Misc_feature (24)..(24): any amino acid other than asparagine
PAGLLDLRQGMFAQLVAQNVLLIXGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 166 - protein, 174 aa, Artificial sequence
  Synthetic polypeptide
  Misc_feature (25)..(25): any amino acid other than glycine
PAGLLDLRQGMFAQLVAQNVLLIDXPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 167 - protein, 174 aa, Artificial sequence
  Synthetic polypeptide
  Misc_feature (26)..(26): any amino acid other than proline
PAGLLDLRQGMFAQLVAQNVLLIGGXLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 168 - protein, 174 aa, Artificial sequence
  Synthetic polypeptide
  Misc_feature (27)..(27): any amino acid other than leucine
PAGLLDLRQGMFAQLVAQNVLLIGGPXSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 169 - protein, 174 aa, Artificial sequence
  Synthetic polypeptide
  Misc_feature (28)..(28): any amino acid other than serine
PAGLLDLRQGMFAQLVAQNVLLIGGPLXWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 170 - protein, 174 aa, Artificial sequence
  Synthetic polypeptide
  Misc_feature (29)..(29): any amino acid other than tryptophan
PAGLLDLRQGMFAQLVAQNVLLIGGPLSXYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 171 - protein, 174 aa, Artificial sequence
  Synthetic polypeptide
  Misc_feature (30)..(30): any amino acid other than tyrosine
PAGLLDLRQGMFAQLVAQNVLLIGGPLSWXSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 172 - protein, 174 aa, Artificial sequence
  Synthetic polypeptide
  Misc_feature (31)..(31): any amino acid other than serine
PAGLLDLRQGMFAQLVAQNVLLIGGPLSWYXDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 173 - protein, 174 aa, Artificial sequence
  Synthetic polypeptide
  Misc_feature (32)..(32): any amino acid other than aspartic acid
PAGLLDLRQGMFAQLVAQNVLLIGGPLSWYSXPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 174 - protein, 174 aa, Artificial sequence
  Synthetic polypeptide
  Misc_feature (33)..(33): any amino acid other than proline
PAGLLDLRQGMFAQLVAQNVLLIGGPLSWYSDXGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 175 - protein, 174 aa, Artificial sequence
  Synthetic polypeptide
  Misc_feature (34)..(34): any amino acid other than glycine
PAGLLDLRQGMFAQLVAQNVLLIGGPLSWYSDPXLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 176 - protein, 174 aa, Artificial sequence
  Synthetic polypeptide
  Misc_feature (35)..(35): any amino acid other than leucine
PAGLLDLRQGMFAQLVAQNVLLIGGPLSWYSDPGXAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 177 - protein, 174 aa, Artificial sequence
  Synthetic polypeptide
  Misc_feature (37)..(37): any amino acid other than glycine
PAGLLDLRQGMFAQLVAQNVLLIGGPLSWYSDPGLAXVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 178 - protein, 174 aa, Artificial sequence
  Synthetic polypeptide
  Misc_feature (38)..(38): any amino acid other than valine
PAGLLDLRQGMFAQLVAQNVLLIGGPLSWYSDPGLAGXSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 179 - protein, 174 aa, Artificial sequence
  Synthetic polypeptide
  Misc_feature (39)..(39): any amino acid other than serine
PAGLLDLRQGMFAQLVAQNVLLIGGPLSWYSDPGLAGVXLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 180 - protein, 174 aa, Artificial sequence
  Synthetic polypeptide
  Misc_feature (40)..(40): any amino acid other than leucine
PAGLLDLRQGMFAQLVAQNVLLIGGPLSWYSDPGLAGVSXTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 181 - protein, 174 aa, Artificial sequence
  Synthetic polypeptide
  Misc_feature (41)..(41): any amino acid other than threonine
PAGLLDLRQGMFAQLVAQNVLLIGGPLSWYSDPGLAGVSLXGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 182 - protein, 174 aa, Artificial sequence
  Synthetic polypeptide
  Misc_feature (42)..(42): any amino acid other than glycine
PAGLLDLRQGMFAQLVAQNVLLIGGPLSWYSDPGLAGVSLTXGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 183 - protein, 174 aa, Artificial sequence
  Synthetic polypeptide
  Misc_feature (43)..(43): any amino acid other than glycine
PAGLLDLRQGMFAQLVAQNVLLIGGPLSWYSDPGLAGVSLTGXLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 184 - protein, 174 aa, Artificial sequence
  Synthetic polypeptide
  Misc_feature (44)..(44): any amino acid other than leucine
PAGLLDLRQGMFAQLVAQNVLLIGGPLSWYSDPGLAGVSLTGGXSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 185 - protein, 174 aa, Artificial sequence
  Synthetic polypeptide
  Misc_feature (45)..(45): any amino acid other than serine
PAGLLDLRQGMFAQLVAQNVLLIGGPLSWYSDPGLAGVSLTGGLXYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 186 - protein, 174 aa, Artificial sequence
  Synthetic polypeptide
  Misc_feature (46)..(46): any amino acid other than tyrosine
PAGLLDLRQGMFAQLVAQNVLLIGGPLSWYSDPGLAGVSLTGGLSXKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 187 - protein, 174 aa, Artificial sequence
  Synthetic polypeptide
  Misc_feature (48)..(48): any amino acid other than glutamic acid
PAGLLDLRQGMFAQLVAQNVLLIGGPLSWYSDPGLAGVSLTGGLSYKXDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 188 - protein, 174 aa, Artificial sequence
  Synthetic polypeptide
  Misc_feature (49)..(49): any amino acid other than aspartic acid
PAGLLDLRQGMFAQLVAQNVLLIGGPLSWYSDPGLAGVSLTGGLSYKEXTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 189 - protein, 174 aa, Artificial sequence
  Synthetic polypeptide
  Misc_feature (50)..(50): any amino acid other than threonine
PAGLLDLRQGMFAQLVAQNVLLIGGPLSWYSDPGLAGVSLTGGLSYKEDXKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 190 - protein, 174 aa, Artificial sequence
  Synthetic polypeptide
  Misc_feature (51)..(51): any amino acid other than lysine
PAGLLDLRQGMFAQLVAQNVLLIGGPLSWYSDPGLAGVSLTGGLSYKEDTXELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 191 - protein, 174 aa, Artificial sequence
  Synthetic polypeptide
  Misc_feature (52)..(52): any amino acid other than glutamic acid
PAGLLDLRQGMFAQLVAQNVLLIGGPLSWYSDPGLAGVSLTGGLSYKEDTKXLVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 192 - protein, 174 aa, Artificial sequence
  Synthetic polypeptide
  Misc_feature (64)..(64): any amino acid other than phenylalanine
PAGLLDLRQGMFAQLVAQNVLLIGGPLSWYSDPGLAGVSLTGGLSYKEDTKELVVAKAGVYYVXFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 193 - protein, 174 aa, Artificial sequence
  Synthetic polypeptide
  Misc_feature (65)..(65): any amino acid other than phenylalanine
PAGLLDLRQGMFAQLVAQNVLLIGGPLSWYSDPGLAGVSLTGGLSYKEDTKELVVAKAGVYYVFXQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 194 - protein, 174 aa, Artificial sequence
  Synthetic polypeptide
  Misc_feature (66)..(66): any amino acid other than glutamine
PAGLLDLRQGMFAQLVAQNVLLIGGPLSWYSDPGLAGVSLTGGLSYKEDTKELVVAKAGVYYVFFXLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 195 - protein, 174 aa, Artificial sequence
  Synthetic polypeptide
  Misc_feature (67)..(67): any amino acid other than leucine
PAGLLDLRQGMFAQLVAQNVLLIGGPLSWYSDPGLAGVSLTGGLSYKEDTKELVVAKAGVYYVFFQXELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 196 - protein, 174 aa, Artificial sequence
  Synthetic polypeptide
  Misc_feature (68)..(68): any amino acid other than glutamic acid
PAGLLDLRQGMFAQLVAQNVLLIGGPLSWYSDPGLAGVSLTGGLSYKEDTKELVVAKAGVYYVFFQLXLRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 197 - protein, 174 aa, Artificial sequence
  Synthetic polypeptide
  Misc_feature (69)..(69): any amino acid other than leucine
PAGLLDLRQGMFAQLVAQNVLLIGGPLSWYSDPGLAGVSLTGGLSYKEDTKELVVAKAGVYYVFFQLEXRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 198 - protein, 174 aa, Artificial sequence
  Synthetic polypeptide
  Misc_feature (70)..(70): any amino acid other than arginine
PAGLLDLRQGMFAQLVAQNVLLIGGPLSWYSDPGLAGVSLTGGLSYKEDTKELVVAKAGVYYVFFQLELXRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 199 - protein, 174 aa, Artificial sequence
  Synthetic polypeptide
  Misc_feature (71)..(71): any amino acid other than arginine
PAGLLDLRQGMFAQLVAQNVLLIGGPLSWYSDPGLAGVSLTGGLSYKEDTKELVVAKAGVYYVFFQLELRXVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 200 - protein, 174 aa, Artificial sequence
  Synthetic polypeptide
  Misc_feature (72)..(72): any amino acid other than valine
PAGLLDLRQGMFAQLVAQNVLLIGGPLSWYSDPGLAGVSLTGGLSYKEDTKELVVAKAGVYYVFFQLELRRXVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 201 - protein, 174 aa, Artificial sequence
  Synthetic polypeptide
  Misc_feature (73)..(73): any amino acid other than valine
PAGLLDLRQGMFAQLVAQNVLLIGGPLSWYSDPGLAGVSLTGGLSYKEDTKELVVAKAGVYYVFFQLELRRVXAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 202 - protein, 174 aa, Artificial sequence
  Synthetic polypeptide
  Misc_feature (75)..(75): any amino acid other than glycine
PAGLLDLRQGMFAQLVAQNVLLIGGPLSWYSDPGLAGVSLTGGLSYKEDTKELVVAKAGVYYVFFQLELRRVVAXEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 203 - protein, 174 aa, Artificial sequence
  Synthetic polypeptide
  Misc_feature (76)..(76): any amino acid other than gluamic acid
PAGLLDLRQGMFAQLVAQNVLLIGGPLSWYSDPGLAGVSLTGGLSYKEDTKELVVAKAGVYYVFFQLELRRVVAGXGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 204 - protein, 174 aa, Artificial sequence
  Synthetic polypeptide
  Misc_feature (77)..(77): any amino acid other than glycine
PAGLLDLRQGMFAQLVAQNVLLIGGPLSWYSDPGLAGVSLTGGLSYKEDTKELVVAKAGVYYVFFQLELRRVVAGEXSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 205 - protein, 174 aa, Artificial sequence
  Synthetic polypeptide
  Misc_feature (78)..(78): any amino acid other than serine
PAGLLDLRQGMFAQLVAQNVLLIGGPLSWYSDPGLAGVSLTGGLSYKEDTKELVVAKAGVYYVFFQLELRRVVAGEGXGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 206 - protein, 174 aa, Artificial sequence
  Synthetic polypeptide
  Misc_feature (104)..(104): any amino acid other than aspartic acid
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVXLPPASSEARNSAFGFQGRLLHLSAGQRLGVHLHTEARARHAWQLTQGATVLGLFRVTPEIPAGLPSPRSE

SEQ ID NO: 207 - protein, 174 aa, Artificial sequence
  Synthetic polypeptide
  Misc_feature (105)..(105): any amino acid other than leucine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDXPPASSEARNSAFGFQGRLLHLSAGQRLGVHLHTEARARHAWQLTQGATVLGLFRVTPEIPAGLPSPRSE

SEQ ID NO: 208 - protein, 174 aa, Artificial sequence
  Synthetic polypeptide
  Misc_feature (106)..(106): any amino acid other than proline
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLXPASSEARNSAFGFQGRLLHLSAGQRLGVHLHTEARARHAWQLTQGATVLGLFRVTPEIPAGLPSPRSE

SEQ ID NO: 209 - protein, 174 aa, Artificial sequence
  Synthetic polypeptide
  Misc_feature (109)..(109): any amino acid other than serine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPAXSEARNSAFGFQGRLLHLSAGQRLGVHLHTEARARHAWQLTQGATVLGLFRVTPEIPAGLPSPRSE

SEQ ID NO: 210 - protein, 174 aa, Artificial sequence
  Synthetic polypeptide
  Misc_feature (110)..(110): any amino acid other than serine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASXEARNSAFGFQGRLLHLSAGQRLGVHLHTEARARHAWQLTQGATVLGLFRVTPEIPAGLPSPRSE

SEQ ID NO: 211 - protein, 174 aa, Artificial sequence
  Synthetic polypeptide
  Misc_feature (111)..(111): any amino acid other than glutamic acid
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSXARNSAFGFQGRLLHLSAGQRLGVHLHTEARARHAWQLTQGATVLGLFRVTPEIPAGLPSPRSE

SEQ ID NO: 212 - protein, 174 aa, Artificial sequence
  Synthetic polypeptide
  Misc_feature (113)..(113): any amino acid other than arginine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEAXNSAFGFQGRLLHLSAGQRLGVHLHTEARARHAWQLTQGATVLGLFRVTPEIPAGLPSPRSE

SEQ ID NO: 213 - protein, 174 aa, Artificial sequence
  Synthetic polypeptide
  Misc_feature (114)..(114): any amino acid other than asparagine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARXSAFGFQGRLLHLSAGQRLGVHLHTEARARHAWQLTQGATVLGLFRVTPEIPAGLPSPRSE

SEQ ID NO: 214 - protein, 174 aa, Artificial sequence
  Synthetic polypeptide
  Misc_feature (115)..(115): any amino acid other than serine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNXAFGFQGRLLHLSAGQRLGVHLHTEARARHAWQLTQGATVLGLFRVTPEIPAGLPSPRSE

SEQ ID NO: 215 - protein, 174 aa, Artificial sequence
  Synthetic polypeptide
  Misc_feature (117)..(117): any amino acid other than phenylalanine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAXGFQGRLLHLSAGQRLGVHLHTEARARHAWQLTQGATVLGLFRVTPEIPAGLPSPRSE

SEQ ID NO: 216 - protein, 174 aa, Artificial sequence
  Synthetic polypeptide
  Misc_feature (130)..(130): any amino acid other than glutamine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGXRLGVHLHTEARARHAWQLTQGATVLGLFRVTPEIPAGLPSPRSE

SEQ ID NO: 217 - protein, 174 aa, Artificial sequence
  Synthetic polypeptide
  misc_feature (131)..(131): any amino acid other than arginine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQXLGVHLHTEARARHAWQLTQGATVLGLFRVTPEIPAGLPSPRSE

SEQ ID NO: 218 - protein, 174 aa, Artificial sequence
  Synthetic polypeptide
  Misc_feature (132)..(132): any amino acid other than leucine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRXGVHLHTEARARHAWQLTQGATVLGLFRVTPEIPAGLPSPRSE

SEQ ID NO: 219 - protein, 174 aa, Artificial sequence
  Synthetic polypeptide
  Misc_feature (133)..(133): any amino acid other than glycine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLXVHLHTEARARHAWQLTQGATVLGLFRVTPEIPAGLPSPRSE

SEQ ID NO: 220 - protein, 174 aa, Artificial sequence
  Synthetic polypeptide
  Misc_feature (134)..(134): any amino acid other than valine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGXHLHTEARARHAWQLTQGATVLGLFRVTPEIPAGLPSPRSE

SEQ ID NO: 221 - protein, 174 aa, Artificial sequence
  Synthetic polypeptide
  Misc_feature (135)..(135): any amino acid other than histidine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVXLHTEARARHAWQLTQGATVLGLFRVTPEIPAGLPSPRSE

SEQ ID NO: 222 - protein, 174 aa, Artificial sequence
  Synthetic polypeptide
  Misc_feature (136)..(136): any amino acid other than leucine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHXHTEARARHAWQLTQGATVLGLFRVTPEIPAGLPSPRSE

SEQ ID NO: 223 - protein, 174 aa, Artificial sequence
  Synthetic polypeptide
  Misc_feature (137)..(137): any amino acid other than histidine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLXTEARARHAWQLTQGATVLGLFRVTPEIPAGLPSPRSE

SEQ ID NO: 224 - protein, 174 aa, Artificial sequence
  Synthetic polypeptide
  Misc_feature (138)..(138): any amino acid other than threonine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHXEARARHAWQLTQGATVLGLFRVTPEIPAGLPSPRSE

SEQ ID NO: 225 - protein, 174 aa, Artificial sequence
  Synthetic polypeptide
  Misc_feature (139)..(139): any amino acid other than glutamic acid
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTXARARHAWQLTQGATVLGLFRVTPEIPAGLPSPRSE

SEQ ID NO: 226 - protein, 174 aa, Artificial sequence
  Synthetic polypeptide
  Misc_feature (141)..(141): any amino acid other than arginine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEAXARHAWQLTQGATVLGLFRVTPEIPAGLPSPRSE

SEQ ID NO: 227 - protein, 174 aa, Artificial sequence
  Synthetic polypeptide
  Misc_feature (143)..(143): any amino acid other than arginine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARAXHAWQLTQGATVLGLFRVTPEIPAGLPSPRSE

SEQ ID NO: 228 - protein, 174 aa, Artificial sequence
  Synthetic polypeptide
  Misc_feature (144)..(144): any amino acid other than histidine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARXAWQLTQGATVLGLFRVTPEIPAGLPSPRSE

SEQ ID NO: 229 - protein, 174 aa, Artificial sequence
  Synthetic polypeptide
  Misc_feature (146)..(146): any amino acid other than tryptophan
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAXQLTQGATVLGLFRVTPEIPAGLPSPRSE

SEQ ID NO: 230 - protein, 174 aa, Artificial sequence
  Synthetic polypeptide
  Misc_feature (148)..(148): any amino acid other than leucine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQXTQGATVLGLFRVTPEIPAGLPSPRSE

SEQ ID NO: 231 - protein, 174 aa, Artificial sequence
  Synthetic polypeptide
  Misc_feature (149)..(149): any amino acid other than threonine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLXQGATVLGLFRVTPEIPAGLPSPRSE

SEQ ID NO: 232 - protein, 174 aa, Artificial sequence
  Synthetic polypeptide
  Misc_feature (150)..(150): any amino acid other than glutamine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTXGATVLGLFRVTPEIPAGLPSPRSE

SEQ ID NO: 233 - protein, 174 aa, Artificial sequence
  Synthetic polypeptide
  Misc_feature (151)..(151): any amino acid other than glycine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQXATVLGLFRVTPEIPAGLPSPRSE

SEQ ID NO: 234 - protein, 173 aa, Artificial sequence
  Synthetic polypeptide
  Misc_feature (153)..(153): any amino acid other than threonine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGAXVLGLFRVTPEIPAGLPSPRS

SEQ ID NO: 235 - protein, 174 aa, Artificial sequence
  Synthetic polypeptide
  Misc_feature (154)..(154): any amino acid other than valine
PAGLLDLRQGMFAQLVAQNVLLIGGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATXLGLFRVTPEIPAGLPSPRSE

SEQ ID NO: 236 - protein, 133 aa, Artificial sequence
  Synthetic polypeptide
  Misc_feature (42)..(42): any amino acid other than phenylalanine
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 237 - protein, 133 aa, Artificial sequence
  Synthetic polypeptide
  Misc_feature (20)..(20): any amino acid other than aspartic acid
APTSSSTKKTQLQLEHLLLXLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 238 - protein, 133 aa, Artificial sequence
  Synthetic polypeptide
  Misc_feature (15)..(15): any amino acid other than glutamic acid
APTSSSTKKTQLQLXHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 239 - protein, 133 aa, Artificial sequence
  Synthetic polypeptide
  Misc_feature (16)..(16): any amino acid other than histidine
APTSSSTKKTQLQLEXLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 240 - protein, 133 aa, Artificial sequence
  Synthetic polypeptide
  Misc_feature (45)..(45): any amino acid other than tyrosine
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 241 - protein, 133 aa, Artificial sequence
  Synthetic polypeptide
  Misc_feature (126)..(126): any amino acid other than glutamine
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 242 - protein, 133 aa, Artificial sequence
  Synthetic polypeptide
  Misc_feature (16)..(16): any amino acid other than histidine
  Misc_feature (42)..(42): any amino acid other than phenylalanine
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 243 - protein, 133 aa, Artificial sequence
  Synthetic polypeptide
  Misc_feature (20)..(20): any amino acid other than aspartic acid
  Misc_feature (42)..(42): any amino acid other than phenylalanine
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 244 - protein, 133 aa, Artificial sequence
  Synthetic polypeptide
  Misc_feature (15)..(15): any amino acid other than glutamic acid
  Misc_feature (20)..(20): any amino acid other than aspartic acid
  Misc_feature (42)..(42): any amino acid other than phenylalanine
APTSSSTKKTQLQLXHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 245 - protein, 133 aa, Artificial sequence
  Synthetic polypeptide
  Misc_feature (16)..(16): any amino acid other than histidine
  Misc_feature (20)..(20): any amino acid other than aspartic acid
  Misc_feature (42)..(42): any amino acid other than phenylalanine
APTSSSTKKTQLQLEXLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 246 - protein, 133 aa, Artificial sequence
  Synthetic polypeptide
  Misc_feature (20)..(20): any amino acid other than aspratic aicd
  Misc_feature (42)..(42): any amino acid other than phenylalanine
  Misc_feature (126)..(126): any amino acid other than glutamine
APTSSSTKKTQLQLEHLLLXLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 247 - protein, 133 aa, Artificial sequence
  Synthetic polypeptide
  Misc_feature (20)..(20): any amino acid other than aspartic acid
  Misc_feature (42)..(42): any amino acid other than phenylalanine
  Misc_feature (45)..(45): any amino acid other than tyrosine
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 248 - protein, 133 aa, Artificial sequence
  Synthetic polypeptide
  Misc_feature (16)..(16): any amino acid other than histidine
  Misc_feature (20)..(20): any amino acid other than aspartic acid
  Misc_feature (42)..(42): any amino acid other than phenylalanine
  Misc_feature (45)..(45): any amino acid other than tyrosine
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 249 - protein, 133 aa, Artificial sequence
  Synthetic polypeptide
  misc_feature (20)..(20): any amino acid other than aspartic acid
  misc_feature (42)..(42): any amino acid other than phenylalanine
  misc_feature (45)..(45): any amino acid other than tyrosine
  misc_feature (126)..(126): any amino acid other than glutamine
APTSSSTKKTQLQLEHLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 250 - protein, 133 aa, Artificial sequence
  Synthetic polypeptide
  Misc_feature (16)..(16): any amino acid other than histidine
  Misc_feature (20)..(20): any amino acid other than aspartic acid
  Misc_feature (42)..(42): any amino acid other than phenylalanine
  Misc_feature (45)..(45): any amino acid other than tyrosine
  Misc_feature (126)..(126): any amino acid other than glutamine
APTSSSTKKTQLQLEXLLLXLQMILNGINNYKNPKLTRMLTXKFXMPKKATELKHLQCLEEELKPLEEVLNLAQSKNFHLRPRDLISNINVIVLELKGSETTFMCEYADETATIVEFLNRWITFCXSIISTLT

SEQ ID NO: 251 - protein, 133 aa, Artificial sequence
  Synthetic polypeptide
  Misc_feature (16)..(16): any amino acid other than histidine
  Misc_feature (42)..(42): any amino acid other than phenylalanine
  Misc_feature (126)..(126): any amino acid other than glutamine
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCXSIISTLT

SEQ ID NO: 252 - protein, 33 aa, Artificial sequence
  Synthetic polypeptide
RMKQIEDKIEEILSKIYHIENEIARIKKLIGER

SEQ ID NO: 253 - protein, 32 aa, Artificial sequence
  Synthetic polypeptide
LSSIEKKQEEQTSWLIWISNELTLIRNELAQS

SEQ ID NO: 254 - protein, 31 aa, Artificial sequence
  Synthetic polypeptide
LSSIEKKLEEITSQLIQISNELTLIRNELAQ

SEQ ID NO: 255 - protein, 31 aa, Artificial sequence
  Synthetic polypeptide
LSSIEKKLEEITSQLIQIRNELTLIRNELAQ

SEQ ID NO: 256 - protein, 31 aa, Artificial sequence
  Synthetic polypeptide
LSSIEKKLEEITSQLQQIRNELTLIRNELAQ

SEQ ID NO: 257 - protein, 31 aa, Artificial sequence
  Synthetic polypeptide
LSSLEKKLEELTSQLIQLRNELTLLRNELAQ

SEQ ID NO: 258 - protein, 31 aa, Artificial sequence
  Synthetic polypeptide
ISSLEKKIEELTSQIQQLRNEITLLRNEIAQ

SEQ ID NO: 259 - protein, 47 aa, Artificial sequence; identical to SEQ ID NO: 99
  Synthetic polypeptide

SEQ ID NO: 260 - protein, 64 aa, Artificial sequence
  Synthetic polypeptide
VTAFSNMDDMLQKAHLVIEGTFIYLRDSTEFFIRVRDGWKKLQLGELIPIPADSPPPPALSSNP

SEQ ID NO: 261 - protein, 31 aa, Artificial sequence
  Synthetic polypeptide
LKSVENRLAVVENQLKTVIEELKTVKDLLSN

SEQ ID NO: 262 - protein, 31 aa, Artificial sequence
  Synthetic polypeptide
LARIEEKLKTIKAQLSEIASTLNMIREQLAQ

SEQ ID NO: 263 - protein, 31 aa, Artificial sequence
  Synthetic polypeptide
VSRLEEKVKTLKSQVTELASTVSLLREQVAQ

SEQ ID NO: 264 - protein, 30 aa, Artificial sequence
  Synthetic polypeptide
IQSEKKIEDISSLIGQIQSEITLIRNEIAQ

SEQ ID NO: 265 - protein, 31 aa, Artificial sequence
  Synthetic polypeptide
LMSLEKKLEELTQTLMQLQNELSMLKNELAQ

SEQ ID NO: 266 - protein, 18 aa, Artificial sequence
  Synthetic polypeptide
VDLEGSTSNGRQCAGIRL

SEQ ID NO: 267 - protein, 27 aa, Artificial sequence
  Synthetic polypeptide
EDDVTTTEELAPALVPPPKGTCAGWMA

SEQ ID NO: 268 - protein, 27 aa, Artificial sequence
  Synthetic polypeptide
GHDQETTTQGPGVLLPLPKGACTGQMA

SEQ ID NO: 269 - protein, 9 aa, Artificial sequence
  Synthetic polypeptide
YPYDVPDYA

SEQ ID NO: 270 - protein, 8 aa, Artificial sequence
  Synthetic polypeptide
DYKDDDDK

SEQ ID NO: 271 - protein, 10 aa, Artificial sequence
  Synthetic polypeptide
EQKLISEEDL

SEQ ID NO: 272 - protein, 5 aa, Artificial sequence
  Synthetic polypeptide
HHHHH

SEQ ID NO: 273 - protein, 6 aa, Artificial sequence
  Synthetic polypeptide
HHHHHH

SEQ ID NO: 274 - protein, 8 aa, Artificial sequence
  Synthetic polypeptide
WSHPQFEK

SEQ ID NO: 275 - protein, 5 aa, Artificial sequence
  Synthetic polypeptide
RYIRS

SEQ ID NO: 276 - protein, 4 aa, Artificial sequence
  Synthetic polypeptide
FHHT

SEQ ID NO: 277 - protein, 17 aa, Artificial sequence
  Synthetic polypeptide
WEAAAREACCRECCARA

SEQ ID NO: 278 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 279 - protein, 266 aa, Homo sapiens [sequence omitted]

SEQ ID NO: 280 - protein, 266 aa, Homo sapiens [sequence omitted]

SEQ ID NO: 281 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 282 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 283 - protein, 266 aa, Homo sapiens [sequence omitted]

SEQ ID NO: 284 - protein, 266 aa, Homo sapiens [sequence omitted]

SEQ ID NO: 285 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 286 - protein, 266 aa, Homo sapiens [sequence omitted]

SEQ ID NO: 287 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 288 - protein, 266 aa, Homo sapiens [sequence omitted]

SEQ ID NO: 289 - protein, 266 aa, Homo sapiens
MVCLKLPGGSSLAALTVTLMVLSSRLAFAGDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSKMLSGVGGFVLGLLFLGAGLFIYFRNQKGHSGLQPTGFLS

SEQ ID NO: 290 - protein, 266 aa, Homo sapiens [sequence omitted]

SEQ ID NO: 291 - protein, 266 aa, Homo sapiens [sequence omitted]

SEQ ID NO: 292 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 293 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 294 - protein, 261 aa, Homo sapiens
MGHEQNQGAALLQMLPLLWLLPHSWAVPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 295 - protein, 263 aa, Homo sapiens
MITFLPLLLGLSLGCTGAGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHTGAPEPILRDWTPGLSPMQTLKVSVSAVTLGLGLIIFSLGVISWRRAGHSSYTPLPGSNYSEGWHIS

SEQ ID NO: 296 - protein, 250 aa, Homo sapiens
MALRAGLVLGFHTLMTLLSPQEAGATKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHWELQVPIPPPDAMETLVCALGLAIGLVGFLVGTVLIIMGTYVSSVPR

SEQ ID NO: 297 - protein, 273 aa, Homo sapiens
MGSGWVPWVVALLVNLTRLDSSMTQGTDSPEDFVIQAKADCYFTNGTEKVQFVVRFIFNLEEYVRFDSDVGMFVALTKLGQPDAEQWNSRLDLLERSRQAVDGVCRHNYRLGAPFTVGRKVQPEVTVYPERTPLLHQHNLLHCSVTGFYPGDIKIKWFLNGQEERAGVMSTGPIRNGDWTFQTVVMLEMTPELGHVYTCLVDHSSLLSPVSVEWRAQSEYSWRKMLSGIAAFLLGLIFLLVGIVIQLRAQKGYVRTQMSGNEVSRAVLLPQSC

SEQ ID NO: 298 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 299 - protein, 258 aa, Homo sapiens
MMVLQVSAAPRTVALTALLMVLLTSVVQGRATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 300 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 301 - protein, 255 aa, Homo sapiens
MILNKALLLGALALTAVMSPCGGEDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETLVCALGLSVGLMGIVVGTVFIIQGLRSVGASRHQGLL

SEQ ID NO: 302 - protein, 269 aa, Homo sapiens
MSWKKALRIPGDLRVATVTLMLAMLSSLLAEGRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGPQGPPPAGLLH

SEQ ID NO: 303 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 304 - protein, 227 aa, Homo sapiens
MALQIPGGFWAAAVTVMLVMLSTPVAEARDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRPRGPPPAGLLH

SEQ ID NO: 305 - protein, 264 aa, Homo sapiens
MALQIPGGFWAAAVTVMLVMLSTPVAEARDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRAQSESAQSKMLSGIGGFVLGLIFLGLGLIIRHRGQKGPRGPPPAGLLH

SEQ ID NO: 306 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 308 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 310 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 311 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 312 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 313 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 314 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 315 - protein, 227 aa, Artificial sequence
  Synthetic polypeptide
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 316 - protein, 227 aa, Artificial sequence; identical to SEQ ID NO: 93
  Synthetic polypeptide

SEQ ID NO: 317 - protein, 227 aa, Artificial sequence; identical to SEQ ID NO: 105
  Synthetic polypeptide

SEQ ID NO: 318 - protein, 493 aa, Artificial sequence
  Synthetic polypeptide
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMGGGGSGGGGSGGGGSLEIEAAFLERENTALETRVAELRQRVQRLRNRVSQYRTRYGPLGGGK

SEQ ID NO: 319 - DNA, 1485 bp, Artificial sequence
  synthetic polynucleotide
atgtacaggatgcaactcctgtcttgcattgcactaagtcttgcacttgtcacaaacagtgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgggtggaggtggctcaggaggcggcggcagcggtggaggagggagcctggagatcgaggccgccttcctggagcgggagaacaccgccctggagacccgggtggccgagctgcggcagcgggtgcagcggctgcggaaccgggtgtcccagtaccggacccggtacggccccctgggcggcggcaagtagtga

SEQ ID NO: 320 - protein, 639 aa, Artificial sequence
  Synthetic polypeptide
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGGGSGGGGSLEIRAAFLRQRNTALRTEVAELEQEVQRLENEVSQYETRYGPLGGGKGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 321 - DNA, 1923 bp, Artificial sequence
  synthetic polynucleotide
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactggtggaggtggctcaggaggcggcggcagcctggagatccgggccgccttcctgcggcagcggaacaccgccctgcggaccgaggtggccgagctggagcaggaggtgcagcggctggagaacgaggtgtcccagtacgagacccggtacggccccctgggcggcggcaagggcggatcagcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 322 - protein, 711 aa, Artificial sequence
  Synthetic polypeptide
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRRVEPGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAGGGGSGGGGSKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSGGGGSGGGGSGGGGSGGGGSGGGGSGGGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 323 - DNA, 2139 bp, Artificial sequence
  synthetic polynucleotide
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctggaggcggaggatctggaggcggaggatctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctggaggcggaggatctggaggcggaggatctggtggtggaggttctggtggtgggggatctggaggcggaggatctggaggcggaggatctgcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 324 - protein, 992 aa, Artificial sequence
  Synthetic polypeptide
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRRVEPGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAGGGGSGGGGSKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 325 - DNA, 2982 bp, Artificial sequence
  synthetic polynucleotide
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctggaggcggaggatctggaggcggaggatctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctgcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 326 - protein, 887 aa, Artificial sequence
  Synthetic polypeptide
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRRVEPGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 327 - DNA, 2667 bp, Artificial sequence
  synthetic polynucleotide
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctgcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 328 - protein, 640 aa, Artificial sequence
  Synthetic polypeptide
MYRMQLLSCIALSLALVTNSLPLKMLNIPSINVHGGGGSGSGGGGSGGGGGDTRPRFLWQHKFECHFFNGTERVRLLERCIYNQEESVRFDSDVGEYRAVTELGRPAAEYWNSQKDLLEQRRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGGGSGGGGSLEIRAAFLRQRNTALRTEVAELEQEVQRLENEVSQYETRYGPLGGGKGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 329 - DNA, 1926 bp, Artificial sequence
  synthetic polynucleotide
atgtacaggatgcaactcctgtcttgcattgcactaagtcttgcacttgtcacaaacagtcttccgctcaaaatgcttaacataccttccattaatgtccacggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgcagccgagtactggaacagccagaaggacctcctggagcagaggcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactggtggaggtggctcaggaggcggcggcagcctggagatccgggccgccttcctgcggcagcggaacaccgccctgcggaccgaggtggccgagctggagcaggaggtgcagcggctggagaacgaggtgtcccagtacgagacccggtacggccccctgggcggcggcaagggcggatcagcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 330 - protein, 644 aa, Artificial sequence
  Synthetic polypeptide
MYRMQLLSCIALSLALVTNSGAGSLQPLALEGSLQKRGGGGGSGSGGGGSGGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGGGSGGGGSLEIRAAFLRQRNTALRTEVAELEQEVQRLENEVSQYETRYGPLGGGKGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 331 - DNA, 1938 bp, Artificial sequence
  synthetic polynucleotide
atgtacaggatgcaactcctgtcttgcattgcactaagtcttgcacttgtcacaaacagtggggcagggtcactccaaccgttggcgctggaaggctctcttcagaagcgaggtggaggtggcggatccggctccggtggaggtggctcaggaggcggcggcggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactggtggaggtggctcaggaggcggcggcagcctggagatccgggccgccttcctgcggcagcggaacaccgccctgcggaccgaggtggccgagctggagcaggaggtgcagcggctggagaacgaggtgtcccagtacgagacccggtacggccccctgggcggcggcaagggcggatcagcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 332 - protein, 493 aa, Artificial sequence
  Synthetic polypeptide
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSLEIEAAFLERENTALETRVAELRQRVQRLRNRVSQYRTRYGPLGGGK

SEQ ID NO: 333 - DNA, 1485 bp, Artificial sequence
  synthetic polynucleotide
atgtacaggatgcaactcctgtcttgcattgcactaagtcttgcacttgtcacaaacagtgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgtctatcctgaggtgactgtgtatcctgcaaagacccagcccctgcagcaccacaacctcctggtctgctctgtgaatggtttctatccagccagcattgaagtcaggtggttccggaacggccaggaagagaagactggggtggtgtccacaggcctgatccagaatggagactggaccttccagaccctggtgatgctggaaacagttcctcggagtggagaggtttacacctgccaagtggagcacccaagcctgacgagccctctcacagtggaatggagagcacggtctgaatctgcacagagcaagatgggtggaggtggctcaggaggcggcggcagcggtggaggagggagcctggagatcgaggccgccttcctggagcgggagaacaccgccctggagacccgggtggccgagctgcggcagcgggtgcagcggctgcggaaccgggtgtcccagtaccggacccggtacggccccctgggcggcggcaagtagtga

SEQ ID NO: 334 - protein, 602 aa, Artificial sequence
  Synthetic polypeptide
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAGGGGSGGGGSGGGGSGGGGSGGGGSGGGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 335 - DNA, 1812 bp, Artificial sequence
  synthetic polynucleotide
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctggaggcggaggatctggaggcggaggatctggtggtggaggttctggtggtgggggatctggaggcggaggatctggaggcggaggatctgcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 336 - protein, 491 aa, Artificial sequence
  Synthetic polypeptide
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMGGGGSGGGGSGGGGSLEIEAAFLERENTALETRVAELRQRVQRLRNRVSQYRTRYGPLGGGK

SEQ ID NO: 337 - DNA, 1479 bp, Artificial sequence
  synthetic polynucleotide
atgtacaggatgcaactcctgtcttgcattgcactaagtcttgcacttgtcacaaacagtgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgggtggaggtggctcaggaggcggcggcagcggtggaggagggagcctggagatcgaggccgccttcctggagcgggagaacaccgccctggagacccgggtggccgagctgcggcagcgggtgcagcggctgcggaaccgggtgtcccagtaccggacccggtacggccccctgggcggcggcaagtagtga

SEQ ID NO: 338 - protein, 429 aa, Artificial sequence
  Synthetic polypeptide
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKM

SEQ ID NO: 339 - DNA, 1293 bp, Artificial sequence
  synthetic polynucleotide
atgtacaggatgcaactcctgtcttgcattgcactaagtcttgcacttgtcacaaacagtgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgtagtga

SEQ ID NO: 340 - protein, 328 aa, Artificial sequence
  Synthetic polypeptide
MYRMQLLSCIALSLALVTNSPKYVKQNTLKLATGGGGSGGGGSGGGGSGDTRPRFLWQHKFECHFFNGTERVRLLERCIYNQEESVRFDSDVGEYRAVTELGRPDAEYWNSQKDLLEQRRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

SEQ ID NO: 341 - DNA, 984 bp, Artificial sequence
  synthetic polynucleotide
atgtacaggatgcaactcctgtcttgcattgcactaagtcttgcacttgtcacaaacagtccgaaatatgtaaaacagaataccctgaaattggcaacaggaggtggcggatccggtggaggtggctcaggaggcggcggctctggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgct

SEQ ID NO: 342 - protein, 688 aa, Artificial sequence
  Synthetic polypeptide
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - DNA, 2064 bp, Artificial sequence
  synthetic polynucleotide
atgtacaggatgcaactcctgtcttgcattgcactaagtcttgcacttgtcacaaacagtgcccctacttccagctccaccaagaagacgcagcttcagctggaagcactgctgctcgatctgcagatgatactgaatggcattaacaactacaaaaaccccaagctcactcgcatgctgaccgctaaattctacatgcccaagaaggctacggaactgaagcacctgcagtgccttgaggaggaactcaagccactcgaggaggtgctgaacctggcacagtcaaagaactttcacctgcggccaagagacctgatttcgaacatcaacgtgattgtgctggaattgaagggctcagaaactacgttcatgtgcgagtacgccgacgaaactgctactatcgtggagttcttgaaccgctggatcacgttctgccagagcattatttcaactcttaccggtggaggtggttctggaggtggtggatcaggaggaggtggctccgggggtggaggtagcgctcccacgtcatcctccactaaaaagacccagctgcaactcgaggcactgttgctggacctccagatgattctgaacggaatcaacaactataagaacccgaagctgactagaatgttgactgccaaattttatatgccaaagaaggcaactgagttgaagcatctgcaatgcctggaagaggagctgaagccactggaagaggtgcttaacctcgctcagtccaagaacttccatctgcgcccacgggaccttatctccaacattaacgtgatcgtgctggaactgaagggatccgaaaccacttttatgtgcgaatacgctgacgaaaccgccactatcgtcgagttcctgaacaggtggatcaccttctgccagtccattatctccaccctcaccggtggaggtggttctggaggtggtggatcaggaggaggtggctccgggggtggaggtagcgttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgggaggcggaggatctggaggcggaggatctggtggtggaggttctggtggtgggggatctggaggcggaggatctggaggcggaggatctgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaa

SEQ ID NO: 344 - protein, 5 aa, Artificial Sequence
  Synthetic sequence
  Misc_feature (2)..(5): This stretch of amino acid residues may be repeated
GSSSS

SEQ ID NO: 345 - protein, 10 aa, Artificial Sequence
  Synthetic sequence
GGGGSGGGGS

SEQ ID NO: 346 - protein, 15 aa, Artificial Sequence
  Synthetic sequence
GGGGSGGGGSGGGGS

SEQ ID NO: 347 - protein, 20 aa, Artificial Sequence
  Synthetic sequence
GGGGSGGGGSGGGGSGGGGS

SEQ ID NO: 348 - protein, 25 aa, Artificial Sequence
  Synthetic sequence
GGGGSGGGGSGGGGSGGGGSGGGGS

SEQ ID NO: 349 - protein, 30 aa, Artificial Sequence
  Synthetic sequence
GGGGSGGGGSGGGGSGGGGSGGGGSGGGGS

SEQ ID NO: 350 - protein, 35 aa, Artificial Sequence
  Synthetic sequence
GGGGSGGGGSGGGGSGGGGSGGGGSGGGGSGGGGS

SEQ ID NO: 351 - protein, 40 aa, Artificial Sequence
  Synthetic sequence
GGGGSGGGGSGGGGSGGGGSGGGGSGGGGSGGGGSGGGGS

SEQ ID NO: 352 - protein, 45 aa, Artificial Sequence
  Synthetic sequence
GGGGSGGGGSGGGGSGGGGSGGGGSGGGGSGGGGSGGGGSGGGGS

SEQ ID NO: 353 - protein, 50 aa, Artificial Sequence
  Synthetic sequence
GGGGSGGGGSGGGGSGGGGSGGGGSGGGGSGGGGSGGGGSGGGGSGGGGS

SEQ ID NO: 354 - protein, 8 aa, Artificial Sequence
  Synthetic sequence
TPITNVPP

SEQ ID NO: 355 - protein, 20 aa, Artificial Sequence
  Synthetic sequence
MYRMQLLSCIALSLALVTNS

SEQ ID NO: 356 - protein, 105 aa, Artificial Sequence; identical to SEQ ID NO: 98
  Synthetic sequence

SEQ ID NO: 357 - protein, 227 aa, Artificial Sequence; identical to SEQ ID NO: 105
  Synthetic sequence